SEQUENCE LISTING (80 sequences)

SEQ ID NO: 1 - RNA, 36 nt, synthetic construct
  misc_feature 1..36: note = Synthetic
  source 1..36: mol_type = other RNA
ggtggatgaaactcagtttagcagccgaaaggctgc

SEQ ID NO: 2 - RNA, 20 nt, synthetic construct
  misc_feature 1..20: note = Synthetic
  source 1..20: mol_type = other RNA
ggtggatgaaactcagttta

SEQ ID NO: 3 - RNA, 36 nt, synthetic construct
  misc_feature 1..36: note = Synthetic
  source 1..36: mol_type = other RNA
atattttcccatctgtattagcagccgaaaggctgc

SEQ ID NO: 4 - RNA, 20 nt, synthetic construct
  misc_feature 1..20: note = Synthetic
  source 1..20: mol_type = other RNA
atattttcccatctgtatta

SEQ ID NO: 5 - RNA, 18 nt, synthetic construct
  misc_feature 1..18: note = Synthetic
  source 1..18: mol_type = other RNA
ggtggatgaaactcagtt

SEQ ID NO: 6 - RNA, 15 nt, synthetic construct
  modified_base 12: mod_base = OTHER; note = thymine
  misc_feature 1..15: note = Synthetic
  source 1..15: mol_type = other RNA
atgaaactcagttta

SEQ ID NO: 7 - RNA, 15 nt, synthetic construct
  misc_feature 1..15: note = Synthetic
  source 1..15: mol_type = other RNA
atgaaactcagttta

SEQ ID NO: 8 - RNA, 15 nt, synthetic construct
  modified_base 1: mod_base = OTHER; note = thymine
  modified_base 11: mod_base = OTHER; note = thymine
  misc_feature 1..15: note = Synthetic
  source 1..15: mol_type = other RNA
ttcccatctgtatta

SEQ ID NO: 9 - RNA, 13 nt, synthetic construct
  misc_feature 1..13: note = Synthetic
  source 1..13: mol_type = other RNA
atgaaactcagtt

SEQ ID NO: 10 - RNA, 16 nt, synthetic construct
  misc_feature 1..16: note = Synthetic
  source 1..16: mol_type = other RNA
aaaatgaaactcagtt

SEQ ID NO: 11 - RNA, 17 nt, synthetic construct
  misc_feature 1..17: note = Synthetic
  source 1..17: mol_type = other RNA
aaaaatgaaactcagtt

SEQ ID NO: 12 - RNA, 13 nt, synthetic construct
  modified_base 1: mod_base = OTHER; note = thymine
  modified_base 11: mod_base = OTHER; note = thymine
  misc_feature 1..13: note = Synthetic
  source 1..13: mol_type = other RNA
ttcccatctgtat

SEQ ID NO: 13 - RNA, 16 nt, synthetic construct
  modified_base 4: mod_base = OTHER; note = thymine
  modified_base 14: mod_base = OTHER; note = thymine
  misc_feature 1..16: note = Synthetic
  source 1..16: mol_type = other RNA
aaattcccatctgtat

SEQ ID NO: 14 - RNA, 17 nt, synthetic construct
  modified_base 5: mod_base = OTHER; note = thymine
  modified_base 15: mod_base = OTHER; note = thymine
  misc_feature 1..17: note = Synthetic
  source 1..17: mol_type = other RNA
aaaattcccatctgtat

SEQ ID NO: 15 - RNA, 22 nt, synthetic construct
  misc_feature 1..22: note = Synthetic
  source 1..22: mol_type = other RNA
taaactgagtttcatccaccgg

SEQ ID NO: 16 - RNA, 22 nt, synthetic construct
  misc_feature 1..22: note = Synthetic
  source 1..22: mol_type = other RNA
taatacagatgggaaaatatgg

SEQ ID NO: 17 - RNA, 21 nt, synthetic construct
  misc_feature 1..21: note = Synthetic
  source 1..21: mol_type = other RNA
taaactgagtttcatccaccg

SEQ ID NO: 18 - RNA, 20 nt, synthetic construct
  misc_feature 1..20: note = Synthetic
  source 1..20: mol_type = other RNA
taaactgagtttcatccacc

SEQ ID NO: 19 - RNA, 19 nt, synthetic construct
  misc_feature 1..19: note = Synthetic
  source 1..19: mol_type = other RNA
taaactgagtttcatccac

SEQ ID NO: 20 - RNA, 18 nt, synthetic construct
  misc_feature 1..18: note = Synthetic
  source 1..18: mol_type = other RNA
taaactgagtttcatcca

SEQ ID NO: 21 - RNA, 36 nt, synthetic construct; identical to SEQ ID NO: 1
  misc_feature 1..36: note = Synthetic
  modified_base 1: mod_base = OTHER; note = 2'-O-methyl-guanosine linked via phosphorothioate
  modified_base 2: mod_base = OTHER; note = 2'-O-methyl-guanosine
  modified_base 3: mod_base = OTHER; note = 2'-O-methyl-uridine
  modified_base 4: mod_base = OTHER; note = 2'-O-methyl-guanosine
  modified_base 5: mod_base = OTHER; note = 2'-O-methyl-guanosine
  modified_base 6: mod_base = OTHER; note = 2'-O-methyl-adenosine
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-Fluoro-adenosine
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O-methyl-cytidine
  modified_base 15: mod_base = OTHER; note = 2'-O-methyl-adenosine
  modified_base 16: mod_base = OTHER; note = 2'-O-methyl-guanosine
  modified_base 17: mod_base = OTHER; note = 2'-O-methyl-uridine
  modified_base 18: mod_base = OTHER; note = 2'-O-methyl-uridine
  modified_base 19: mod_base = OTHER; note = 2'-O-methyl-uridine
  modified_base 20: mod_base = OTHER; note = 2'-O-methyl-adenosine
  modified_base 21: mod_base = OTHER; note = 2'-O-methyl-guanosine
  modified_base 22: mod_base = OTHER; note = 2'-O-methyl-cytidine
  modified_base 23: mod_base = OTHER; note = 2'-O-methyl-adenosine
  modified_base 24: mod_base = OTHER; note = 2'-O-methyl-guanosine
  modified_base 25: mod_base = OTHER; note = 2'-O-methyl-cytidine
  modified_base 26: mod_base = OTHER; note = 2'-O-methyl-cytidine
  modified_base 27: mod_base = OTHER; note = 2'-O-methyl-guanosine
  modified_base 28: mod_base = OTHER; note = aminodiethoxymethanol-adenosine-N-acetylgalactosami ne
  modified_base 29: mod_base = OTHER; note = aminodiethoxymethanol-adenosine-N-acetylgalactosami ne
  modified_base 30: mod_base = OTHER; note = aminodiethoxymethanol-adenosine-N-acetylgalactosami ne
  modified_base 31: mod_base = OTHER; note = 2'-O-methyl-guanosine
  modified_base 32: mod_base = OTHER; note = 2'-O-methyl-guanosine
  modified_base 33: mod_base = OTHER; note = 2'-O-methyl-cytidine
  modified_base 34: mod_base = OTHER; note = 2'-O-methyl-uridine
  modified_base 35: mod_base = OTHER; note = 2'-O-methyl-guanosine
  modified_base 36: mod_base = OTHER; note = 2'-O-methyl-cytidine
  source 1..36: mol_type = other RNA

SEQ ID NO: 22 - RNA, 20 nt, synthetic construct
  misc_feature 1..20: note = Synthetic
  modified_base 1: mod_base = OTHER; note = aminodiethoxymethanol-guanosine-N-acetylgalactosami ne linked via phosphorothioate
  modified_base 2: mod_base = OTHER; note = aminodiethoxymethanol-guanosine-N-acetylgalactosami ne
  modified_base 3: mod_base = OTHER; note = aminodiethoxymethanol-uridine-N-acetylgalactosamine
  modified_base 4: mod_base = OTHER; note = 2'-O-methyl-guanosine
  modified_base 5: mod_base = OTHER; note = 2'-O-methyl-guanosine
  modified_base 6: mod_base = OTHER; note = 2'-O-methyl-adenosine
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-Fluoro-adenosine
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O-methyl-cytidine
  modified_base 15: mod_base = OTHER; note = 2'-O-methyl-adenosine
  modified_base 16: mod_base = OTHER; note = 2'-O-methyl-guanosine
  modified_base 17: mod_base = OTHER; note = 2'-O-methyl-uridine
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
ggtggatgaaactcagttta

SEQ ID NO: 23 - RNA, 20 nt, synthetic construct
  misc_feature 1..20: note = Synthetic
  modified_base 1: mod_base = OTHER; note = 2'-O-methyl-guanosine linked via phosphorothioate
  modified_base 2: mod_base = OTHER; note = 2'-O-methyl-guanosine
  modified_base 3: mod_base = OTHER; note = 2'-O-methyl-uridine
  modified_base 4: mod_base = OTHER; note = aminodiethoxymethanol-guanosine-N-acetylgalactosami ne
  modified_base 5: mod_base = OTHER; note = aminodiethoxymethanol-guanosine-N-acetylgalactosami ne
  modified_base 6: mod_base = OTHER; note = aminodiethoxymethanol-adenosine-N-acetylgalactosami ne
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-Fluoro-adenosine
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O-methyl-cytidine
  modified_base 15: mod_base = OTHER; note = 2'-O-methyl-adenosine
  modified_base 16: mod_base = OTHER; note = 2'-O-methyl-guanosine
  modified_base 17: mod_base = OTHER; note = 2'-O-methyl-uridine
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
ggtggatgaaactcagttta

SEQ ID NO: 24 - RNA, 20 nt, synthetic construct
  misc_feature 1..20: note = Synthetic
  modified_base 1: mod_base = OTHER; note = 2'-O-methyl-guanosine linked via phosphorothioate
  modified_base 2: mod_base = OTHER; note = 2'-O-methyl-guanosine
  modified_base 3: mod_base = OTHER; note = 2'-O-methyl-uridine
  modified_base 4: mod_base = OTHER; note = 2'-O-methyl-guanosine
  modified_base 5: mod_base = OTHER; note = 2'-O-methyl-guanosine
  modified_base 6: mod_base = OTHER; note = 2'-O-methyl-adenosine
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = aminodiethoxymethanol-adenosine-N-acetylgalactosami ne
  modified_base 10: mod_base = OTHER; note = aminodiethoxymethanol-adenosine-N-acetylgalactosami ne
  modified_base 11: mod_base = OTHER; note = aminodiethoxymethanol-adenosine-N-acetylgalactosami ne
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O-methyl-cytidine
  modified_base 15: mod_base = OTHER; note = 2'-O-methyl-adenosine
  modified_base 16: mod_base = OTHER; note = 2'-O-methyl-guanosine
  modified_base 17: mod_base = OTHER; note = 2'-O-methyl-uridine
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
ggtggatgaaactcagttta

SEQ ID NO: 25 - RNA, 20 nt, synthetic construct
  misc_feature 1..20: note = Synthetic
  modified_base 1: mod_base = OTHER; note = 2'-O-methyl-guanosine linked via phosphorothioate
  modified_base 2: mod_base = OTHER; note = 2'-O-methyl-guanosine
  modified_base 3: mod_base = OTHER; note = 2'-O-methyl-uridine
  modified_base 4: mod_base = OTHER; note = 2'-O-methyl-guanosine
  modified_base 5: mod_base = OTHER; note = 2'-O-methyl-guanosine
  modified_base 6: mod_base = OTHER; note = 2'-O-methyl-adenosine
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-Fluoro-adenosine
  modified_base 12: mod_base = OTHER; note = aminodiethoxymethanol-cytidine-N-acetylgalactosamin e
  modified_base 13: mod_base = OTHER; note = aminodiethoxymethanol-uridine-N-acetylgalactosamine
  modified_base 14: mod_base = OTHER; note = aminodiethoxymethanol-cytidine-N-acetylgalactosamin e
  modified_base 15: mod_base = OTHER; note = 2'-O-methyl-adenosine
  modified_base 16: mod_base = OTHER; note = 2'-O-methyl-guanosine
  modified_base 17: mod_base = OTHER; note = 2'-O-methyl-uridine
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
ggtggatgaaactcagttta

SEQ ID NO: 26 - RNA, 20 nt, synthetic construct
  misc_feature 1..20: note = Synthetic
  modified_base 1: mod_base = OTHER; note = 2'-O-methyl-guanosine linked via phosphorothioate
  modified_base 2: mod_base = OTHER; note = 2'-O-methyl-guanosine
  modified_base 3: mod_base = OTHER; note = 2'-O-methyl-uridine
  modified_base 4: mod_base = OTHER; note = 2'-O-methyl-guanosine
  modified_base 5: mod_base = OTHER; note = 2'-O-methyl-guanosine
  modified_base 6: mod_base = OTHER; note = 2'-O-methyl-adenosine
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-Fluoro-adenosine
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O-methyl-cytidine
  modified_base 15: mod_base = OTHER; note = aminodiethoxymethanol-adenosine-N-acetylgalactosami ne
  modified_base 16: mod_base = OTHER; note = aminodiethoxymethanol-guanosine-N-acetylgalactosami ne
  modified_base 17: mod_base = OTHER; note = aminodiethoxymethanol-uridine-N-acetylgalactosamine
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
ggtggatgaaactcagttta

SEQ ID NO: 27 - RNA, 20 nt, synthetic construct
  misc_feature 1..20: note = Synthetic
  modified_base 1: mod_base = OTHER; note = 2'-O-methyl-guanosine linked via phosphorothioate
  modified_base 2: mod_base = OTHER; note = 2'-O-methyl-guanosine
  modified_base 3: mod_base = OTHER; note = 2'-O-methyl-uridine
  modified_base 4: mod_base = OTHER; note = 2'-O-methyl-guanosine
  modified_base 5: mod_base = OTHER; note = 2'-O-methyl-guanosine
  modified_base 6: mod_base = OTHER; note = 2'-O-methyl-adenosine
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-Fluoro-adenosine
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O-methyl-cytidine
  modified_base 15: mod_base = OTHER; note = 2'-O-methyl-adenosine
  modified_base 16: mod_base = OTHER; note = 2'-O-methyl-guanosine
  modified_base 17: mod_base = OTHER; note = 2'-O-methyl-uridine
  modified_base 18: mod_base = OTHER; note = aminodiethoxymethanol-uridine-N-acetylgalactosamine linked via phosphorothioate
  modified_base 19: mod_base = OTHER; note = aminodiethoxymethanol-uridine-N-acetylgalactosamine linked via phosphorothioate
  modified_base 20: mod_base = OTHER; note = aminodiethoxymethanol-adenosine-N-acetylgalactosami ne
  source 1..20: mol_type = other RNA
ggtggatgaaactcagttta

SEQ ID NO: 28 - RNA, 20 nt, synthetic construct
  misc_feature 1..20: note = Synthetic
  modified_base 1: mod_base = OTHER; note = 2'-O-methyl-guanosine linked via phosphorothioate
  modified_base 2: mod_base = OTHER; note = 2'-O-methyl-guanosine
  modified_base 3: mod_base = OTHER; note = 2'-O-methyl-uridine
  modified_base 4: mod_base = OTHER; note = 2'-O-methyl-guanosine
  modified_base 5: mod_base = OTHER; note = 2'-O-methyl-guanosine
  modified_base 6: mod_base = OTHER; note = 2'-O-methyl-adenosine
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-Fluoro-adenosine
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O-methyl-cytidine
  modified_base 15: mod_base = OTHER; note = 2'-O-methyl-adenosine
  modified_base 16: mod_base = OTHER; note = 2'-O-methyl-guanosine
  modified_base 17: mod_base = OTHER; note = 2'-O-methyl-uridine
  modified_base 18: mod_base = OTHER; note = 2'-O-methyl-uridine linked via phosphorothioate
  modified_base 19: mod_base = OTHER; note = aminodiethoxymethanol-uridine-N-acetylgalactosamine linked via phosphorothioate
  modified_base 20: mod_base = OTHER; note = aminodiethoxymethanol-adenosine-N-acetylgalactosami ne
  source 1..20: mol_type = other RNA
ggtggatgaaactcagttta

SEQ ID NO: 29 - RNA, 36 nt, synthetic construct; identical to SEQ ID NO: 3
  misc_feature 1..36: note = Synthetic
  modified_base 1: mod_base = OTHER; note = 2'-O-methyl-adenosine linked via phosphorothioate
  modified_base 2: mod_base = OTHER; note = 2'-O-methyl-uridine
  modified_base 3: mod_base = OTHER; note = 2'-O-methyl-adenosine
  modified_base 4: mod_base = OTHER; note = 2'-O-methyl-uridine
  modified_base 5: mod_base = OTHER; note = 2'-O-methyl-uridine
  modified_base 6: mod_base = OTHER; note = 2'-O-methyl-uridine
  modified_base 7: mod_base = OTHER; note = 2'-O-methyl-uridine
  modified_base 8: mod_base = OTHER; note = 2'-Fluoro-cytidine
  modified_base 9: mod_base = OTHER; note = 2'-Fluoro-cytidine
  modified_base 10: mod_base = OTHER; note = 2'-Fluoro-cytidine
  modified_base 11: mod_base = OTHER; note = 2'-Fluoro-adenosine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-O-methyl-uridine
  modified_base 15: mod_base = OTHER; note = 2'-O-methyl-guanosine
  modified_base 16: mod_base = OTHER; note = 2'-O-methyl-uridine
  modified_base 17: mod_base = OTHER; note = 2'-O-methyl-adenosine
  modified_base 18: mod_base = OTHER; note = 2'-O-methyl-uridine
  modified_base 19: mod_base = OTHER; note = 2'-O-methyl-uridine
  modified_base 20: mod_base = OTHER; note = 2'-O-methyl-adenosine
  modified_base 21: mod_base = OTHER; note = 2'-O-methyl-guanosine
  modified_base 22: mod_base = OTHER; note = 2'-O-methyl-cytidine
  modified_base 23: mod_base = OTHER; note = 2'-O-methyl-adenosine
  modified_base 24: mod_base = OTHER; note = 2'-O-methyl-guanosine
  modified_base 25: mod_base = OTHER; note = 2'-O-methyl-cytidine
  modified_base 26: mod_base = OTHER; note = 2'-O-methyl-cytidine
  modified_base 27: mod_base = OTHER; note = 2'-O-methyl-guanosine
  modified_base 28: mod_base = OTHER; note = aminodiethoxymethanol-adenosine-N-acetylgalactosami ne
  modified_base 29: mod_base = OTHER; note = aminodiethoxymethanol-adenosine-N-acetylgalactosami ne
  modified_base 30: mod_base = OTHER; note = aminodiethoxymethanol-adenosine-N-acetylgalactosami ne
  modified_base 31: mod_base = OTHER; note = 2'-O-methyl-guanosine
  modified_base 32: mod_base = OTHER; note = 2'-O-methyl-guanosine
  modified_base 33: mod_base = OTHER; note = 2'-O-methyl-cytidine
  modified_base 34: mod_base = OTHER; note = 2'-O-methyl-uridine
  modified_base 35: mod_base = OTHER; note = 2'-O-methyl-guanosine
  modified_base 36: mod_base = OTHER; note = 2'-O-methyl-cytidine
  source 1..36: mol_type = other RNA

SEQ ID NO: 30 - RNA, 20 nt, synthetic construct
  misc_feature 1..20: note = Synthetic
  modified_base 1: mod_base = OTHER; note = aminodiethoxymethanol-adenosine-N-acetylgalactosami ne linked via phosphorothioate
  modified_base 2: mod_base = OTHER; note = aminodiethoxymethanol-uridine-N-acetylgalactosamine
  modified_base 3: mod_base = OTHER; note = aminodiethoxymethanol-adenosine-N-acetylgalactosami ne
  modified_base 4: mod_base = OTHER; note = 2'-O-methyl-uridine
  modified_base 5: mod_base = OTHER; note = 2'-O-methyl-uridine
  modified_base 6: mod_base = OTHER; note = 2'-O-methyl-uridine
  modified_base 7: mod_base = OTHER; note = 2'-O-methyl-uridine
  modified_base 8: mod_base = OTHER; note = 2'-Fluoro-cytidine
  modified_base 9: mod_base = OTHER; note = 2'-Fluoro-cytidine
  modified_base 10: mod_base = OTHER; note = 2'-Fluoro-cytidine
  modified_base 11: mod_base = OTHER; note = 2'-Fluoro-adenosine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-O-methyl-uridine
  modified_base 15: mod_base = OTHER; note = 2'-O-methyl-guanosine
  modified_base 16: mod_base = OTHER; note = 2'-O-methyl-uridine
  modified_base 17: mod_base = OTHER; note = 2'-O-methyl-adenosine
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
atattttcccatctgtatta

SEQ ID NO: 31 - RNA, 20 nt, synthetic construct
  misc_feature 1..20: note = Synthetic
  modified_base 1: mod_base = OTHER; note = 2'-O-methyl-adenosine linked via phosphorothioate
  modified_base 2: mod_base = OTHER; note = 2'-O-methyl-uridine
  modified_base 3: mod_base = OTHER; note = 2'-O-methyl-adenosine
  modified_base 4: mod_base = OTHER; note = aminodiethoxymethanol-uridine-N-acetylgalactosamine
  modified_base 5: mod_base = OTHER; note = aminodiethoxymethanol-uridine-N-acetylgalactosamine
  modified_base 6: mod_base = OTHER; note = aminodiethoxymethanol-uridine-N-acetylgalactosamine
  modified_base 7: mod_base = OTHER; note = 2'-O-methyl-uridine
  modified_base 8: mod_base = OTHER; note = 2'-Fluoro-cytidine
  modified_base 9: mod_base = OTHER; note = 2'-Fluoro-cytidine
  modified_base 10: mod_base = OTHER; note = 2'-Fluoro-cytidine
  modified_base 11: mod_base = OTHER; note = 2'-Fluoro-adenosine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-O-methyl-uridine
  modified_base 15: mod_base = OTHER; note = 2'-O-methyl-guanosine
  modified_base 16: mod_base = OTHER; note = 2'-O-methyl-uridine
  modified_base 17: mod_base = OTHER; note = 2'-O-methyl-adenosine
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
atattttcccatctgtatta

SEQ ID NO: 32 - RNA, 20 nt, synthetic construct
  misc_feature 1..20: note = Synthetic
  modified_base 1: mod_base = OTHER; note = 2'-O-methyl-adenosine linked via phosphorothioate
  modified_base 2: mod_base = OTHER; note = 2'-O-methyl-uridine
  modified_base 3: mod_base = OTHER; note = 2'-O-methyl-adenosine
  modified_base 4: mod_base = OTHER; note = 2'-O-methyl-uridine
  modified_base 5: mod_base = OTHER; note = 2'-O-methyl-uridine
  modified_base 6: mod_base = OTHER; note = 2'-O-methyl-uridine
  modified_base 7: mod_base = OTHER; note = 2'-O-methyl-uridine
  modified_base 8: mod_base = OTHER; note = 2'-Fluoro-cytidine
  modified_base 9: mod_base = OTHER; note = aminodiethoxymethanol-cytidine-N-acetylgalactosamin e
  modified_base 10: mod_base = OTHER; note = aminodiethoxymethanol-cytidine-N-acetylgalactosamin e
  modified_base 11: mod_base = OTHER; note = aminodiethoxymethanol-adenosine-N-acetylgalactosami ne
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-O-methyl-uridine
  modified_base 15: mod_base = OTHER; note = 2'-O-methyl-guanosine
  modified_base 16: mod_base = OTHER; note = 2'-O-methyl-uridine
  modified_base 17: mod_base = OTHER; note = 2'-O-methyl-adenosine
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
atattttcccatctgtatta

SEQ ID NO: 33 - RNA, 20 nt, synthetic construct
  misc_feature 1..20: note = Synthetic
  modified_base 1: mod_base = OTHER; note = 2'-O-methyl-adenosine linked via phosphorothioate
  modified_base 2: mod_base = OTHER; note = 2'-O-methyl-uridine
  modified_base 3: mod_base = OTHER; note = 2'-O-methyl-adenosine
  modified_base 4: mod_base = OTHER; note = 2'-O-methyl-uridine
  modified_base 5: mod_base = OTHER; note = 2'-O-methyl-uridine
  modified_base 6: mod_base = OTHER; note = 2'-O-methyl-uridine
  modified_base 7: mod_base = OTHER; note = 2'-O-methyl-uridine
  modified_base 8: mod_base = OTHER; note = 2'-Fluoro-cytidine
  modified_base 9: mod_base = OTHER; note = 2'-Fluoro-cytidine
  modified_base 10: mod_base = OTHER; note = 2'-Fluoro-cytidine
  modified_base 11: mod_base = OTHER; note = 2'-Fluoro-adenosine
  modified_base 12: mod_base = OTHER; note = aminodiethoxymethanol-uridine-N-acetylgalactosamine
  modified_base 13: mod_base = OTHER; note = aminodiethoxymethanol-cytidine-N-acetylgalactosamin e
  modified_base 14: mod_base = OTHER; note = aminodiethoxymethanol-uridine-N-acetylgalactosamine
  modified_base 15: mod_base = OTHER; note = 2'-O-methyl-guanosine
  modified_base 16: mod_base = OTHER; note = 2'-O-methyl-uridine
  modified_base 17: mod_base = OTHER; note = 2'-O-methyl-adenosine
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
atattttcccatctgtatta

SEQ ID NO: 34 - RNA, 20 nt, synthetic construct
  misc_feature 1..20: note = Synthetic
  modified_base 1: mod_base = OTHER; note = 2'-O-methyl-adenosine linked via phosphorothioate
  modified_base 2: mod_base = OTHER; note = 2'-O-methyl-uridine
  modified_base 3: mod_base = OTHER; note = 2'-O-methyl-adenosine
  modified_base 4: mod_base = OTHER; note = 2'-O-methyl-uridine
  modified_base 5: mod_base = OTHER; note = 2'-O-methyl-uridine
  modified_base 6: mod_base = OTHER; note = 2'-O-methyl-uridine
  modified_base 7: mod_base = OTHER; note = 2'-O-methyl-uridine
  modified_base 8: mod_base = OTHER; note = 2'-Fluoro-cytidine
  modified_base 9: mod_base = OTHER; note = 2'-Fluoro-cytidine
  modified_base 10: mod_base = OTHER; note = 2'-Fluoro-cytidine
  modified_base 11: mod_base = OTHER; note = 2'-Fluoro-adenosine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-O-methyl-uridine
  modified_base 15: mod_base = OTHER; note = aminodiethoxymethanol-guanosine-N-acetylgalactosami ne
  modified_base 16: mod_base = OTHER; note = aminodiethoxymethanol-uridine-N-acetylgalactosamine
  modified_base 17: mod_base = OTHER; note = aminodiethoxymethanol-adenosine-N-acetylgalactosami ne
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
atattttcccatctgtatta

SEQ ID NO: 35 - RNA, 20 nt, synthetic construct
  misc_feature 1..20: note = Synthetic
  modified_base 1: mod_base = OTHER; note = 2'-O-methyl-adenosine linked via phosphorothioate
  modified_base 2: mod_base = OTHER; note = 2'-O-methyl-uridine
  modified_base 3: mod_base = OTHER; note = 2'-O-methyl-adenosine
  modified_base 4: mod_base = OTHER; note = 2'-O-methyl-uridine
  modified_base 5: mod_base = OTHER; note = 2'-O-methyl-uridine
  modified_base 6: mod_base = OTHER; note = 2'-O-methyl-uridine
  modified_base 7: mod_base = OTHER; note = 2'-O-methyl-uridine
  modified_base 8: mod_base = OTHER; note = 2'-Fluoro-cytidine
  modified_base 9: mod_base = OTHER; note = 2'-Fluoro-cytidine
  modified_base 10: mod_base = OTHER; note = 2'-Fluoro-cytidine
  modified_base 11: mod_base = OTHER; note = 2'-Fluoro-adenosine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-O-methyl-uridine
  modified_base 15: mod_base = OTHER; note = 2'-O-methyl-guanosine
  modified_base 16: mod_base = OTHER; note = 2'-O-methyl-uridine
  modified_base 17: mod_base = OTHER; note = 2'-O-methyl-adenosine
  modified_base 18: mod_base = OTHER; note = aminodiethoxymethanol-uridine-N-acetylgalactosamine linked via phosphorothioate
  modified_base 19: mod_base = OTHER; note = aminodiethoxymethanol-uridine-N-acetylgalactosamine linked via phosphorothioate
  modified_base 20: mod_base = OTHER; note = aminodiethoxymethanol-adenosine-N-acetylgalactosami ne
  source 1..20: mol_type = other RNA
atattttcccatctgtatta

SEQ ID NO: 36 - RNA, 20 nt, synthetic construct
  misc_feature 1..20: note = Synthetic
  modified_base 1: mod_base = OTHER; note = 2'-O-methyl-adenosine linked via phosphorothioate
  modified_base 2: mod_base = OTHER; note = 2'-O-methyl-uridine
  modified_base 3: mod_base = OTHER; note = 2'-O-methyl-adenosine
  modified_base 4: mod_base = OTHER; note = 2'-O-methyl-uridine
  modified_base 5: mod_base = OTHER; note = 2'-O-methyl-uridine
  modified_base 6: mod_base = OTHER; note = 2'-O-methyl-uridine
  modified_base 7: mod_base = OTHER; note = 2'-O-methyl-uridine
  modified_base 8: mod_base = OTHER; note = 2'-Fluoro-cytidine
  modified_base 9: mod_base = OTHER; note = 2'-Fluoro-cytidine
  modified_base 10: mod_base = OTHER; note = 2'-Fluoro-cytidine
  modified_base 11: mod_base = OTHER; note = 2'-Fluoro-adenosine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-O-methyl-uridine
  modified_base 15: mod_base = OTHER; note = 2'-O-methyl-guanosine
  modified_base 16: mod_base = OTHER; note = 2'-O-methyl-uridine
  modified_base 17: mod_base = OTHER; note = 2'-O-methyl-adenosine
  modified_base 18: mod_base = OTHER; note = 2'-O-methyl-uridine linked via phosphorothioate
  modified_base 19: mod_base = OTHER; note = aminodiethoxymethanol-uridine-N-acetylgalactosamine linked via phosphorothioate
  modified_base 20: mod_base = OTHER; note = aminodiethoxymethanol-adenosine-N-acetylgalactosami ne
  source 1..20: mol_type = other RNA
atattttcccatctgtatta

SEQ ID NO: 37 - RNA, 18 nt, synthetic construct
  misc_feature 1..18: note = Synthetic
  modified_base 1: mod_base = OTHER; note = 2'-O-methyl-guanosine linked via phosphorothioate
  modified_base 2: mod_base = OTHER; note = 2'-O-methyl-guanosine
  modified_base 3: mod_base = OTHER; note = 2'-O-methyl-uridine
  modified_base 4: mod_base = OTHER; note = 2'-O-methyl-guanosine
  modified_base 5: mod_base = OTHER; note = 2'-O-methyl-guanosine
  modified_base 6: mod_base = OTHER; note = 2'-O-methyl-adenosine
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-Fluoro-adenosine
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O-methyl-cytidine
  modified_base 15: mod_base = OTHER; note = 2'-O-methyl-adenosine
  modified_base 16: mod_base = OTHER; note = aminodiethoxymethanol-guanosine-N-acetylgalactosami ne linked via phosphorothioate
  modified_base 17: mod_base = OTHER; note = aminodiethoxymethanol-uridine-N-acetylgalactosamine linked via phosphorothioate
  modified_base 18: mod_base = OTHER; note = aminodiethoxymethanol-uridine-N-acetylgalactosamine
  source 1..18: mol_type = other RNA
ggtggatgaaactcagtt

SEQ ID NO: 38 - RNA, 15 nt, synthetic construct
  misc_feature 1..15: note = Synthetic
  modified_base 1: mod_base = OTHER; note = 2'-O,4'-C-Methyleneadenosine linked via phosphorothioate
  modified_base 2: mod_base = OTHER; note = 2'-O-methyl-uridine
  modified_base 3: mod_base = OTHER; note = 2'-Fluoro-guanosine
  modified_base 4: mod_base = OTHER; note = 2'-Fluoro-adenosine
  modified_base 5: mod_base = OTHER; note = 2'-Fluoro-adenosine
  modified_base 6: mod_base = OTHER; note = 2'-Fluoro-adenosine
  modified_base 7: mod_base = OTHER; note = 2'-O-methyl-cytidine
  modified_base 8: mod_base = OTHER; note = 2'-O-methyl-uridine
  modified_base 9: mod_base = OTHER; note = 2'-O-methyl-cytidine
  modified_base 10: mod_base = OTHER; note = 2'-O-methyl-adenosine
  modified_base 11: mod_base = OTHER; note = 2'-O,4'-C-Methyleneguanosine
  modified_base 12: mod_base = OTHER; note = 2'-O,4'-C-Methylenethymine
  modified_base 13: mod_base = OTHER; note = aminodiethoxymethanol-uridine-N-acetylgalactosamine linked via phosphorothioate
  modified_base 14: mod_base = OTHER; note = aminodiethoxymethanol-uridine-N-acetylgalactosamine linked via phosphorothioate
  modified_base 15: mod_base = OTHER; note = aminodiethoxymethanol-adenosine-N-acetylgalactosami ne
  source 1..15: mol_type = other RNA
atgaaactcagttta

SEQ ID NO: 39 - RNA, 20 nt, synthetic construct
  misc_feature 1..20: note = Synthetic
  modified_base 1: mod_base = OTHER; note = 2'-O-methyl-guanosine linked via phosphorothioate
  modified_base 2: mod_base = OTHER; note = 2'-O-methyl-guanosine
  modified_base 3: mod_base = OTHER; note = 2'-O-methyl-uridine
  modified_base 4: mod_base = OTHER; note = 2'-O-methyl-guanosine
  modified_base 5: mod_base = OTHER; note = 2'-O-methyl-guanosine
  modified_base 6: mod_base = OTHER; note = 2'-O-methyl-adenosine
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-Fluoro-adenosine
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O-methyl-cytidine
  modified_base 15: mod_base = OTHER; note = 2'-O-methyl-adenosine
  modified_base 16: mod_base = OTHER; note = 2'-O-methyl-guanosine
  modified_base 17: mod_base = OTHER; note = 2'-O-methyl-uridine
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
ggtggatgaaactcagttta

SEQ ID NO: 40 - RNA, 15 nt, synthetic construct
  misc_feature 1..15: note = Synthetic
  modified_base 1: mod_base = OTHER; note = 2'-O,4'-C-Methyleneadenosine linked via phosphorothioate
  modified_base 2: mod_base = OTHER; note = 2'-O-methyl-uridine
  modified_base 3: mod_base = OTHER; note = 2'-Fluoro-guanosine
  modified_base 4: mod_base = OTHER; note = 2'-Fluoro-adenosine
  modified_base 5: mod_base = OTHER; note = 2'-Fluoro-adenosine
  modified_base 6: mod_base = OTHER; note = 2'-Fluoro-adenosine
  modified_base 7: mod_base = OTHER; note = 2'-O-methyl-cytidine
  modified_base 8: mod_base = OTHER; note = 2'-O-methyl-uridine
  modified_base 9: mod_base = OTHER; note = 2'-O-methyl-cytidine
  modified_base 10: mod_base = OTHER; note = 2'-O-methyl-adenosine
  modified_base 11: mod_base = OTHER; note = 2'-O,4'-C-Methyleneguanosine
  modified_base 12: mod_base = OTHER; note = 2'-O,4'-C-Methylenethymine
  modified_base 13: mod_base = OTHER; note = 2'-O-methyl-uridine linked via phosphorothioate
  modified_base 14: mod_base = OTHER; note = 2'-O-methyl-uridine linked via phosphorothioate
  modified_base 15: mod_base = OTHER; note = 2'-O-methyl-adenosine
  source 1..15: mol_type = other RNA
atgaaactcagttta

SEQ ID NO: 41 - RNA, 20 nt, synthetic construct
  misc_feature 1..20: note = Synthetic
  modified_base 1: mod_base = OTHER; note = aminodiethoxymethanol-guanosine-N-acetylgalactosami ne linked via phosphorothioate
  modified_base 2: mod_base = OTHER; note = 2'-O-methyl-guanosine
  modified_base 3: mod_base = OTHER; note = 2'-O-methyl-uridine
  modified_base 4: mod_base = OTHER; note = 2'-O-methyl-guanosine
  modified_base 5: mod_base = OTHER; note = 2'-O-methyl-guanosine
  modified_base 6: mod_base = OTHER; note = 2'-O-methyl-adenosine
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-Fluoro-adenosine
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O-methyl-cytidine
  modified_base 15: mod_base = OTHER; note = 2'-O-methyl-adenosine
  modified_base 16: mod_base = OTHER; note = 2'-O-methyl-guanosine
  modified_base 17: mod_base = OTHER; note = 2'-O-methyl-uridine
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
ggtggatgaaactcagttta

SEQ ID NO: 42 - RNA, 15 nt, synthetic construct
  misc_feature 1..15: note = Synthetic
  modified_base 1: mod_base = OTHER; note = aminodiethoxymethanol-adenosine-N-acetylgalactosami ne linked via phosphorothioate
  modified_base 2: mod_base = OTHER; note = 2'-O-methyl-uridine
  modified_base 3: mod_base = OTHER; note = 2'-Fluoro-guanosine
  modified_base 4: mod_base = OTHER; note = 2'-Fluoro-adenosine
  modified_base 5: mod_base = OTHER; note = 2'-Fluoro-adenosine
  modified_base 6: mod_base = OTHER; note = 2'-Fluoro-adenosine
  modified_base 7: mod_base = OTHER; note = 2'-O-methyl-cytidine
  modified_base 8: mod_base = OTHER; note = 2'-O-methyl-uridine
  modified_base 9: mod_base = OTHER; note = 2'-O-methyl-cytidine
  modified_base 10: mod_base = OTHER; note = 2'-O-methyl-adenosine
  modified_base 11: mod_base = OTHER; note = 2'-O-methyl-guanosine
  modified_base 12: mod_base = OTHER; note = 2'-O-methyl-uridine
  modified_base 13: mod_base = OTHER; note = 2'-O-methyl-uridine linked via phosphorothioate
  modified_base 14: mod_base = OTHER; note = 2'-O-methyl-uridine linked via phosphorothioate
  modified_base 15: mod_base = OTHER; note = 2'-O-methyl-adenosine
  source 1..15: mol_type = other RNA
atgaaactcagttta

SEQ ID NO: 43 - RNA, 20 nt, synthetic construct
  misc_feature 1..20: note = Synthetic
  modified_base 1: mod_base = OTHER; note = 2'-O-methyl-guanosine linked via phosphorothioate
  modified_base 2: mod_base = OTHER; note = 2'-O-methyl-guanosine
  modified_base 3: mod_base = OTHER; note = 2'-O-methyl-uridine
  modified_base 4: mod_base = OTHER; note = 2'-O-methyl-guanosine
  modified_base 5: mod_base = OTHER; note = 2'-O-methyl-guanosine
  modified_base 6: mod_base = OTHER; note = 2'-O-methyl-adenosine
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-Fluoro-adenosine
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O-methyl-cytidine
  modified_base 15: mod_base = OTHER; note = 2'-O-methyl-adenosine
  modified_base 16: mod_base = OTHER; note = 2'-O-methyl-guanosine
  modified_base 17: mod_base = OTHER; note = 2'-O-methyl-uridine
  modified_base 18: mod_base = OTHER; note = aminodiethoxymethanol-uridine-N-acetylgalactosamine linked via phosphorothioate
  modified_base 19: mod_base = OTHER; note = aminodiethoxymethanol-uridine-N-acetylgalactosamine linked via phosphorothioate
  modified_base 20: mod_base = OTHER; note = 2'-O-methyl-adenosine
  source 1..20: mol_type = other RNA
ggtggatgaaactcagttta

SEQ ID NO: 44 - RNA, 20 nt, synthetic construct
  misc_feature 1..20: note = Synthetic
  modified_base 1: mod_base = OTHER; note = aminodiethoxymethanol-guanosine-N-acetylgalactosami ne linked via phosphorothioate
  modified_base 2: mod_base = OTHER; note = aminodiethoxymethanol-guanosine-N-acetylgalactosami ne
  modified_base 3: mod_base = OTHER; note = 2'-O-methyl-uridine
  modified_base 4: mod_base = OTHER; note = 2'-O-methyl-guanosine
  modified_base 5: mod_base = OTHER; note = 2'-O-methyl-guanosine
  modified_base 6: mod_base = OTHER; note = 2'-O-methyl-adenosine
  modified_base 7: mod_base = OTHER; note = 2'-O-methyl-uridine
  modified_base 8: mod_base = OTHER; note = 2'-Fluoro-guanosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-Fluoro-adenosine
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O-methyl-cytidine
  modified_base 15: mod_base = OTHER; note = 2'-O-methyl-adenosine
  modified_base 16: mod_base = OTHER; note = 2'-O-methyl-guanosine
  modified_base 17: mod_base = OTHER; note = 2'-O-methyl-uridine
  modified_base 18: mod_base = OTHER; note = 2'-O-methyl-uridine linked via phosphorothioate
  modified_base 19: mod_base = OTHER; note = 2'-O-methyl-uridine linked via phosphorothioate
  modified_base 20: mod_base = OTHER; note = 2'-O-methyl-adenosine
  source 1..20: mol_type = other RNA
ggtggatgaaactcagttta

SEQ ID NO: 45 - RNA, 15 nt, synthetic construct
  misc_feature 1..15: note = Synthetic
  modified_base 1: mod_base = OTHER; note = 2'-O,4'-C-Methyleneadenosine linked via phosphorothioate
  modified_base 2: mod_base = OTHER; note = aminodiethoxymethanol-uridine-N-acetylgalactosamine
  modified_base 3: mod_base = OTHER; note = aminodiethoxymethanol-guanosine-N-acetylgalactosami ne
  modified_base 4: mod_base = OTHER; note = aminodiethoxymethanol-adenosine-N-acetylgalactosami ne
  modified_base 5: mod_base = OTHER; note = 2'-Fluoro-adenosine
  modified_base 6: mod_base = OTHER; note = 2'-Fluoro-adenosine
  modified_base 7: mod_base = OTHER; note = 2'-O-methyl-cytidine
  modified_base 8: mod_base = OTHER; note = 2'-O-methyl-uridine
  modified_base 9: mod_base = OTHER; note = 2'-O-methyl-cytidine
  modified_base 10: mod_base = OTHER; note = 2'-O-methyl-adenosine
  modified_base 11: mod_base = OTHER; note = 2'-O,4'-C-Methyleneguanosine
  modified_base 12: mod_base = OTHER; note = 2'-O-methyl-uridine
  modified_base 13: mod_base = OTHER; note = 2'-O-methyl-uridine linked via phosphorothioate
  modified_base 14: mod_base = OTHER; note = 2'-O-methyl-uridine linked via phosphorothioate
  modified_base 15: mod_base = OTHER; note = 2'-O-methyl-adenosine
  source 1..15: mol_type = other RNA
atgaaactcagttta

SEQ ID NO: 46 - RNA, 15 nt, synthetic construct
  misc_feature 1..15: note = Synthetic
  modified_base 1: mod_base = OTHER; note = 2'-O,4'-C-Methyleneadenosine linked via phosphorothioate
  modified_base 2: mod_base = OTHER; note = 2'-O-methyl-uridine
  modified_base 3: mod_base = OTHER; note = 2'-Fluoro-guanosine
  modified_base 4: mod_base = OTHER; note = 2'-Fluoro-adenosine
  modified_base 5: mod_base = OTHER; note = 2'-Fluoro-adenosine
  modified_base 6: mod_base = OTHER; note = 2'-Fluoro-adenosine
  modified_base 7: mod_base = OTHER; note = 2'-O-methyl-cytidine
  modified_base 8: mod_base = OTHER; note = aminodiethoxymethanol-uridine-N-acetylgalactosamine
  modified_base 9: mod_base = OTHER; note = aminodiethoxymethanol-cytidine-N-acetylgalactosamin e
  modified_base 10: mod_base = OTHER; note = aminodiethoxymethanol-adenosine-N-acetylgalactosami ne
  modified_base 11: mod_base = OTHER; note = 2'-O,4'-C-Methyleneguanosine
  modified_base 12: mod_base = OTHER; note = 2'-O-methyl-uridine
  modified_base 13: mod_base = OTHER; note = 2'-O-methyl-uridine linked via phosphorothioate
  modified_base 14: mod_base = OTHER; note = 2'-O-methyl-uridine linked via phosphorothioate
  modified_base 15: mod_base = OTHER; note = 2'-O-methyl-adenosine
  source 1..15: mol_type = other RNA
atgaaactcagttta

SEQ ID NO: 47 - RNA, 15 nt, synthetic construct
  misc_feature 1..15: note = Synthetic
  modified_base 1: mod_base = OTHER; note = 2'-O,4'-C-Methyleneadenosine linked via phosphorothioate
  modified_base 2: mod_base = OTHER; note = 2'-O-methyl-uridine
  modified_base 3: mod_base = OTHER; note = 2'-Fluoro-guanosine
  modified_base 4: mod_base = OTHER; note = 2'-Fluoro-adenosine
  modified_base 5: mod_base = OTHER; note = 2'-Fluoro-adenosine
  modified_base 6: mod_base = OTHER; note = 2'-Fluoro-adenosine
  modified_base 7: mod_base = OTHER; note = 2'-O-methyl-cytidine
  modified_base 8: mod_base = OTHER; note = 2'-O-methyl-uridine
  modified_base 9: mod_base = OTHER; note = 2'-O-methyl-cytidine
  modified_base 10: mod_base = OTHER; note = 2'-O-methyl-adenosine
  modified_base 11: mod_base = OTHER; note = 2'-O,4'-C-Methyleneguanosine
  modified_base 12: mod_base = OTHER; note = 2'-O-methyl-uridine
  modified_base 13: mod_base = OTHER; note = aminodiethoxymethanol-uridine-N-acetylgalactosamine linked via phosphorothioate
  modified_base 14: mod_base = OTHER; note = aminodiethoxymethanol-uridine-N-acetylgalactosamine linked via phosphorothioate
  modified_base 15: mod_base = OTHER; note = aminodiethoxymethanol-adenosine-N-acetylgalactosami ne
  source 1..15: mol_type = other RNA
atgaaactcagttta

SEQ ID NO: 48 - RNA, 15 nt, synthetic construct
  misc_feature 1..15: note = Synthetic
  modified_base 1: mod_base = OTHER; note = 2'-O,4'-C-Methyleneadenosine linked via phosphorothioate
  modified_base 2: mod_base = OTHER; note = 2'-O-methyl-uridine
  modified_base 3: mod_base = OTHER; note = 2'-Fluoro-guanosine
  modified_base 4: mod_base = OTHER; note = 2'-Fluoro-adenosine
  modified_base 5: mod_base = OTHER; note = 2'-Fluoro-adenosine
  modified_base 6: mod_base = OTHER; note = 2'-Fluoro-adenosine
  modified_base 7: mod_base = OTHER; note = 2'-O-methyl-cytidine
  modified_base 8: mod_base = OTHER; note = 2'-O-methyl-uridine
  modified_base 9: mod_base = OTHER; note = 2'-O-methyl-cytidine
  modified_base 10: mod_base = OTHER; note = 2'-O-methyl-adenosine
  modified_base 11: mod_base = OTHER; note = 2'-O,4'-C-Methyleneguanosine
  modified_base 12: mod_base = OTHER; note = 2'-O-methyl-uridine
  modified_base 13: mod_base = OTHER; note = 2'-O-methyl-uridine linked via phosphorothioate
  modified_base 14: mod_base = OTHER; note = aminodiethoxymethanol-uridine-N-acetylgalactosamine linked via phosphorothioate
  modified_base 15: mod_base = OTHER; note = aminodiethoxymethanol-adenosine-N-acetylgalactosami ne
  source 1..15: mol_type = other RNA
atgaaactcagttta

SEQ ID NO: 49 - RNA, 15 nt, synthetic construct
  misc_feature 1..15: note = Synthetic
  modified_base 1: mod_base = OTHER; note = 2'-O,4'-C-Methylenethymine linked via phosphorothioate
  modified_base 2: mod_base = OTHER; note = aminodiethoxymethanol-uridine-N-acetylgalactosamine
  modified_base 3: mod_base = OTHER; note = aminodiethoxymethanol-cytidine-N-acetylgalactosamin e
  modified_base 4: mod_base = OTHER; note = aminodiethoxymethanol-cytidine-N-acetylgalactosamin e
  modified_base 5: mod_base = OTHER; note = 2'-Fluoro-cytidine
  modified_base 6: mod_base = OTHER; note = 2'-Fluoro-adenosine
  modified_base 7: mod_base = OTHER; note = 2'-O-methyl-uridine
  modified_base 8: mod_base = OTHER; note = 2'-O-methyl-cytidine
  modified_base 9: mod_base = OTHER; note = 2'-O-methyl-uridine
  modified_base 10: mod_base = OTHER; note = 2'-O-methyl-guanosine
  modified_base 11: mod_base = OTHER; note = 2'-O,4'-C-Methylenethymine
  modified_base 12: mod_base = OTHER; note = 2'-O-methyl-adenosine
  modified_base 13: mod_base = OTHER; note = 2'-O-methyl-uridine linked via phosphorothioate
  modified_base 14: mod_base = OTHER; note = 2'-O-methyl-uridine linked via phosphorothioate
  modified_base 15: mod_base = OTHER; note = 2'-O-methyl-adenosine
  source 1..15: mol_type = other RNA
ttcccatctgtatta

SEQ ID NO: 50 - RNA, 15 nt, synthetic construct
  misc_feature 1..15: note = Synthetic
  modified_base 1: mod_base = OTHER; note = 2'-O,4'-C-Methylenethymine linked via phosphorothioate
  modified_base 2: mod_base = OTHER; note = 2'-O-methyl-uridine
  modified_base 3: mod_base = OTHER; note = 2'-Fluoro-cytidine
  modified_base 4: mod_base = OTHER; note = 2'-Fluoro-cytidine
  modified_base 5: mod_base = OTHER; note = 2'-Fluoro-cytidine
  modified_base 6: mod_base = OTHER; note = 2'-Fluoro-adenosine
  modified_base 7: mod_base = OTHER; note = 2'-O-methyl-uridine
  modified_base 8: mod_base = OTHER; note = aminodiethoxymethanol-cytidine-N-acetylgalactosamin e
  modified_base 9: mod_base = OTHER; note = aminodiethoxymethanol-uridine-N-acetylgalactosamine
  modified_base 10: mod_base = OTHER; note = aminodiethoxymethanol-guanosine-N-acetylgalactosami ne
  modified_base 11: mod_base = OTHER; note = 2'-O,4'-C-Methylenethymine
  modified_base 12: mod_base = OTHER; note = 2'-O-methyl-adenosine
  modified_base 13: mod_base = OTHER; note = 2'-O-methyl-uridine linked via phosphorothioate
  modified_base 14: mod_base = OTHER; note = 2'-O-methyl-uridine linked via phosphorothioate
  modified_base 15: mod_base = OTHER; note = 2'-O-methyl-adenosine
  source 1..15: mol_type = other RNA
ttcccatctgtatta

SEQ ID NO: 51 - RNA, 15 nt, synthetic construct
  misc_feature 1..15: note = Synthetic
  modified_base 1: mod_base = OTHER; note = 2'-O,4'-C-Methylenethymine linked via phosphorothioate
  modified_base 2: mod_base = OTHER; note = 2'-O-methyl-uridine
  modified_base 3: mod_base = OTHER; note = 2'-Fluoro-cytidine
  modified_base 4: mod_base = OTHER; note = 2'-Fluoro-cytidine
  modified_base 5: mod_base = OTHER; note = 2'-Fluoro-cytidine
  modified_base 6: mod_base = OTHER; note = 2'-Fluoro-adenosine
  modified_base 7: mod_base = OTHER; note = 2'-O-methyl-uridine
  modified_base 8: mod_base = OTHER; note = 2'-O-methyl-cytidine
  modified_base 9: mod_base = OTHER; note = 2'-O-methyl-uridine
  modified_base 10: mod_base = OTHER; note = 2'-O-methyl-guanosine
  modified_base 11: mod_base = OTHER; note = 2'-O,4'-C-Methylenethymine
  modified_base 12: mod_base = OTHER; note = 2'-O-methyl-adenosine
  modified_base 13: mod_base = OTHER; note = aminodiethoxymethanol-uridine-N-acetylgalactosamine linked via phosphorothioate
  modified_base 14: mod_base = OTHER; note = aminodiethoxymethanol-uridine-N-acetylgalactosamine linked via phosphorothioate
  modified_base 15: mod_base = OTHER; note = aminodiethoxymethanol-adenosine-N-acetylgalactosami ne
  source 1..15: mol_type = other RNA
ttcccatctgtatta

SEQ ID NO: 52 - RNA, 15 nt, synthetic construct
  misc_feature 1..15: note = Synthetic
  modified_base 1: mod_base = OTHER; note = 2'-O,4'-C-Methylenethymine linked via phosphorothioate
  modified_base 2: mod_base = OTHER; note = 2'-O-methyl-uridine
  modified_base 3: mod_base = OTHER; note = 2'-Fluoro-cytidine
  modified_base 4: mod_base = OTHER; note = 2'-Fluoro-cytidine
  modified_base 5: mod_base = OTHER; note = 2'-Fluoro-cytidine
  modified_base 6: mod_base = OTHER; note = 2'-Fluoro-adenosine
  modified_base 7: mod_base = OTHER; note = 2'-O-methyl-uridine
  modified_base 8: mod_base = OTHER; note = 2'-O-methyl-cytidine
  modified_base 9: mod_base = OTHER; note = 2'-O-methyl-uridine
  modified_base 10: mod_base = OTHER; note = 2'-O-methyl-guanosine
  modified_base 11: mod_base = OTHER; note = 2'-O,4'-C-Methylenethymine
  modified_base 12: mod_base = OTHER; note = 2'-O-methyl-adenosine
  modified_base 13: mod_base = OTHER; note = 2'-O-methyl-uridine linked via phosphorothioate
  modified_base 14: mod_base = OTHER; note = aminodiethoxymethanol-uridine-N-acetylgalactosamine linked via phosphorothioate
  modified_base 15: mod_base = OTHER; note = aminodiethoxymethanol-adenosine-N-acetylgalactosami ne
  source 1..15: mol_type = other RNA
ttcccatctgtatta

SEQ ID NO: 53 - RNA, 13 nt, synthetic construct
  misc_feature 1..13: note = Synthetic
  modified_base 1: mod_base = OTHER; note = 2'-O,4'-C-Methyleneadenosine linked via phosphorothioate
  modified_base 2: mod_base = OTHER; note = aminodiethoxymethanol-uridine-N-acetylgalactosamine
  modified_base 3: mod_base = OTHER; note = aminodiethoxymethanol-guanosine-N-acetylgalactosami ne
  modified_base 4: mod_base = OTHER; note = aminodiethoxymethanol-adenosine-N-acetylgalactosami ne
  modified_base 5: mod_base = OTHER; note = 2'-Fluoro-adenosine
  modified_base 6: mod_base = OTHER; note = 2'-Fluoro-adenosine
  modified_base 7: mod_base = OTHER; note = 2'-O-methyl-cytidine
  modified_base 8: mod_base = OTHER; note = 2'-O-methyl-uridine
  modified_base 9: mod_base = OTHER; note = 2'-O-methyl-cytidine
  modified_base 10: mod_base = OTHER; note = 2'-O,4'-C-Methyleneadenosine
  modified_base 11: mod_base = OTHER; note = 2'-O,4'-C-Methyleneguanosine linked via phosphorothioate
  modified_base 12: mod_base = OTHER; note = 2'-O-methyl-uridine linked via phosphorothioate
  modified_base 13: mod_base = OTHER; note = 2'-O-methyl-uridine
  source 1..13: mol_type = other RNA
atgaaactcagtt

SEQ ID NO: 54 - RNA, 13 nt, synthetic construct
  misc_feature 1..13: note = Synthetic
  modified_base 1: mod_base = OTHER; note = 2'-O,4'-C-Methyleneadenosine linked via phosphorothioate
  modified_base 2: mod_base = OTHER; note = 2'-O-methyl-uridine
  modified_base 3: mod_base = OTHER; note = 2'-Fluoro-guanosine
  modified_base 4: mod_base = OTHER; note = 2'-Fluoro-adenosine
  modified_base 5: mod_base = OTHER; note = aminodiethoxymethanol-adenosine-N-acetylgalactosami ne
  modified_base 6: mod_base = OTHER; note = aminodiethoxymethanol-adenosine-N-acetylgalactosami ne
  modified_base 7: mod_base = OTHER; note = aminodiethoxymethanol-cytidine-N-acetylgalactosamin e
  modified_base 8: mod_base = OTHER; note = 2'-O-methyl-uridine
  modified_base 9: mod_base = OTHER; note = 2'-O-methyl-cytidine
  modified_base 10: mod_base = OTHER; note = 2'-O,4'-C-Methyleneadenosine
  modified_base 11: mod_base = OTHER; note = 2'-O,4'-C-Methyleneguanosine linked via phosphorothioate
  modified_base 12: mod_base = OTHER; note = 2'-O-methyl-uridine linked via phosphorothioate
  modified_base 13: mod_base = OTHER; note = 2'-O-methyl-uridine
  source 1..13: mol_type = other RNA
atgaaactcagtt

SEQ ID NO: 55 - RNA, 13 nt, synthetic construct
  misc_feature 1..13: note = Synthetic
  modified_base 1: mod_base = OTHER; note = 2'-O,4'-C-Methyleneadenosine linked via phosphorothioate
  modified_base 2: mod_base = OTHER; note = 2'-O-methyl-uridine
  modified_base 3: mod_base = OTHER; note = 2'-Fluoro-guanosine
  modified_base 4: mod_base = OTHER; note = 2'-Fluoro-adenosine
  modified_base 5: mod_base = OTHER; note = 2'-Fluoro-adenosine
  modified_base 6: mod_base = OTHER; note = 2'-Fluoro-adenosine
  modified_base 7: mod_base = OTHER; note = aminodiethoxymethanol-cytidine-N-acetylgalactosamin e
  modified_base 8: mod_base = OTHER; note = aminodiethoxymethanol-uridine-N-acetylgalactosamine
  modified_base 9: mod_base = OTHER; note = aminodiethoxymethanol-cytidine-N-acetylgalactosamin e
  modified_base 10: mod_base = OTHER; note = 2'-O,4'-C-Methyleneadenosine
  modified_base 11: mod_base = OTHER; note = 2'-O,4'-C-Methyleneguanosine linked via phosphorothioate
  modified_base 12: mod_base = OTHER; note = 2'-O-methyl-uridine linked via phosphorothioate
  modified_base 13: mod_base = OTHER; note = 2'-O-methyl-uridine
  source 1..13: mol_type = other RNA
atgaaactcagtt

SEQ ID NO: 56 - RNA, 13 nt, synthetic construct
  misc_feature 1..13: note = Synthetic
  modified_base 1: mod_base = OTHER; note = 2'-O,4'-C-Methyleneadenosine linked via phosphorothioate
  modified_base 2: mod_base = OTHER; note = 2'-O-methyl-uridine
  modified_base 3: mod_base = OTHER; note = 2'-Fluoro-guanosine
  modified_base 4: mod_base = OTHER; note = 2'-Fluoro-adenosine
  modified_base 5: mod_base = OTHER; note = 2'-Fluoro-adenosine
  modified_base 6: mod_base = OTHER; note = 2'-Fluoro-adenosine
  modified_base 7: mod_base = OTHER; note = 2'-O-methyl-cytidine
  modified_base 8: mod_base = OTHER; note = aminodiethoxymethanol-uridine-N-acetylgalactosamine
  modified_base 9: mod_base = OTHER; note = aminodiethoxymethanol-cytidine-N-acetylgalactosamin e
  modified_base 10: mod_base = OTHER; note = 2'-O,4'-C-Methyleneadenosine
  modified_base 11: mod_base = OTHER; note = 2'-O,4'-C-Methyleneguanosine linked via phosphorothioate
  modified_base 12: mod_base = OTHER; note = 2'-O-methyl-uridine linked via phosphorothioate
  modified_base 13: mod_base = OTHER; note = 2'-O-methyl-uridine
  source 1..13: mol_type = other RNA
atgaaactcagtt

SEQ ID NO: 57 - RNA, 13 nt, synthetic construct
  misc_feature 1..13: note = Synthetic
  modified_base 1: mod_base = OTHER; note = 2'-O,4'-C-Methyleneadenosine linked via phosphorothioate
  modified_base 2: mod_base = OTHER; note = 2'-O-methyl-uridine
  modified_base 3: mod_base = OTHER; note = 2'-Fluoro-guanosine
  modified_base 4: mod_base = OTHER; note = 2'-Fluoro-adenosine
  modified_base 5: mod_base = OTHER; note = 2'-Fluoro-adenosine
  modified_base 6: mod_base = OTHER; note = 2'-Fluoro-adenosine
  modified_base 7: mod_base = OTHER; note = 2'-O-methyl-cytidine
  modified_base 8: mod_base = OTHER; note = 2'-O-methyl-uridine
  modified_base 9: mod_base = OTHER; note = 2'-O-methyl-cytidine
  modified_base 10: mod_base = OTHER; note = 2'-O,4'-C-Methyleneadenosine
  modified_base 11: mod_base = OTHER; note = 2'-O,4'-C-Methyleneguanosine linked via phosphorothioate
  modified_base 12: mod_base = OTHER; note = aminodiethoxymethanol-uridine-N-acetylgalactosamine linked via phosphorothioate
  modified_base 13: mod_base = OTHER; note = aminodiethoxymethanol-uridine-N-acetylgalactosamine
  source 1..13: mol_type = other RNA
atgaaactcagtt

SEQ ID NO: 58 - RNA, 16 nt, synthetic construct
  misc_feature 1..16: note = Synthetic
  modified_base 1: mod_base = OTHER; note = aminodiethoxymethanol-adenosine-N-acetylgalactosami ne linked via phosphorothioate
  modified_base 2: mod_base = OTHER; note = aminodiethoxymethanol-adenosine-N-acetylgalactosami ne
  modified_base 3: mod_base = OTHER; note = aminodiethoxymethanol-adenosine-N-acetylgalactosami ne
  modified_base 4: mod_base = OTHER; note = 2'-O,4'-C-Methyleneadenosine
  modified_base 5: mod_base = OTHER; note = 2'-O-methyl-uridine
  modified_base 6: mod_base = OTHER; note = 2'-Fluoro-guanosine
  modified_base 7: mod_base = OTHER; note = 2'-Fluoro-adenosine
  modified_base 8: mod_base = OTHER; note = 2'-Fluoro-adenosine
  modified_base 9: mod_base = OTHER; note = 2'-Fluoro-adenosine
  modified_base 10: mod_base = OTHER; note = 2'-O-methyl-cytidine
  modified_base 11: mod_base = OTHER; note = 2'-O-methyl-uridine
  modified_base 12: mod_base = OTHER; note = 2'-O-methyl-cytidine
  modified_base 13: mod_base = OTHER; note = 2'-O,4'-C-Methyleneadenosine
  modified_base 14: mod_base = OTHER; note = 2'-O,4'-C-Methyleneguanosine linked via phosphorothioate
  modified_base 15: mod_base = OTHER; note = 2'-O-methyl-uridine linked via phosphorothioate
  modified_base 16: mod_base = OTHER; note = 2'-O-methyl-uridine
  source 1..16: mol_type = other RNA
aaaatgaaactcagtt

SEQ ID NO: 59 - RNA, 17 nt, synthetic construct
  misc_feature 1..17: note = Synthetic
  modified_base 1: mod_base = OTHER; note = aminodiethoxymethanol-adenosine-N-acetylgalactosami ne linked via phosphorothioate
  modified_base 2: mod_base = OTHER; note = aminodiethoxymethanol-adenosine-N-acetylgalactosami ne
  modified_base 3: mod_base = OTHER; note = aminodiethoxymethanol-adenosine-N-acetylgalactosami ne
  modified_base 4: mod_base = OTHER; note = 2'-O-methyl-adenosine
  modified_base 5: mod_base = OTHER; note = 2'-O,4'-C-Methyleneadenosine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-Fluoro-adenosine
  modified_base 9: mod_base = OTHER; note = 2'-Fluoro-adenosine
  modified_base 10: mod_base = OTHER; note = 2'-Fluoro-adenosine
  modified_base 11: mod_base = OTHER; note = 2'-O-methyl-cytidine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-O,4'-C-Methyleneadenosine
  modified_base 15: mod_base = OTHER; note = 2'-O,4'-C-Methyleneguanosine linked via phosphorothioate
  modified_base 16: mod_base = OTHER; note = 2'-O-methyl-uridine linked via phosphorothioate
  modified_base 17: mod_base = OTHER; note = 2'-O-methyl-uridine
  source 1..17: mol_type = other RNA
aaaaatgaaactcagtt

SEQ ID NO: 60 - RNA, 13 nt, synthetic construct
  misc_feature 1..13: note = Synthetic
  modified_base 1: mod_base = OTHER; note = 2'-O,4'-C-Methylenethymine linked via phosphorothioate
  modified_base 2: mod_base = OTHER; note = aminodiethoxymethanol-uridine-N-acetylgalactosamine
  modified_base 3: mod_base = OTHER; note = aminodiethoxymethanol-cytidine-N-acetylgalactosamin e
  modified_base 4: mod_base = OTHER; note = aminodiethoxymethanol-cytidine-N-acetylgalactosamin e
  modified_base 5: mod_base = OTHER; note = 2'-Fluoro-cytidine
  modified_base 6: mod_base = OTHER; note = 2'-Fluoro-adenosine
  modified_base 7: mod_base = OTHER; note = 2'-O-methyl-uridine
  modified_base 8: mod_base = OTHER; note = 2'-O-methyl-cytidine
  modified_base 9: mod_base = OTHER; note = 2'-O-methyl-uridine
  modified_base 10: mod_base = OTHER; note = 2'-O,4'-C-Methyleneguanosine
  modified_base 11: mod_base = OTHER; note = 2'-O,4'-C-Methylenethymine linked via phosphorothioate
  modified_base 12: mod_base = OTHER; note = 2'-O-methyl-adenosine linked via phosphorothioate
  modified_base 13: mod_base = OTHER; note = 2'-O-methyl-uridine
  source 1..13: mol_type = other RNA
ttcccatctgtat

SEQ ID NO: 61 - RNA, 13 nt, synthetic construct
  misc_feature 1..13: note = Synthetic
  modified_base 1: mod_base = OTHER; note = 2'-O,4'-C-Methylenethymine linked via phosphorothioate
  modified_base 2: mod_base = OTHER; note = 2'-O-methyl-uridine
  modified_base 3: mod_base = OTHER; note = 2'-Fluoro-cytidine
  modified_base 4: mod_base = OTHER; note = 2'-Fluoro-cytidine
  modified_base 5: mod_base = OTHER; note = aminodiethoxymethanol-cytidine-N-acetylgalactosamin e
  modified_base 6: mod_base = OTHER; note = aminodiethoxymethanol-adenosine-N-acetylgalactosami ne
  modified_base 7: mod_base = OTHER; note = aminodiethoxymethanol-uridine-N-acetylgalactosamine
  modified_base 8: mod_base = OTHER; note = 2'-O-methyl-cytidine
  modified_base 9: mod_base = OTHER; note = 2'-O-methyl-uridine
  modified_base 10: mod_base = OTHER; note = 2'-O,4'-C-Methyleneguanosine
  modified_base 11: mod_base = OTHER; note = 2'-O,4'-C-Methylenethymine linked via phosphorothioate
  modified_base 12: mod_base = OTHER; note = 2'-O-methyl-adenosine linked via phosphorothioate
  modified_base 13: mod_base = OTHER; note = 2'-O-methyl-uridine
  source 1..13: mol_type = other RNA
ttcccatctgtat

SEQ ID NO: 62 - RNA, 13 nt, synthetic construct
  misc_feature 1..13: note = Synthetic
  modified_base 1: mod_base = OTHER; note = 2'-O,4'-C-Methylenethymine linked via phosphorothioate
  modified_base 2: mod_base = OTHER; note = 2'-O-methyl-uridine
  modified_base 3: mod_base = OTHER; note = 2'-Fluoro-cytidine
  modified_base 4: mod_base = OTHER; note = 2'-Fluoro-cytidine
  modified_base 5: mod_base = OTHER; note = 2'-Fluoro-cytidine
  modified_base 6: mod_base = OTHER; note = 2'-Fluoro-adenosine
  modified_base 7: mod_base = OTHER; note = aminodiethoxymethanol-uridine-N-acetylgalactosamine
  modified_base 8: mod_base = OTHER; note = aminodiethoxymethanol-cytidine-N-acetylgalactosamin e
  modified_base 9: mod_base = OTHER; note = aminodiethoxymethanol-uridine-N-acetylgalactosamine
  modified_base 10: mod_base = OTHER; note = 2'-O,4'-C-Methyleneguanosine
  modified_base 11: mod_base = OTHER; note = 2'-O,4'-C-Methylenethymine linked via phosphorothioate
  modified_base 12: mod_base = OTHER; note = 2'-O-methyl-adenosine linked via phosphorothioate
  modified_base 13: mod_base = OTHER; note = 2'-O-methyl-uridine
  source 1..13: mol_type = other RNA
ttcccatctgtat

SEQ ID NO: 63 - RNA, 13 nt, synthetic construct
  misc_feature 1..13: note = Synthetic
  modified_base 1: mod_base = OTHER; note = 2'-O,4'-C-Methylenethymine linked via phosphorothioate
  modified_base 2: mod_base = OTHER; note = 2'-O-methyl-uridine
  modified_base 3: mod_base = OTHER; note = 2'-Fluoro-cytidine
  modified_base 4: mod_base = OTHER; note = 2'-Fluoro-cytidine
  modified_base 5: mod_base = OTHER; note = 2'-Fluoro-cytidine
  modified_base 6: mod_base = OTHER; note = 2'-Fluoro-adenosine
  modified_base 7: mod_base = OTHER; note = 2'-O-methyl-uridine
  modified_base 8: mod_base = OTHER; note = aminodiethoxymethanol-cytidine-N-acetylgalactosamin e
  modified_base 9: mod_base = OTHER; note = aminodiethoxymethanol-uridine-N-acetylgalactosamine
  modified_base 10: mod_base = OTHER; note = 2'-O,4'-C-Methyleneguanosine
  modified_base 11: mod_base = OTHER; note = 2'-O,4'-C-Methylenethymine linked via phosphorothioate
  modified_base 12: mod_base = OTHER; note = 2'-O-methyl-adenosine linked via phosphorothioate
  modified_base 13: mod_base = OTHER; note = 2'-O-methyl-uridine
  source 1..13: mol_type = other RNA
ttcccatctgtat

SEQ ID NO: 64 - RNA, 13 nt, synthetic construct
  misc_feature 1..13: note = Synthetic
  modified_base 1: mod_base = OTHER; note = 2'-O,4'-C-Methylenethymine linked via phosphorothioate
  modified_base 2: mod_base = OTHER; note = 2'-O-methyl-uridine
  modified_base 3: mod_base = OTHER; note = 2'-Fluoro-cytidine
  modified_base 4: mod_base = OTHER; note = 2'-Fluoro-cytidine
  modified_base 5: mod_base = OTHER; note = 2'-Fluoro-cytidine
  modified_base 6: mod_base = OTHER; note = 2'-Fluoro-adenosine
  modified_base 7: mod_base = OTHER; note = 2'-O-methyl-uridine
  modified_base 8: mod_base = OTHER; note = 2'-O-methyl-cytidine
  modified_base 9: mod_base = OTHER; note = 2'-O-methyl-uridine
  modified_base 10: mod_base = OTHER; note = 2'-O,4'-C-Methyleneguanosine
  modified_base 11: mod_base = OTHER; note = 2'-O,4'-C-Methylenethymine linked via phosphorothioate
  modified_base 12: mod_base = OTHER; note = aminodiethoxymethanol-adenosine-N-acetylgalactosami ne linked via phosphorothioate
  modified_base 13: mod_base = OTHER; note = aminodiethoxymethanol-uridine-N-acetylgalactosamine
  source 1..13: mol_type = other RNA
ttcccatctgtat

SEQ ID NO: 65 - RNA, 16 nt, synthetic construct
  misc_feature 1..16: note = Synthetic
  modified_base 1: mod_base = OTHER; note = aminodiethoxymethanol-adenosine-N-acetylgalactosami ne linked via phosphorothioate
  modified_base 2: mod_base = OTHER; note = aminodiethoxymethanol-adenosine-N-acetylgalactosami ne
  modified_base 3: mod_base = OTHER; note = aminodiethoxymethanol-adenosine-N-acetylgalactosami ne
  modified_base 4: mod_base = OTHER; note = 2'-O,4'-C-Methylenethymine
  modified_base 5: mod_base = OTHER; note = 2'-O-methyl-uridine
  modified_base 6: mod_base = OTHER; note = 2'-Fluoro-cytidine
  modified_base 7: mod_base = OTHER; note = 2'-Fluoro-cytidine
  modified_base 8: mod_base = OTHER; note = 2'-Fluoro-cytidine
  modified_base 9: mod_base = OTHER; note = 2'-Fluoro-adenosine
  modified_base 10: mod_base = OTHER; note = 2'-O-methyl-uridine
  modified_base 11: mod_base = OTHER; note = 2'-O-methyl-cytidine
  modified_base 12: mod_base = OTHER; note = 2'-O-methyl-uridine
  modified_base 13: mod_base = OTHER; note = 2'-O,4'-C-Methyleneguanosine
  modified_base 14: mod_base = OTHER; note = 2'-O,4'-C-Methylenethymine linked via phosphorothioate
  modified_base 15: mod_base = OTHER; note = 2'-O-methyl-adenosine linked via phosphorothioate
  modified_base 16: mod_base = OTHER; note = 2'-O-methyl-uridine
  source 1..16: mol_type = other RNA
aaattcccatctgtat

SEQ ID NO: 66 - RNA, 17 nt, synthetic construct
  misc_feature 1..17: note = Synthetic
  modified_base 1: mod_base = OTHER; note = aminodiethoxymethanol-adenosine-N-acetylgalactosami ne linked via phosphorothioate
  modified_base 2: mod_base = OTHER; note = aminodiethoxymethanol-adenosine-N-acetylgalactosami ne
  modified_base 3: mod_base = OTHER; note = aminodiethoxymethanol-adenosine-N-acetylgalactosami ne
  modified_base 4: mod_base = OTHER; note = 2'-O-methyl-adenosine
  modified_base 5: mod_base = OTHER; note = 2'-O,4'-C-Methylenethymine
  modified_base 6: mod_base = OTHER; note = 2'-O-methyl-uridine
  modified_base 7: mod_base = OTHER; note = 2'-Fluoro-cytidine
  modified_base 8: mod_base = OTHER; note = 2'-Fluoro-cytidine
  modified_base 9: mod_base = OTHER; note = 2'-Fluoro-cytidine
  modified_base 10: mod_base = OTHER; note = 2'-Fluoro-adenosine
  modified_base 11: mod_base = OTHER; note = 2'-O-methyl-uridine
  modified_base 12: mod_base = OTHER; note = 2'-O-methyl-cytidine
  modified_base 13: mod_base = OTHER; note = 2'-O-methyl-uridine
  modified_base 14: mod_base = OTHER; note = 2'-O,4'-C-Methyleneguanosine
  modified_base 15: mod_base = OTHER; note = 2'-O,4'-C-Methylenethymine linked via phosphorothioate
  modified_base 16: mod_base = OTHER; note = 2'-O-methyl-adenosine linked via phosphorothioate
  modified_base 17: mod_base = OTHER; note = 2'-O-methyl-uridine
  source 1..17: mol_type = other RNA
aaaattcccatctgtat

SEQ ID NO: 67 - RNA, 22 nt, synthetic construct
  misc_feature 1..22: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = 2'-O-methyl-uridine
  modified_base 16: mod_base = OTHER; note = 2'-O-methyl-cytidine
  modified_base 17: mod_base = OTHER; note = 2'-O-methyl-cytidine
  modified_base 18: mod_base = OTHER; note = 2'-O-methyl-adenosine
  modified_base 19: mod_base = OTHER; note = 2'-O-methyl-cytidine
  modified_base 20: mod_base = OTHER; note = 2'-O-methyl-cytidine linked via phosphorothioate
  modified_base 21: mod_base = OTHER; note = 2'-O-methyl-guanosine linked via phosphorothioate
  modified_base 22: mod_base = OTHER; note = 2'-O-methyl-guanosine
  source 1..22: mol_type = other RNA
taaactgagtttcatccaccgg

SEQ ID NO: 68 - RNA, 22 nt, synthetic construct
  misc_feature 1..22: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine
  modified_base 4: mod_base = OTHER; note = 2'-Fluoro-uridine
  modified_base 5: mod_base = OTHER; note = 2'-Fluoro-adenosine
  modified_base 6: mod_base = OTHER; note = 2'-O-methyl-cytidine
  modified_base 7: mod_base = OTHER; note = 2'-Fluoro-adenosine
  modified_base 8: mod_base = OTHER; note = 2'-O-methyl-guanosine
  modified_base 9: mod_base = OTHER; note = 2'-O-methyl-adenosine
  modified_base 10: mod_base = OTHER; note = 2'-Fluoro-uridine
  modified_base 11: mod_base = OTHER; note = 2'-O-methyl-guanosine
  modified_base 12: mod_base = OTHER; note = 2'-O-methyl-guanosine
  modified_base 13: mod_base = OTHER; note = 2'-O-methyl-guanosine
  modified_base 14: mod_base = OTHER; note = 2'-Fluoro-adenosine
  modified_base 15: mod_base = OTHER; note = 2'-O-methyl-adenosine
  modified_base 16: mod_base = OTHER; note = 2'-O-methyl-adenosine
  modified_base 17: mod_base = OTHER; note = 2'-O-methyl-adenosine
  modified_base 18: mod_base = OTHER; note = 2'-O-methyl-uridine
  modified_base 19: mod_base = OTHER; note = 2'-O-methyl-adenosine
  modified_base 20: mod_base = OTHER; note = 2'-O-methyl-uridine linked via phosphorothioate
  modified_base 21: mod_base = OTHER; note = 2'-O-methyl-guanosine linked via phosphorothioate
  modified_base 22: mod_base = OTHER; note = 2'-O-methyl-guanosine
  source 1..22: mol_type = other RNA
taatacagatgggaaaatatgg

SEQ ID NO: 69 - RNA, 22 nt, synthetic construct
  misc_feature 1..22: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine linked via phosphorothioate
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = 2'-O-methyl-uridine
  modified_base 16: mod_base = OTHER; note = 2'-O-methyl-cytidine
  modified_base 17: mod_base = OTHER; note = 2'-O-methyl-cytidine
  modified_base 18: mod_base = OTHER; note = 2'-O-methyl-adenosine
  modified_base 19: mod_base = OTHER; note = 2'-O-methyl-cytidine
  modified_base 20: mod_base = OTHER; note = 2'-O-methyl-cytidine linked via phosphorothioate
  modified_base 21: mod_base = OTHER; note = 2'-O-methyl-guanosine linked via phosphorothioate
  modified_base 22: mod_base = OTHER; note = 2'-O-methyl-guanosine
  source 1..22: mol_type = other RNA
taaactgagtttcatccaccgg

SEQ ID NO: 70 - RNA, 21 nt, synthetic construct
  misc_feature 1..21: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine linked via phosphorothioate
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = 2'-O-methyl-uridine
  modified_base 16: mod_base = OTHER; note = 2'-O-methyl-cytidine
  modified_base 17: mod_base = OTHER; note = 2'-O-methyl-cytidine
  modified_base 18: mod_base = OTHER; note = 2'-O-methyl-adenosine
  modified_base 19: mod_base = OTHER; note = 2'-O-methyl-cytidine linked via phosphorothioate
  modified_base 20: mod_base = OTHER; note = 2'-O-methyl-cytidine linked via phosphorothioate
  modified_base 21: mod_base = OTHER; note = 2'-O-methyl-guanosine
  source 1..21: mol_type = other RNA
taaactgagtttcatccaccg

SEQ ID NO: 71 - RNA, 20 nt, synthetic construct
  misc_feature 1..20: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine linked via phosphorothioate
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = 2'-O-methyl-uridine
  modified_base 16: mod_base = OTHER; note = 2'-O-methyl-cytidine
  modified_base 17: mod_base = OTHER; note = 2'-O-methyl-cytidine
  modified_base 18: mod_base = OTHER; note = 2'-O-methyl-adenosine linked via phosphorothioate
  modified_base 19: mod_base = OTHER; note = 2'-O-methyl-cytidine linked via phosphorothioate
  modified_base 20: mod_base = OTHER; note = 2'-O-methyl-cytidine
  source 1..20: mol_type = other RNA
taaactgagtttcatccacc

SEQ ID NO: 72 - RNA, 19 nt, synthetic construct
  misc_feature 1..19: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine linked via phosphorothioate
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = 2'-O-methyl-uridine
  modified_base 16: mod_base = OTHER; note = 2'-O-methyl-cytidine
  modified_base 17: mod_base = OTHER; note = 2'-O-methyl-cytidine linked via phosphorothioate
  modified_base 18: mod_base = OTHER; note = 2'-O-methyl-adenosine linked via phosphorothioate
  modified_base 19: mod_base = OTHER; note = 2'-O-methyl-cytidine
  source 1..19: mol_type = other RNA
taaactgagtttcatccac

SEQ ID NO: 73 - RNA, 18 nt, synthetic construct
  misc_feature 1..18: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine linked via phosphorothioate
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = 2'-O-methyl-uridine
  modified_base 16: mod_base = OTHER; note = 2'-O-methyl-cytidine linked via phosphorothioate
  modified_base 17: mod_base = OTHER; note = 2'-O-methyl-cytidine linked via phosphorothioate
  modified_base 18: mod_base = OTHER; note = 2'-O-methyl-adenosine
  source 1..18: mol_type = other RNA
taaactgagtttcatcca

SEQ ID NO: 74 - RNA, 22 nt, synthetic construct
  misc_feature 1..22: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine linked via phosphorothioate
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = 2'-O-methyl-uridine
  modified_base 16: mod_base = OTHER; note = 2'-O-methyl-cytidine
  modified_base 17: mod_base = OTHER; note = 2'-O-methyl-cytidine
  modified_base 18: mod_base = OTHER; note = 2'-O-methyl-adenosine
  modified_base 19: mod_base = OTHER; note = 2'-O-methyl-cytidine
  modified_base 20: mod_base = OTHER; note = aminodiethoxymethanol-cytidine-N-acetylgalactosamin e linked via phosphorothioate
  modified_base 21: mod_base = OTHER; note = aminodiethoxymethanol-guanosine-N-acetylgalactosami ne linked via phosphorothioate
  modified_base 22: mod_base = OTHER; note = 2'-O-methyl-guanosine
  source 1..22: mol_type = other RNA
taaactgagtttcatccaccgg

SEQ ID NO: 75 - RNA, 22 nt, synthetic construct
  misc_feature 1..22: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine linked via phosphorothioate
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = 2'-O-methyl-uridine
  modified_base 16: mod_base = OTHER; note = 2'-O-methyl-cytidine
  modified_base 17: mod_base = OTHER; note = 2'-O-methyl-cytidine
  modified_base 18: mod_base = OTHER; note = 2'-O-methyl-adenosine
  modified_base 19: mod_base = OTHER; note = aminodiethoxymethanol-cytidine-N-acetylgalactosamin e
  modified_base 20: mod_base = OTHER; note = aminodiethoxymethanol-cytidine-N-acetylgalactosamin e linked via phosphorothioate
  modified_base 21: mod_base = OTHER; note = aminodiethoxymethanol-guanosine-N-acetylgalactosami ne linked via phosphorothioate
  modified_base 22: mod_base = OTHER; note = 2'-O-methyl-guanosine
  source 1..22: mol_type = other RNA
taaactgagtttcatccaccgg

SEQ ID NO: 76 - RNA, 22 nt, synthetic construct
  misc_feature 1..22: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine linked via phosphorothioate
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = 2'-O-methyl-uridine
  modified_base 16: mod_base = OTHER; note = 2'-O-methyl-cytidine
  modified_base 17: mod_base = OTHER; note = aminodiethoxymethanol-cytidine-N-acetylgalactosamin e
  modified_base 18: mod_base = OTHER; note = aminodiethoxymethanol-adenosine-N-acetylgalactosami ne
  modified_base 19: mod_base = OTHER; note = aminodiethoxymethanol-cytidine-N-acetylgalactosamin e
  modified_base 20: mod_base = OTHER; note = 2'-O-methyl-cytidine linked via phosphorothioate
  modified_base 21: mod_base = OTHER; note = 2'-O-methyl-guanosine linked via phosphorothioate
  modified_base 22: mod_base = OTHER; note = 2'-O-methyl-guanosine
  source 1..22: mol_type = other RNA
taaactgagtttcatccaccgg

SEQ ID NO: 77 - RNA, 22 nt, synthetic construct
  misc_feature 1..22: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine linked via phosphorothioate
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = aminodiethoxymethanol-uridine-N-acetylgalactosamine
  modified_base 16: mod_base = OTHER; note = aminodiethoxymethanol-cytidine-N-acetylgalactosamin e
  modified_base 17: mod_base = OTHER; note = 2'-O-methyl-cytidine
  modified_base 18: mod_base = OTHER; note = 2'-O-methyl-adenosine
  modified_base 19: mod_base = OTHER; note = 2'-O-methyl-cytidine
  modified_base 20: mod_base = OTHER; note = 2'-O-methyl-cytidine linked via phosphorothioate
  modified_base 21: mod_base = OTHER; note = 2'-O-methyl-guanosine linked via phosphorothioate
  modified_base 22: mod_base = OTHER; note = 2'-O-methyl-guanosine
  source 1..22: mol_type = other RNA
taaactgagtttcatccaccgg

SEQ ID NO: 78 - RNA, 22 nt, synthetic construct
  misc_feature 1..22: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = aminodiethoxymethanol-adenosine-N-acetylgalactosami ne linked via phosphorothioate
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = 2'-O-methyl-uridine
  modified_base 16: mod_base = OTHER; note = 2'-O-methyl-cytidine
  modified_base 17: mod_base = OTHER; note = 2'-O-methyl-cytidine
  modified_base 18: mod_base = OTHER; note = 2'-O-methyl-adenosine
  modified_base 19: mod_base = OTHER; note = 2'-O-methyl-cytidine
  modified_base 20: mod_base = OTHER; note = 2'-O-methyl-cytidine linked via phosphorothioate
  modified_base 21: mod_base = OTHER; note = 2'-O-methyl-guanosine linked via phosphorothioate
  modified_base 22: mod_base = OTHER; note = 2'-O-methyl-guanosine
  source 1..22: mol_type = other RNA
taaactgagtttcatccaccgg

SEQ ID NO: 79 - RNA, 22 nt, synthetic construct
  misc_feature 1..22: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine linked via phosphorothioate
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = 2'-O-methyl-uridine
  modified_base 16: mod_base = OTHER; note = 2'-O-methyl-cytidine
  modified_base 17: mod_base = OTHER; note = 2'-O-methyl-cytidine
  modified_base 18: mod_base = OTHER; note = 2'-O-methyl-adenosine
  modified_base 19: mod_base = OTHER; note = aminodiethoxymethanol-cytidine-N-acetylgalactosamin e
  modified_base 20: mod_base = OTHER; note = aminodiethoxymethanol-cytidine-N-acetylgalactosamin e linked via phosphorothioate
  modified_base 21: mod_base = OTHER; note = 2'-O-methyl-guanosine linked via phosphorothioate
  modified_base 22: mod_base = OTHER; note = 2'-O-methyl-guanosine
  source 1..22: mol_type = other RNA
taaactgagtttcatccaccgg

SEQ ID NO: 80 - RNA, 22 nt, synthetic construct
  misc_feature 1..22: note = Synthetic
  modified_base 1: mod_base = OTHER; note = 4'-O-monomethylphosphonate-2'-O-methylmethyluridine
  modified_base 2: mod_base = OTHER; note = 2'-Fluoro-adenosine linked via phosphorothioate
  modified_base 3: mod_base = OTHER; note = 2'-Fluoro-adenosine linked via phosphorothioate
  modified_base 4: mod_base = OTHER; note = 2'-Fluoro-adenosine
  modified_base 5: mod_base = OTHER; note = 2'-Fluoro-cytidine
  modified_base 6: mod_base = OTHER; note = 2'-O-methyl-uridine
  modified_base 7: mod_base = OTHER; note = 2'-Fluoro-guanosine
  modified_base 8: mod_base = OTHER; note = 2'-O-methyl-adenosine
  modified_base 9: mod_base = OTHER; note = 2'-O-methyl-guanosine
  modified_base 10: mod_base = OTHER; note = 2'-Fluoro-uridine
  modified_base 11: mod_base = OTHER; note = 2'-O-methyl-uridine
  modified_base 12: mod_base = OTHER; note = 2'-O-methyl-uridine
  modified_base 13: mod_base = OTHER; note = 2'-O-methyl-cytidine
  modified_base 14: mod_base = OTHER; note = 2'-Fluoro-adenosine
  modified_base 15: mod_base = OTHER; note = 2'-O-methyl-uridine
  modified_base 16: mod_base = OTHER; note = 2'-O-methyl-cytidine
  modified_base 17: mod_base = OTHER; note = 2'-O-methyl-cytidine
  modified_base 18: mod_base = OTHER; note = 2'-O-methyl-adenosine
  modified_base 19: mod_base = OTHER; note = 2'-O-methyl-cytidine
  modified_base 20: mod_base = OTHER; note = aminodiethoxymethanol-cytidine-N-acetylgalactosamin e linked via phosphorothioate
  modified_base 21: mod_base = OTHER; note = 2'-O-methyl-guanosine linked via phosphorothioate
  modified_base 22: mod_base = OTHER; note = 2'-O-methyl-guanosine
  source 1..22: mol_type = other RNA
taaactgagtttcatccaccgg

(19) United States
(12) Patent Application Publication
(10) Pub. No.: US 2026/0317763 A1
(43) Pub. Date: Oct. 15, 2026

(54) GALNAC-CONJUGATED RNAI OLIGONUCLEOTIDES

(71) Applicant: NOVO NORDISK A/S, Bagsværd (DK)

(72) Inventors: Martin Lee Koser, Westford, MA (US); Cristina Grande, Lexington, MA (US)

(21) Appl. No.: 19/482,272

(22) PCT Filed: May 10, 2024
(86) PCT No.: PCT/US2024/028729

§ 371 Date: Nov. 7, 2025

Related U.S. Application Data
(60) Provisional application No. 63/501,333, filed on May 10, 2023.

(51) Int. Cl. C12N 15/113; A61P 1/16

(52) U.S. Cl. CPC C12N 15/113; A61P 1/16; C12N 2310/14; C12N 2310/312; C12N 2310/315; C12N 2310/321; C12N 2310/322; C12N 2310/351

(57) ABSTRACT

Ligand-conjugated oligonucleotide are provided herein that inhibit or reduce expression of target genes. Also provided are compositions including the same and uses thereof, particularly uses relating to treating diseases, disorders and/or conditions associated with an RNAi trigger induced decrease in target gene expression.

### CROSS-REFERENCE TO RELATED APPLICATIONS

This application claims the benefit of U.S. Provisional Patent Application No. 63/501,333 filed May 10, 2023 which is incorporated by reference herein in its entirety.

### TECHNICAL FIELD

The disclosure relates to oligonucleotides linked to ligand moieties useful in the inhibition of target genes in a variety of tissues. Specifically, the present disclosure relates to oligonucleotide-ligand conjugates, methods to prepare them, their chemical configuration, and methods to modulate (e.g., inhibit or reduce) the expression of a target gene using the conjugated nucleic acids and oligonucleotides according to the description provided herein. The disclosure also provides pharmaceutically acceptable compositions comprising the conjugates of the present description and methods of using said compositions in the treatment of various diseases or disorders.

### BACKGROUND OF THE DISCLOSURE

Regulation of gene expression by modified nucleic acids shows great potential as both a research tool in the laboratory and a therapeutic approach in the clinic. Several classes of oligonucleotide or nucleic acid-based therapeutics have been under the clinical investigation, including antisense oligonucleotides (ASO), short interfering RNA (siRNA), double-stranded nucleic acids (dsNA), aptamers, ribozymes, exon-skipping and splice-altering oligonucleotides, immunomodulatory oligonucleotides, mRNAs, and CRISPR. Chemical modifications in the relevant molecules to allow functionality in various tissues, organs and/or cell types play a key role in overcoming challenges of oligonucleotide therapeutics, including improving nuclease stability, RNA-binding affinity, and pharmacokinetics. Various chemical modification strategies for oligonucleotides have been developed in the past three decades including modification of the sugars, nucleobases, and phosphodiester backbone to improve and optimize performance and therapeutic efficacy (Deleavey and Darma, CHEM. BIOL. 2012, 19(8):937-54; Wan and Seth, J. MED. CHEM. 2016, 59(21):9645-67; and Egli and Manoharan, ACC. CHEM. RES. 2019, 54(4):1036-47).

However, the in vivo use of RNAi or siRNA molecules as pharmaceuticals has remained difficult due to obstacles encountered such as low biostability and unacceptable toxicity possibly caused by off-target effects. Various types of chemical modifications to improve the pharmacokinetics and to overcome bio-instability problems have been investigated over the years to improve the stability and specificity of the RNAi duplexes. In some cases, the chemical modification in siRNAs has improved the serum stability of siRNAs. However, often RNAi activity was lost, but the careful placement of some specific modified residues enables enhanced siRNA biostability without loss of siRNA potency. Some of these modifications have reduced siRNA side effects, such as the induction of recipient immune responses and inherent off-targeting effects and have even enhanced siRNA potency. Various chemically modified siRNAs have been investigated, among them were bridged nucleic acids (BNA's) such as 2′,4′-methylene bridged nucleic acid 2′,4′-BNAs, also known as locked nucleic acid or LNA's. Some of these modified siRNAs showed promising effects.

Therapeutic gene silencing mediated by RNAi oligonucleotide-based therapeutics comprising siRNAs or double-stranded nucleic acids (dsNAs) offer the potential for considerable expansion of the druggable target space and the possibility for treating orphan diseases that may be therapeutically unapproachable by other drug modalities (e.g., antibodies and/or small molecules). RNAi oligonucleotide-based therapeutics that inhibit or reduce expression of specific target genes in the liver have been developed and are currently in clinical use (Sehgal et al., (2013) JOURNAL OF HEPATOLOGY 59:1354-59). Technological hurdles remain for the development and clinical use of RNAi oligonucleotides in extrahepatic cells, tissues, and organs. Thus, an ongoing need exists in the art for the successful development of new and effective RNAi oligonucleotides to modulate the expression of a target genes in extrahepatic cells, tissues, and/or organs. This is complicated by the variant nature of the cell types in extrahepatic as well as concerns about circulatory patterns and cell membrane constituents such as receptor types.

Over the past decade, synthetic RNAi triggers such as double stranded RNAs have become ubiquitous tools in biological research, and extensive basic and clinical development efforts have recently culminated in the FDA approval of ONPATTRO™, the first RNAi drug. Despite a burgeoning drug development pipeline and an extensive compendium of excipients targeting ligands and delivery techniques, the difficulty of delivering RNAi agents to specific populations of disease related cells and or tissues, particularly outside the liver continues to limit the potential of RNAi therapy. Repeated attempts over the past several years to develop useful, active, and persistent RNAi agents and structures for use based on known liver delivery technology have not convincingly demonstrated the intended effects outside the liver. Thus, new dsRNA's with variant structures have been developed to overcome the limitations in the field.

### BRIEF SUMMARY OF THE DISCLOSURE

The disclosure is based, in part, on the discovery of low-molecular weight oligonucleotides that target mRNA in the liver and reduce expression. Low-molecular weight molecules are expected to reduce the cost of goods required for manufacturing. Additionally, the small size of the molecules decreases the time required for synthesis. Without wishing to be bound by theory, the small molecule size enables more active pharmaceutical ingredient to be incorporated within a composition for administration and thus is expected to allow for lower but more potent doses. Further, the small molecule size may facilitate increased bioavailability as the molecule enables formulation for different routes of administration to enhance knockdown activity within a tissue of interest.

Accordingly, in some aspects, the disclosure provides a double-stranded oligonucleotide comprising an antisense strand of about 18-22 nucleotides in length and a sense strand of about 13-20 nucleotides in length, wherein the antisense and sense strands form a duplex region of about 13-20 base pairs, wherein the antisense strand comprises a 3′ overhang of at least one nucleotide, wherein the antisense strand comprises a region of complementarity to a liver mRNA target sequence, and wherein the sense strand and/or antisense strand comprise at least one nucleotide conjugated to a GalNAc molecule.

In some or any of the foregoing or related embodiments, the 3′ overhang is 1-7 nucleotides. In embodiments, the 3′ overhang is 3-7 nucleotides.

In some or any of the foregoing or related embodiments, the oligonucleotide comprises a blunt end. In some embodiments, the blunt end comprises the 3′ end of the sense strand and the 5′ end of the antisense strand.

In some or any of the foregoing or related embodiments, the antisense strand comprises a 5′ overhang of at least one nucleotide. In some embodiments, the 3′ overhang is 2 nucleotides and the 5′ overhang is 2 nucleotides.

In some or any of the foregoing or related embodiments,
(i) the antisense strand is 22 nucleotides, the sense strand is 20 nucleotides, the duplex region is 20 base pairs, and the 3′ overhang is two nucleotides;
(ii) the antisense strand is 22 nucleotides, the sense strand is 18 nucleotides, the duplex region is 18 base pairs, and the 3′ overhang is two nucleotides;
(iii) the antisense strand is 22 nucleotides, the sense strand is 15 nucleotides, the duplex region is 15 base pairs, and the 3′ overhang is 7 nucleotides;
(iv) the antisense strand is 22 nucleotides, the sense strand is 13 nucleotides, the duplex region is 13 base pairs, and the 3′ overhang is 7 nucleotides;
(v) the antisense strand is 22 nucleotides, the sense strand is 16 nucleotides, the duplex region is 16 base pairs, and the 3′ overhang is 4 nucleotides; or
(vi) the antisense strand is 22 nucleotides, the sense strand is 17 nucleotides, the duplex region is 17 base pairs, and the 3′ overhang is 4 nucleotides.

In some or any of the foregoing or related embodiments, the three 5′ terminal nucleotides of the sense strand are each conjugated to a GalNAc molecule.

In some or any of the foregoing or related embodiments, the three 3′ terminal nucleotides of the sense strand are each conjugated to a GalNAc molecule. In some or any of the foregoing or related embodiments, the two 3′ terminal nucleotides of the sense strand are each conjugated to a GalNAc molecule. In some or any of the foregoing or related embodiments, two to four, or three consecutive nucleotides of the sense strand are each conjugated to a GalNAc molecule.

In some or any of the foregoing or related embodiments, the three consecutive nucleotides of the sense strand are selected from (i) nucleotides at positions 4, 5 and 6; (ii) nucleotides at positions 9, 10 and 11; (iii) nucleotides at positions 12, 13 and 15; and (iv) nucleotides at positions 15, 16 and 17.

In some or any of the foregoing or related embodiments, the three consecutive nucleotides of the sense strand are selected from (i) nucleotides at positions 2, 3 and 4; (ii) nucleotides at positions 5, 6 and 7; and (iii) nucleotides at positions 7, 8 and 9.

In some or any of the foregoing or related embodiments, the two 5′ terminal nucleotides of the sense strand are each conjugated to a GalNAc molecule.

In some or any of the foregoing or related embodiments, two consecutive nucleotides of the sense strand are each conjugated to a GalNAc molecule. In some embodiments, the two consecutive nucleotides of the sense strand are selected from (i) nucleotides at positions 8 and 9; and (ii) nucleotides at positions 12 and 13.

In some or any of the foregoing or related aspects, two to four consecutive nucleotides of the antisense strand are each conjugated to a GalNAc molecule. In some embodiments, nucleotides at positions 20 and 21 of the antisense strand are each conjugated to a GalNAc molecule.

In some or any of the foregoing or related embodiments, the 5′ terminal nucleotide of the sense strand is conjugated to a GalNAc molecule, and wherein two consecutive nucleotides of the antisense strand are each conjugated to a GalNAc molecule.

In some or any of the foregoing or related embodiments, the two 5′ terminal nucleotides of the sense strand are each conjugated to a GalNAc molecule, and wherein a nucleotide of the antisense strand is conjugated to a GalNAc molecule.

In some or any of the foregoing or related embodiments, the nucleotide of the antisense strand conjugated to the GalNAc molecule is the nucleotide at position 20 of the antisense strand.

In some or any of the foregoing or related embodiments, the two 5′ terminal nucleotides of the sense strand are each conjugated to a GalNAc molecule, and wherein positions 19 and 20 of the antisense strand are each conjugated to a GalNAc molecule.

In some or any of the foregoing or related embodiments,
(i) the antisense strand is 21 nucleotides, the sense strand is 15 nucleotides, the duplex region is 15 base pairs, and the 3′ overhang is 6 nucleotides;
(ii) the antisense strand is 20 nucleotides, the sense strand is 15 nucleotides, the duplex region is 15 base pairs, and the 3′ overhang is 5 nucleotides;
(iii) the antisense strand is 19 nucleotides, the sense strand is 15 nucleotides, the duplex region is 15 base pairs, and the 3′ overhang is 4 nucleotides; or
(iv) the antisense strand is 18 nucleotides, the sense strand is 15 nucleotides, the duplex region is 15 base pairs, and the 3′ overhang is 3 nucleotides.

In some or any of the foregoing or related embodiments, two to four consecutive nucleotides of the sense strand are each conjugated to a GalNAc molecule.

In some or any of the foregoing or related embodiments, the three 3′ terminal nucleotides of the sense strand are each conjugated to a GalNAc molecule.

In some or any of the foregoing or related embodiments, the GalNAc molecule is a monovalent GalNAc moiety, a bivalent GalNAc moiety, a trivalent GalNAc moiety, or a tetravalent GalNAc moiety. In some embodiments, the GalNAc molecule is a monovalent GalNAc moiety.

In some aspects, the disclosure provides a double-stranded oligonucleotide comprising an antisense strand of about 20-22 nucleotides in length and a sense strand of about 18-20 nucleotides in length, wherein the antisense and sense strands form an asymmetric duplex region of about 18-20 base pairs comprising a 3′ terminal overhang of at least 2 nucleotides of the antisense strand, wherein the antisense strand comprises a region of complementarity to a liver mRNA target sequence, and wherein the sense strand comprises two to four consecutive nucleotides at the 5′ or 3′ terminus each conjugated to a monovalent GalNAc molecule.

In some or any of the foregoing or related embodiments, the antisense strand is 22 nucleotides in length, the sense strand is 20 nucleotides in length, the duplex region is 20 base pairs, and the 3′ terminal overhang is 2 nucleotides.

In some or any of the foregoing or related embodiments, the oligonucleotide comprises a blunt end comprising the 3′ end of the sense strand and the 5′ end of the antisense strand.

In some aspects, the disclosure provides double-stranded oligonucleotide comprising an antisense strand of about 18-22 nucleotides in length and a sense strand of about 15 nucleotides in length, wherein the antisense and sense strands form an asymmetric duplex region of about 15 base pairs comprising a 3′ terminal overhang of at least 2 nucleotides of the antisense strand, wherein the antisense strand comprises a region of complementarity to an mRNA target sequence, and wherein the sense strand comprises three consecutive nucleotides at the 5′ or 3′ terminus each conjugated to a monovalent GalNAc molecule.

In some or any of the foregoing or related embodiments,
(i) the antisense strand is 21 nucleotides, the sense strand is 15 nucleotides, the duplex region is 15 base pairs, and the 3′ terminal overhang is 6 nucleotides;
(ii) the antisense strand is 20 nucleotides, the sense strand is 15 nucleotides, the duplex region is 15 base pairs, and the 3′ terminal overhang is 5 nucleotides;
(iii) the antisense strand is 19 nucleotides, the sense strand is 15 nucleotides, the duplex region is 15 base pairs, and the 3′ terminal overhang is 4 nucleotides; or
(iv) the antisense strand is 18 nucleotides, the sense strand is 15 nucleotides, the duplex region is 15 base pairs, and the 3′ terminal overhang is 3 nucleotides.

In some or any of the foregoing or related embodiments, the oligonucleotide comprises a blunt end comprising the 3′ end of the sense strand and the 5′ end of the antisense strand.

In some embodiments, the disclosure provides a double-stranded oligonucleotide comprising an antisense strand of about 20-22 nucleotides in length and a sense strand of about 18-20 nucleotides in length, wherein the antisense and sense strands form an asymmetric duplex region of about 18-20 base pairs comprising a 3′ terminal overhang of at least 2 nucleotides of the antisense strand, wherein the antisense strand comprises a region of complementarity to an mRNA target sequence, and wherein the antisense strand comprises two to four nucleotides each conjugated to a monovalent GalNAc molecule.

In some or any of the foregoing or related embodiments, the antisense strand is 22 nucleotides, and wherein (i) nucleotides at positions 20 and 21 are each conjugated to the monovalent GalNAc molecule, (ii) nucleotides at positions 19 and 20 are each conjugated to the monovalent GalNAc molecule, or (iii) the nucleotide at position 20 is conjugated to the monovalent GalNAc molecule.

In some or any of the foregoing or related embodiments, the sense strand comprises a monovalent GalNAc molecule conjugated to the 5′ terminal nucleotide.

In some or any of the foregoing or related embodiments, the two 5′ terminal nucleotides of the sense strand are each conjugated to a monovalent GalNAc molecule.

In some aspects, the disclosure provides a double-stranded oligonucleotide comprising an antisense strand of about 20-22 nucleotides in length and a sense strand of about 13-18 nucleotides in length, wherein the antisense and sense strands form a duplex region of about 13-18 base pairs, wherein the antisense strand comprises an orientation of 5′ to 3′, wherein the antisense strand comprises a 3′ overhang of at least one nucleotide and a 5′ overhang of at least one nucleotide, wherein the antisense strand comprises a region of complementarity to an mRNA target sequence, and wherein the sense strand comprises two to four nucleotides each conjugated to a monovalent GalNAc molecule.

In some or any of the foregoing or related embodiments, the antisense strand is 22 nucleotides, the sense strand is 18 nucleotides, the duplex region is 18 base pairs, the 3′ overhang is 2 nucleotides, and the 5′ overhang is two nucleotides.

In some or any of the foregoing or related embodiments, the three consecutive 3′ terminal nucleotides of the sense strand are each conjugated to the monovalent GalNAc molecule.

In some or any of the foregoing or related embodiments, the antisense strand is 22 nucleotides, the sense strand is 13 nucleotides, the duplex region is 13 base pairs, the 3′ overhang is 7 nucleotides, and the 5′ overhang is 2 nucleotides.

In some or any of the foregoing or related embodiments, two to three consecutive nucleotides of the sense strand are each conjugated to the monovalent GalNAc molecule.

In some or any of the foregoing or related embodiments, the antisense strand is 22 nucleotides, the sense strand is 16 nucleotides, the duplex region is 16 base pairs, the 3′ overhang is 4 nucleotides, and the 5′ overhang is 2 nucleotides.

In some or any of the foregoing or related embodiments, the antisense strand is 20 nucleotides, the sense strand is 16 nucleotides, the duplex region is 16 base pairs, the 3′ overhang is 2 nucleotides, and the 5′ overhang is 2 nucleotides.

In some or any of the foregoing or related embodiments, the antisense strand is 22 nucleotides, the sense strand is 20 nucleotides, the duplex region is 18 base pairs, the 3′ overhang of the antisense strand is 2 nucleotides, and the 5′ end of the antisense strand is blunt.

In some or any of the foregoing or related embodiments, the antisense strand is 22 nucleotides, the sense strand is 17 nucleotides, the duplex region is 17 base pairs, the 3′ overhang is 3 nucleotides, and the 5′ overhang is 2 nucleotides.

In some or any of the foregoing or related embodiments, the three consecutive 5′ terminal nucleotides of the sense strand are each conjugated to the monovalent GalNAc molecule.

In some aspects, the disclosure provides a double-stranded oligonucleotide comprising an antisense strand of about 18-22 nucleotides in length and a sense strand of about 13-18 nucleotides in length, wherein the antisense and sense strands form a duplex region of about 13-18 base pairs, wherein the antisense strand comprises an orientation of 5′ to 3′, wherein the antisense strand comprises a 3′ overhang of at least one nucleotide and a 5′ overhang of at least one nucleotide, wherein the antisense strand comprises a region of complementarity to an mRNA target sequence, and wherein the sense strand comprises two to four nucleotides each conjugated to a monovalent GalNAc molecule.

In some or any of the foregoing or related embodiments, the oligonucleotide comprises at least one modified nucleotide. In some or any of the foregoing or related aspects, the modified nucleotide comprises a 2′-modification.

In some or any of the foregoing or related embodiments, each of the nucleotides of the sense strand and the antisense strand comprise a 2′-modification. In some or any of the foregoing or related aspects, the 2′-modification is a modification selected from 2′-aminoethyl, 2′-fluoro, 2′-O-methyl, 2′-O-methoxyethyl, and 2′-deoxy-2′-fluoro-β-d-arabinonucleic acid. In some embodiments, the sense strand comprises nucleotide positions numbered 5′ to 3′, and wherein position 8 comprises a 2′-fluor modification, or each of positions (i) 8-11; (ii) 3-6; (iii) 5-6; (iv) 3-4; (v) 6-9; or (vi) 7-10, comprise a 2′-fluoro modification. In some embodiments, the sense strand comprises a 2′-fluoro modification at each of nucleotides forming a base pair with nucleotides at positions 10-13, 12-13, or 10-11, of the antisense strand, wherein positions are numbered 5′ to 3′. In some embodiments, the antisense strand comprises positions numbered 5′ to 3′, and wherein each of positions 2, 3, 4, 5, 7, 10 and 14 comprise a 2′-fluoro modification. In some embodiments, the remaining nucleotides comprise a 2′-O-methyl modification, provided that the nucleotides conjugated to the GalNAc molecule does not comprise a 2′-O-methyl modification.

In some or any of the foregoing or related embodiments, the oligonucleotide comprises at least one modified internucleotide linkage. In some or any of the foregoing or related aspects, the at least one modified internucleotide linkage is a phosphorothioate linkage. In some embodiments, the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′. In some embodiments, the antisense strand comprises a phosphorothioate linkage between the penultimate nucleotide and third nucleotide from the 3′ end, and between the penultimate nucleotide and ultimate nucleotide. In some embodiments, the antisense strand is 18 nucleotides in length, and wherein the antisense strand comprises a phosphorothioate linkage between positions 16 and 17 and between positions 17 and 18, wherein positions are numbered 1-18 from 5′ to 3′. In some embodiments, the sense strand comprises a phosphorothioate linkage between positions 1 and 2, between the penultimate nucleotide and third nucleotide from the 3′ end, and between the penultimate nucleotide and ultimate nucleotide.

In some or any of the foregoing or related embodiments, the sense strand comprises at least one Tm-increasing nucleotide. In some embodiments, the sense strand comprises up to three Tm-increasing nucleotides. In some embodiments, the sense strand comprises three Tm-increasing nucleotides. In some embodiments, the Tm-increasing nucleotide is a bicyclic nucleotide. In some embodiments, the Tm-increasing nucleotide is a locked nucleic acid.

In some or any of the foregoing or related embodiments, the antisense strand comprises a phosphorylated nucleotide at the 5′ terminus, wherein the phosphorylated nucleotide is selected from uridine and adenosine. In some embodiments, the phosphorylated nucleotide is uridine. In some embodiments, the phosphorylated nucleotide is 4′-O-monomethylphosphonate-2′-O-methyl uridine.

In some or any of the foregoing or related embodiments, the 4′-carbon of the sugar of the 5′-nucleotide of the antisense strand comprises a phosphate analog. In some embodiments, the phosphate analog is oxymethyl phosphonate, vinyl phosphonate or malonyl phosphonate.

In some or any of the foregoing or related embodiments, the region of complementarity is fully complementary to the liver mRNA target sequence. In some or any of the foregoing or related embodiments, the region of complementarity is partially complementary to the liver mRNA target sequence. In some embodiments, the region of complementarity comprises no more than four mismatches to the liver mRNA target sequence.

In some or any of the foregoing or related embodiments, the region of complementarity is fully complementary to the mRNA target sequence. In some or any of the foregoing or related embodiments, the region of complementarity is partially complementary to the mRNA target sequence.

In some embodiments, the region of complementarity comprises no more than four mismatches to the mRNA target sequence

In some or any of the foregoing or related embodiments, the oligonucleotide reduces expression of the liver mRNA target sequence or mRNA target sequence in a cell or population of cells in vitro and/or in vivo.

In some aspects, the disclosure provides a pharmaceutical composition comprising the oligonucleotide of any of the foregoing or related aspects, and a pharmaceutically acceptable carrier, delivery agent or excipient.

In some aspects, the disclosure provides a method for treating a subject having a disease, disorder or condition associated with expression of a liver mRNA or target mRNA, the method comprising administering to the subject a therapeutically effective amount of the double-stranded oligonucleotide of any of the foregoing or related embodiments, or the pharmaceutical composition of any of the foregoing or related embodiments.

In some aspects, the disclosure provides a method of reducing expression of a liver mRNA or target mRNA in a subject, comprising administering to the subject the double-stranded oligonucleotide of any of the foregoing or related aspects, or the pharmaceutical composition of any of the foregoing or related aspects.

In some aspects, the disclosure provides a kit comprising the double-stranded oligonucleotide of some or any of the foregoing or related embodiments, an optional pharmaceutically acceptable carrier, and a package insert comprising instructions for administration to a subject having a disease, disorder or condition associated with the overexpression or reduction in expression of a liver mRNA or target mRNA.

In some aspects, the disclosure provides for use of the double-stranded oligonucleotide of some or any of the foregoing or related embodiments, or the pharmaceutical composition of any of the foregoing or related aspects, in the manufacture of a medicament for the treatment of a disease, disorder, or condition associated with the overexpression or reduction in expression of a liver mRNA or a target mRNA.

In some aspects, the disclosure provides a double-stranded oligonucleotide of any of the foregoing or related aspects, or the pharmaceutical composition of some or any of the foregoing or related embodiments, for use, or adaptable for use, in the treatment of a disease, disorder, or condition associated with overexpression or reduction in expression of a liver mRNA or target mRNA.

In some aspects, the disclosure provides a method of activating target-specific RNA interference (RNAi) in an organism comprising administering to said organism the double-stranded oligonucleotide of some or any of the foregoing or related embodiments, said double-stranded oligonucleotide being administered in an amount sufficient for degradation of the liver mRNA or target mRNA to occur, thereby activating target-specific RNAi in the organism.

In some or any of the foregoing or related embodiments, the liver mRNA or target mRNA specifies the amino acid sequence of a protein involved or predicted to be involved in a human disease or disorder.

In some or any of the foregoing or related embodiments, the disease or disorder is selected from the group consisting of viral infections, bacterial infections, parasitic infections, cancers, allergies, autoimmune diseases, immunodeficiencies, and immunosuppression.

In some aspects, the disclosure provides a double-stranded oligonucleotide comprising an antisense strand of about 20-22 nucleotides in length and a sense strand of about 16-20 nucleotides in length, wherein:
(i) the antisense and sense strands form a duplex region of about 16-20 base pairs,
(ii) the antisense strand comprises a 3′ overhang of at least two nucleotides,
(iii) the antisense strand comprises a region of complementarity to a liver mRNA target sequence,
(iv) the sense strand and antisense strand each comprise at least two nucleotides modified with a 2′-modification, wherein the 2′-modification is 2′-fluoro and 2′-O-methyl, and
(v) the sense strand comprises one to four nucleotides conjugated to a GalNAc molecule.

In some or any of the foregoing or related embodiments, the antisense strand comprises a 5′ overhang of at least two nucleotides.

In some or any of the foregoing or related embodiments, the oligonucleotide comprises a blunt end comprising the 5′ end of the antisense strand and the 3′ end of the sense strand.

In some or any of the foregoing or related embodiments, the antisense strand is 22 nucleotides and the sense strand is 18-20 nucleotides.

In some or any of the foregoing or related embodiments, the antisense strand comprises nucleotide positions numbered 5′ to 3′, and wherein positions 2, 3, 4, 5, 7 and 14 each comprise a 2′-fluoro modification.

In some or any of the foregoing or related embodiments, positions 6, 8, 9, 11-13 and 15-22 each comprise a 2′-O-methyl modification.

In some or any of the foregoing or related embodiments, the sense strand is 18 nucleotides, and wherein the antisense comprises a 5′ overhang of at least two nucleotides.

In some or any of the foregoing or related embodiments, the sense strand is 20 nucleotides, and wherein the oligonucleotide comprises a blunt end comprising the 5′ end of the antisense strand and the 3′ end of the sense strand.

In some or any of the foregoing or related embodiments, the sense strand comprises three nucleotides conjugated to a GalNAc molecule.

In some or any of the foregoing or related embodiments, the three nucleotides are at the 3′ end of the sense strand.

In some or any of the foregoing or related embodiments, the sense strand comprises nucleotide positions numbered 5′ to 3′, and wherein positions 8-11 each comprise a 2′-fluoro modification.

In some or any of the foregoing or related embodiments, the remaining nucleotides of the sense strand comprise a 2′-O-methyl modification, provided the nucleotide is not conjugated to the GalNAc molecule.

In some or any of the foregoing or related embodiments, the antisense strand is 20 nucleotides and the sense strand is 16-18 nucleotides.

In some or any of the foregoing or related embodiments, the antisense strand comprises nucleotide positions numbered 5′ to 3′, and wherein positions 2, 3, 4, 5, 7 and 14 each comprise a 2′-fluoro modification.

In some or any of the foregoing or related embodiments, positions 6, 8, 9, 11-13 and 15-20 each comprise a 2′-O-methyl modification.

In some or any of the foregoing or related embodiments, the sense strand is 16 nucleotides, and wherein the antisense comprises a 5′ overhang of at least two nucleotides.

In some or any of the foregoing or related embodiments, the sense strand comprises three nucleotides conjugated to a GalNAc molecule.

In some or any of the foregoing or related embodiments, the three nucleotides are at the 3′ end of the sense strand.

In some or any of the foregoing or related embodiments, the sense strand comprises nucleotide positions numbered 5′ to 3′, and wherein positions 6-9 each comprise a 2′-fluoro modification.

In some or any of the foregoing or related embodiments, the remaining nucleotides of the sense strand comprise a 2′-O-methyl modification, provided the nucleotide is not conjugated to the GalNAc molecule.

In some or any of the foregoing or related embodiments, the antisense strand comprises a phosphorylated nucleotide at the 5′ terminus, wherein the phosphorylated nucleotide is selected from uridine and adenosine.

In some or any of the foregoing or related embodiments, the phosphorylated nucleotide is uridine.

In some or any of the foregoing or related embodiments, the phosphorylated nucleotide is 4′-O-monomethylphosphonate-2′-O-methyl uridine.

In some or any of the foregoing or related embodiments, the 4′-carbon of the sugar of the 5′-nucleotide of the antisense strand comprises a phosphate analog.

In some or any of the foregoing or related embodiments, the phosphate analog is oxymethyl phosphonate, vinyl phosphonate or malonyl phosphonate.

In some or any of the foregoing or related embodiments, the oligonucleotide comprises at least one modified internucleotide linkage.

In some or any of the foregoing or related embodiments, the at least one modified internucleotide linkage is a phosphorothioate linkage.

In some or any of the foregoing or related embodiments, the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′.

In some or any of the foregoing or related embodiments, the antisense strand comprises a phosphorothioate linkage between the penultimate nucleotide and third nucleotide from the 3′ end, and between the penultimate nucleotide and ultimate nucleotide.

In some or any of the foregoing or related embodiments, the sense strand comprises a phosphorothioate linkage between positions 1 and 2, between the penultimate nucleotide and third nucleotide from the 3′ end, and between the penultimate nucleotide and ultimate nucleotide.

In some or any of the foregoing or related embodiments, the oligonucleotide reduces expression of the liver mRNA target sequence in a cell or population of cells in vitro and/or in vivo.

In some aspects, the disclosure provides a pharmaceutical composition comprising the oligonucleotide of some or any of the foregoing or related embodiments, and a pharmaceutically acceptable carrier, delivery agent or excipient.

In some aspects, the disclosure provides a method for treating a subject having a disease, disorder or condition associated with expression of a liver mRNA, the method comprising administering to the subject a therapeutically effective amount of the double-stranded oligonucleotide of some or any of the foregoing or related embodiments, or the pharmaceutical composition of some or any of the foregoing or related embodiments.

In some aspects, the disclosure provides a method of reducing expression of a liver mRNA in a subject, comprising administering to the subject the double-stranded oligonucleotide of some or any of the foregoing or related embodiments, or the pharmaceutical composition of some or any of the foregoing or related embodiments.

### BRIEF DESCRIPTION OF FIGURES

FIG. 1 provides schematics of RNAi oligonucleotide-ligand conjugates having the structures and sequences of Compounds 1-16.

FIG. 2 provides a graph measuring percent (%) murine Aldh2 and Hao1 mRNA remaining in liver of mice 5 days after administration of PBS or 0.5 mg/kg of Compounds 1-16 via subcutaneous injection.

FIG. 3 provides schematics of RNAi oligonucleotide-ligand conjugates having the structures and sequences of Compounds 17-23.

FIG. 4 provides a graph measuring percent (%) murine Aldh2 mRNA remaining in liver of mice 7 days after administration of PBS or 0.5 mg/kg of Compounds 17-23 via subcutaneous injection.

FIG. 5 provides schematics of RNAi oligonucleotide-ligand conjugates having the structures and sequences of Compounds 17-18 and 24-27.

FIG. 6 provides a graph measuring percent (%) murine Aldh2 mRNA remaining in liver of mice 7 days after administration of PBS or 0.5 mg/kg of Compounds 17-18 and 24-27 via subcutaneous injection.

FIG. 7 provides schematics of RNAi oligonucleotide-ligand conjugates having the structures and sequences of Compounds 17-18 and 28-34.

FIG. 8 provides a graph measuring percent (%) murine Aldh2 mRNA remaining in liver of mice 7 days after administration of PBS or 0.5 mg/kg of Compounds 17-18 and 28-34 via subcutaneous injection.

FIG. 9 provides schematics of RNAi oligonucleotide-ligand conjugates having the structures and sequences of Compounds 1, 9, and 35-42.

FIG. 10 provides a graph measuring percent (%) murine Aldh2 and Hao1 mRNA remaining in liver of mice 5 days after administration of PBS or 0.5 mg/kg of Compounds 1, 9, and 35-42 via subcutaneous injection.

FIG. 11 provides schematics of RNAi oligonucleotide-ligand conjugates having the structures and sequences of Compounds 1, 9, and 43-56.

FIG. 12 provides a graph measuring percent (%) murine Aldh2 and Hao1 mRNA remaining in liver of mice 5 days after administration of PBS or 0.5 mg/kg of Compounds 1, 9, and 43-56 via subcutaneous injection.

FIG. 13 provides schematics of RNAi oligonucleotide-ligand conjugates having the structures and sequences of Compounds 1, 17, 18, 33, and 24.

FIG. 14 provides a graph measuring percent (%) murine Aldh2 mRNA remaining in liver of mice 7 days after administration of PBS or Compounds 1, 9, and 35-42 at either 0.25 mg/kg or 0.5 mg/kg via subcutaneous injection.

FIG. 15 provides schematics of RNAi oligonucleotide-ligand conjugates having the structures and sequences of Compounds 1, 2, 7-10, 15, and 16.

FIG. 16A provides a graph measuring percent (%) murine Aldh2 mRNA remaining in liver of mice 7, 14, and 21 days after administration of PBS or 0.5 mg/kg of Compounds 1, 2, 7, and 8, and 9 via subcutaneous injection.

FIG. 16B provides a graph measuring percent (%) murine Hao1 mRNA remaining in liver of mice 7, 14, and 21 days after administration of PBS or 0.5 mg/kg of Compounds 1, 9, 10, 15, and 16 via subcutaneous injection.

FIG. 17 provides schematics of RNAi oligonucleotide-ligand conjugates having the structures and sequences of Compounds 1 and 18.

FIGS. 18A-18B provide graphs measuring percent (%) non-human primate (NHP) Aldh2 mRNA remaining in liver of NHPs 28, 56, 84, and 112 days after administration of PBS or 3 mg/kg of Compounds 1 and 18 via subcutaneous injection. FIG. 18A shows the average expression for each treatment group and FIG. 18B shows expression in individual animals.

FIG. 19 provides a schematic of an RNAi oligonucleotide-ligand conjugate having the structure of Compound 57.

### DETAILED DESCRIPTION

In some aspects, the disclosure provides oligonucleotide-ligand conjugates (e.g., RNAi oligonucleotide-ligand conjugates) that reduce expression of a target gene. In other aspects, the disclosure provides methods of treating a disease or disorder associated with expression of a target gene. In other aspects, the disclosure provides methods of treating a disease or disorder associated with expression of a target gene using the ligand-conjugated RNAi oligonucleotides, or pharmaceutically acceptable compositions thereof, described herein. In other aspects, the disclosure provides methods of using the ligand-conjugated RNAi oligonucleotides described herein in the manufacture of a medicament for treating a disease or disorder associated with expression of a target gene.

### Ligand-Conjugated RNAi Oligonucleotides

The disclosure provides, inter alia, ligand-conjugated RNAi oligonucleotides (e.g., RNAi oligonucleotide-ligand conjugates) that reduce expression of a target gene. In some embodiments, a ligand-conjugated RNAi oligonucleotide provided by the disclosure is targeted to an mRNA encoding the target gene. Messenger RNA (mRNA) that encodes a target gene and is targeted by a ligand-conjugated RNAi oligonucleotide of the disclosure is referred to herein as “target mRNA”.

mRNA Target Sequences

In some embodiments, the ligand-conjugated RNAi oligonucleotide is targeted to a target sequence comprising a target mRNA. In some embodiments, the ligand-conjugated RNAi oligonucleotide is targeted to a target sequence within a target mRNA. In some embodiments, the ligand-conjugated RNAi oligonucleotide, or a portion, fragment, or strand thereof (e.g., an antisense strand or a guide strand of a double-stranded oligonucleotide) binds or anneals to a target sequence comprising a target mRNA, thereby reducing target gene expression. In some embodiments, the ligand-conjugated RNAi oligonucleotide is targeted to a target sequence comprising target mRNA for the purpose of reducing expression of a target gene in vivo. In some embodiments, the amount or extent of reduction of target gene expression by a ligand-conjugated RNAi oligonucleotide targeted to a specific target sequence correlates with the potency of the ligand-conjugated RNAi oligonucleotide. In some embodiments, the amount or extent of reduction of target gene expression by a ligand-conjugated RNAi oligonucleotide targeted to a specific target sequence correlates with the amount or extent of therapeutic benefit in a subject or patient having a disease, disorder or condition associated with target gene expression treated with the ligand-conjugated RNAi oligonucleotide.

Through examination of the nucleotide sequence of mRNAs encoding target genes, including mRNAs of multiple different species (e.g., human, cynomolgus monkey, mouse, and rat) and as a result of in vitro and in vivo testing, it has been discovered that certain nucleotide sequences and certain systemic modifications to those oligonucleotides are more amenable than others to RNAi oligonucleotide-mediated reduction and are thus useful as part of oligonucleotides that are otherwise targeted to specific gene target sequences. In some embodiments, a sense strand of a ligand-conjugated RNAi oligonucleotide, or a portion or fragment thereof, described herein, comprises a nucleotide sequence that is similar (e.g., having no more than 4 mismatches) or is identical to a target sequence comprising a target mRNA. In some embodiments, a portion or region of the sense strand of a double-stranded oligonucleotide described herein comprises a target sequence comprising a target mRNA.

In some embodiments, the target mRNA is expressed in a tissue or cell of a subject. In some embodiments, the target mRNA is expressed in more than one tissue or cell of a subject, wherein the tissues or cells are different. In some embodiments, the target mRNA is differentially expressed in a tissue or cell. In some embodiments, the target mRNA is expressed throughout multiple tissue types and/or cell types. In some embodiments, the target mRNA is expressed in liver tissue. In some embodiments, the target mRNA is expressed in a hepatocyte

### RNAi Oligonucleotide Targeting Sequences

In some embodiments, the ligand-conjugated RNAi oligonucleotides provided by the disclosure comprise a targeting sequence. As used herein, the term “targeting sequence” refers to a nucleotide sequence having a region of complementarity to a specific nucleotide sequence comprising an mRNA. In some embodiments, the ligand-conjugated RNAi oligonucleotides provided by the disclosure comprise a gene targeting sequence having a region of complementarity to a nucleotide sequence comprising a target sequence of a target mRNA.

The targeting sequence imparts the ligand-conjugated RNAi oligonucleotide with the ability to specifically target an mRNA by binding or annealing to a target sequence comprising a target mRNA by complementary (Watson-Crick) base pairing. In some embodiments, the ligand-conjugated RNAi oligonucleotides herein (or a strand thereof, e.g., an antisense strand or a guide strand of a double-stranded oligonucleotide) comprise a targeting sequence having a region of complementarity that binds or anneals to a target sequence comprising a target mRNA by complementary (Watson-Crick) base pairing. In some embodiments, the ligand-conjugated RNAi oligonucleotides herein (or a strand thereof, e.g., an antisense strand or a guide strand of a double-stranded oligonucleotide) comprise a targeting sequence having a region of complementarity that binds or anneals to a target sequence within a target mRNA by complementary (Watson-Crick) base pairing. The targeting sequence is generally of suitable length and base content to enable binding or annealing of the ligand-conjugated RNAi oligonucleotide (or a strand thereof) to a specific target mRNA for purposes of inhibiting target gene expression. In some embodiments, the targeting sequence is at least 16, at least 17, at least 18, at least 19, at least 20, at least 21, or at least 22 nucleotides in length. In some embodiments, the targeting sequence is about 16 to about 22 (e.g., 16 to 22, 16 to 21, 16 to 20, 16 to 19, 16 to 17, or 16 to 17) nucleotides in length. In some embodiments, the targeting sequence is about 16, 17, 18, 19, 20, 21, or 22 nucleotides in length. In some embodiments, the targeting sequence is 12 nucleotides in length. In some embodiments, the targeting sequence is 13 nucleotides in length. In some embodiments, the targeting sequence is 14 nucleotides in length. In some embodiments, the targeting sequence is 15 nucleotides in length. In some embodiments, the targeting sequence is 16 nucleotides in length. In some embodiments, the targeting sequence is 17 nucleotides in length. In some embodiments, the targeting sequence is 18 nucleotides in length. In some embodiments, the targeting sequence is 19 nucleotides in length. In some embodiments, the targeting sequence is 20 nucleotides in length. In some embodiments, the targeting sequence is 21 nucleotides in length. In some embodiments, the targeting sequence is 22 nucleotides in length.

In some embodiments, the ligand-conjugated RNAi oligonucleotides herein comprise a targeting sequence that is fully complementary to a target sequence comprising a target mRNA. In some embodiments, the ligand-conjugated RNAi oligonucleotides herein comprise a targeting sequence that is fully complementary to a target sequence within a target mRNA. In some embodiments, the targeting sequence is partially complementary to a target sequence comprising a target mRNA. In some embodiments, the targeting sequence is partially complementary to a target sequence within a target mRNA. In some embodiments, the targeting sequence comprises a region of contiguous nucleotides comprising the antisense strand.

In some embodiments, the ligand-conjugated RNAi oligonucleotides herein comprise a targeting sequence that is complementary to a contiguous sequence of nucleotides comprising a target mRNA, wherein the contiguous sequence of nucleotides is about 12 to about 20 nucleotides in length (e.g., 12 to 20, 12 to 18, 12 to 16, 15 to 18, 15 to 20, 12 to 16, 14 to 20, 16 to 20, 18 to 20 or 18 to 19 nucleotides in length). In some embodiments, the ligand-conjugated RNAi oligonucleotides comprise a targeting sequence that is complementary to a contiguous sequence of nucleotides comprising a target mRNA, wherein the contiguous sequence of nucleotides is 10, 11, 12, 13, 14, 15, 16, 17, 18, 19 or 20 nucleotides in length. In some embodiments, the ligand-conjugated RNAi oligonucleotides comprise a targeting sequence that is complementary to a contiguous sequence of nucleotides comprising a target mRNA, wherein the contiguous sequence of nucleotides is 13 nucleotides in length. In some embodiments, the ligand-conjugated RNAi oligonucleotides comprise a targeting sequence that is complementary to a contiguous sequence of nucleotides comprising a target mRNA, wherein the contiguous sequence of nucleotides is 14 nucleotides in length. In some embodiments, the ligand-conjugated RNAi oligonucleotides comprise a targeting sequence that is complementary to a contiguous sequence of nucleotides comprising a target mRNA, wherein the contiguous sequence of nucleotides is 15 nucleotides in length. In some embodiments, the ligand-conjugated RNAi oligonucleotides comprise a targeting sequence that is complementary to a contiguous sequence of nucleotides comprising a target mRNA, wherein the contiguous sequence of nucleotides is 16 nucleotides in length. In some embodiments, the ligand-conjugated RNAi oligonucleotides comprise a targeting sequence that is complementary to a contiguous sequence of nucleotides comprising a target mRNA, wherein the contiguous sequence of nucleotides is 17 nucleotides in length. In some embodiments, the ligand-conjugated RNAi oligonucleotides comprise a targeting sequence that is complementary to a contiguous sequence of nucleotides comprising a target mRNA, wherein the contiguous sequence of nucleotides is 18 nucleotides in length. In some embodiments, the ligand-conjugated RNAi oligonucleotides comprise a targeting sequence that is complementary to a contiguous sequence of nucleotides comprising a target mRNA, wherein the contiguous sequence of nucleotides is 19 nucleotides in length. In some embodiments, the ligand-conjugated RNAi oligonucleotides comprise a targeting sequence that is complementary to a contiguous sequence of nucleotides comprising a target mRNA, wherein the contiguous sequence of nucleotides is 20 nucleotides in length.

In some embodiments, a targeting sequence of a ligand-conjugated RNAi oligonucleotide herein is fully complementary (e.g., having no mismatches) to a target sequence comprising a target mRNA and comprises the entire length of an antisense strand. In some embodiments, a targeting sequence of a ligand-conjugated RNAi oligonucleotide herein is fully complementary (e.g., having no mismatches) to a target sequence comprising a target mRNA and comprises a portion of the entire length of an antisense strand. In some embodiments, a targeting sequence of a ligand-conjugated RNAi oligonucleotide herein is fully complementary (e.g., having no mismatches) to a target sequence comprising a target mRNA and comprises 10 to 20 nucleotides of the antisense strand. In some embodiments, a targeting sequence of a ligand-conjugated RNAi oligonucleotide herein is fully complementary (e.g., having no mismatches) to a target sequence comprising a target mRNA and comprises 15 to 19 nucleotides of the antisense strand. In some embodiments, a targeting sequence of a ligand-conjugated RNAi oligonucleotide herein is fully complementary (e.g., having no mismatches) to a target sequence comprising a target mRNA and comprises 13 nucleotides, 14 nucleotides, 15 nucleotides, 16 nucleotides, 17 nucleotides, 18 nucleotides, 19 nucleotides, 20 nucleotides, 21 nucleotides, or 22 nucleotides of the antisense strand. In some embodiments, a targeting sequence of a ligand-conjugated RNAi oligonucleotide herein is fully complementary (e.g., having no mismatches) to a target sequence comprising a target mRNA and comprises 20 nucleotides of the antisense strand. In some embodiments, a targeting sequence of a ligand-conjugated RNAi oligonucleotide herein is fully complementary (e.g., having no mismatches) to a target sequence comprising a target mRNA and comprises 19 nucleotides of the antisense strand. In some embodiments, a targeting sequence of a ligand-conjugated RNAi oligonucleotide herein is fully complementary (e.g., having no mismatches) to a target sequence comprising a target mRNA and comprises 18 nucleotides of the antisense strand. In some embodiments, a targeting sequence of a ligand-conjugated RNAi oligonucleotide herein is fully complementary (e.g., having no mismatches) to a target sequence comprising a target mRNA and comprises 17 nucleotides of the antisense strand. In some embodiments, a targeting sequence of a ligand-conjugated RNAi oligonucleotide herein is fully complementary (e.g., having no mismatches) to a target sequence comprising a target mRNA and comprises 16 nucleotides of the antisense strand.

In some embodiments, a targeting sequence of a ligand-conjugated RNAi oligonucleotide herein is partially complementary (e.g., having no more than 4 mismatches) to a target sequence comprising a target mRNA and comprises the entire length of an antisense strand. In some embodiments, a targeting sequence of a ligand-conjugated RNAi oligonucleotide herein is partially complementary (e.g., having no more than 4 mismatches) to a target sequence comprising a target mRNA and comprises a portion of the entire length of an antisense strand. In some embodiments, a targeting sequence of a ligand-conjugated RNAi oligonucleotide herein is partially complementary (e.g., having no more than 4 mismatches) to a target sequence comprising a target mRNA and comprises 15 to 20 nucleotides of the antisense strand. In some embodiments, a targeting sequence of a ligand-conjugated RNAi oligonucleotide herein is partially complementary (e.g., having no more than 4 mismatches) to a target sequence comprising a target mRNA and comprises 15 to 19 nucleotides of the antisense strand. In some embodiments, a targeting sequence of a ligand-conjugated RNAi oligonucleotide herein is partially complementary (e.g., having no more than 4 mismatches) to a target sequence comprising a target mRNA and comprises 15 nucleotides, 16 nucleotides, 17 nucleotides, 18 nucleotides, 19 nucleotides, 20 nucleotides, 21 nucleotides, or 22 nucleotides of the antisense strand. In some embodiments, a targeting sequence of a ligand-conjugated RNAi oligonucleotide herein is partially complementary (e.g., having no more than 4 mismatches) to a target sequence comprising a target mRNA and comprises 20 nucleotides of the antisense strand. In some embodiments, a targeting sequence of a ligand-conjugated RNAi oligonucleotide herein is partially complementary (e.g., having no more than 4 mismatches) to a target sequence comprising a target mRNA and comprises 19 nucleotides of the antisense strand. In some embodiments, a targeting sequence of a ligand-conjugated RNAi oligonucleotide herein is partially complementary (e.g., having no more than 4 mismatches) to a target sequence comprising a target mRNA and comprises 18 nucleotides of the antisense strand. In some embodiments, a targeting sequence of a ligand-conjugated RNAi oligonucleotide herein is partially complementary (e.g., having no more than 4 mismatches) to a target sequence comprising a target mRNA and comprises 17 nucleotides of the antisense strand. In some embodiments, a targeting sequence of a ligand-conjugated RNAi oligonucleotide herein is partially complementary (e.g., having no more than 4 mismatches) to a target sequence comprising a target mRNA and comprises 16 nucleotides of the antisense strand. In some embodiments, a targeting sequence of a ligand-conjugated RNAi oligonucleotide herein is partially complementary (e.g., having no more than 4 mismatches) to a target sequence comprising a target mRNA and comprises 15 nucleotides of the antisense strand.

In some embodiments, a ligand-conjugated RNAi oligonucleotide herein comprises a targeting sequence having one or more base pair (bp) mismatches with the corresponding target sequence comprising a target mRNA. In some embodiments, the targeting sequence has a 1 bp mismatch, a 2 bp mismatch, a 3 bp mismatch, a 4 bp mismatch, or a 5 bp mismatch with the corresponding target sequence comprising a target mRNA provided that the ability of the targeting sequence to bind or anneal to the target sequence under appropriate hybridization conditions and/or the ability of the ligand-conjugated RNAi oligonucleotide to inhibit or reduce target gene expression is maintained (e.g., under physiological conditions). Alternatively, in some embodiments, the targeting sequence comprises no more than 1, no more than 2, no more than 3, no more than 4, or no more than 5 bp mismatches with the corresponding target sequence comprising a target mRNA provided that the ability of the targeting sequence to bind or anneal to the target sequence under appropriate hybridization conditions and/or the ability of the ligand-conjugated RNAi oligonucleotide to inhibit or reduce target gene expression is maintained. In some embodiments, the ligand-conjugated RNAi oligonucleotide comprises a targeting sequence having 1 mismatch with the corresponding target sequence. In some embodiments, the ligand-conjugated RNAi oligonucleotide comprises a targeting sequence having 2 mismatches with the corresponding target sequence. In some embodiments, the ligand-conjugated RNAi oligonucleotide comprises a targeting sequence having 3 mismatches with the corresponding target sequence. In some embodiments, the ligand-conjugated RNAi oligonucleotide comprises a targeting sequence having 4 mismatches with the corresponding target sequence. In some embodiments, the ligand-conjugated RNAi oligonucleotide comprises a targeting sequence having 5 mismatches with the corresponding target sequence. In some embodiments, the ligand-conjugated RNAi oligonucleotide comprises a targeting sequence having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein the mismatches are interspersed in any position throughout the targeting sequence. In some embodiments, the ligand-conjugated RNAi oligonucleotide comprises a targeting sequence having more than one mismatch (e.g., 2, 3, 4, 5 or more mismatches) with the corresponding target sequence, wherein at least 2 (e.g., all) of the mismatches are positioned consecutively (e.g., 2, 3, 4, 5 or more mismatches in a row), or wherein at least one or more non-mismatched base pair is located between the mismatches, or a combination thereof.

### Types of Oligonucleotides

A variety of RNAi oligonucleotide types and/or structures are useful for reducing target gene expression in the methods herein. Any of the RNAi oligonucleotide types described herein or elsewhere are contemplated for use as a framework to incorporate a targeting sequence herein for the purposes of inhibiting or reducing corresponding target gene expression.

In some embodiments, the RNAi oligonucleotides conjugates herein engage with the RNAi pathway downstream of the involvement of Dicer (e.g., Dicer cleavage). In some embodiments, the oligonucleotides described herein are Dicer substrates. In some embodiments, the oligonucleotides herein interact with Dicer and are loaded into RISC.

In some embodiments, the RNAi oligonucleotides conjugates disclosed herein comprise sense and antisense strands that are both in the range of about 13 to 22 (e.g., 13 to 22, or 13 to 18) nucleotides in length. In some embodiments, the RNAi oligonucleotides conjugates disclosed herein comprise sense and antisense strands that are both in the range of about 13 to 18 (e.g., 13 to 18, 14 to 18, 15 to 18, or 16 to 18) nucleotides in length. In some embodiments, the ligand-conjugated RNAi oligonucleotides disclosed herein comprise a sense and antisense strand that are both in the range of about 19-22 nucleotides in length. In some embodiments, the sense and antisense strands are of equal length. In some embodiments, the ligand-conjugated RNAi oligonucleotides disclosed herein comprise sense and antisense strands, such that there is a 3′-overhang on either the sense strand or the antisense strand, or both the sense and antisense strand.

### Antisense Strands

In some embodiments, an antisense strand of a ligand-conjugated RNAi oligonucleotide is referred to as a “guide strand.” For example, an antisense strand that engages with RNA-induced silencing complex (RISC) and binds to an Argonaute protein such as Ago2, or engages with or binds to one or more similar factors, and directs silencing of a target gene, the antisense strand is referred to as a guide strand. In some embodiments, a sense strand complementary to a guide strand is referred to as a “passenger strand.”

In some embodiments, a ligand-conjugated RNAi oligonucleotide herein comprises an antisense strand of up to about 22 nucleotides in length (e.g., up to 22, up to 21, up to 20, up to 19, up to 18, up to 17, or up to 16 nucleotides in length). In some embodiments, a ligand-conjugated RNAi oligonucleotide herein comprises an antisense strand of at least about 16 nucleotides in length (e.g., at least 16, at least 17, at least 18, at least 19, at least 20, at least 21, or at least 22 nucleotides in length). In some embodiments, a ligand-conjugated RNAi oligonucleotide herein comprises an antisense strand in a range of about 16 to about 22 (e.g., 16 to 22, 16 to 21, 16 to 20, 16 to 19, 16 to 18, or 16 to 17) nucleotides in length. In some embodiments, an antisense strand of any one of the ligand-conjugated RNAi oligonucleotide disclosed herein is 16, 17, 18, 19, 20, 21, or 22 nucleotides in length. In some embodiments, a ligand-conjugated RNAi oligonucleotide herein comprises an antisense strand of 16 to 22 nucleotides in length. In some embodiments, a ligand-conjugated RNAi oligonucleotide herein comprises an antisense strand of 18 to 19 nucleotides in length. In some embodiments, a ligand-conjugated RNAi oligonucleotide herein comprises an antisense strand of 18 to 22 nucleotides in length. In some embodiments, a ligand-conjugated RNAi oligonucleotide herein comprises an antisense strand of 16 to 18 nucleotides in length. In some embodiments, a ligand-conjugated RNAi oligonucleotide comprises an antisense strand of 16 nucleotides in length. In some embodiments, a ligand-conjugated RNAi oligonucleotide comprises an antisense strand of 17 nucleotides in length. In some embodiments, a ligand-conjugated RNAi oligonucleotide comprises an antisense strand of 18 nucleotides in length. In some embodiments, a ligand-conjugated RNAi oligonucleotide comprises an antisense strand of 19 nucleotides in length. In some embodiments, a ligand-conjugated RNAi oligonucleotide comprises an antisense strand of 20 nucleotides in length. In some embodiments, a ligand-conjugated RNAi oligonucleotide comprises an antisense strand of 21 nucleotides in length. In some embodiments, a ligand-conjugated RNAi oligonucleotide comprises an antisense strand of 22 nucleotides in length.

### Sense Strands

In some embodiments, a ligand-conjugated RNAi oligonucleotide disclosed herein comprises a sense strand (or passenger strand) of up to about 21 nucleotides in length (e.g., up to 21, up to 20, up to 19, up to 18, up to 17, up to 16, up to 15, up to 14, up to 13 up to 12, up to 11, or up to 10 nucleotides in length). In some embodiments, a ligand-conjugated RNAi oligonucleotide herein comprises a sense strand of at least about 12 nucleotides in length (e.g., at least 12, at least 15, at least 19, at least 20, or at least 21 nucleotides in length). In some embodiments, a ligand-conjugated RNAi oligonucleotide herein comprises a sense strand of at least about 10 nucleotides in length (e.g., at least 10, at least 12, at least 15, at least 19, at least 20, or at least 21, nucleotides in length). In some embodiments, a ligand-conjugated RNAi oligonucleotide herein comprises a sense strand in a range of about 13 to about 21 (e.g., 13 to 21, 13 to 20, 13 to 19, 13 to 18, 13 to 17, 13 to 16, 13 to 15, or 13 to 14) nucleotides in length. In some embodiments, a ligand-conjugated RNAi oligonucleotide herein comprises a sense strand 13 to 21 nucleotides in length. In some embodiments, a ligand-conjugated RNAi oligonucleotide herein comprises a sense strand 15 to 21 nucleotides in length. In some embodiments, a ligand-conjugated RNAi oligonucleotide herein comprises a sense strand 15 to 18 nucleotides in length. In some embodiments, a ligand-conjugated RNAi oligonucleotide herein comprises a sense strand of 13, 14, 15, 16, 17, 18, 19, 20, or 21 nucleotides in length. In some embodiments, a ligand-conjugated RNAi oligonucleotide herein comprises a sense strand of 15, 16, 17, 18, 19, 20, or 21, nucleotides in length. In some embodiments, a ligand-conjugated RNAi oligonucleotide herein comprises a sense strand of 13 nucleotides in length. In some embodiments, a ligand-conjugated RNAi oligonucleotide herein comprises a sense strand of 14 nucleotides in length. In some embodiments, a ligand-conjugated RNAi oligonucleotide herein comprises a sense strand of 15 nucleotides in length. In some embodiments, a ligand-conjugated RNAi oligonucleotide herein comprises a sense strand of 16 nucleotides in length. In some embodiments, a ligand-conjugated RNAi oligonucleotide herein comprises a sense strand of 17 nucleotides in length. In some embodiments, a ligand-conjugated RNAi oligonucleotide herein comprises a sense strand of 18 nucleotides in length. In some embodiments, a ligand-conjugated RNAi oligonucleotide herein comprises a sense strand of 19 nucleotides in length. In some embodiments, a ligand-conjugated RNAi oligonucleotide herein comprises a sense strand of 20 nucleotides in length. In some embodiments, a ligand-conjugated RNAi oligonucleotide herein comprises a sense strand of 21 nucleotides in length

### Duplex Length

In some embodiments, a duplex formed between a sense and antisense strand is at least 13 (e.g., at least 13, at least 14, at least 15, or at least 16) nucleotides in length. In some embodiments, a duplex formed between a sense and antisense strand is in the range of 13-21 nucleotides in length (e.g., 13 to 21, 13 to 20, 13 to 19, 13 to 18, 13 to 17, 13 to 16, 13 to 15, or 13 to 14, nucleotides in length). In some embodiments, a duplex formed between a sense and antisense strand is in the range of 15-21 nucleotides in length (e.g., 15 to 21, 15 to 20, 15 to 19, 15 to 18, 15 to 17, or 15 to 16 nucleotides in length). In some embodiments, a duplex formed between a sense and antisense strand is in the range of 18-21 nucleotides in length (e.g., 18 to 21, 18 to 20, or 18 to 19 nucleotides in length). In some embodiments, a duplex formed between a sense and antisense strand is 13, 14, 15, 16, 17, 18, 19, or 20 nucleotides in length. In some embodiments, a duplex formed between a sense and antisense strand is 13-21 base pairs in length. In some embodiments, a duplex formed between a sense and antisense strand is 15-20 base pairs in length. In some embodiments, a duplex formed between a sense and antisense strand is 18-21 base pairs in length. In some embodiments, a duplex formed between a sense and antisense strand is 13 base pairs in length. In some embodiments, a duplex formed between a sense and antisense strand is 14 base pairs in length. In some embodiments, a duplex formed between a sense and antisense strand is 15 base pairs in length. In some embodiments, a duplex formed between a sense and antisense strand is 16 base pairs in length. In some embodiments, a duplex formed between a sense and antisense strand is 17 base pairs in length. In some embodiments, a duplex formed between a sense and antisense strand is 18 base pairs in length. In some embodiments, a duplex formed between a sense and antisense strand is 19 base pairs in length. In some embodiments, a duplex formed between a sense and antisense strand is 20 base pairs in length. In some embodiments, a duplex formed between a sense and antisense strand is 21 base pairs in length. In some embodiments, a duplex formed between a sense and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand.

### Oligonucleotide Ends

In some embodiments, a ligand-conjugated RNAi oligonucleotide disclosed herein comprises sense and antisense strands, such that there is a 3′-overhang on either the sense strand or the antisense strand, or both the sense and antisense strand. In some embodiments, a ligand-conjugated RNAi oligonucleotide herein has one 5′ end that is thermodynamically less stable compared to the other 5′ end. In some embodiments, an asymmetric ligand-conjugated RNAi oligonucleotide conjugate is provided that includes a blunt end at the 3′ end of a sense strand and overhang at the 3′ end of the antisense strand. In some embodiments, a 3′ overhang on an antisense strand is 1~4 nucleotides in length (e.g., 1, 2, 3, or 4 nucleotides in length).

In some embodiments, the 3′-overhang is about one to ten nucleotides in length (e.g., about 1, 2, 3, 4, 5, 6, or about 7 nucleotides in length). In some embodiments, the 3′ overhang is about one to seven, one to six, one to five, one to four, one to three, or about one to two nucleotides in length. In some embodiments, the 3′-overhang is about two to about ten nucleotides in length. In some embodiments, the 3′-overhang is one nucleotide in length. In some embodiments, the 3′-overhang is two nucleotides in length. In some embodiments, the 3′-overhang is three nucleotides in length. In some embodiments, the 3′-overhang is four nucleotides in length. In some embodiments, the 3′-overhang is five nucleotides in length. In some embodiments, the 3′-overhang is six nucleotides in length. In some embodiments, the 3′-overhang is seven nucleotides in length.

In some embodiments, an overhang is a 3′ overhang comprising a length of between one and seven nucleotides, optionally one to seven, one to six, one to five, one to four, one to three, one to two, two to seven, two to six, two to five, two to four, two to three, or one, two, three, four, five, six, or seven nucleotides. In some embodiments, the overhang is a 5′ overhang comprising a length of between one and two nucleotides.

In some embodiments, an oligonucleotide herein comprises a sense strand and an antisense strand, wherein the 5′ terminus of either or both strands comprise a 5′-overhang comprising one or more nucleotides. In some embodiments, an oligonucleotide herein comprises a sense strand and an antisense strand, wherein the sense strand comprises a 5′-overhang comprising one or more nucleotides. In some embodiments, an oligonucleotide herein comprises a sense strand and an antisense strand, wherein the antisense strand comprises a 5′-overhang comprising one or more nucleotides. In some embodiments, an oligonucleotide herein comprises a sense strand and an antisense strand, wherein both the sense strand and the antisense strand comprises a 5′-overhang comprising one or more nucleotides.

In some embodiments, the 5′-overhang is about one to two nucleotides in length (e.g., about 1 or about 2 nucleotides in length). In some embodiments, the 5′-overhang is one nucleotide in length. In some embodiments, the 5′-overhang is two nucleotides in length.

In some embodiments, the 5′ end is blunt and the and the 3′ overhang is about 1-7 nucleotides. In some embodiments, the 5′ end is blunt and the 3′ overhang is 1 nucleotide. In some embodiments, the 5′ end is blunt and the 3′ overhang is 2 nucleotides. In some embodiments, the 5′ end is blunt and the 3′ overhang is 3 nucleotides. In some embodiments, the 5′ end is blunt and the 3′ overhang is 4 nucleotides. In some embodiments, the 5′ end is blunt and the 3′ overhang is 5 nucleotides. In some embodiments, the 5′ end is blunt and the 3′ overhang is 6 nucleotides. In some embodiments, the 5′ end is blunt and the 3′ overhang is 7 nucleotides.

In some embodiments, the 5′ overhang is 1 nucleotide and the 3′ overhang is about 2-7 nucleotides. In some embodiments, the 5′ overhang is 1 nucleotide and the 3′ overhang is 1 nucleotide. In some embodiments, the 5′ overhang is 1 nucleotide and the 3′ overhang is 2 nucleotides. In some embodiments, the 5′ overhang is 1 nucleotide and the 3′ overhang is 3 nucleotides. In some embodiments, the 5′ overhang is 1 nucleotide and the 3′ overhang is 4 nucleotides. In some embodiments, the 5′ overhang is 1 nucleotide and the 3′ overhang is 5 nucleotides. In some embodiments, the 5′ overhang is 1 nucleotide and the 3′ overhang is 6 nucleotides. In some embodiments, the 5′ overhang is 1 nucleotide and the 3′ overhang is 7 nucleotides.

In some embodiments, the 5′ overhang is 2 nucleotides and the 3′ overhang is about 3-7 nucleotides. In some embodiments, the 5′ overhang is 2 nucleotides and the 3′ overhang is 3 nucleotides. In some embodiments, the 5′ overhang is 2 nucleotides and the 3′ overhang is 4 nucleotides. In some embodiments, the 5′ overhang is 2 nucleotides and the 3′ overhang is 5 nucleotides. In some embodiments, the 5′ overhang is 2 nucleotides and the 3′ overhang is 6 nucleotides. In some embodiments, the 5′ overhang is 2 nucleotides and the 3′ overhang is 7 nucleotides.

In some embodiments, one or more (e.g., 2, 3, or 4) terminal nucleotides of the 3′ end or 5′ end of a sense and/or antisense strand are modified. For example, in some embodiments, one or two terminal nucleotides of the 3′ end of the antisense strand are modified. In some embodiments, the last nucleotide at the 3′ end of an antisense strand is modified, e.g., comprises 2′ modification, e.g., a 2′-O-methoxyethyl. In some embodiments, the last one or two terminal nucleotides at the 3′ end of an antisense strand are complementary with the target. In some embodiments, the last one or two nucleotides at the 3′ end of the antisense strand are not complementary with the target.

In some embodiments, an RNAi oligonucleotide conjugate disclosed herein comprises an overhang of 1-8 nucleotides at the 5′ end of the sense strand and comprises an overhang of 1-8 nucleotides at the 5′ end of the antisense strand.

In some embodiments, the two terminal nucleotides of the 3′ overhang is selected from AA, GG, AG, and GA. In some embodiments, the overhang is AA. In some embodiments, the overhang is AG. In some embodiments, the overhang is GA. In some embodiments, the two terminal overhang nucleotides are GG. Typically, one or both of the two terminal GG nucleotides of the antisense strand are not complementary with the target.

In some embodiments, the 5′ end and/or the 3′ end of a sense or antisense strand has an inverted cap nucleotide.

In some embodiments, one or more (e.g., 2, 3, 4, 5, 6) modified internucleotide linkages are provided between terminal nucleotides of the 3′ end or 5′ end of a sense and/or antisense strand. In some embodiments, modified internucleotide linkages are provided between overhang nucleotides at the 3′ end or 5′ end of a sense and/or antisense strand.

In some embodiments, the antisense strand is 22 nucleotides in length and comprises a blunt end at the 5′ terminus and a 3′ overhang of 1-7 nucleotides. In some embodiments, the antisense strand is 22 nucleotides in length and comprises a blunt end at the 5′ terminus and a 3′ overhang of 1 nucleotide. In some embodiments, the antisense strand is 22 nucleotides in length and comprises a blunt end at the 5′ terminus and a 3′ overhang of 2 nucleotides. In some embodiments, the antisense strand is 22 nucleotides in length and comprises a blunt end at the 5′ terminus and a 3′ overhang of 3 nucleotides. In some embodiments, the antisense strand is 22 nucleotides in length and comprises a blunt end at the 5′ terminus and a 3′ overhang of 4 nucleotides. In some embodiments, the antisense strand is 22 nucleotides in length and comprises a blunt end at the 5′ terminus and a 3′ overhang of 5 nucleotides. In some embodiments, the antisense strand is 22 nucleotides in length and comprises a blunt end at the 5′ terminus and a 3′ overhang of 6 nucleotides. In some embodiments, the antisense strand is 22 nucleotides in length and comprises a blunt end at the 5′ terminus and a 3′ overhang of 7 nucleotides.

In some embodiments, the antisense strand is 21 nucleotides in length and comprises a blunt end at the 5′ terminus and a 3′ overhang of 1-6 nucleotides. In some embodiments, the antisense strand is 21 nucleotides in length and comprises a blunt end at the 5′ terminus and a 3′ overhang of 1 nucleotide. In some embodiments, the antisense strand is 21 nucleotides in length and comprises a blunt end at the 5′ terminus and a 3′ overhang of 2 nucleotides. In some embodiments, the antisense strand is 21 nucleotides in length and comprises a blunt end at the 5′ terminus and a 3′ overhang of 3 nucleotides. In some embodiments, the antisense strand is 21 nucleotides in length and comprises a blunt end at the 5′ terminus and a 3′ overhang of 4 nucleotides. In some embodiments, the antisense strand is 21 nucleotides in length and comprises a blunt end at the 5′ terminus and a 3′ overhang of 5 nucleotides. In some embodiments, the antisense strand is 21 nucleotides in length and comprises a blunt end at the 5′ terminus and a 3′ overhang of 6 nucleotides.

In some embodiments, the antisense strand is 20 nucleotides in length and comprises a blunt end at the 5′ terminus and a 3′ overhang of 1-5 nucleotides. In some embodiments, the antisense strand is 20 nucleotides in length and comprises a blunt end at the 5′ terminus and a 3′ overhang of 1 nucleotide. In some embodiments, the antisense strand is 20 nucleotides in length and comprises a blunt end at the 5′ terminus and a 3′ overhang of 2 nucleotides. In some embodiments, the antisense strand is 20 nucleotides in length and comprises a blunt end at the 5′ terminus and a 3′ overhang of 3 nucleotides. In some embodiments, the antisense strand is 20 nucleotides in length and comprises a blunt end at the 5′ terminus and a 3′ overhang of 4 nucleotides. In some embodiments, the antisense strand is 20 nucleotides in length and comprises a blunt end at the 5′ terminus and a 3′ overhang of 5 nucleotides.

In some embodiments, the antisense strand is 19 nucleotides in length and comprises a blunt end at the 5′ terminus and a 3′ overhang of 1-4 nucleotides. In some embodiments, the antisense strand is 19 nucleotides in length and comprises a blunt end at the 5′ terminus and a 3′ overhang of 1 nucleotide. In some embodiments, the antisense strand is 19 nucleotides in length and comprises a blunt end at the 5′ terminus and a 3′ overhang of 2 nucleotides. In some embodiments, the antisense strand is 19 nucleotides in length and comprises a blunt end at the 5′ terminus and a 3′ overhang of 3 nucleotides. In some embodiments, the antisense strand is 19 nucleotides in length and comprises a blunt end at the 5′ terminus and a 3′ overhang of 4 nucleotides.

In some embodiments, the antisense strand is 18 nucleotides in length and comprises a blunt end at the 5′ terminus and a 3′ overhang of 1-3 nucleotides. In some embodiments, the antisense strand is 18 nucleotides in length and comprises a blunt end at the 5′ terminus and a 3′ overhang of 1 nucleotide. In some embodiments, the antisense strand is 18 nucleotides in length and comprises a blunt end at the 5′ terminus and a 3′ overhang of 2 nucleotides. In some embodiments, the antisense strand is 18 nucleotides in length and comprises a blunt end at the 5′ terminus and a 3′ overhang of 3 nucleotides.

In some embodiments, the antisense strand is 22 nucleotides in length and comprises a 5′ overhang of 1-7 nucleotides and a 3′ overhang of 0-2 nucleotides. In some embodiments, the antisense strand is 20 nucleotides in length and comprises a 5′ overhang of 1-7 nucleotides and a 3′ overhang of 1-2 nucleotides. In some embodiments, the antisense strand is 22 nucleotides in length and comprises a 5′ overhang of 1 nucleotide and a 3′ overhang of 1 nucleotide. In some embodiments, the antisense strand is 22 nucleotides in length and comprises a 5′ overhang of 2 nucleotides and a 3′ overhang of 1 nucleotide. In some embodiments, the antisense strand is 22 nucleotides in length and comprises a 5′ overhang of 3 nucleotides and a 3′ overhang of 1 nucleotide. In some embodiments, the antisense strand is 22 nucleotides in length and comprises a 5′ overhang of 4 nucleotides and a 3′ overhang of 1 nucleotide. In some embodiments, the antisense strand is 22 nucleotides in length and comprises a 5′ overhang of 5 nucleotides and a 3′ overhang of 1 nucleotide. In some embodiments, the antisense strand is 22 nucleotides in length and comprises a 5′ overhang of 6 nucleotides and a 3′ overhang of 1 nucleotide. In some embodiments, the antisense strand is 22 nucleotides in length and comprises a 5′ overhang of 7 nucleotides and a 3′ overhang of 1 nucleotide. In some embodiments, the antisense strand is 22 nucleotides in length and comprises a 5′ overhang of 1 nucleotide and a 3′ overhang of 2 nucleotides. In some embodiments, the antisense strand is 22 nucleotides in length and comprises a 5′ overhang of 2 nucleotides and a 3′ overhang of 2 nucleotides. In some embodiments, the antisense strand is 22 nucleotides in length and comprises a 5′ overhang of 3 nucleotides and a 3′ overhang of 2 nucleotides. In some embodiments, the antisense strand is 22 nucleotides in length and comprises a 5′ overhang of 4 nucleotides and a 3′ overhang of 2 nucleotides. In some embodiments, the antisense strand is 22 nucleotides in length and comprises a 5′ overhang of 5 nucleotides and a 3′ overhang of 2 nucleotides. In some embodiments, the antisense strand is 22 nucleotides in length and comprises a 5′ overhang of 6 nucleotides and a 3′ overhang of 2 nucleotides. In some embodiments, the antisense strand is 22 nucleotides in length and comprises a 5′ overhang of 7 nucleotides and a 3′ overhang of 2 nucleotides.

In some embodiments, the antisense strand is 21 nucleotides in length and comprises a 5′ overhang of 1-6 nucleotides and a 3′ overhang of 0-2 nucleotides. In some embodiments, the antisense strand is 21 nucleotides in length and comprises a 5′ overhang of 1-6 nucleotides and a 3′ overhang of 1-2 nucleotides. In some embodiments, the antisense strand is 21 nucleotides in length and comprises a 5′ overhang of 1 nucleotide and a 3′ overhang of 1 nucleotide. In some embodiments, the antisense strand is 21 nucleotides in length and comprises a 5′ overhang of 2 nucleotides and a 3′ overhang of 1 nucleotide. In some embodiments, the antisense strand is 21 nucleotides in length and comprises a 5′ overhang of 3 nucleotides and a 3′ overhang of 1 nucleotide. In some embodiments, the antisense strand is 21 nucleotides in length and comprises a 5′ overhang of 4 nucleotides and a 3′ overhang of 1 nucleotide. In some embodiments, the antisense strand is 21 nucleotides in length and comprises a 5′ overhang of 5 nucleotides and a 3′ overhang of 1 nucleotide. In some embodiments, the antisense strand is 21 nucleotides in length and comprises a 5′ overhang of 6 nucleotides and a 3′ overhang of 1 nucleotide. In some embodiments, the antisense strand is 21 nucleotides in length and comprises a 5′ overhang of 1 nucleotide and a 3′ overhang of 2 nucleotides. In some embodiments, the antisense strand is 21 nucleotides in length and comprises a 5′ overhang of 2 nucleotides and a 3′ overhang of 2 nucleotides. In some embodiments, the antisense strand is 21 nucleotides in length and comprises a 5′ overhang of 3 nucleotides and a 3′ overhang of 2 nucleotides. In some embodiments, the antisense strand is 21 nucleotides in length and comprises a 5′ overhang of 4 nucleotides and a 3′ overhang of 2 nucleotides. In some embodiments, the antisense strand is 21 nucleotides in length and comprises a 5′ overhang of 5 nucleotides and a 3′ overhang of 2 nucleotides. In some embodiments, the antisense strand is 21 nucleotides in length and comprises a 5′ overhang of 6 nucleotides and a 3′ overhang of 2 nucleotides.

In some embodiments, the antisense strand is 20 nucleotides in length and comprises a 5′ overhang of 1-5 nucleotides and a 3′ overhang of 0-2 nucleotides. In some embodiments, the antisense strand is 20 nucleotides in length and comprises a 5′ overhang of 1-5 nucleotides and a 3′ overhang of 1-2 nucleotides. In some embodiments, the antisense strand is 20 nucleotides in length and comprises a 5′ overhang of 1 nucleotide and a 3′ overhang of 1 nucleotide. In some embodiments, the antisense strand is 20 nucleotides in length and comprises a 5′ overhang of 2 nucleotides and a 3′ overhang of 1 nucleotide. In some embodiments, the antisense strand is 20 nucleotides in length and comprises a 5′ overhang of 3 nucleotides and a 3′ overhang of 1 nucleotide. In some embodiments, the antisense strand is 20 nucleotides in length and comprises a 5′ overhang of 4 nucleotides and a 3′ overhang of 1 nucleotide. In some embodiments, the antisense strand is 20 nucleotides in length and comprises a 5′ overhang of 5 nucleotides and a 3′ overhang of 1 nucleotide. In some embodiments, the antisense strand is 20 nucleotides in length and comprises a 5′ overhang of 1 nucleotide and a 3′ overhang of 2 nucleotides. In some embodiments, the antisense strand is 20 nucleotides in length and comprises a 5′ overhang of 2 nucleotides and a 3′ overhang of 2 nucleotides. In some embodiments, the antisense strand is 20 nucleotides in length and comprises a 5′ overhang of 3 nucleotides and a 3′ overhang of 2 nucleotides. In some embodiments, the antisense strand is 20 nucleotides in length and comprises a 5′ overhang of 4 nucleotides and a 3′ overhang of 2 nucleotides. In some embodiments, the antisense strand is 20 nucleotides in length and comprises a 5′ overhang of 5 nucleotides and a 3′ overhang of 2 nucleotides.

In some embodiments, the antisense strand is 19 nucleotides in length and comprises a 5′ overhang of 1-4 nucleotides and a 3′ overhang of 0-2 nucleotides. In some embodiments, the antisense strand is 19 nucleotides in length and comprises a 5′ overhang of 1-4 nucleotides and a 3′ overhang of 1-2 nucleotides. In some embodiments, the antisense strand is 19 nucleotides in length and comprises a 5′ overhang of 1 nucleotide and a 3′ overhang of 1 nucleotide. In some embodiments, the antisense strand is 19 nucleotides in length and comprises a 5′ overhang of 2 nucleotides and a 3′ overhang of 1 nucleotide. In some embodiments, the antisense strand is 19 nucleotides in length and comprises a 5′ overhang of 3 nucleotides and a 3′ overhang of 1 nucleotide. In some embodiments, the antisense strand is 19 nucleotides in length and comprises a 5′ overhang of 4 nucleotides and a 3′ overhang of 1 nucleotide. In some embodiments, the antisense strand is 19 nucleotides in length and comprises a 5′ overhang of 1 nucleotide and a 3′ overhang of 2 nucleotides. In some embodiments, the antisense strand is 19 nucleotides in length and comprises a 5′ overhang of 2 nucleotides and a 3′ overhang of 2 nucleotides. In some embodiments, the antisense strand is 19 nucleotides in length and comprises a 5′ overhang of 3 nucleotides and a 3′ overhang of 2 nucleotides. In some embodiments, the antisense strand is 19 nucleotides in length and comprises a 5′ overhang of 4 nucleotides and a 3′ overhang of 2 nucleotides.

In some embodiments, the antisense strand is 18 nucleotides in length and comprises a 5′ overhang of 1-3 nucleotides and a 3′ overhang of 0-2 nucleotides. In some embodiments, the antisense strand is 18 nucleotides in length and comprises a 5′ overhang of 1-3 nucleotides and a 3′ overhang of 1-2 nucleotides. In some embodiments, the antisense strand is 18 nucleotides in length and comprises a 5′ overhang of 1 nucleotide and a 3′ overhang of 1 nucleotide. In some embodiments, the antisense strand is 18 nucleotides in length and comprises a 5′ overhang of 2 nucleotides and a 3′ overhang of 1 nucleotide. In some embodiments, the antisense strand is 18 nucleotides in length and comprises a 5′ overhang of 3 nucleotides and a 3′ overhang of 1 nucleotide. In some embodiments, the antisense strand is 18 nucleotides in length and comprises a 5′ overhang of 1 nucleotide and a 3′ overhang of 2 nucleotides. In some embodiments, the antisense strand is 18 nucleotides in length and comprises a 5′ overhang of 2 nucleotides and a 3′ overhang of 2 nucleotides. In some embodiments, the antisense strand is 18 nucleotides in length and comprises a 5′ overhang of 3 nucleotides and a 3′ overhang of 2 nucleotides.

### Oligonucleotide Modifications

In some embodiments, an RNAi oligonucleotide conjugate disclosed herein comprises one or more modifications. Oligonucleotides (e.g., RNAi oligonucleotides) may be modified in various ways to improve or control specificity, stability, delivery, bioavailability, resistance from nuclease degradation, immunogenicity, base-pairing properties, RNA distribution and cellular uptake and other features relevant to therapeutic research use.

In some embodiments, the modification is a modified sugar. In some embodiments, the modification is a 5′-terminal phosphate group. In some embodiments, the modification is a modified internucleoside linkage. In some embodiments, the modification is a modified base. In some embodiments, an oligonucleotide described herein can comprise any one of the modifications described herein or any combination thereof. For example, in some embodiments, an oligonucleotide described herein comprises at least one modified sugar, a 5′-terminal phosphate group, at least one modified internucleoside linkage, and at least one modified base.

The number of modifications on an oligonucleotide (e.g., an RNAi oligonucleotide) and the position of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of the nucleotides to be modified. Accordingly, in some embodiments, all or substantially all of the nucleotides of an oligonucleotides are modified. In some embodiments, more than half of the nucleotides are modified. In some embodiments, less than half of the nucleotides are modified. In some embodiments, the sugar moiety of all nucleotides comprising the oligonucleotide is modified at the 2′ position. The modifications may be reversible or irreversible. In some embodiments, an oligonucleotide as disclosed herein has a number and type of modified nucleotides sufficient to cause the desired characteristics (e.g., protection from enzymatic degradation, capacity to target a desired cell after in vivo administration, and/or thermodynamic stability).

### Sugar Modifications

In some embodiments, a nucleotide modification in a sugar comprises a 2′-modification. In some embodiments, a 2′-modification may be 2′-O-propargyl, 2′-O-propylamine, 2′-amino, 2′-ethyl, 2′-fluoro (2′-F), 2′-aminoethyl (EA), 2′-O-methyl(2′-OMe), 2′-O-methoxyethyl(2′-MOE), 2′-O-[2-(methylamino)-2-oxoethyl] (2′-O-NMA) or 2′-deoxy-2′-fluoro-β-d-arabinonucleic acid (2′-FANA). In some embodiments, the modification is 2′-F, 2′-OMe or 2′-MOE. In some embodiments, a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, a ligand-conjugated RNAi oligonucleotide described herein comprises at least about 1 modified nucleotide (e.g., at least 1, at least 5, at least 10, at least 15, at least 20, at least 25, at least 30, at least 35, at least 40, or at least 45, or more). In some embodiments, the sense strand of the ligand-conjugated RNAi oligonucleotide comprises at least about 1 modified nucleotide (e.g., at least 1, at least 5, at least 10, at least 15, or at least 20, or more). In some embodiments, the antisense strand of the ligand-conjugated RNAi oligonucleotide comprises at least about 1 modified nucleotide (e.g., at least 1, at least 5, at least 10, at least 15, at least 20, or more).

In some embodiments, all the nucleotides of the sense strand of the ligand-conjugated RNAi oligonucleotide are modified. In some embodiments, all the nucleotides of the antisense strand of the ligand-conjugated RNAi oligonucleotide are modified. In some embodiments, all the nucleotides of the ligand-conjugated RNAi oligonucleotide (i.e., both the sense strand and the antisense strand) are modified. In some embodiments, the modified nucleotide comprises a 2′-modification (e.g., a 2′-F or 2′-OMe, 2′-MOE, and 2′-deoxy-2′-fluoro-β-d-arabinonucleic acid).

In some embodiments, the disclosure provides ligand-conjugated RNAi oligonucleotides having different modification patterns. In some embodiments, the modified ligand-conjugated RNAi oligonucleotides comprise a sense strand sequence having a modification pattern as set forth in the Examples and Sequence Listing and an antisense strand having a modification pattern as set forth in the Examples and Sequence Listing.

In some embodiments, a ligand-conjugated RNAi oligonucleotide disclosed herein comprises an antisense strand having nucleotides that are modified with 2′-F. In some embodiments, a ligand-conjugated RNAi oligonucleotide disclosed herein comprises an antisense strand comprising nucleotides that are modified with 2′-F and 2′-OMe. In some embodiments, a ligand-conjugated RNAi oligonucleotide disclosed herein comprises a sense strand having nucleotides that are modified with 2′-F. In some embodiments, a ligand-conjugated RNAi oligonucleotide disclosed herein comprises a sense strand comprising nucleotides that are modified with 2′-F and 2′-OMe. In some embodiments, a ligand-conjugated RNAi oligonucleotide disclosed herein comprises a sense strand comprising nucleotides that are modified with 2′-F and 2′-OMe, provided that a nucleotide conjugated to a ligand is not modified with 2′-F or 2′-OMe.

In some embodiments, an oligonucleotide described herein comprises a sense strand with about 10-25%, 10%, 11%, 12%, 13%, 14% 15%, 16%, 17%, 18%, 19% or 20% of the nucleotides of the sense strand comprising a 2′-fluoro modification. In some embodiments, an oligonucleotide described herein comprises a sense strand with about 10-40%, 10%, 11%, 12%, 13%, 14% 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% of the nucleotides of the sense strand comprising a 2′-fluoro modification. In some embodiments, about 11% of the nucleotides of the sense strand comprise a 2-fluoro modification. In some embodiments, about 20% of the nucleotides of the sense strand comprise a 2-fluoro modification. In some embodiments, about 30% of the nucleotides of the sense strand comprise a 2-fluoro modification. In some embodiments, an oligonucleotide described herein comprises an antisense strand with about 25-35%, 25%, 26%, 27%, 28%, 29%, 30%, 31%, 32%, 33%, 34% or 35% of the nucleotides of the antisense strand comprising a 2′-fluoro modification. In some embodiments, an oligonucleotide described herein comprises an antisense strand with about 25-50%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% of the nucleotides of the antisense strand comprising a 2′-fluoro modification. In some embodiments, about 32% of the nucleotides of the antisense strand comprise a 2′-fluoro modification. In some embodiments, about 40% of the nucleotides of the antisense strand comprise a 2′-fluoro modification. In some embodiments, the oligonucleotide has about 15-25%, 15%, 16%, 17%, 18%, 19%, 20%, 21%, 22%, 23%, 24%, or 25% of its nucleotides comprising a 2′-fluoro modification. In some embodiments, the oligonucleotide has about 15-50%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% of its nucleotides comprising a 2′-fluoro modification. In some embodiments, about 19% of the nucleotides in the oligonucleotide comprise a 2′-fluoro modification. In some embodiments, about 26% of the nucleotides in the oligonucleotide comprise a 2′-fluoro modification.

In some embodiments, at least one internal position (e.g., a position other than the 5′ and 3′ terminal nucleotides) of the sense strand is modified with a 2′-F group. In some embodiments, there is no 2′-F group at a terminal nucleotide of the sense strand. In some embodiments, at least one internal position (e.g., a position other than the 5′ and 3′ terminal nucleotides) of the antisense strand is modified with a 2′-F group. In some embodiments, there is no 2′-F group at a terminal nucleotide of the antisense strand.

In some embodiments, one or more of positions 8, 9, 10 or 11 of the sense strand is modified with a 2′-F group. In some embodiments, one or more nucleotides forming a base pair with a nucleotide at one or more of positions 10-13 of the antisense strand, is modified with a 2′-F group. In some embodiments, the sugar moiety at each of nucleotides not modified with a 2′-F group or conjugated to a ligand in the sense strand is modified with a 2′-OMe. In some embodiments, the sugar moiety at each of nucleotides at positions 1-7 and 12-20 in the sense strand is modified with a 2′-OMe.

In some embodiments, a ligand-conjugated RNAi oligonucleotide provided herein comprises an antisense strand 22 nucleotides in length, with positions 1-22 numbered 5′ to 3′, and a sense strand having a 2′-fluoro modification at each of the nucleotides forming a base pair with nucleotides at one or more of positions 10, 11, 12, and 13 of the antisense strand. In some embodiments, a ligand-conjugated RNAi oligonucleotide provided herein comprises an antisense strand 22 nucleotides in length, with positions 1-22 numbered 5′ to 3′, and a sense strand having a 2′-fluoro modification at each of the nucleotides forming a base pair with nucleotides at positions 10, 11, 12, 13, or any combination thereof, of the antisense strand. In some embodiments, a ligand-conjugated RNAi oligonucleotide provided herein comprises an antisense strand 22 nucleotides in length, with positions 1-22 numbered 5′ to 3′, and a sense strand having a 2′-fluoro modification at each of the nucleotides forming a base pair with nucleotides at positions 10, 11, 12, and 13 of the antisense strand.

In some embodiments, a ligand-conjugated RNAi oligonucleotide provided herein comprises an antisense strand 21 nucleotides in length, with positions 1-21 numbered 5′ to 3′, and a sense strand having a 2′-fluoro modification at each of the nucleotides forming a base pair with nucleotides at one or more of positions 10, 11, 12, and 13 of the antisense strand. In some embodiments, a ligand-conjugated RNAi oligonucleotide provided herein comprises an antisense strand 21 nucleotides in length, with positions 1-21 numbered 5′ to 3′, and a sense strand having a 2′-fluoro modification at each of the nucleotides forming a base pair with nucleotides at positions 10, 11, 12, 13, or any combination thereof, of the antisense strand. In some embodiments, a ligand-conjugated RNAi oligonucleotide provided herein comprises an antisense strand 21 nucleotides in length, with positions 1-21 numbered 5′ to 3′, and a sense strand having a 2′-fluoro modification at each of the nucleotides forming a base pair with nucleotides at positions 10, 11, 12, and 13 of the antisense strand.

In some embodiments, a ligand-conjugated RNAi oligonucleotide provided herein comprises an antisense strand 20 nucleotides in length, with positions 1-20 numbered 5′ to 3′, and a sense strand having a 2′-fluoro modification at each of the nucleotides forming a base pair with nucleotides at one or more of positions 10, 11, 12, and 13 of the antisense strand. In some embodiments, a ligand-conjugated RNAi oligonucleotide provided herein comprises an antisense strand 20 nucleotides in length, with positions 1-20 numbered 5′ to 3′, and a sense strand having a 2′-fluoro modification at each of the nucleotides forming a base pair with nucleotides at positions 10, 11, 12, 13, or any combination thereof, of the antisense strand. In some embodiments, a ligand-conjugated RNAi oligonucleotide provided herein comprises an antisense strand 20 nucleotides in length, with positions 1-20 numbered 5′ to 3′, and a sense strand having a 2′-fluoro modification at each of the nucleotides forming a base pair with nucleotides at positions 10, 11, 12, and 13 of the antisense strand.

In some embodiments, a ligand-conjugated RNAi oligonucleotide provided herein comprises an antisense strand 19 nucleotides in length, with positions 1-19 numbered 5′ to 3′, and a sense strand having a 2′-fluoro modification at each of the nucleotides forming a base pair with nucleotides at one or more of positions 10, 11, 12, and 13 of the antisense strand. In some embodiments, a ligand-conjugated RNAi oligonucleotide provided herein comprises an antisense strand 19 nucleotides in length, with positions 1-19 numbered 5′ to 3′, and a sense strand having a 2′-fluoro modification at each of the nucleotides forming a base pair with nucleotides at positions 10, 11, 12, 13, or any combination thereof, of the antisense strand. In some embodiments, a ligand-conjugated RNAi oligonucleotide provided herein comprises an antisense strand 19 nucleotides in length, with positions 1-19 numbered 5′ to 3′, and a sense strand having a 2′-fluoro modification at each of the nucleotides forming a base pair with nucleotides at positions 10, 11, 12, and 13 of the antisense strand.

In some embodiments, a ligand-conjugated RNAi oligonucleotide provided herein comprises an antisense strand 18 nucleotides in length, with positions 1-18 numbered 5′ to 3′, and a sense strand having a 2′-fluoro modification at each of the nucleotides forming a base pair with nucleotides at one or more of positions 10, 11, 12, and 13 of the antisense strand. In some embodiments, a ligand-conjugated RNAi oligonucleotide provided herein comprises an antisense strand 18 nucleotides in length, with positions 1-18 numbered 5′ to 3′, and a sense strand having a 2′-fluoro modification at each of the nucleotides forming a base pair with nucleotides at positions 10, 11, 12, 13, or any combination thereof, of the antisense strand. In some embodiments, a ligand-conjugated RNAi oligonucleotide provided herein comprises an antisense strand 18 nucleotides in length, with positions 1-18 numbered 5′ to 3′, and a sense strand having a 2′-fluoro modification at each of the nucleotides forming a base pair with nucleotides at positions 10, 11, 12, and 13 of the antisense strand.

In some embodiments, the sense strand comprises at least one 2′-F modified nucleotide wherein the remaining nucleotides not modified with a 2′-F group or conjugated to a ligand are modified with a 2′-OMe.

In some embodiments, a ligand-conjugated RNAi oligonucleotide provided herein comprises an antisense strand having the sugar moiety of each of the nucleotides at positions 2, 3, 4, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamine, 2′-amino, 2′-ethyl, 2′-aminoethyl (EA), 2′-O-methyl(2′-OMe), 2′-O-methoxyethyl(2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a ligand-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-F.

In some embodiments, a ligand-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-OMe.

In some embodiments, a ligand-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′-O-propargyl, 2′-O-propylamin, 2′-amino, 2′-ethyl, 2′-aminoethyl (EA), 2′-O-methyl(2′-OMe), 2′-O-methoxyethyl(2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a ligand-conjugated RNAi oligonucleotide provided herein comprises a sense strand having the sugar moiety at positions 8-11 modified with 2′-F. In some embodiments, a ligand-conjugated RNAi oligonucleotide provided herein comprises a sense strand having the sugar moiety at positions 5-6 modified with 2′-F. In some embodiments, a ligand-conjugated RNAi oligonucleotide provided herein comprises a sense strand having the sugar moiety at positions 3-4 modified with 2′-F. In some embodiments, a ligand-conjugated RNAi oligonucleotide provided herein comprises a sense strand having the sugar moiety at positions 3-6 modified with 2′-F. In some embodiments, a ligand-conjugated RNAi oligonucleotide provided herein comprises a sense strand having the sugar moiety at positions 6-9 modified with 2′-F. In some embodiments, a ligand-conjugated RNAi oligonucleotide provided herein comprises a sense strand having the sugar moiety at positions 7-10 modified with 2′-F. In some embodiments, a ligand-conjugated RNAi oligonucleotide provided herein comprises a sense strand having the sugar moiety at positions 5-6 modified with 2′-F.

In some embodiments, a ligand-conjugated RNAi oligonucleotide provided herein comprises a sense strand having the sugar moiety at positions 1, 2, 3, 7, and 12-20 modified with 2′OMe. In some embodiments, a ligand-conjugated RNAi oligonucleotide provided herein comprises a sense strand having the sugar moiety at positions 2-7 and 12-20 modified with 2′OMe. In some embodiments, a ligand-conjugated RNAi oligonucleotide provided herein comprises a sense strand having the sugar moiety at positions 3-7 and 12-20 modified with 2′OMe. In some embodiments, a ligand-conjugated RNAi oligonucleotide provided herein comprises a sense strand having the sugar moiety at positions 4-7 and 12-20 modified with 2′OMe. In some embodiments, a ligand-conjugated RNAi oligonucleotide provided herein comprises a sense strand having the sugar moiety at positions 1-7 and 12-20 modified with 2′OMe. In some embodiments, a ligand-conjugated RNAi oligonucleotide provided herein comprises a sense strand having the sugar moiety at positions 1-7 and 12-15 modified with 2′OMe. In some embodiments, a ligand-conjugated RNAi oligonucleotide provided herein comprises a sense strand having the sugar moiety at positions 1-7 and 12-17 modified with 2′OMe. In some embodiments, a ligand-conjugated RNAi oligonucleotide provided herein comprises a sense strand having the sugar moiety at positions 1-7 and 15-20 modified with 2′OMe. In some embodiments, a ligand-conjugated RNAi oligonucleotide provided herein comprises a sense strand having the sugar moiety at positions 1-7, 12-14, and 18-20 modified with 2′OMe. In some embodiments, a ligand-conjugated RNAi oligonucleotide provided herein comprises a sense strand having the sugar moiety at positions 1-7 and 12-17 modified with 2′OMe. In some embodiments, a ligand-conjugated RNAi oligonucleotide provided herein comprises a sense strand having the sugar moiety at positions 1-7 and 12-18 modified with 2′OMe. In some embodiments, a ligand-conjugated RNAi oligonucleotide provided herein comprises a sense strand having the sugar moiety at positions 2 and 7-10 modified with 2′OMe. In some embodiments, a ligand-conjugated RNAi oligonucleotide provided herein comprises a sense strand having the sugar moiety at positions 2 and 7-15 modified with 2′OMe. In some embodiments, a ligand-conjugated RNAi oligonucleotide provided herein comprises a sense strand having the sugar moiety at positions 2, 7-10, and 13-15 modified with 2′OMe. In some embodiments, a ligand-conjugated RNAi oligonucleotide provided herein comprises a sense strand having the sugar moiety at positions 1-7, 12-17, and 20 modified with 2′OMe. In some embodiments, a ligand-conjugated RNAi oligonucleotide provided herein comprises a sense strand having the sugar moiety at positions 2-7 and 12-20 modified with 2′OMe. In some embodiments, a ligand-conjugated RNAi oligonucleotide provided herein comprises a sense strand having the sugar moiety at positions 3-7 and 12-20 modified with 2′OMe. In some embodiments, a ligand-conjugated RNAi oligonucleotide provided herein comprises a sense strand having the sugar moiety at 7-10 and 12-15 modified with 2′OMe. In some embodiments, a ligand-conjugated RNAi oligonucleotide provided herein comprises a sense strand having the sugar moiety at positions 2, 7, and 12-15 modified with 2′OMe. In some embodiments, a ligand-conjugated RNAi oligonucleotide provided herein comprises a sense strand having the sugar moiety at positions 2, 7-10, and 12 modified with 2′OMe. In some embodiments, a ligand-conjugated RNAi oligonucleotide provided herein comprises a sense strand having the sugar moiety at positions 2, 7-10, and 12-13 modified with 2′OMe. In some embodiments, a ligand-conjugated RNAi oligonucleotide provided herein comprises a sense strand having the sugar moiety at positions 7, 8, 9, 12, and 13 modified with 2′OMe. In some embodiments, a ligand-conjugated RNAi oligonucleotide provided herein comprises a sense strand having the sugar moiety at positions 2, 8, 9, 12, and 13 modified with 2′OMe. In some embodiments, a ligand-conjugated RNAi oligonucleotide provided herein comprises a sense strand having the sugar moiety at positions 2, 7, 12, and 13 modified with 2′OMe. In some embodiments, a ligand-conjugated RNAi oligonucleotide provided herein comprises a sense strand having the sugar moiety at positions 2, 7, 8, and 9 modified with 2′OMe. In some embodiments, a ligand-conjugated RNAi oligonucleotide provided herein comprises a sense strand having the sugar moiety at positions 5, 10-12, and 15-16 modified with 2′OMe. In some embodiments, a ligand-conjugated RNAi oligonucleotide provided herein comprises a sense strand having the sugar moiety at positions 4, 6, 11-13, and 16-17 modified with 2′OMe.

In some embodiments, a ligand-conjugated RNAi oligonucleotide provided herein comprises a sense strand having the sugar moiety of each of the nucleotides at positions 1-7 and 12-20 of the sense strand modified with a modification selected from the group consisting of 2′-O-propargyl, 2′-O-propylamin, 2′-amino, 2′-ethyl, 2′-aminoethyl (EA), 2′-O-methyl(2′-OMe), 2′-O-methoxyethyl(2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a ligand-conjugated RNAi oligonucleotide provided herein comprises a sense strand having the sugar moiety of each of the nucleotides at positions 72, and 7-10 of the sense strand modified with a modification selected from the group consisting of 2′-O-propargyl, 2′-O-propylamin, 2′-amino, 2′-ethyl, 2′-aminoethyl (EA), 2′-O-methyl(2′-OMe), 2′-O-methoxyethyl(2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a ligand-conjugated RNAi oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, or position 20 modified with 2′-F.

In some embodiments, a ligand-conjugated RNAi oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, or position 20 with 2′-OMe.

In some embodiments, a ligand-conjugated RNAi oligonucleotide provided herein comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, or position 20 modified with a modification selected from the group consisting of 2′-O-propargyl, 2′-O-propylamin, 2′-amino, 2′-ethyl, 2′-aminoethyl (EA), 2′-O-methyl(2′-OMe), 2′-O-methoxyethyl(2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a ligand-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, or position 18, position 19, position 20, position 21, or position 22 with 2′-F.

In some embodiments, a ligand-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, or position 18, position 19, position 20, position 21, or position 22 modified with 2′-OMe.

In some embodiments, a ligand-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, or position 18, position 19, position 20, position 21, or position 22 modified with a modification selected from the group consisting of 2′-O-propargyl, 2′-O-propylamine, 2′-amino, 2′-ethyl, 2′-aminoethyl (EA), 2′-O-methyl(2′-OMe), 2′-O-methoxyethyl(2′-MOE), 2′-O-[2-(methylamino)-2-oxoethyl] (2′-O-NMA), and 2′-deoxy-2′-fluoro-β-d-arabinonucleic acid (2′-FANA).

In some embodiments, a ligand-conjugated RNAi oligonucleotide provided herein comprises an antisense strand having the sugar moiety at position 1, position 2, position 3, position 4, position 5, position 6, position 7, position 8, position 9, position 10, position 11, position 12, position 13, position 14, position 15, position 16, position 17, or position 18, position 19, position 20, position 21, or position 22 modified with a modification selected from the group consisting of 2′-O-propargyl, 2′-O-propylamine, 2′-amino, 2′-ethyl, 2′-aminoethyl (EA), 2′-O-methyl(2′-OMe), 2′-O-methoxyethyl(2′-MOE), 2′-O-[2-(methylamino)-2-oxoethyl] (2′-O-NMA), and 2′-deoxy-2′-fluoro-β-d-arabinonucleic acid (2′-FANA).

### 5′-Terminal Phosphate

In some embodiments, a ligand-conjugated RNAi oligonucleotide described herein comprises a 5′-terminal phosphate. In some embodiments, the 5′-terminal phosphate groups of the ligand-conjugated RNAi oligonucleotide enhance the interaction with Ago2. However, oligonucleotides comprising a 5′-phosphate group may be susceptible to degradation via phosphatases or other enzymes, which can limit their bioavailability in vivo. In some embodiments, a ligand-conjugated RNAi oligonucleotide herein comprises analogs of 5′ phosphates that are resistant to such degradation. In some embodiments, the phosphate analog is oxymethyl phosphonate, vinyl phosphonate or malonyl phosphonate, or a combination thereof. In some embodiments, the 5′ end of a ligand-conjugated RNAi oligonucleotide strand is attached to chemical moiety that mimics the electrostatic and steric properties of a natural 5′-phosphate group (“phosphate mimic”).

In some embodiments, a ligand-conjugated RNAi oligonucleotide herein has a phosphate analog at a 4′-carbon position of the sugar (referred to as a “4′-phosphate analog”). See, e.g., Intl. Patent Application Publication No. WO 2018/045317. In some embodiments, a ligand-conjugated RNAi oligonucleotide herein comprises a 4′-phosphate analog at a 5′-terminal nucleotide. In some embodiments, a phosphate analog is an oxymethyl phosphonate, in which the oxygen atom of the oxymethyl group is bound to the sugar moiety (e.g., at its 4′-carbon) or analog thereof. In other embodiments, a 4′-phosphate analog is a thiomethyl phosphonate or an aminomethyl phosphonate, in which the sulfur atom of the thiomethyl group or the nitrogen atom of the amino methyl group is bound to the 4′-carbon of the sugar moiety or analog thereof. In some embodiments, a 4′-phosphate analog is an oxymethyl phosphonate. In some embodiments, an oxymethyl phosphonate is represented by the formula —O—CH₂—PO(OH)₂, —O—CH₂—PO(OR)₂, or —O—CH₂—POOH(R), in which R is independently selected from H, CH₃, an alkyl group, CH₂CH₂CN, CH₂OCOC(CH₃)₃, CH₂OCH₂CH₂Si(CH₃)₃ or a protecting group. In some embodiments, the alkyl group is CH₂CH₃. More typically, R is independently selected from H, CH₃ or CH₂CH₃. In some embodiment, R is CH3. In some embodiments, the 4′-phosphate analog is 5′-methoxyphosphonate-4′-oxy. In some embodiments, the 4′-phosphate analog is 4′-oxymethyl phosphonate.

In some embodiments, a ligand-conjugated RNAi oligonucleotide provided herein comprises an antisense strand comprising a 4′-phosphate analog at the 5′-terminal nucleotide, wherein 5′-terminal nucleotide comprises the following structure:

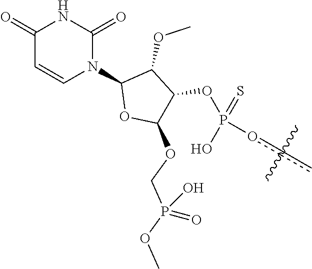

### 4′-O-monomethylphosphonate-2′-O-methyluridine phosphorothioate [MePhosphonate-40-mUs, alternatively referred to as “MeMOP”]

### Modified Internucleotide Linkage

In some embodiments, a ligand-conjugated RNAi oligonucleotide herein comprises a modified internucleoside linkage. In some embodiments, phosphate modifications or substitutions result in an oligonucleotide that comprises at least about 1 (e.g., at least 1, at least 2, at least 3 or at least 5) modified internucleotide linkage. In some embodiments, any one of the oligonucleotides disclosed herein comprises about 1 to about 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9 or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, the oligonucleotide described herein has a phosphorothioate linkage between each of positions 1 and 2 of the sense strand and the penultimate position and ultimate position of the sense strand.

In some embodiments, the oligonucleotide described herein has a phosphorothioate linkage between each of positions 1 and 2 of the sense strand, the third to last position and penultimate position of the sense strand, and the penultimate position and ultimate position of the sense strand.

In some embodiments, the oligonucleotide described herein has a phosphorothioate linkage between each of positions 1 and 2 of the antisense strand, positions 2 and 3 of the antisense strand, the third to last position and penultimate position of the antisense strand, and the penultimate position and ultimate position of the antisense strand.

In some embodiments, the oligonucleotide described herein has a phosphorothioate linkage between each of positions 1 and 2 of the antisense strand, positions 2 and 3 of the antisense strand, positions 3 and 4 of the antisense strand, the third to last position and penultimate position of the antisense strand, and the penultimate position and ultimate position of the antisense strand.

In some embodiments, the oligonucleotide described herein has a phosphorothioate linkage between each of positions 1 and 2 of the sense strand, the third to last position and penultimate position of the sense strand, the penultimate position and ultimate position of the sense strand, positions 1 and 2 of the antisense strand, positions 2 and 3 of the antisense strand, the third to last position and penultimate position of the antisense strand, and the penultimate position and ultimate position of the antisense strand.

In some embodiments, the oligonucleotide described herein has a phosphorothioate linkage between each of positions 1 and 2 of the sense strand, the third to last position and penultimate position of the sense strand, the penultimate position and ultimate position of the sense strand, positions 1 and 2 of the antisense strand, positions 2 and 3 of the antisense strand, positions 3 and 4 of the antisense strand, the third to last position and penultimate position of the antisense strand, and the penultimate position and ultimate position of the antisense strand.

In some embodiments, the oligonucleotide described herein has a phosphorothioate linkage between each of positions 1 and 2 of the sense strand, the third to last position and penultimate position of the sense strand, the penultimate position and ultimate position of the sense strand, positions 1 and 2 of the antisense strand, positions 2 and 3 of the antisense strand, positions 20 and 21 of the antisense strand, and positions 21 and 22 of the antisense strand.

In some embodiments, the oligonucleotide described herein has a phosphorothioate linkage between each of positions 1 and 2 of the sense strand, the third to last position and penultimate position of the sense strand, the penultimate position and ultimate position of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand.

In some embodiments, the oligonucleotide described herein has a phosphorothioate linkage between each of positions 1 and 2 of the sense strand, the third to last position and penultimate position of the sense strand, the penultimate position and ultimate position of the sense strand, positions 1 and 2 of the antisense strand, positions 2 and 3 of the antisense strand, positions 3 and 4 of the antisense strand, positions 19 and 20 of the antisense strand, and positions 20 and 21 of the antisense strand.

In some embodiments, the oligonucleotide described herein has a phosphorothioate linkage between each of positions 1 and 2 of the sense strand, the third to last position and penultimate position of the sense strand, the penultimate position and ultimate position of the sense strand, positions 1 and 2 of the antisense strand, positions 2 and 3 of the antisense strand, positions 3 and 4 of the antisense strand, positions 18 and 19 of the antisense strand, and positions 19 and 20 of the antisense strand.

In some embodiments, the oligonucleotide described herein has a phosphorothioate linkage between each of positions 1 and 2 of the sense strand, the third to last position and penultimate position of the sense strand, the penultimate position and ultimate position of the sense strand, positions 1 and 2 of the antisense strand, positions 2 and 3 of the antisense strand, positions 3 and 4 of the antisense strand, positions 17 and 18 of the antisense strand, and positions 18 and 19 of the antisense strand.

In some embodiments, the oligonucleotide described herein has a phosphorothioate linkage between each of positions 1 and 2 of the sense strand, the third to last position and penultimate position of the sense strand, the penultimate position and ultimate position of the sense strand, positions 1 and 2 of the antisense strand, positions 2 and 3 of the antisense strand, positions 3 and 4 of the antisense strand, positions 16 and 17 of the antisense strand, and positions 17 and 18 of the antisense strand.

In some embodiments, the oligonucleotide described herein has a phosphorothioate linkage between each of positions 1 and 2 of the sense strand, the penultimate position and the ultimate position of the sense strand, positions 1 and 2 of the antisense strand, positions 2 and 3 of the antisense strand, positions 20 and 21 of the antisense strand, and positions 21 and 22 of the antisense strand.

In some embodiments, an oligonucleotide conjugate described herein comprises a peptide nucleic acid (PNA). PNAs are oligonucleotide mimics in which the sugar-phosphate backbone has been replaced by a pseudopeptide skeleton, composed of N-(2-aminoethyl)glycine units. Nucleobases are linked to this skeleton through a two-atom carboxymethyl spacer. In some embodiments, an oligonucleotide conjugate described herein comprises a morpholino oligomer (PMO) comprising an internucleotide linkage backbone of methylene morpholine rings linked through phosphorodiamidate groups.

### Base Modifications

In some embodiments, a ligand-conjugated RNAi oligonucleotide herein comprises one or more modified nucleobases. In some embodiments, modified nucleobases (also referred to herein as base analogs) are linked at the 1′ position of a nucleotide sugar moiety. In some embodiments, a modified nucleobase is a nitrogenous base. In some embodiments, a modified nucleobase does not contain nitrogen atom. See, e.g., US Patent Application Publication No. 2008/0274462. In some embodiments, a modified nucleotide comprises a universal base. In some embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. In some embodiments, when compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, US Patent Application Publication No. 2007/0254362; Van Aerschot et al. (1995) NUCLEIC ACIDS RES. 23:4363-70; Loakes et al. (1995) NUCLEIC ACIDS RES. 23:2361-66; and Loakes & Brown (1994) NUCLEIC ACIDS RES. 22:4039-43).

### Tm-Increasing Nucleotides

In some embodiments, the oligonucleotide described herein comprises at least one Tm-increasing nucleotide in the sense strand. In some embodiments, the oligonucleotide has one Tm-increasing nucleotide in the sense strand. In some embodiments, the oligonucleotide has up to two Tm-increasing nucleotides in the sense strand. In some embodiments, the oligonucleotide has up to three Tm-increasing nucleotides in the sense strand. In some embodiments, the oligonucleotide has up to four Tm-increasing nucleotides in the sense strand. In some embodiments, the oligonucleotide has up to five Tm-increasing nucleotides in the sense strand. In some embodiments, the oligonucleotide has up to six Tm-increasing nucleotides in the sense strand. In some embodiments, the oligonucleotide has up to seven Tm-increasing nucleotides in the sense strand. In some embodiments, the oligonucleotide has up to eight Tm-increasing nucleotides in the sense strand. In some embodiments, the oligonucleotide has up to nine Tm-increasing nucleotides in the sense strand. In some embodiments, the oligonucleotide has up to ten Tm-increasing nucleotides in the sense strand.

In some embodiments, the oligonucleotide has 1 to 2 Tm-increasing nucleotides in the sense strand. In some embodiments, the oligonucleotide has 1 to 3 Tm-increasing nucleotides in the sense strand. In some embodiments, the oligonucleotide has 1 to 4 Tm-increasing nucleotides in the sense strand. In some embodiments, the oligonucleotide has 1 to 5 Tm-increasing nucleotides in the sense strand.

Tₘ-increasing nucleotides include, but are not limited to, bicyclic nucleotides, tricyclic nucleotides, a G-clamp, and analogues thereof, hexitol nucleotides, or a modified nucleotide. In some embodiments, the Tm-increasing nucleotide is a bicyclic nucleotide. In some embodiments, the Tm-increasing nucleotide is a locked nucleic acid (LNA).

In some embodiments, the sense strand of the oligonucleotide comprises a Tm-increasing nucleotide at one or more of positions 1, 4, 5, 10, 11, 12, 13, 14, 15, 16, and 18. In some embodiments, the sense strand of the oligonucleotide comprises a Tm-increasing nucleotide at position 1. In some embodiments, the sense strand of the oligonucleotide comprises a Tm-increasing nucleotide at position 4. In some embodiments, the sense strand of the oligonucleotide comprises a Tm-increasing nucleotide at position 5. In some embodiments, the sense strand of the oligonucleotide comprises a Tm-increasing nucleotide at position 10. In some embodiments, the sense strand of the oligonucleotide comprises a Tm-increasing nucleotide at position 11. In some embodiments, the sense strand of the oligonucleotide comprises a Tm-increasing nucleotide at position 12. In some embodiments, the sense strand of the oligonucleotide comprises a Tm-increasing nucleotide at position 13. In some embodiments, the sense strand of the oligonucleotide comprises a Tm-increasing nucleotide at position 14. In some embodiments, the sense strand of the oligonucleotide comprises a Tm-increasing nucleotide at position 15. In some embodiments, the sense strand of the oligonucleotide comprises a Tm-increasing nucleotide at position 16. In some embodiments, the sense strand of the oligonucleotide comprises a Tm-increasing nucleotide at position 18.

In some embodiments, a 20-nucleotide sense strand, with nucleotides numbered 5′ to 3′, comprises a Tm-increasing nucleotide at one or more of positions 1, 11, and 12. In some embodiments, a 15-nucleotide sense strand, with nucleotides numbered 5′ to 3′, comprises a Tm-increasing nucleotide at position 1. In some embodiments, a 15-nucleotide sense strand, with nucleotides numbered 5′ to 3′, comprises a Tm-increasing nucleotide at positions 1 and 11. In some embodiments, a 15-nucleotide sense strand, with nucleotides numbered 5′ to 3′, comprises a Tm-increasing nucleotide at positions 1 and 12. In some embodiments, a 15-nucleotide sense strand, with nucleotides numbered 5′ to 3′, comprises a Tm-increasing nucleotide at 11 and 12. In some embodiments, a 15-nucleotide sense strand, with nucleotides numbered 5′ to 3′, comprises a Tm-increasing nucleotide at positions 1, 11, and 12.

In some embodiments, a 13-nucleotide sense strand, with nucleotides numbered 5′ to 3′, comprises a Tm-increasing nucleotide at one or more of positions 1, 10, and 11. In some embodiments, a 13-nucleotide sense strand, with nucleotides numbered 5′ to 3′, comprises a Tm-increasing nucleotide at position 1. In some embodiments, a 13-nucleotide sense strand, with nucleotides numbered 5′ to 3′, comprises a Tm-increasing nucleotide at position 1 and position 10. In some embodiments, a 12-nucleotide sense strand, with nucleotides numbered 5′ to 3′, comprises a Tm-increasing nucleotide at position 1 and position 11. In some embodiments, a 13-nucleotide sense strand, with nucleotides numbered 5′ to 3′, comprises a Tm-increasing nucleotide at position 10 and position 11. In some embodiments, the sense strand comprises a Tm-increasing nucleotide at position 1, position 10, and position 11.

In some embodiments, a 16-nucleotide sense strand, with nucleotides numbered 5′ to 3′, comprises a Tm-increasing nucleotide at one or more of positions 4, 13, and 14. In some embodiments, a 16-nucleotide sense strand, with nucleotides numbered 5′ to 3′, comprises a Tm-increasing nucleotide at position 4. In some embodiments, a 16-nucleotide sense strand, with nucleotides numbered 5′ to 3′, comprises a Tm-increasing nucleotide at positions 13 and 14. In some embodiments, a 16-nucleotide sense strand, with nucleotides numbered 5′ to 3′, comprises a Tm-increasing nucleotide at positions 4 and 14. In some embodiments, a 16-nucleotide sense strand, with nucleotides numbered 5′ to 3′, comprises a Tm-increasing nucleotide at 13 and 14. In some embodiments, a 15-nucleotide sense strand, with nucleotides numbered 5′ to 3′, comprises a Tm-increasing nucleotide at positions 4, 13, and 14.

In some embodiments, a 17-nucleotide sense strand, with nucleotides numbered 5′ to 3′, comprises a Tm-increasing nucleotide at one or more of positions 5, 14, and 15. In some embodiments, a 17-nucleotide sense strand, with nucleotides numbered 5′ to 3′, comprises a Tm-increasing nucleotide at position 5. In some embodiments, a 17-nucleotide sense strand, with nucleotides numbered 5′ to 3′, comprises a Tm-increasing nucleotide at positions 14 and 15. In some embodiments, a 17-nucleotide sense strand, with nucleotides numbered 5′ to 3′, comprises a Tm-increasing nucleotide at positions 5 and 15. In some embodiments, a 17-nucleotide sense strand, with nucleotides numbered 5′ to 3′, comprises a Tm-increasing nucleotide at 14 and 15. In some embodiments, a 17-nucleotide sense strand, with nucleotides numbered 5′ to 3′, comprises a Tm-increasing nucleotide at positions 5, 14, and 15.

In some embodiments, the disclosure provides an RNAi oligonucleotide for reducing target gene expression by the RNAi pathway comprising a combination of one or more Tm-increasing nucleotides and one or more nucleotides (e.g., a modified nucleotide) having a lower binding affinity, wherein the duplex region comprising the RNAi oligonucleotide is maintained under physiological conditions and the ability of the RNAi oligonucleotide to inhibit or reduce target gene expression is maintained.

### Bicyclic Nucleotides

Bicyclic nucleotides typically have a sugar moiety with a 4 to 7 membered ring (including but not limited to a furanosyl) comprising a bridge connecting two atoms of the 4 to 7 membered ring to form a second ring, resulting in a bicyclic structure. Such bicyclic nucleotides have various names including BNA's and LNA's for bicyclic nucleic acids and locked nucleic acids, respectively. The synthesis of bicyclic nucleotides and their incorporation into nucleic acid compounds has also been reported in the literature, including, for example, Singh et al., Chem. Commun., 1998, 4, 455-56; Koshkin et al., TETRAHEDRON, 1998, 54, 3607-30; Wahlestedt et al., PROC. NATL. ACAD. SCI. U.S.A., 2000, 97, 5633-38; Kumar et al., BIOORG. MED. CHEM. LETT., 1998, 8, 2219-22; Singh et al., J. ORG. CHEM., 1998, 63, 10035-039; U.S. Pat. Nos. 7,427,672, 7,053,207, 6,794,499, 6,770,748, 6,268,490 and 6,794,499; and published U.S. applications 20040219565, 20040014959, 20030207841, 20040192918, 20030224377, 20040143114 and 20030082807; each of which is incorporated by reference herein, in its entirety.

In some embodiments, the Tₘ-increasing nucleotide is a bicyclic nucleotide that comprises a bicyclic sugar moiety. In certain embodiments, the bicyclic sugar moiety comprises a first ring of 4 to 7 members and a bridge forming a North-type sugar confirmation that connects any two atoms of the first ring of the sugar moiety to form a second ring. In certain embodiments, the bridge connects the 2′-carbon and the 4′-carbon of the first ring to form a second ring.

Typically, the bridge contains 2 to 8 atoms. In certain embodiments, the bridge contains 3 atoms. In certain embodiments, the bridge contains 4 atoms. In certain embodiments, the bridge contains 5 atoms. In certain embodiments, the bridge contains 6 atoms. In certain embodiments, the bridge contains 7 atoms. In certain embodiments, the bridge contains 8 atoms. In certain embodiments, the bridge contains more than 8 atoms.

In certain embodiments, the bicyclic sugar moiety is a substituted furanosyl comprising a bridge that connects the 2′-carbon and the 4′-carbon of the furanosyl to form the second ring. In certain embodiments, the bicyclic nucleotide has the structure of Formula I:

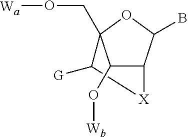

wherein B is a nucleobase;
wherein G is H, OH, NH₂, C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol, or substituted thio;
wherein X is O, S, or NR₁, wherein R₁ is H, C₁-C₆ alkyl, C₁-C₆ alkoxy, benzene or pyrene; and
wherein Wₐ and W_{b} are each independently, H, OH, a hydroxyl protecting group, a phosphorous moiety, or an internucleotide linking group attaching the nucleotide represented by Formula I to another nucleotide or to an oligonucleotide and wherein at least one of Wₐᵣ or W_{b} is an internucleotide linking group attaching the nucleotide represented by Formula I to an oligonucleotide.

In certain embodiments of Formula I, G is H and X is NR₁, wherein R₁ is benzene or pyrene. In certain embodiments, of Formula I, G is H and X is S.

In certain embodiments of Formula I, G is H and X is O:

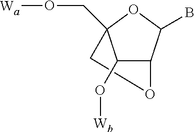

In certain embodiments of Formula I, G is H and X is NR₁, wherein R₁ is H, CH₃, or OCH₃:

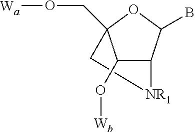

In certain embodiments of Formula I, G is OH or NH₂ and X is O.

In certain embodiments of Formula I, G is OH and X is O:

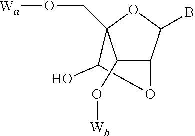

In certain embodiments of Formula I, G is NH₂ and X is O:

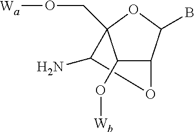

In certain embodiments, of Formula I, G is CH₃ or CH₂OCH₃ and X is O. In certain embodiments, of Formula I, G is CH₃ and X is O:

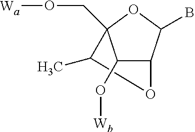

In certain embodiments, of Formula I, G is CH₂OCH₃ and X is O:

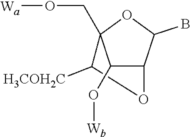

In certain embodiments, the bicyclic nucleotide has the structure of Formula II:

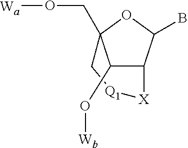

wherein B is a nucleobase;
wherein Q₁ is CH₂ or O;
wherein X is CH₂, O, S, or NR₁, wherein R₁ is H, C₁-C₆ alkyl, C₁-C₆ alkoxy, benzene or pyrene;
wherein if Q₁ is O, X is CH₂;
wherein if Q₁ is CH₂, X is CH₂, O, S, or NR₁, wherein R₁ is H, C₁-C₆ alkyl, C₁-C₆ alkoxy, benzene or pyrene;
wherein Wₐ and W_{b} are each independently, H, OH, a hydroxyl protecting group, a phosphorous moiety, or an internucleotide linking group attaching the nucleotide represented by Formula II to another nucleotide or to an oligonucleotide and wherein at least one of Wₐ or W_{b} is an internucleotide linking group attaching the nucleotide represented by Formula II to an oligonucleotide.

In certain embodiments of Formula II, Q₁ is O and X is CH₂:

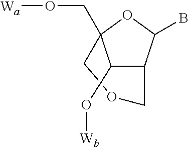

In certain embodiments of Formula II, Q₁ is CH₂ and X is O:

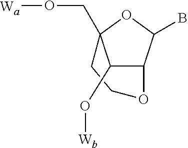

In certain embodiments of Formula II, Q₁ is CH₂ and X is NR₁, wherein R₁ is H, CH₃ or OCH₃:

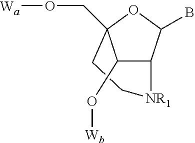

In certain embodiments of Formula II, Q₁ is CH₂ and X is NH:

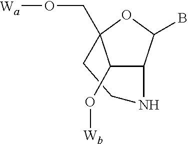

In certain embodiments, the bicyclic nucleotide has the structure of Formula III:

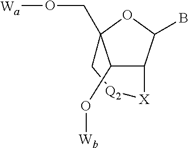

wherein B is a nucleobase;
wherein Q₂ is O or NR₁, wherein R₁ is H, C₁-C₆ alkyl, C₁-C₆ alkoxy, benzene or pyrene;
wherein X is CH₂, O, S, or NR₁, wherein R₁ is H, C₁-C₆ alkyl, C₁-C₆ alkoxy, benzene or pyrene;
wherein if Q₂ is O, X is NR₁;
wherein if Q₂ is NR₁, X is O or S;
wherein Wₐ and W_{b} are each independently, H, OH, a hydroxyl protecting group, a phosphorous moiety, or an internucleotide linking group attaching the nucleotide represented by Formula III to another nucleotide or to an oligonucleotide and wherein at least one of Wₐ or W_{b} is an internucleotide linking group attaching the nucleotide represented by Formula III to an oligonucleotide.

In certain embodiments of Formula III, Q₂ is O and X is NR₁. In certain embodiments of Formula III, Q₂ is O and X is NR₁, wherein R₁ is C₁-C₆ alkyl. In certain embodiments of Formula III, Q₂ is O and X is NR₁ and R₁ is H or CH₃.

In certain embodiments of Formula III, Q₂ is O and X is NR₁ and R₁ is CH₃:

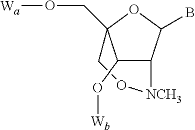

In certain embodiments of Formula III, Q₂ is NR₁ and X is O. In certain embodiments of Formula III, Q₂ is NR₁, wherein R₁ is C₁-C₆ alkyl and X is O.

In certain embodiments of Formula III, Q₂ is NCH₃ and X is O:

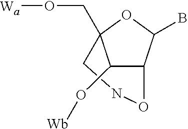

In certain embodiments, the bicyclic nucleotide has the structure of Formula IV:

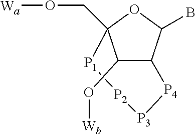

wherein B is a nucleobase;
wherein P₁ and P₃ are CH₂, P₂ is CH₂ or O and P₄ is O; and
wherein Wₐ and W_{b} are each independently, H, OH, a hydroxyl protecting group, a phosphorous moiety, or an internucleotide linking group attaching the nucleotide represented by Formula IV to another nucleotide or to an oligonucleotide and wherein at least one of Wₐ or W_{b} is an internucleotide linking group attaching the nucleotide represented by Formula IV to an oligonucleotide.

In certain embodiments of Formula IV, P₁, P₂, and P₃ are CH₂, and P₄ is O:

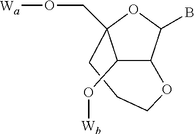

In certain embodiments of Formula IV, P₁ and P₃ are CH₂, P₂ is O and P₄ is O:

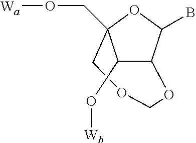

In certain embodiments, the bicyclic nucleotide has the structure of Formula Va or Vb:

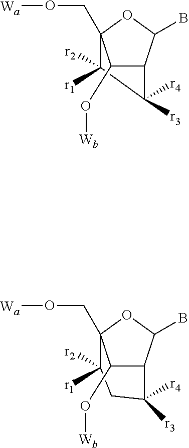

wherein B is a nucleobase;
wherein r1, r2, r3, and r4 are each independently H, halogen, C₁-C₁₂ alkyl, substituted C₁-C₁₂ alkyl, C₂-C₁₂ alkenyl, substituted C₂-C₁₂ alkenyl, C₂-C₁₂ alkynyl; substituted C₂-C₁₂ alkynyl; C₁-C₁₂ alkoxy; substituted C₁-C₁₂ alkoxy, OT₁, ST₁, SOT₁, SO₂T₁, NT₁T₂, N3, CN, C(═O)OT₁, C(═O)NT₁T₂, C(═O)T₁, O—C(═O)NT₁T2, N(H)C(═NH)NT₁T₂, N(H)C(═O)NT₁T₂ or N(H)C(═S)NT₁T₂, wherein each of T1 and T2 is independently H, C₁-C₆ alkyl, or substituted C₁-C₁₆ alkyl; or
r1 and r2 or r3 and r4 together are ═C (r5)(r6), wherein r5 and r6 are each independently H, halogen, C₁-C₁₂ alkyl, or substituted C₁-C₁₂ alkyl; and
wherein Wₐ and W_{b} are each independently, H, OH, a hydroxyl protecting group, a phosphorous moiety, or an internucleotide linking group attaching the nucleotide represented by Formula V to another nucleotide or to an oligonucleotide and wherein at least one of Wₐ or W_{b} is an internucleotide linking group attaching the nucleotide represented by Formula V to an oligonucleotide.

In certain embodiments, the bicyclic sugar moiety is a substituted furanosyl comprising a bridge that connects the 2′-carbon and the 4′-carbon of the furanosyl to form the second ring, wherein the bridge that connects the 2′-carbon and the 4′-carbon of the furanosyl includes, but is not limited to:
a) 4′-CH₂—O—N(R)-2′ and 4′-CH₂—N(R)—O-2′, wherein R is H, C₁-C₁₂ alkyl, or a protecting group, including, for example, 4′-CH₂—NH—O-2′ (also known as BNA^{NC}), 4′-CH₂—N(CH₃)—O-2′ (also known as BNA^{NC}[NMe]), (as described in U.S. Pat. No. 7,427,672, which is hereby incorporated by reference in its entirety);
b) 4′-CH₂-2′; 4′-(CH₂)₂-2′; 4′-(CH₂)₃-2′; 4′-(CH₂)—O-2′ (also known as LNA); 4′-(CH₂)—S-2′; 4′-(CH₂)₂—O-2′ (also known as ENA); 4′-CH(CH₃)—O-2′ (also known as cEt); and 4′-CH(CH₂OCH₃)—O-2′ (also known as cMOE), and analogs thereof (as described in U.S. Pat. No. 7,399,845, which is hereby incorporated by reference in its entirety);
c) 4′-C(CH₃)(CH₃)—O-2′ and analogs thereof (as described in U.S. Pat. No. 8,278,283, which is hereby incorporated by reference in its entirety);
d) 4′-CH₂—N(OCH₃)-2′ and analogs thereof (as described in U.S. Pat. No. 8,278,425, which is hereby incorporated by reference in its entirety);
e) 4′-CH₂—O—N(CH₃)-2′ and analogs thereof (as described in U.S. Patent Publication No. 2004/0171570, which is hereby incorporated by reference in its entirety);
f) 4′-CH₂—C(H)(CH₃)-2′ and analogs thereof (as described in Chattopadhyaya et al., J. ORG. CHEM., 2009, 74, 118-34, which is hereby incorporated by reference in its entirety); and
g) 4′-CH₂—C(—CH₂)-2′ and analogs thereof as described in U.S. Pat. No. 8,278,426, which is hereby incorporated by reference in its entirety).

In certain embodiments, the bicyclic nucleotide (BN) is one or more of the following: (a) methyleneoxy BN, (b) ethyleneoxy BN, (c) aminooxy BN; (d) oxyamino BN, (e) methyl(methyleneoxy) BN (also known as constrained ethyl or cET), (f) methylene-thio BN, (g) methylene amino BN, (h) methyl carbocyclic BN, and (i) propylene carbocyclic BN, as shown below.

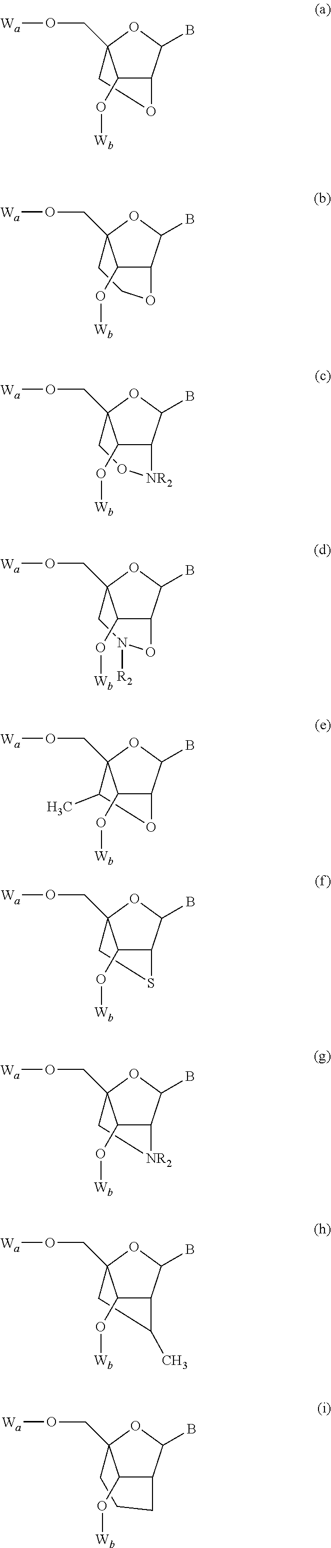

In the bicyclic nucleotides of (a) to (i) above, B is a nucleobase, R₂ is H or CH₃ and Wₐ and W_{b} are each independently, H, OH, a hydroxyl protecting group, a phosphorous moiety, or an internucleotide linking group attaching the bicyclic nucleotide to another nucleotide or to an oligonucleotide and wherein at least one of Wₐ or W_{b} is an internucleotide linking group attaching the bicyclic nucleotide to an oligonucleotide.

In one embodiment of the oxyamino BN (d), R₂ is CH₃, as follows (also known as BNA^{NC}[NMe]):

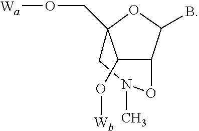

In certain embodiments, bicyclic sugar moieties and bicyclic nucleotides incorporating such bicyclic sugar moieties are further defined by isomeric configuration. In certain embodiments, the bicyclic sugar moiety or nucleotide is in the α-L configuration. In certain embodiments, the bicyclic sugar moiety or nucleotide is in the β-D configuration. For example, in certain embodiments, the bicyclic sugar moiety or nucleotide comprises a 2′O,4′-C-methylene bridge (2′-O—CH₂-4′) in the α-L configuration (α-L LNA). In certain embodiments, the bicyclic sugar moiety or nucleotide is in the R configuration. In certain embodiments, the bicyclic sugar moiety or nucleotide is in the S configuration. For example, in certain embodiments, the bicyclic sugar moiety or nucleotide comprises a 4′-CH(CH₃)—O-2′ bridge (i.e., cEt) in the S-configuration.

### Tricyclic Nucleotides

In some embodiments, the Tₘ-increasing nucleotide is a tricyclic nucleotide. The synthesis of tricyclic nucleotides and their incorporation into nucleic acid compounds has also been reported in the literature, including, for example, Steffens et al., J. AM. CHEM. SOC. 1997; 119:11548-549; Steffens et al., J. ORG. CHEM. 1999; 121(14):3249-55; Renneberg et al., J. AM. CHEM. SOC. 2002; 124:5993-6002; Ittig et al., NUCLEIC ACIDS RES. 2004; 32(1):346-53; Scheidegger et al., CHEMISTRY 2006; 12:8014-23; Ivanova et al., OLIGONUCLEOTIDES 2007; 17:54-65; each of which is each hereby incorporated by reference in its entirety.

In certain embodiments, the tricyclic nucleotide is a tricyclo nucleotide (also called tricyclo DNA) in which the 3′-carbon and 5′-carbon centers are connected by an ethylene that is fused to a cyclopropane ring, as discussed for example in Leumann C J, BIOORG. MED. CHEM. 2002; 10:841-54 and published U.S. Applications 2015/0259681 and 2018/0162897, which are each hereby incorporated by reference. In certain embodiments, the tricyclic nucleotide comprises a substituted furanosyl ring comprising a bridge that connects the 2′-carbon and the 4′-carbon of the furanosyl to form a second ring, and a third fused ring resulting from a group connecting the 5′-carbon to the methylene group of the bridge that connects the 2′-carbon and the 4′-carbon of the furanosyl, as discussed, for example, in published U.S. Application 2015/0112055, which is hereby incorporated by reference.

### Other Tₘ-Increasing Nucleotides

In addition to bicyclic and tricyclic nucleotides, other Tₘ-increasing nucleotides can be used in the RNAi oligonucleotides described herein. For example, in certain embodiments, the Tₘ-increasing nucleotide is a G-clamp, guanidine G-clamp or analogue thereof (Wilds et al., CHEM, 2002; 114:123 and Wilds et al., CHIM ACTA 2003; 114:123), a hexitol nucleotide (Herdewijn, CHEM. BIODIVERSITY 2010; 7:1-59), or a modified nucleotide. The modified nucleotide can have a modified nucleobase, as described herein, including for example, 5-bromo-uracil, 5-iodo-uracil, 5-propynyl-modified pyrimidines, or 2-amino adenine (also called 2,6-diaminopurine) (Deleavey et al., CHEM. & BIOL. 2012; 19:937-54) or 2-thio uridine, 5 Me-thio uridine, and pseudo uridine. The modified nucleotide can also have a modified sugar moiety, as described for example, in U.S. Pat. No. 8,975,389, which is hereby incorporated by reference, or as described herein, except that the Tₘ-increasing nucleotide is not modified at the 2′-carbon of the sugar moiety with a 2′-F or a 2′-OMe.

In certain embodiments, the Tₘ-increasing nucleotide is a bicyclic nucleotide. In certain embodiments, the Tₘ-increasing nucleotide is a tricyclic nucleotide. In certain embodiments, the Tₘ-increasing nucleotide a G-clamp, guanidine G-clamp or analogue thereof. In certain embodiments, the Tₘ-increasing nucleotide is a hexitol nucleotide. In certain embodiments, the Tₘ-increasing nucleotide is a bicyclic or tricyclic nucleotide. In certain embodiments, the Tₘ-increasing nucleotide is a bicyclic nucleotide, a tricyclic nucleotide, or a G-clamp, guanidine G-clamp or analogue thereof. In certain embodiments, the Tₘ-increasing nucleotide is a bicyclic nucleotide, a tricyclic nucleotide, a G-clamp, guanidine G-clamp or analogue thereof, or a hexitol nucleotide.

In certain embodiments, the Tₘ-increasing nucleotide increases the Tₘ of the nucleic acid inhibitor molecule by at least 2° C. per incorporation. In certain embodiments, the Tₘ-increasing nucleotide increases the Tₘ of nucleic acid inhibitor molecule by at least 3° C. per incorporation. In certain embodiments, the Tₘ-increasing nucleotide increases the Tₘ of nucleic acid inhibitor molecule by at least 4° C. per incorporation. In certain embodiments, the Tₘ-increasing nucleotide increases the Tₘ of nucleic acid inhibitor molecule by at least 5° C. per incorporation.

### Targeting Ligands

In some embodiments, it is desirable to target the oligonucleotides of the disclosure (e.g., ligand-conjugated RNAi oligonucleotides) to one or more cells or tissues. In some embodiments, it is desirable to target the oligonucleotides of the disclosure (e.g., ligand-conjugated RNAi oligonucleotides) to one or more cells or tissues of the liver.

Such a strategy can help to avoid undesirable effects in other organs or avoid undue loss of the oligonucleotide to cells, tissue or organs that would not benefit from the oligonucleotide. Accordingly, in some embodiments, a ligand-conjugated RNAi oligonucleotide disclosed herein is modified to facilitate targeting and/or delivery to a particular tissue, cell, or organ (e.g., to facilitate delivery of the conjugate to the liver). In some embodiments, a ligand-conjugated RNAi oligonucleotide comprises at least one nucleotide (e.g., 1, 2, 3, 4, 5, 6 or more nucleotides) conjugated to one or more GalNAc moieties.

### GalNAc Ligands

GalNAc is a high affinity ligand for the ASGPR, which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to oligonucleotide of the instant disclosure can be used to target these oligonucleotides to the ASGPR expressed on cells. In some embodiments, an oligonucleotide of the instant disclosure is conjugated to at least one or more GalNAc moieties, wherein the GalNAc moieties target the oligonucleotide to an ASGPR expressed on human liver cells (e.g., human hepatocytes). In some embodiments, the GalNAc moiety target the oligonucleotide to the liver.

In some embodiments, an oligonucleotide of the disclosure is conjugated directly or indirectly to a monovalent GalNAc. In some embodiments, the oligonucleotide is conjugated directly or indirectly to more than one monovalent GalNAc (i.e., is conjugated to 2, 3 or 4 monovalent GalNAc moieties, and is typically conjugated to 3 or 4 monovalent GalNAc moieties). In some embodiments, an oligonucleotide is conjugated to one or more bivalent GalNAc, trivalent GalNAc or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of an oligonucleotide are each conjugated to a separate GalNAc. In some embodiments, 1 to 3 nucleotides of an oligonucleotide are each conjugated to a separate GalNAc. In some embodiments, GalNAc moieties are conjugated to 2 to 4 consecutive nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. In some embodiments, 2 to 4 consecutive nucleotides of a sense strand are each conjugated to a GalNAc moiety. In some embodiments, 1 to 3 consecutive nucleotides of a sense strand are each conjugated to a GalNAc moiety.

In some embodiments, the sense strand comprises three consecutive nucleotides conjugated with a GalNAc moiety. In some embodiments, the three 3′ terminal nucleotides of the sense strand are conjugated with a GalNAc moiety. In some embodiments, the three 3′ terminal nucleotides of the sense strand are conjugated with a GalNAc. In some embodiments, the two 2′ terminal nucleotides of the sense strand are conjugated with a GalNAc moiety. In some embodiments, the two 3′ terminal nucleotides of the sense strand are conjugated with a GalNAc.

In some embodiments, any of the GalNAc moieties described herein are conjugated to a nucleotide of the sense strand of the oligonucleotide. In some embodiments, a GalNAc moiety is conjugated to a terminal position of the oligonucleotide. In some embodiments, the GalNAc moiety is conjugated to the 5′ terminal nucleotide of the sense strand. In some embodiments, the GalNAc moiety is conjugated to the 3′ terminal nucleotide of the sense strand.

In some embodiments, the GalNAc moiety is conjugated to an internal nucleotide on the sense strand. An internal position is any nucleotide position other than the two terminal positions from each end of the sense strand. In some embodiments, the GalNAc moiety is conjugated to one or more internal positions of the sense strand. In some embodiments, the GalNAc moiety is conjugated to position 1, position 2, position 3, position 4, position 5, position 6, position 7, position 8, position 9, position 10, position 11, position 12, position 13, position 14, position 15, position 16, position 17, position 18, position 19, position 20, or position 21, of a sense strand. In some embodiments, the GalNAc moiety is conjugated to position 1 of the sense strand. In some embodiments, the GalNAc moiety is conjugated to position 2 of the sense strand. In some embodiments, the GalNAc moiety is conjugated to position 3 of the sense strand. In some embodiments, the GalNAc moiety is conjugated to position 4 of the sense strand. In some embodiments, the GalNAc moiety is conjugated to position 5 of the sense strand. In some embodiments, the GalNAc moiety is conjugated to position 6 of the sense strand. In some embodiments, the GalNAc moiety is conjugated to position 7 of the sense strand. In some embodiments, the GalNAc moiety is conjugated to position 8 of the sense strand. In some embodiments, the GalNAc moiety is conjugated to position 9 of the sense strand. In some embodiments, the GalNAc moiety is conjugated to position 10 of the sense strand. In some embodiments, the GalNAc moiety is conjugated to position 11 of the sense strand. In some embodiments, the GalNAc moiety is conjugated to position 12 of the sense strand. In some embodiments, the GalNAc moiety is conjugated to position 13 of the sense strand. In some embodiments, the GalNAc moiety is conjugated to position 14 of the sense strand. In some embodiments, the GalNAc moiety is conjugated to position 15 of the sense strand. In some embodiments, the GalNAc moiety is conjugated to position 16 of the sense strand. In some embodiments, the GalNAc moiety is conjugated to position 17 of the sense strand. In some embodiments, the GalNAc moiety is conjugated to position 18 of the sense strand. In some embodiments, the GalNAc moiety is conjugated to position 19 of the sense strand. In some embodiments, the GalNAc moiety is conjugated to position 20 of the sense strand.

In some embodiments, the ligan moiety is conjugated to position 3 of the antisense strand. In some embodiments, the ligan moiety is conjugated to position 15 of the antisense strand. In some embodiments, the ligan moiety is conjugated to position 16 of the antisense strand. In some embodiments, the ligan moiety is conjugated to position 19 of the antisense strand. In some embodiments, the ligan moiety is conjugated to position 20 of the antisense strand. In some embodiments, the ligan moiety is conjugated to position 21 of the antisense strand. In some embodiments, the ligan moiety is conjugated to position 22 of the antisense strand.

In some embodiments, the GalNAc moiety is conjugated to a nucleotide of the sense strand that forms a base pair with a nucleotide at position 20, position 19, position 18, position 17, position 16, position 15, position 14, position 13, position 12, position 11, position 10, position 9, position 8, position 7, position 6, position 5, position 4, position 3, position 2, or position 1 of the antisense strand.

In some embodiments, a ligand-conjugated RNAi oligonucleotide described herein comprises at least one nucleotide conjugated with one or more GalNAc moieties. In some embodiments, the one or more GalNAc moieties are conjugated to the same nucleotide. In some embodiments, the one or more GalNAc moieties are conjugated to different nucleotides. In some embodiments, one, two, three, four, five, or six GalNAc moieties are conjugated to the oligonucleotide. In some embodiments, one or more GalNAc moieties are conjugated to an adenine nucleotide. In some embodiments, one or more GalNAc moieties are conjugated to a guanine nucleotide. In some embodiments, one or more GalNAc moieties are conjugated to a cytosine nucleotide. In some embodiments, one or more GalNAc moieties are conjugated to a thymine nucleotide. In some embodiments, one or more GalNAc moieties are conjugated to a uracil nucleotide.

In some embodiments, a ligand-conjugated RNAi oligonucleotide described herein comprises at least two nucleotides conjugated with one or more GalNAc moieties. In some embodiments, a ligand-conjugated RNAi oligonucleotide described herein comprises at least three nucleotides conjugated with one or more GalNAc moieties. In some embodiments, the sense strand of a ligand-conjugated RNAi oligonucleotide described herein comprises at least one nucleotide conjugated with one or more GalNAc moieties. In some embodiments, the sense strand of a ligand-conjugated RNAi oligonucleotide described herein comprises at least two nucleotides conjugated with one or more GalNAc moieties. In some embodiments, the sense strand of a ligand-conjugated RNAi oligonucleotide described herein comprises at least three nucleotides conjugated with one or more GalNAc moieties.

In some embodiments, a ligand-conjugated RNAi oligonucleotide comprises a sense strand, wherein the sense strand comprises 1-2 GalNAc moieties. In some embodiments, a ligand-conjugated RNAi oligonucleotide comprises a sense strand, wherein the sense strand comprises 1-3 GalNAc moieties. In some embodiments, a ligand-conjugated RNAi oligonucleotide comprises a sense strand, wherein the sense strand comprises 1~4 GalNAc moieties. In some embodiments, a ligand-conjugated RNAi oligonucleotide comprises a sense strand, wherein the sense strand comprises 2-3 GalNAc moieties. In some embodiments, a ligand-conjugated RNAi oligonucleotide comprises a sense strand, wherein the sense strand comprises 2-4 GalNAc moieties. In some embodiments, a ligand-conjugated RNAi oligonucleotide comprises a sense strand, wherein the sense strand comprises 3-4 GalNAc moieties.

In some embodiments, one to two consecutive nucleotides of the sense strand are each conjugated to a monovalent GalNAc. In some embodiments, one to three consecutive nucleotides of the sense strand are each conjugated to a monovalent GalNAc. In some embodiments, one to four consecutive nucleotides of the sense strand are each conjugated to a monovalent GalNAc. In some embodiments, two to three consecutive nucleotides of the sense strand are each conjugated to a monovalent GalNAc. In some embodiments, two to four consecutive nucleotides of the sense strand are each conjugated to a monovalent GalNAc. In some embodiments, three to four consecutive nucleotides of the sense strand are each conjugated to a monovalent GalNAc. In some embodiments, two consecutive nucleotides of the sense strand are each conjugated to a monovalent GalNAc. In some embodiments, three consecutive nucleotides of the sense strand are each conjugated to a monovalent GalNAc. In some embodiments, four consecutive nucleotides of the sense strand are each conjugated to a monovalent GalNAc. In some embodiments, a ligand-conjugated RNAi oligonucleotide comprises an antisense strand, wherein the antisense strand comprises 1-3 GalNAc moieties. In some embodiments, a ligand-conjugated RNAi oligonucleotide comprises an antisense strand, wherein the antisense strand comprises 2-3 GalNAc moieties.

In some embodiments, a ligand-conjugated RNAi oligonucleotide comprises a sense strand and antisense strand, wherein the sense strand comprises 1-2 GalNAc moieties, and the antisense strand comprises 1-2 GalNAc moieties. In some embodiments, a ligand-conjugated RNAi oligonucleotide comprises a sense strand and antisense strand, wherein the sense strand comprises 1-2 GalNAc moieties, and the antisense strand comprises 1-3 GalNAc moieties. In some embodiments, a ligand-conjugated RNAi oligonucleotide comprises a sense strand and antisense strand, wherein the sense strand comprises 1-3 GalNAc moieties, and the antisense strand comprises 1-2 GalNAc moieties. In some embodiments, a ligand-conjugated RNAi oligonucleotide comprises a sense strand and antisense strand, wherein the sense strand comprises 1-3 GalNAc moieties, and the antisense strand comprises 1-3 GalNAc moieties. In some embodiments, a ligand-conjugated RNAi oligonucleotide comprises a sense strand and antisense strand, wherein the sense strand comprises 1~4 GalNAc moieties, and the antisense strand comprises 1-2 GalNAc moieties. In some embodiments, a ligand-conjugated RNAi oligonucleotide comprises a sense strand and antisense strand, wherein the sense strand comprises 1~4 GalNAc moieties, and the antisense strand comprises 1-3 GalNAc moieties.

In some embodiments, a ligand-conjugated RNAi oligonucleotide comprises a 20-nucleotide sense strand, wherein the sense strand comprises at least two nucleotides conjugated with one or more GalNAc moieties. In some embodiments, a ligand-conjugated RNAi oligonucleotide comprises a 20-nucleotide sense strand, wherein the sense strand comprises at least three nucleotides conjugated with one or more GalNAc moieties. In some embodiments, a ligand-conjugated RNAi oligonucleotide comprises a 20-nucleotide sense strand, wherein the sense strand comprises at least three nucleotides conjugated with one or more GalNAc moieties. In some embodiments, a ligand-conjugated RNAi oligonucleotide comprises a 19-nucleotide sense strand, wherein the sense strand comprises at least two nucleotides conjugated with one or more GalNAc moieties. In some embodiments, a ligand-conjugated RNAi oligonucleotide comprises a 19-nucleotide sense strand, wherein the sense strand comprises at least three nucleotides conjugated with one or more GalNAc moieties. In some embodiments, a ligand-conjugated RNAi oligonucleotide comprises a 18-nucleotide sense strand, wherein the sense strand comprises at least two nucleotides conjugated with one or more GalNAc moieties. In some embodiments, a ligand-conjugated RNAi oligonucleotide comprises a 18-nucleotide sense strand, wherein the sense strand comprises at least three nucleotides conjugated with one or more GalNAc moieties. In some embodiments, a ligand-conjugated RNAi oligonucleotide comprises a 17-nucleotide sense strand, wherein the sense strand comprises at least two nucleotides conjugated with one or more GalNAc moieties. In some embodiments, a ligand-conjugated RNAi oligonucleotide comprises a 17-nucleotide sense strand, wherein the sense strand comprises at least three nucleotides conjugated with one or more GalNAc moieties. In some embodiments, a ligand-conjugated RNAi oligonucleotide comprises a 16-nucleotide sense strand, wherein the sense strand comprises at least two nucleotides conjugated with one or more GalNAc moieties. In some embodiments, a ligand-conjugated RNAi oligonucleotide comprises a 16-nucleotide sense strand, wherein the sense strand comprises at least three nucleotides conjugated with one or more GalNAc moieties. In some embodiments, a ligand-conjugated RNAi oligonucleotide comprises a 15-nucleotide sense strand, wherein the sense strand comprises at least two nucleotides conjugated with one or more GalNAc moieties. In some embodiments, a ligand-conjugated RNAi oligonucleotide comprises a 15-nucleotide sense strand, wherein the sense strand comprises at least three nucleotides conjugated with one or more GalNAc moieties. In some embodiments, a ligand-conjugated RNAi oligonucleotide comprises a 14-nucleotide sense strand, wherein the sense strand comprises at least two nucleotides conjugated with one or more GalNAc moieties. In some embodiments, a ligand-conjugated RNAi oligonucleotide comprises a 14-nucleotide sense strand, wherein the sense strand comprises at least three nucleotides conjugated with one or more GalNAc moieties. In some embodiments, a ligand-conjugated RNAi oligonucleotide comprises a 13-nucleotide sense strand, wherein the sense strand comprises at least two nucleotides conjugated with one or more GalNAc moieties. In some embodiments, a ligand-conjugated RNAi oligonucleotide comprises a 13-nucleotide sense strand, wherein the sense strand comprises at least three nucleotides conjugated with one or more GalNAc moieties.

In some embodiments, the sense strand comprises a GalNac moiety conjugated to a nucleotide at position 1 and a nucleotide at position 2. In some embodiments, the sense strand comprises a GalNac moiety conjugated to a nucleotide at position 2, a nucleotide at position 3, and a nucleotide at position 4. In some embodiments, the sense strand comprises a GalNac moiety conjugated to a nucleotide at position 5, a nucleotide at position 6, and a nucleotide at position 7. In some embodiments, the sense strand comprises a GalNac moiety conjugated to a nucleotide at position 7, a nucleotide at position 8, and a nucleotide at position 9. In some embodiments, the sense strand comprises a GalNac moiety conjugated to a nucleotide at position 8 and a nucleotide at position 9. In some embodiments, the sense strand comprises a GalNac moiety conjugated to a nucleotide at position 12 and a nucleotide at position 13. In some embodiments, the sense strand comprises a GalNac moiety conjugated to a nucleotide at position 1, a nucleotide at position 2, and a nucleotide at position 3. In some embodiments, the sense strand comprises a GalNac moiety conjugated to a nucleotide at position 4, a nucleotide at position 5, and a nucleotide at position 6. In some embodiments, the sense strand comprises a GalNac moiety conjugated to a nucleotide at position 9, a nucleotide at position 10, and a nucleotide at position 11. In some embodiments, the sense strand comprises a GalNac moiety conjugated to a nucleotide at position 12, a nucleotide at position 13, and a nucleotide at position 14. In some embodiments, the sense strand comprises a GalNac moiety conjugated to a nucleotide at position 8, a nucleotide at position 9, and a nucleotide at position 10. In some embodiments, the sense strand comprises a GalNac moiety conjugated to a nucleotide at position 15, a nucleotide at position 16, and a nucleotide at position 17. In some embodiments, the sense strand comprises a GalNac moiety conjugated to a nucleotide at position 18, a nucleotide at position 19, and a nucleotide at position 20. In some embodiments, the sense strand comprises a GalNac moiety conjugated to a nucleotide at position 19 and a nucleotide at position 20. In some embodiments, the sense strand comprises a GalNac moiety conjugated to a nucleotide at position 16, a nucleotide at position 17, and a nucleotide at position 18. In some embodiments, the sense strand comprises a GalNac moiety conjugated to a nucleotide at position 13, a nucleotide at position 14, and a nucleotide at position 15. In some embodiments, the sense strand comprises a GalNac moiety conjugated to a nucleotide at position 14 and a nucleotide at position 15.

In some embodiments, the antisense strand comprises a GalNac moiety conjugated to a nucleotide at position 20 and a nucleotide at position 21. In some embodiments, the antisense strand comprises a GalNac moiety conjugated to a nucleotide at position 21 and a nucleotide at position 22. In some embodiments, the antisense strand comprises a GalNac moiety conjugated to a nucleotide at position 19, a nucleotide at position 20, and a nucleotide at position 21. In some embodiments, the antisense strand comprises a GalNac moiety conjugated to a nucleotide at position 17, a nucleotide at position 18, and a nucleotide at position 19.

In some embodiments, the sense strand comprises a GalNAc moiety conjugated to a nucleotide at position 1 and the antisense strand comprises a GalNac moiety conjugated to a nucleotide at position 15 and a nucleotide at position 16. In some embodiments, the sense strand comprises a GalNAc moiety conjugated to a nucleotide at position 18 and a nucleotide at position 19, and the antisense strand comprises a GalNac moiety conjugated to a nucleotide at position 13. In some embodiments, the sense strand comprises a GalNAc moiety conjugated to a nucleotide at position 1, and the antisense strand comprises a GalNac moiety conjugated to a nucleotide at position 19 and a nucleotide at position 20. In some embodiments, the sense strand comprises a GalNAc moiety conjugated to a nucleotide at position 1 and a nucleotide at position 2, and the antisense strand comprises a GalNac moiety conjugated to a nucleotide at position 19 and a nucleotide at position 20. In some embodiments, the sense strand comprises a GalNAc moiety conjugated to a nucleotide at position 1 and a nucleotide at position 2, and the antisense strand comprises a GalNac moiety conjugated to a nucleotide at position 20.

In some embodiments, the oligonucleotide has a monovalent GalNAc moiety attached to any one or more guanine nucleotides via any linker described herein, as depicted below in Chem 1 (X=heteroatom):

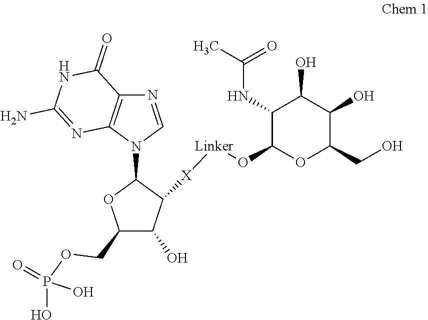

In some embodiments, the oligonucleotide has a monovalent GalNAc attached to any one or more adenine nucleotides via any linker described herein, as depicted below in Chem 2 (X=heteroatom):

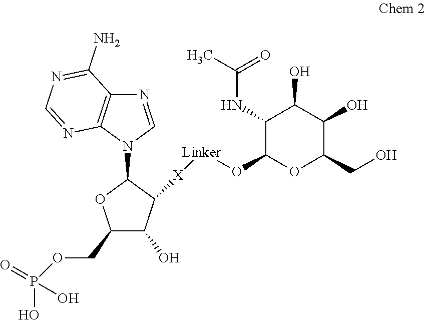

In some embodiments, a ligand-conjugated RNAi oligonucleotide herein comprises a monovalent GalNAc attached to a guanine nucleotide referred to as [ademG-GalNAc] or 2′-aminodiethoxymethanol-Guanine-GalNAc, as depicted below in Chem 3:

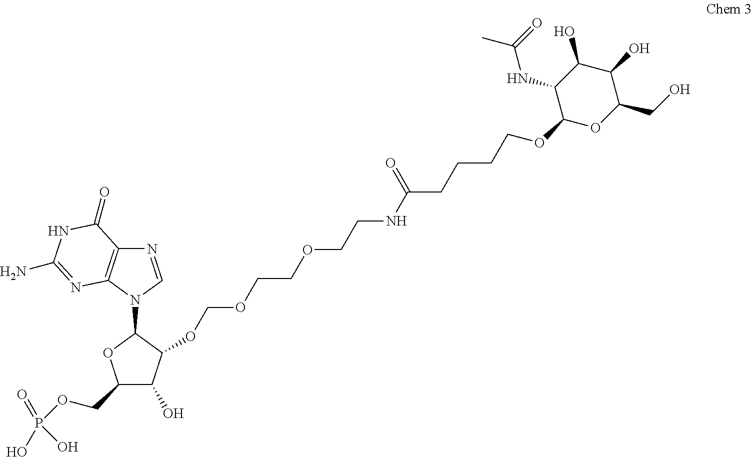 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, a ligand-conjugated RNAi oligonucleotide herein comprises a monovalent GalNAc attached to an adenine nucleotide, referred to as [ademA-GalNAc] or 2′-aminodiethoxymethanol-Adenine-GalNAc, as depicted below in Chem 4:

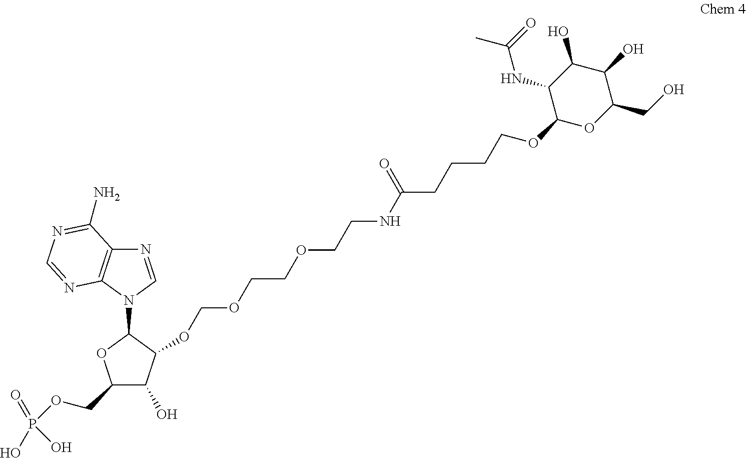 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is stable.

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5 or 6 bp in length) is provided between a targeting ligand (e.g., a GalNAc moiety) and a ligand-conjugated RNAi oligonucleotide. In some embodiments, a ligand-conjugated RNAi oligonucleotide herein does not have a GalNAc conjugated thereto.

### Exemplary Oligonucleotides

In some embodiments, the ligand-conjugated RNAi oligonucleotide comprises a nucleotide conjugated with a fatty acid. In some embodiments, the fatty acid is a saturated fatty acid. In some embodiments, the fatty acid is an unsaturated fatty acid. In some embodiments, ligand-conjugated RNAi oligonucleotide comprises a nucleotide conjugated with a ligand. In some embodiments, ligand-conjugated RNAi oligonucleotide comprises a nucleotide conjugated with GalNAc. In some embodiments, ligand-conjugated RNAi oligonucleotide comprises two nucleotides conjugated with GalNAc. In some embodiments, ligand-conjugated RNAi oligonucleotide comprises three nucleotides conjugated with GalNAc. In some embodiments, ligand-conjugated RNAi oligonucleotide comprises four nucleotides conjugated with GalNAc.

In some embodiments, the 3′ end of the sense strand is a blunt end. In some embodiments, the 5′ end of the antisense strand is a blunt end. In some embodiments, the 3′ end of the antisense strand comprises an overhang. In some embodiments, the 5′ end of the antisense strand comprises an overhang. In some embodiments, the 5′ and 3′ ends of the antisense strand each comprise an overhang.

In some embodiments, the ligand-conjugated RNAi oligonucleotide comprises one or more 2′ modifications. In some embodiments, the 2′ modifications are selected from 2′-fluoro and 2′-methyl.

In some embodiments, the sense strand is 18-nucleotides in length and the antisense strand is 22-nucleotides in length. In some embodiments, the sense strand is 20-nucleotides in length and the antisense strand is 22-nucleotides in length. In some embodiments, the sense strand is 15-nucleotides in length and the antisense strand is 22-nucleotides in length. In some embodiments, the sense strand is 15-nucleotides in length and the antisense strand is 21-nucleotides in length. In some embodiments, the sense strand is 15-nucleotides in length and the antisense strand is 20-nucleotides in length. In some embodiments, the sense strand is 15-nucleotides in length and the antisense strand is 19-nucleotides in length. In some embodiments, the sense strand is 15-nucleotides in length and the antisense strand is 18-nucleotides in length. In some embodiments, the sense strand is 13-nucleotides in length and the antisense strand is 22-nucleotides in length.

In some embodiments, the ligand-conjugated RNAi oligonucleotide comprises an antisense strand 22 nucleotides in length, a sense strand 18 nucleotides in length, a duplex between the antisense and sense strand of 18 base pairs, a 3′ overhang of the antisense strand of 2 nucleotides, a 5′ overhang of the antisense strand of 2 nucleotides, wherein the sense strand comprises one to four nucleotides each conjugated to a monovalent GalNAc molecule, and the oligonucleotide comprises at least one 2′-modification.

In some embodiments, the ligand-conjugated RNAi oligonucleotide comprises an antisense strand 22 nucleotides in length, a sense strand 18 nucleotides in length, a duplex between the antisense and sense strand of 18 base pairs, a 3′ overhang of the antisense strand of 2 nucleotides, a 5′ overhang of the antisense strand of 2 nucleotides, wherein the antisense strand comprises one to four nucleotides each conjugated to a monovalent GalNAc molecule, and the oligonucleotide comprises at least one 2′-modification.

In some embodiments, the ligand-conjugated RNAi oligonucleotide comprises an antisense strand 22 nucleotides in length, a sense strand 18 nucleotides in length, a duplex between the antisense and sense strand of 18 base pairs, a 3′ overhang of the antisense strand of 2 nucleotides, a 5′ overhang of the antisense strand of 2 nucleotides, wherein the sense strand comprises one to four nucleotides each conjugated to a monovalent GalNAc molecule, and the oligonucleotide comprises at least one 2′-modification selected from 2′-aminoethyl, 2′-fluoro, 2′-O-methyl, 2′-O-methoxyethyl, and 2′-deoxy-2′-fluoro-β-d-arabinonucleic acid.

In some embodiments, the ligand-conjugated RNAi oligonucleotide comprises an antisense strand 22 nucleotides in length, a sense strand 18 nucleotides in length, a duplex between the antisense and sense strand of 18 base pairs, a 3′ overhang of the antisense strand of 2 nucleotides, a 5′ overhang of the antisense strand of 2 nucleotides, wherein the antisense strand comprises one to four nucleotides each conjugated to a monovalent GalNAc molecule, and the oligonucleotide comprises at least one 2′-modification selected from 2′-aminoethyl, 2′-fluoro, 2′-O-methyl, 2′-O-methoxyethyl, and 2′-deoxy-2′-fluoro-β-d-arabinonucleic acid.

In some embodiments, the ligand-conjugated RNAi oligonucleotide comprises an antisense strand 22 nucleotides in length, a sense strand 18 nucleotides in length, a duplex between the antisense and sense strand of 18 base pairs, a 3′ overhang of the antisense strand of 2 nucleotides, a 5′ overhang of the antisense strand of 2 nucleotides, wherein the sense strand comprises one to four nucleotides each conjugated to a monovalent GalNAc molecule, the oligonucleotide comprises at least one 2′-modification selected from 2′-aminoethyl, 2′-fluoro, 2′-O-methyl, 2′-O-methoxyethyl, and 2′-deoxy-2′-fluoro-β-d-arabinonucleic acid, and the oligonucleotide comprises at least one internucleotide linkage.

In some embodiments, the ligand-conjugated RNAi oligonucleotide comprises an antisense strand 22 nucleotides in length, a sense strand 18 nucleotides in length, a duplex between the antisense and sense strand of 18 base pairs, a 3′ overhang of the antisense strand of 2 nucleotides, a 5′ overhang of the antisense strand of 2 nucleotides, wherein the antisense strand comprises one to four nucleotides each conjugated to a monovalent GalNAc molecule, the oligonucleotide comprises at least one 2′-modification selected from 2′-aminoethyl, 2′-fluoro, 2′-O-methyl, 2′-O-methoxyethyl, and 2′-deoxy-2′-fluoro-β-d-arabinonucleic acid, and the oligonucleotide comprises at least one internucleotide linkage.

In some embodiments, the ligand-conjugated RNAi oligonucleotide comprises an antisense strand 22 nucleotides in length, a sense strand 18 nucleotides in length, a duplex between the antisense and sense strand of 18 base pairs, a 3′ overhang of the antisense strand of 2 nucleotides, a 5′ overhang of the antisense strand of 2 nucleotides, wherein the sense strand comprises one to four nucleotides each conjugated to a monovalent GalNAc molecule, the oligonucleotide comprises at least one 2′-fluoro modification and at least one 2′-O-methyl modification, and the oligonucleotide comprises at least one internucleotide linkage.

In some embodiments, the ligand-conjugated RNAi oligonucleotide comprises an antisense strand 22 nucleotides in length, a sense strand 18 nucleotides in length, a duplex between the antisense and sense strand of 18 base pairs, a 3′ overhang of the antisense strand of 2 nucleotides, a 5′ overhang of the antisense strand of 2 nucleotides, wherein the antisense strand comprises one to four nucleotides each conjugated to a monovalent GalNAc molecule, the oligonucleotide comprises at least one 2′-fluoro modification and at least one 2′-O-methyl modification, and the oligonucleotide comprises at least one internucleotide linkage.

In some embodiments, the ligand-conjugated RNAi oligonucleotide comprises an antisense strand 20 nucleotides in length, a sense strand 16 nucleotides in length, a duplex between the antisense and sense strand of 16 base pairs, a 3′ overhang of the antisense strand of 2 nucleotides, a 5′ overhang of the antisense strand of 2 nucleotides, wherein the sense strand comprises one to four nucleotides each conjugated to a monovalent GalNAc molecule, and the oligonucleotide comprises at least one 2′-modification.

In some embodiments, the ligand-conjugated RNAi oligonucleotide comprises an antisense strand 20 nucleotides in length, a sense strand 16 nucleotides in length, a duplex between the antisense and sense strand of 16 base pairs, a 3′ overhang of the antisense strand of 2 nucleotides, a 5′ overhang of the antisense strand of 2 nucleotides, wherein the antisense strand comprises one to four nucleotides each conjugated to a monovalent GalNAc molecule, and the oligonucleotide comprises at least one 2′-modification.

In some embodiments, the ligand-conjugated RNAi oligonucleotide comprises an antisense strand 20 nucleotides in length, a sense strand 16 nucleotides in length, a duplex between the antisense and sense strand of 16 base pairs, a 3′ overhang of the antisense strand of 2 nucleotides, a 5′ overhang of the antisense strand of 2 nucleotides, wherein the sense strand comprises one to four nucleotides each conjugated to a monovalent GalNAc molecule, and the oligonucleotide comprises at least one 2′-modification selected from 2′-aminoethyl, 2′-fluoro, 2′-O-methyl, 2′-O-methoxyethyl, and 2′-deoxy-2′-fluoro-β-d-arabinonucleic acid.

In some embodiments, the ligand-conjugated RNAi oligonucleotide comprises an antisense strand 20 nucleotides in length, a sense strand 16 nucleotides in length, a duplex between the antisense and sense strand of 16 base pairs, a 3′ overhang of the antisense strand of 2 nucleotides, a 5′ overhang of the antisense strand of 2 nucleotides, wherein the antisense strand comprises one to four nucleotides each conjugated to a monovalent GalNAc molecule, and the oligonucleotide comprises at least one 2′-modification selected from 2′-aminoethyl, 2′-fluoro, 2′-O-methyl, 2′-O-methoxyethyl, and 2′-deoxy-2′-fluoro-β-d-arabinonucleic acid.

In some embodiments, the ligand-conjugated RNAi oligonucleotide comprises an antisense strand 20 nucleotides in length, a sense strand 16 nucleotides in length, a duplex between the antisense and sense strand of 16 base pairs, a 3′ overhang of the antisense strand of 2 nucleotides, a 5′ overhang of the antisense strand of 2 nucleotides, wherein the sense strand comprises one to four nucleotides each conjugated to a monovalent GalNAc molecule, the oligonucleotide comprises at least one 2′-modification selected from 2′-aminoethyl, 2′-fluoro, 2′-O-methyl, 2′-O-methoxyethyl, and 2′-deoxy-2′-fluoro-β-d-arabinonucleic acid, and the oligonucleotide comprises at least one internucleotide linkage.

In some embodiments, the ligand-conjugated RNAi oligonucleotide comprises an antisense strand 20 nucleotides in length, a sense strand 16 nucleotides in length, a duplex between the antisense and sense strand of 16 base pairs, a 3′ overhang of the antisense strand of 2 nucleotides, a 5′ overhang of the antisense strand of 2 nucleotides, wherein the antisense strand comprises one to four nucleotides each conjugated to a monovalent GalNAc molecule, the oligonucleotide comprises at least one 2′-modification selected from 2′-aminoethyl, 2′-fluoro, 2′-O-methyl, 2′-O-methoxyethyl, and 2′-deoxy-2′-fluoro-β-d-arabinonucleic acid, and the oligonucleotide comprises at least one internucleotide linkage.

In some embodiments, the ligand-conjugated RNAi oligonucleotide comprises an antisense strand 20 nucleotides in length, a sense strand 16 nucleotides in length, a duplex between the antisense and sense strand of 16 base pairs, a 3′ overhang of the antisense strand of 2 nucleotides, a 5′ overhang of the antisense strand of 2 nucleotides, wherein the sense strand comprises one to four nucleotides each conjugated to a monovalent GalNAc molecule, the oligonucleotide comprises at least one 2′-fluoro modification and at least one 2′-O-methyl modification, and the oligonucleotide comprises at least one internucleotide linkage.

In some embodiments, the ligand-conjugated RNAi oligonucleotide comprises an antisense strand 20 nucleotides in length, a sense strand 16 nucleotides in length, a duplex between the antisense and sense strand of 16 base pairs, a 3′ overhang of the antisense strand of 2 nucleotides, a 5′ overhang of the antisense strand of 2 nucleotides, wherein the antisense strand comprises one to four nucleotides each conjugated to a monovalent GalNAc molecule, the oligonucleotide comprises at least one 2′-fluoro modification and at least one 2′-O-methyl modification, and the oligonucleotide comprises at least one internucleotide linkage.

In some embodiments, the ligand-conjugated RNAi oligonucleotide comprises an antisense strand 22 nucleotides in length, a sense strand 20 nucleotides in length, a duplex between the antisense and sense strand of 20 base pairs, a 3′ overhang of the antisense strand of 2 nucleotides, a 5′ blunt end the antisense strand, wherein the sense strand comprises one to four nucleotides each conjugated to a monovalent GalNAc molecule, and the oligonucleotide comprises at least one 2′-modification.

In some embodiments, the ligand-conjugated RNAi oligonucleotide comprises an antisense strand 22 nucleotides in length, a sense strand 20 nucleotides in length, a duplex between the antisense and sense strand of 20 base pairs, a 3′ overhang of the antisense strand of 2 nucleotides, a 5′ blunt end the antisense strand, wherein the antisense strand comprises one to four nucleotides each conjugated to a monovalent GalNAc molecule, and the oligonucleotide comprises at least one 2′-modification.

In some embodiments, the ligand-conjugated RNAi oligonucleotide comprises an antisense strand 22 nucleotides in length, a sense strand 20 nucleotides in length, a duplex between the antisense and sense strand of 20 base pairs, a 3′ overhang of the antisense strand of 2 nucleotides, a 5′ blunt end the antisense strand, wherein the sense strand comprises one to four nucleotides each conjugated to a monovalent GalNAc molecule, and the oligonucleotide comprises at least one 2′-modification selected from 2′-aminoethyl, 2′-fluoro, 2′-O-methyl, 2′-O-methoxyethyl, and 2′-deoxy-2′-fluoro-β-d-arabinonucleic acid.

In some embodiments, the ligand-conjugated RNAi oligonucleotide comprises an antisense strand 22 nucleotides in length, a sense strand 20 nucleotides in length, a duplex between the antisense and sense strand of 20 base pairs, a 3′ overhang of the antisense strand of 2 nucleotides, a 5′ blunt end the antisense strand, wherein the antisense strand comprises one to four nucleotides each conjugated to a monovalent GalNAc molecule, and the oligonucleotide comprises at least one 2′-modification selected from 2′-aminoethyl, 2′-fluoro, 2′-O-methyl, 2′-O-methoxyethyl, and 2′-deoxy-2′-fluoro-β-d-arabinonucleic acid.

In some embodiments, the ligand-conjugated RNAi oligonucleotide comprises an antisense strand 22 nucleotides in length, a sense strand 20 nucleotides in length, a duplex between the antisense and sense strand of 20 base pairs, a 3′ overhang of the antisense strand of 2 nucleotides, a 5′ blunt end the antisense strand, wherein the sense strand comprises one to four nucleotides each conjugated to a monovalent GalNAc molecule, the oligonucleotide comprises at least one 2′-modification selected from 2′-aminoethyl, 2′-fluoro, 2′-O-methyl, 2′-O-methoxyethyl, and 2′-deoxy-2′-fluoro-β-d-arabinonucleic acid, and the oligonucleotide comprises at least one internucleotide linkage.

In some embodiments, the ligand-conjugated RNAi oligonucleotide comprises an antisense strand 22 nucleotides in length, a sense strand 20 nucleotides in length, a duplex between the antisense and sense strand of 20 base pairs, a 3′ overhang of the antisense strand of 2 nucleotides, a 5′ blunt end the antisense strand, wherein the antisense strand comprises one to four nucleotides each conjugated to a monovalent GalNAc molecule, the oligonucleotide comprises at least one 2′-modification selected from 2′-aminoethyl, 2′-fluoro, 2′-O-methyl, 2′-O-methoxyethyl, and 2′-deoxy-2′-fluoro-β-d-arabinonucleic acid, and the oligonucleotide comprises at least one internucleotide linkage.

In some embodiments, the ligand-conjugated RNAi oligonucleotide comprises an antisense strand 22 nucleotides in length, a sense strand 20 nucleotides in length, a duplex between the antisense and sense strand of 20 base pairs, a 3′ overhang of the antisense strand of 2 nucleotides, a 5′ blunt end the antisense strand, wherein the sense strand comprises one to four nucleotides each conjugated to a monovalent GalNAc molecule, the oligonucleotide comprises at least one 2′-fluoro modification and at least one 2′-O-methyl modification, and the oligonucleotide comprises at least one internucleotide linkage.

In some embodiments, the ligand-conjugated RNAi oligonucleotide comprises an antisense strand 22 nucleotides in length, a sense strand 20 nucleotides in length, a duplex between the antisense and sense strand of 20 base pairs, a 3′ overhang of the antisense strand of 2 nucleotides, a 5′ blunt end the antisense strand, wherein the antisense strand comprises one to four nucleotides each conjugated to a monovalent GalNAc molecule, the oligonucleotide comprises at least one 2′-fluoro modification and at least one 2′-O-methyl modification, and the oligonucleotide comprises at least one internucleotide linkage.

In some embodiments, a ligand-conjugated RNAi oligonucleotide for reducing expression of a target gene comprises the modification pattern of

Sense Strand:
5′-[ademXs-GalNAc][ademX-GalNAc][ademX-GalNAc][mX]
[mX][mX][mX][fX][fX][fX][fX][mX][mX][mX][mX][mX][m
X][mXs][mXs][mX]-3′
Hybridized to:
Antisense Strand:
5′-[MePhosphonate-4O-mXs][fXs][fX][X][fX][mX][fX][
mX][mX][fX][mX][mX][mX][fX][X][mX][mX][mX][mX][mXs
][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, and [ademX-GalNAc]=GalNAc conjugated nucleotide, and [ademXs-GalNAc]=GalNAc attached to a nucleotide with a phosphorothioate linkage to the neighboring nucleotide.

In some embodiments, a ligand-conjugated RNAi oligonucleotide for reducing expression of a target gene comprises the modification pattern of

Sense Strand:
5′-[mXs][mX][X][X][mX][mX][mX][fX][fX][X][fX][X][m
X][mX][mX][ademXs-GalNAc][ademXs-GalNAc][ademX-Gal
NAc]-3′
Hybridized to:
Antisense Strand:
5′-[MePhosphonate-4O-mXs][fXs][fXs][fX][fX][mX][fX
][mX][mX][fX][mX][mX][mX][fX][mX][mX][mX][mX][mX][
mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-GalNAc]=GalNAc conjugated nucleotide, and [ademXs-GalNAc]=GalNAc attached to a nucleotide with a phosphorothioate linkage to the neighboring nucleotide.

In some embodiments, a ligand-conjugated RNAi oligonucleotide for reducing expression of a target gene comprises the modification pattern of

Sense Strand:
5′-[mXs][mX][mX][mX][mX][fX][fX][fX][fX][mX][X][mX
][mX][ademXs-GalNAc][ademXs-GalNAc][ademX-GalNAc]-
3′
Hybridized to:
Antisense Strand:
5′-[MePhosphonate-4O-mXs][fXs][fXs][fX][X][mX][fX]
[mX][mX][fX][mX][mX][mX][fX][mX][mX][mX][mXs][mXs]
[mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-GalNAc]=GalNAc conjugated nucleotide, and [ademXs-GalNAc]=GalNAc attached to a nucleotide with a phosphorothioate linkage to the neighboring nucleotide.

In some embodiments, a ligand-conjugated RNAi oligonucleotide for reducing expression of a target gene comprises the modification pattern of

Sense Strand:
5′-[mXs][mX][mX][mX][mX][mX][mX][fX][fX][fX][X][mX
][mX][mX][mX][mX][X][ademXs-GalNAc][ademXs-GalNAc]
[ademX-GalNAc]-3′
Hybridized to:
Antisense Strand:
5′-[MePhosphonate-4O-mXs][fXs][fXs][fX][fX][mX][fX
][X][mX][fX][mX][mX][mX][fX][mX][mX][mX][mX][mX][m
Xs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-GalNAc]=GalNAc conjugated nucleotide, and [ademXs-GalNAc]=GalNAc attached to a nucleotide with a phosphorothioate linkage to the neighboring nucleotide.

In some embodiments, a ligand-conjugated RNAi oligonucleotide for reducing expression of a target gene comprises the modification pattern of

Sense Strand:
5′-[mXs][mX][mX][mX][X][mX][mX][X][fX][fX][fX][mX]
[mX][mX][mX][X][mX][ademXs-GalNAc][ademXs-GalNAc][
ademX-GalNAc]-3′
Hybridized to:
Antisense Strand:
5′-[MePhosphonate-4O-mXs][fXs][fX][fX][fX][mX][fX]
[mX][mX][fX][mX][mX][mX][fX][mX][mX][X][mX][mX][mX
s][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-GalNAc]=GalNAc conjugated nucleotide, and [ademXs-GalNAc]=GalNAc attached to a nucleotide with a phosphorothioate linkage to the neighboring nucleotide.

In some embodiments, a ligand-conjugated RNAi oligonucleotide for reducing expression of a target gene comprises the modification pattern of

Sense Strand:
5′-[mXs/+Xs][mX][fX][fX][fX][fX][mX][mX][mX][mX][m
X/+X][mX/+X][ademXs-GalNAc][ademXs-GalNAc][ademX-G
alNAc]-3′
Hybridized to:
Antisense Strand:
5′-[MePhosphonate-4O-mXs][fXs][fXs][fX][fX][mX][fX
][mX][mX][fX][mX][mX][X][fX][mX][mX][mX][mX][mX][m
Xs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [+X]=Tm-increasing nucleotide, [mXs/+Xs]=a 2′-O-methyl modified nucleotide with phosphorothioate linkages to neighboring nucleotides or a Tm-increasing nucleotide with phosphorothioate linkage to neighboring nucleotides, [mX/+X]=a 2′-O-methyl modified nucleotide with phosphodiester linkages to neighboring nucleotides or a Tm-increasing nucleotide, [ademX-GalNAc]=GalNAc conjugated nucleotide, and [ademXs-GalNAc]=GalNAc attached to a nucleotide with a phosphorothioate linkage to the neighboring nucleotide.

In some embodiments, a ligand-conjugated RNAi oligonucleotide for reducing expression of a target gene comprises the modification pattern of

Sense Strand:
5′-[mXs/+Xs][mX][fX][fX][fX][fX][mX][mX][mX][mX][m
X/+X][mX/+X][ademXs-GalNAc][ademXs-GalNAc][ademX-G
alNAc]-3′
Hybridized to:
Antisense Strand:
5′-[MePhosphonate-4O-mXs][fXs][fXs][fX][[X][mX][fX
][mX][mX][fX][mX][mX][mX][fX][mX][mXs][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [+X]=Tm-increasing nucleotide, [mXs/+Xs]=a 2′-O-methyl modified nucleotide with phosphorothioate linkages to neighboring nucleotides or a Tm-increasing nucleotide with phosphorothioate linkage to neighboring nucleotides, [mX/+X]=a 2′-O-methyl modified nucleotide with phosphodiester linkages to neighboring nucleotides or a Tm-increasing nucleotide, [ademX-GalNAc]=GalNAc conjugated nucleotide, and [ademXs-GalNAc]=GalNAc attached to a nucleotide with a phosphorothioate linkage to the neighboring nucleotide.

In some embodiments, a ligand-conjugated RNAi oligonucleotide for reducing expression of a target gene comprises the modification pattern of

Sense Strand:
5′-[ademXs-GalNAc][ademX-GalNAc][mX][mX][mX][mX][m
X][fX][fX][fX][fX][mX][mX][mX][mX][mX][mX][mXs][mX
s][mX]-3′
Hybridized to:
Antisense Strand:
5′-[MePhosphonate-4O-mXs][fXs][fXs][fX][fX][mX][fX
][mX][mX][fX][mX][X][mX][fX][mX][mX][mX][mX][mX][a
demXs-GalNAc][mXs][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]-2′-fluoro modified nucleotide with phosphodiester linkages to neighboring nucleotides, [MePhosphonate-4O-mX]=4′-O-monomethylphosphonate-2′-O-methyl modified nucleotide, [ademX-GalNAc]=GalNAc conjugated nucleotide, and [ademXs-GalNAc]=GalNAc attached to a nucleotide with a phosphorothioate linkage to the neighboring nucleotide.

In some embodiments, a ligand-conjugated RNAi oligonucleotide for reducing expression of a target gene comprises the modification pattern of

Sense Strand:
5′-[mXs][mX][mX][mX][mX][X][mX][X][fX][fX][fX][mX]
[X][mX][mX][mX][mX][mXs][mXs][mX]-3′
Hybridized to:
Antisense Strand:
5′-[MePhosphonate-4O-mXs][fXs][fXs][fX][fX][mX][fX
][mX][mX][X][mX][X][mX][fX][mX][mX][X][mX][mX][ade
mXs-GalNAc][ademXs-GalNAc][mX]-3′

wherein [mXs]=2′-O-methyl modified nucleotide with a phosphorothioate linkage to the neighboring nucleotide, [fXs]=2′-fluoro modified nucleotide with a phosphorothioate linkage to the neighboring nucleotide, [mX]=2′-O-methyl modified nucleotide with phosphodiester linkages to neighboring nucleotides, [fX]=2′-fluoro modified nucleotide with phosphodiester linkages to neighboring nucleotides, [MePhosphonate-4O-mX]=4′-O-monomethylphosphonate-2′-O-methyl modified nucleotide, [ademX-GalNAc]=GalNAc conjugated nucleotide, and [ademXs-GalNAc]=GalNAc attached to a nucleotide with a phosphorothioate linkage to the neighboring nucleotide.

In some embodiments, a ligand-conjugated RNAi oligonucleotide for reducing expression of a target gene comprises the modification pattern as set forth in any one of Compounds 1-56 as described herein. In some embodiments, a ligand-conjugated RNAi oligonucleotide for reducing expression of a target gene comprises the modification pattern as set forth in Compound 1 as described herein. In some embodiments, a ligand-conjugated RNAi oligonucleotide for reducing expression of a target gene comprises the modification pattern as set forth in Compound 2 as described herein. In some embodiments, a ligand-conjugated RNAi oligonucleotide for reducing expression of a target gene comprises the modification pattern as set forth in Compound 3 as described herein. In some embodiments, a ligand-conjugated RNAi oligonucleotide for reducing expression of a target gene comprises the modification pattern as set forth in Compound 4 as described herein. In some embodiments, a ligand-conjugated RNAi oligonucleotide for reducing expression of a target gene comprises the modification pattern as set forth in Compound 5 as described herein. In some embodiments, a ligand-conjugated RNAi oligonucleotide for reducing expression of a target gene comprises the modification pattern as set forth in Compound 6 as described herein. In some embodiments, a ligand-conjugated RNAi oligonucleotide for reducing expression of a target gene comprises the modification pattern as set forth in Compound 7 as described herein. In some embodiments, a ligand-conjugated RNAi oligonucleotide for reducing expression of a target gene comprises the modification pattern as set forth in Compound 8 as described herein. In some embodiments, a ligand-conjugated RNAi oligonucleotide for reducing expression of a target gene comprises the modification pattern as set forth in Compound 17 as described herein. In some embodiments, a ligand-conjugated RNAi oligonucleotide for reducing expression of a target gene comprises the modification pattern as set forth in Compound 18 as described herein. In some embodiments, a ligand-conjugated RNAi oligonucleotide for reducing expression of a target gene comprises the modification pattern as set forth in Compound 19 as described herein. In some embodiments, a ligand-conjugated RNAi oligonucleotide for reducing expression of a target gene comprises the modification pattern as set forth in Compound 20 as described herein. In some embodiments, a ligand-conjugated RNAi oligonucleotide for reducing expression of a target gene comprises the modification pattern as set forth in Compound 21 as described herein. In some embodiments, a ligand-conjugated RNAi oligonucleotide for reducing expression of a target gene comprises the modification pattern as set forth in Compound 22 as described herein. In some embodiments, a ligand-conjugated RNAi oligonucleotide for reducing expression of a target gene comprises the modification pattern as set forth in Compound 23 as described herein. In some embodiments, a ligand-conjugated RNAi oligonucleotide for reducing expression of a target gene comprises the modification pattern as set forth in Compound 24 as described herein. In some embodiments, a ligand-conjugated RNAi oligonucleotide for reducing expression of a target gene comprises the modification pattern as set forth in Compound 25 as described herein. In some embodiments, a ligand-conjugated RNAi oligonucleotide for reducing expression of a target gene comprises the modification pattern as set forth in Compound 26 as described herein. In some embodiments, a ligand-conjugated RNAi oligonucleotide for reducing expression of a target gene comprises the modification pattern as set forth in Compound 27 as described herein. In some embodiments, a ligand-conjugated RNAi oligonucleotide for reducing expression of a target gene comprises the modification pattern as set forth in Compound 28 as described herein. In some embodiments, a ligand-conjugated RNAi oligonucleotide for reducing expression of a target gene comprises the modification pattern as set forth in Compound 29 as described herein. In some embodiments, a ligand-conjugated RNAi oligonucleotide for reducing expression of a target gene comprises the modification pattern as set forth in Compound 30 as described herein. In some embodiments, a ligand-conjugated RNAi oligonucleotide for reducing expression of a target gene comprises the modification pattern as set forth in Compound 31 as described herein. In some embodiments, a ligand-conjugated RNAi oligonucleotide for reducing expression of a target gene comprises the modification pattern as set forth in Compound 32 as described herein. In some embodiments, a ligand-conjugated RNAi oligonucleotide for reducing expression of a target gene comprises the modification pattern as set forth in Compound 33 as described herein. In some embodiments, a ligand-conjugated RNAi oligonucleotide for reducing expression of a target gene comprises the modification pattern as set forth in Compound 34 as described herein. In some embodiments, a ligand-conjugated RNAi oligonucleotide for reducing expression of a target gene comprises the modification pattern as set forth in Compound 35 as described herein. In some embodiments, a ligand-conjugated RNAi oligonucleotide for reducing expression of a target gene comprises the modification pattern as set forth in Compound 36 as described herein. In some embodiments, a ligand-conjugated RNAi oligonucleotide for reducing expression of a target gene comprises the modification pattern as set forth in Compound 37 as described herein. In some embodiments, a ligand-conjugated RNAi oligonucleotide for reducing expression of a target gene comprises the modification pattern as set forth in Compound 38 as described herein. In some embodiments, a ligand-conjugated RNAi oligonucleotide for reducing expression of a target gene comprises the modification pattern as set forth in Compound 43 as described herein. In some embodiments, a ligand-conjugated RNAi oligonucleotide for reducing expression of a target gene comprises the modification pattern as set forth in Compound 44 as described herein. In some embodiments, a ligand-conjugated RNAi oligonucleotide for reducing expression of a target gene comprises the modification pattern as set forth in Compound 45 as described herein. In some embodiments, a ligand-conjugated RNAi oligonucleotide for reducing expression of a target gene comprises the modification pattern as set forth in Compound 46 as described herein. In some embodiments, a ligand-conjugated RNAi oligonucleotide for reducing expression of a target gene comprises the modification pattern as set forth in Compound 47 as described herein. In some embodiments, a ligand-conjugated RNAi oligonucleotide for reducing expression of a target gene comprises the modification pattern as set forth in Compound 48 as described herein. In some embodiments, a ligand-conjugated RNAi oligonucleotide for reducing expression of a target gene comprises the modification pattern as set forth in Compound 49 as described herein. In some embodiments, a ligand-conjugated RNAi oligonucleotide for reducing expression of a target gene comprises the modification pattern as set forth in Compound 57 as described herein. In some or any of the foregoing embodiments, reference to Compound numbers refers to the modification pattern (e.g., phosphorothioate linkages, 2′ modifications, conjugation) and not the nucleotide sequences.

### Formulations

Various formulations have been developed to facilitate oligonucleotide use. For example, oligonucleotides (e.g., ligand-conjugated RNAi oligonucleotides) can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., ligand-conjugated RNAi oligonucleotides) reduce the expression of a target mRNA (e.g., a target liver mRNA). In some embodiments, provided herein are compositions comprising oligonucleotides (e.g., ligand-conjugated RNAi oligonucleotides) reduce the expression of a target mRNA expressed in one or more tissues or cells of a subject. Such compositions can be suitably formulated such that when administered to a subject, either into the immediate environment of a target cell or systemically, a sufficient portion of the oligonucleotides enter the cell to reduce target gene expression. Any variety of suitable oligonucleotide formulations can be used to deliver oligonucleotides for the reduction of target gene expression as disclosed herein. In some embodiments, an oligonucleotide is formulated in buffer solutions such as phosphate buffered saline solutions, liposomes, micellar structures, and capsids.

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol or polyvinylpyrrolidone) or a collapse temperature modifier (e.g., dextran, Ficoll™ or gelatin). Likewise, the oligonucleotides herein may be provided in the form of their free acids.

In some embodiments, a pharmaceutical composition is formulated to be compatible with its intended route of administration. Examples of routes of administration include parenteral (e.g., intravenous, intramuscular, intraperitoneal, intradermal, subcutaneous, intrathecal), oral (e.g., inhalation), transdermal (e.g., topical), transmucosal and rectal administration. In some embodiments, a pharmaceutical composition is formulated for delivery to the eye (e.g., ophthalmic, intraocular, subconjunctival, intravitreal, retrobulbar, intracameral).

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and liquid polyethylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., an ligand-conjugated RNAi oligonucleotide herein) or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Structural Modifications

As nucleic acids are polymers of subunits or compounds, many of the modifications described below occur at a position which is repeated within a nucleic acid (e.g., a modification of a base, or a phosphate moiety, or the non-bridging oxygen of a phosphate moiety). In some cases the modification will occur at all of the subject positions in the nucleic acid but in many, and in fact in most cases it will not. By way of example, a modification may only occur at a 3′ or 5′ terminal position, may only occur in the internal unpaired region, may only occur in a terminal regions, e.g. at a position on a terminal nucleotide or in the last 2, 3, 4, 5, or 10 nucleotides of a strand. In some embodiments, a modification occurs at all of the subject positions in the nucleic acid. A modification may occur in a double strand region, a single strand region, or in both. A modification may occur only in the double strand region of an RNA agent or may only occur in a single strand region of an RNA agent. (e.g., a phosphorothioate modification at a non-bridging oxygen position may only occur at one or both termini, may only occur in a terminal regions or at a position on a terminal nucleotide or in the last 2, 3, 4, 5, or 10 nucleotides of a strand, or may occur in double strand and single strand regions, particularly at termini. The 5′ end or ends can be phosphorylated.

Many studies in the art have indicated that modified oligonucleotides and oligonucleotide analogs may be less readily internalized than their natural counterparts. As a result, the activity of many previously available RNAi trigger molecules has not been sufficient for practical therapeutic, research or diagnostic purposes.

Modifications to enhance the effectiveness of the RNAi trigger molecule oligonucleotides and overcome these problems have taken many forms. These modifications include base ring modifications, sugar moiety modifications, and sugar-phosphate backbone modifications, many exemplified herein and used in the current disclosure. Prior sugar-phosphate backbone modifications, particularly on the phosphorus atom, have affected various levels of resistance to nucleases. However, while the ability of an RNAi trigger molecule oligonucleotide to load into the RISC and direct the location of relevant mRNA sequences is fundamental to RNAi trigger molecule methodology, many modifications work at cross purposes with each other to optimize the behavior of the RNAi trigger. It is this balancing act which must be taken into account relative to the development of superior and effective RNAi molecules.

Another key factor is the stereochemical effect that arises in oligomers having P-chiral centers. In general, an oligomer with a length of n nucleosides will constitute a mixture of chirality in successive non-stereospecific chain synthesis. It has been observed that Rp and Sp homochiral chains, whose absolute configuration at all internucleotide methane phosphonate phosphorus atoms are either Rp or Sp, and non-stereoregular chains show different physicochemical properties as well as different capabilities of forming adducts with oligonucleotides of complementary sequence. In addition, phosphorothioate analogs of nucleotides have shown substantial stereoselectivity differences between Oligo-Rp and Oligo-Sp oligonucleotides in resistance to nucleases activity (Potter, BIOCHEMISTRY, 22:1369, (1983); Bryant et al., BIOCHEMISTRY, 18:2825, (1979)). Lesnikowski (NUCL. ACIDS RES., 18:2109, (1990)) observed that diastereomerically pure octathymidine methylphosphonates, in which six out of seven methylphosphonate bonds have defined configuration at the phosphorus atom when complexed with the matrix showed substantial differences in melting temperatures. According to the current disclosure chirally pure nucleotide analogs, or portions thereof, are expected to provide trigger structures with improved characteristics allowing the development of more potent and longer lasting RNAi triggers.

In some embodiments of the current disclosure it is particularly preferred to enhance stability, to include particular bases in overhangs, or to include modified nucleotides or nucleotide surrogates, in single strand overhangs, e.g., in a 5′ or 3′ overhang, or in both. Likewise, it can be desirable to include purine nucleotides in overhangs as they are more resistant to nuclease activity. In some embodiments all or some of the bases in a 3′ or 5′ overhang will be modified, with a modification described herein. Modifications can include the use of modifications at the 2′ OH group of the ribose sugar, deoxythymidine, instead of ribonucleotides, and modifications in the phosphate group, that is, phosphothioate modifications. Overhangs need not be homologous with the target sequence.

### Methods of Use

### Reducing Target Gene Expression

In some embodiments, the disclosure provides methods for contacting or delivering to a cell or population of cells an effective amount of any of the ligand-conjugated RNAi oligonucleotides herein to reduce expression of a target gene.

In some embodiments, expression of a target gene is reduced in one or more tissues or cells in a subject. In some embodiments, expression of a target gene is reduced in the liver.

In some embodiments, a reduction of target gene expression is determined by measuring a reduction in the amount or level of target mRNA, protein encoded by the target mRNA, or target gene (mRNA or protein) activity in a cell. The methods include those described herein and known to one of ordinary skill in the art.

Methods provided herein are useful in any appropriate cell type. In some embodiments, a cell is any cell that expresses the target mRNA. In some embodiments, the cell is a primary cell obtained from a subject. In some embodiments, the primary cell has undergone a limited number of passages such that the cell substantially maintains is natural phenotypic properties. In some embodiments, a cell to which the oligonucleotide is delivered is ex vivo or in vitro (i.e., can be delivered to a cell in culture or to an organism in which the cell resides).

In some embodiments, the ligand-conjugated RNAi oligonucleotides disclosed herein are delivered to a cell or population of cells using a nucleic acid delivery method known in the art including, but not limited to, injection of a solution or pharmaceutical composition containing the ligand-conjugated RNAi oligonucleotide, bombardment by particles covered by the ligand-conjugated RNAi oligonucleotide, exposing the cell or population of cells to a solution containing the ligand-conjugated RNAi oligonucleotide, or electroporation of cell membranes in the presence of the ligand-conjugated RNAi oligonucleotide. Other methods known in the art for delivering oligonucleotides to cells may be used, such as lipid-mediated carrier transport, chemical-mediated transport, and cationic liposome transfection such as calcium phosphate, and others.

In some embodiments, reduction of target gene expression is determined by an assay or technique that evaluates one or more molecules, properties or characteristics of a cell or population of cells associated with target gene expression, or by an assay or technique that evaluates molecules that are directly indicative of target gene expression in a cell or population of cells (e.g., target mRNA or protein). In some embodiments, the extent to which a ligand-conjugated RNAi oligonucleotide provided herein reduces target gene expression in a cell is evaluated by comparing target gene expression in a cell or population of cells contacted with the ligand-conjugated RNAi oligonucleotide to a control cell or population of cells (e.g., a cell or population of cells not contacted with the ligand-conjugated RNAi oligonucleotide or contacted with a control ligand-conjugated RNAi oligonucleotide). In some embodiments, a control amount or level of target gene expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering a ligand-conjugated RNAi oligonucleotide described herein to a cell or a population of cells results in a reduction in expression of a target gene. In some embodiments, the reduction in target gene expression is relative to a control amount or level of target gene expression in cell or population of cells not contacted with the ligand-conjugated RNAi oligonucleotide or contacted with a control ligand-conjugated RNAi oligonucleotide. In some embodiments, the reduction in target gene expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of target gene expression. In some embodiments, the control amount or level of target gene expression is an amount or level of target mRNA and/or protein in a cell or population of cells that has not been contacted with a ligand-conjugated RNAi oligonucleotide herein. In some embodiments, the effect of delivery of a ligand-conjugated RNAi oligonucleotide to a cell or population of cells according to a method herein is assessed after any finite period or amount of time (e.g., minutes, hours, days, weeks, months). For example, in some embodiments, target gene expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the ligand-conjugated RNAi oligonucleotide to the cell or population of cells. In some embodiments, target gene expression is determined in a cell or population of cells at least about 1 month, about 2 months, about 3 months, about 4 months, about 5 months, or about 6 months or more after contacting or delivering the ligand-conjugated RNAi oligonucleotide to the cell or population of cells.

### Treatment Methods

In some embodiments, the disclosure provides methods for treating a disease, disorder, or condition associated with expression of a target gene in one or more tissues or cells. In some embodiments, the disclosure provides methods for treating a disease, disorder, or condition associated with expression of a target gene in the liver.

Methods described herein are typically involve administering to a subject a therapeutically effective amount of a ligand-conjugated RNAi oligonucleotide herein, that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the brain of a subject).

In some embodiments, a ligand-conjugated RNAi oligonucleotide herein, or a composition thereof, is administered once every year, once every 6 months, once every 4 months, quarterly (once every three months), bi-monthly (once every two months), monthly or weekly. In some embodiments, a ligand-conjugated RNAi oligonucleotide herein, or a composition thereof, is administered every week or at intervals of two, or three weeks. In some embodiments, a ligand-conjugated RNAi oligonucleotide herein, or a composition thereof, is administered daily. In some embodiments, a subject is administered one or more loading doses of a ligand-conjugated RNAi oligonucleotide herein, or a composition thereof, followed by one or more maintenance doses of the ligand-conjugated RNAi oligonucleotide, or a composition thereof.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### Treatment Methods in the Liver

The disclosure provides oligonucleotides for use as a medicament, in particular for use in a method for the treatment of diseases, disorders, and conditions associated with the liver. The disclosure also provides oligonucleotides for use, or adaptable for use, to treat a subject (e.g., a human) having a disease, disorder or condition associated with expression of a liver target gene that would benefit from reducing expression of the liver target gene. In some embodiments, the disclosure provides oligonucleotides for use, or adapted for use, to treat a subject having a disease, disorder or condition associated with liver target gene expression. The disclosure also provides oligonucleotides for use, or adaptable for use, in the manufacture of a medicament or pharmaceutical composition for treating a disease, disorder or condition associated with expression of a liver target gene. In some embodiments, the oligonucleotides for use, or adaptable for use, target mRNA and reduce expression of a liver target gene (e.g., via the RNAi pathway). In some embodiments, the oligonucleotides for use, or adaptable for use, target mRNA and reduce the amount or level of liver target mRNA, protein and/or activity.

In addition, in some embodiments of the methods herein, a subject having a disease, disorder or condition associated with expression of a liver target gene or is predisposed to the same is selected for treatment with an oligonucleotide herein. In some embodiments, the method comprises selecting an individual having a marker (e.g., a biomarker) for a disease, disorder or condition associated with expression of a liver target gene, or predisposed to the same, such as, but not limited to, target mRNA, protein, or a combination thereof. Likewise, and as detailed below, some embodiments of the methods provided by the disclosure include steps such as measuring or obtaining a baseline value for a marker of expression of a liver target gene, and then comparing such obtained value to one or more other baseline values or values obtained after the subject is administered the oligonucleotide to assess the effectiveness of treatment.

The disclosure also provides methods of treating a subject having, suspected of having, or at risk of developing a disease, disorder or condition associated with expression of a liver target gene with an oligonucleotide provided herein. In some embodiments, the disclosure provides methods of treating or attenuating the onset or progression of a disease, disorder or condition associated with expression of a liver target gene using the oligonucleotides provided herein. In some embodiments, the disclosure provides methods to achieve one or more therapeutic benefits in a subject having a disease, disorder or condition associated with expression of a liver target gene using the oligonucleotides provided herein.

In some embodiments of the methods herein, the subject is treated by administering a therapeutically effective amount of any one or more of the oligonucleotides provided herein. In some embodiments, treatment comprises reducing expression of a liver target gene. In some embodiments, the subject is treated therapeutically. In some embodiments, the subject is treated prophylactically.

In some embodiments of the methods herein, an oligonucleotide provided herein, or a pharmaceutical composition comprising the oligonucleotide, is administered to a subject having a disease, disorder or condition associated with expression of a liver target gene such that target gene expression is reduced in the subject, thereby treating the subject. In some embodiments, an amount or level of target mRNA is reduced in the subject. In some embodiments, an amount or level of protein encoded by the target mRNA is reduced in the subject.

In some embodiments of the methods herein, an oligonucleotide provided herein, or a pharmaceutical composition comprising the oligonucleotide, is administered to a subject having a disease, disorder or condition associated with expression of a liver target gene such that target gene expression is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression prior to administration of the oligonucleotide or pharmaceutical composition. In some embodiments, expression of a liver target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to target gene expression in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of a liver target gene such that an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target mRNA prior to administration of the oligonucleotide or pharmaceutical composition. In some embodiments, an amount or level of target mRNA is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target mRNA in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of a liver target gene such that an amount or level of protein encoded by the liver target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of protein encoded by the target gene prior to administration of the oligonucleotide or pharmaceutical composition. In some embodiments, an amount or level of protein encoded by a liver target gene is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of protein encoded by the target gene in a subject (e.g., a reference or control subject) not receiving the oligonucleotide or pharmaceutical composition or receiving a control oligonucleotide, pharmaceutical composition or treatment.

In some embodiments of the methods herein, an oligonucleotide herein, or a pharmaceutical composition comprising the oligonucleotide, is administered to a subject having a disease, disorder or condition associated with expression of a liver target gene such that an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to the amount or level of target gene activity prior to administration of the oligonucleotide or pharmaceutical composition. In some embodiments, an amount or level of target gene activity is reduced in the subject by at least about 30%, about 35%, about 40%, about 45%, about 50%, about 55%, about 60%, about 65%, about 70%, about 75%, about 80%, about 85%, about 90%, about 95%, about 99% or greater than 99% when compared to an amount or level of target gene activity in a subject (e.g., a reference or control subject) not receiving the oligonucleotide or pharmaceutical composition or receiving a control oligonucleotide, pharmaceutical composition or treatment.

Suitable methods for determining target gene expression, an amount or level of target mRNA, an amount or level of protein encoded by the target gene, and/or an amount or level of target gene activity, in the subject, or in a sample from the subject, are known in the art. Further, the Examples set forth herein illustrate exemplary methods for determining target gene expression.

In some embodiments, target gene expression, an amount or level of target gene mRNA, an amount or level of protein encoded by a target gene, an amount or level of target gene activity, or any combination thereof, is reduced in a cell, a population or a group of cells (e.g., an organoid), an organ (e.g., liver), blood or a fraction thereof (e.g., plasma), a tissue (e.g., liver tissue), a sample (e.g., liver biopsy sample), or any other biological material obtained or isolated from the subject. In some embodiments, expression of a liver target gene, an amount or level of target gene mRNA, an amount or level of protein encoded by the target gene, an amount or level of target gene activity, or any combination thereof, is reduced in more than one type of cell, more than one groups of cells, more than one organ (e.g., liver and one or more other organ(s)), more than one fraction of blood (e.g., plasma and one or more other blood fraction(s)), more than one type of tissue (e.g., liver tissue and one or more other type(s) of tissue), more than one type of sample (e.g., a liver biopsy sample and one or more other type(s) of biopsy sample) obtained or isolated from the subject.

In some embodiments, the liver target gene may be a target gene from any mammal, such as a human. Any liver gene may be silenced according to the method described herein.

In some embodiments, an oligonucleotide herein, or a composition thereof, is administered via subcutaneous or intravenous administration.

### Kits

In some embodiments, the disclosure provides a kit comprising a ligand-conjugated RNAi oligonucleotide herein, or a composition thereof, described herein, and instructions for use. In some embodiments, the kit comprises a ligand-conjugated RNAi oligonucleotide herein, or a composition thereof, described herein, and a package insert containing instructions for use of the kit and/or any component thereof. In some embodiments, the kit comprises, in a suitable container, a ligand-conjugated RNAi oligonucleotide herein, or a composition thereof, described herein, one or more controls, and various buffers, reagents, enzymes and other standard ingredients well known in the art. In some embodiments, the container comprises at least one vial, well, test tube, flask, bottle, syringe, or other container means, into which the ligand-conjugated RNAi oligonucleotide herein, or a composition thereof, is placed, and in some instances, suitably aliquoted. In some embodiments where an additional component is provided, the kit contains additional containers into which this component is placed. The kits can also include a means for containing a ligand-conjugated RNAi oligonucleotide herein, or a composition thereof, and any other reagent in close confinement for commercial sale. Such containers may include injection or blow-molded plastic containers into which the desired vials are retained. Containers and/or kits can include labeling with instructions for use and/or warnings.

In some embodiments, a kit comprises a ligand-conjugated RNAi oligonucleotide herein, or a composition thereof, described herein, and a pharmaceutically acceptable carrier, or a pharmaceutical composition comprising the ligand-conjugated RNAi oligonucleotide and instructions for treating or delaying progression of a disease, disorder or condition associated with expression of a target gene expressed in one or more tissues or cells in a subject in need thereof.

In some embodiments, a kit comprises a ligand-conjugated RNAi oligonucleotide herein, or a composition thereof, described herein, and a pharmaceutically acceptable carrier, or a pharmaceutical composition comprising the ligand-conjugated RNAi oligonucleotide and instructions for treating or delaying progression of a disease, disorder or condition associated with expression of a target gene expressed in liver tissue in a subject in need thereof.

### Definitions

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, exemplary methods, and materials are described herein.

General texts which describe molecular biological techniques useful herein, including the use of vectors, promoters and many other relevant topics, include Berger and Kimmel, GUIDE TO MOLECULAR CLONING TECHNIQUES, METHODS IN ENZYMOLOGY, volume 152, (Academic Press, Inc., San Diego, Calif.) (“Berger”); Sambrook et al., MOLECULAR CLONING—A LABORATORY MANUAL, 2d ed., Vol. 1-3, Cold Spring Harbor Laboratory, Cold Spring Harbor, 1989 (“Sambrook”) and CURRENT PROTOCOLS IN MOLECULAR BIOLOGY, F. M. Ausubel et al., eds., CURRENT PROTOCOLS, A JOINT VENTURE BETWEEN GREENE PUBLISHING ASSOCIATES, INC. AND JOHN WILEY AND SONS, INC., (supplemented through 1999) (“Ausubel”). Examples of protocols sufficient to direct persons of skill through in vitro amplification methods, including the polymerase chain reaction (PCR), the ligase chain reaction (LCR), Q.beta.-replicase amplification and other RNA polymerase mediated techniques (e.g., NASBA), e.g., for the production of the homologous nucleic acids of the disclosure are found in Berger, Sambrook, and Ausubel, as well as in Mullis et al., (1987) U.S. Pat. No. 4,683,202; Innis et al., eds. (1990); PCR PROTOCOLS: A GUIDE TO METHODS AND APPLICATIONS (Academic Press Inc. San Diego, Calif.) (“Innis”); Arnheim and Levinson (Oct. 1, 1990) CandEN 36-47; J. NIH RES. (1991) 3:81-94; Kwoh et al., (1989) PROC. NATL. ACAD. SCI. USA 86:1173; Guatelli et al (1990) PROC. NAT'L. ACAD. SCI. USA 87:1874; Lomell et al., (1989) J. CLIN. CHEM 35:1826; Landegren et al., (1988) SCIENCE 241:1077-80; Van Brunt (1990) BIOTECHNOLOGY 8:291-94; Wu and Wallace (1989) GENE 4:560; Barringer et al., (1990) GENE 89:117; and, Sooknanan and Malek (1995) BIOTECHNOLOGY 13:563-564. Improved methods for cloning in vitro amplified nucleic acids are described in Wallace et al., U.S. Pat. No. 5,426,039. Improved methods for amplifying large nucleic acids by PCR are summarized in Cheng et al., (1994) NATURE 369:684-85 and the references cited therein, in which PCR amplicons of up to 40 kb are generated.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one value, and/or to “about” another value. When such a range is expressed, another embodiment includes from the one value and/or to the other value. Similarly, when values are expressed as approximations, by use of the antecedent “about,” it will be understood that the value forms another embodiment. It will be further understood that the endpoints of each of the ranges are significant both in relation to the other endpoint, and independently of the other endpoint. It is also understood that there are several values disclosed herein, and that each value is also herein disclosed as “about” that value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in several different formats, and that this data, represents endpoints and starting points, and ranges for any combination of the data points. For example, if a particular datapoint “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used here, the term “amount” refers to an absolute amount (e.g., an absolute amount of mRNA or protein), a relative amount (e.g., a relative amount of target mRNA as measured by PCR assay or protein), or a concentration (e.g. a concentration of ligand-conjugated RNA in a composition), whether the amount referred to in a given instance refers to an absolute amount, concentration, or both, will be clear to the skilled artisan based on the context provided herein.

As used herein, “bicyclic nucleotide” refers to a nucleotide comprising a bicyclic sugar moiety.

As used herein “bicyclic sugar moiety” refers to a modified sugar moiety comprising a 4 to 7 membered ring (including but not limited to a furanosyl) comprising a bridge connecting two atoms of the 4 to 7 membered ring to form a second ring, resulting in a bicyclic structure. Typically, the 4 to 7 membered ring is a sugar. In some embodiments, the 4-to-7-member ring is a furanosyl. In certain embodiments, the bridge connects the 2′-carbon and the 4′-carbon of the furanosyl.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded RNA” or “dsRNA” refers to an RNA oligonucleotide that is substantially in a duplex form. In some embodiments, the complementary base-pairing of duplex region(s) of a dsRNA oligonucleotide is formed between antiparallel sequences of nucleotides of covalently separate nucleic acid strands. In some embodiments, complementary base-pairing of duplex region(s) of a dsRNA formed between antiparallel sequences of nucleotides of nucleic acid strands that are covalently linked. In some embodiments, complementary base-pairing of duplex region(s) of a dsRNA is formed from single nucleic acid strand that is folded (e.g., via a hairpin) to provide complementary antiparallel sequences of nucleotides that base pair together. In some embodiments, a dsRNA comprises two covalently separate nucleic acid strands that are fully duplexed with one another. However, in some embodiments, a dsRNA comprises two covalently separate nucleic acid strands that are partially duplexed (e.g., having overhangs at one or both ends). In some embodiments, a dsRNA comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect. As used herein, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

As used herein, “melting temperature” or “Tm” means the temperature at which the two strands of a duplex nucleic acid separate. Tₘ is often used as a measure of duplex stability or the binding affinity of two strands of complementary nucleic acids or portions thereof. Tm can be measured by using the UV spectrum to determine the formation and breakdown (melting) of hybridization. Base stacking, which occurs during hybridization, is accompanied by a reduction in UV absorption (hypochromicity). Consequently, a reduction in UV absorption indicates a higher Tₘ.

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “oligonucleotide” refers to a short nucleic acid (e.g., less than about 100 nucleotides in length). An oligonucleotide may be single stranded (ss) or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA or ss siRNA. In some embodiments, a double-stranded (dsRNA) is an RNAi oligonucleotide.

The terms “ligand-conjugated RNAi oligonucleotide” and “oligonucleotide-ligand conjugate” are used interchangeably and refer to an oligonucleotide comprising one or more nucleotides conjugated with one or more targeting ligands (e.g., GalNAc).

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a dsRNA. In some embodiments, the overhang is a 3′ or 5′ overhang on the antisense strand or sense strand of a dsRNA.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include, but are not limited to, 5′ phosphonates, such as 5′ methylene phosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethyl phosphonate, in which the oxygen atom of the oxymethyl group is bound to the sugar moiety (e.g., at its 4′-carbon) or analog thereof. See, e.g., U.S. Provisional Patent Application Nos. 62/383,207 (filed on 2 Sep. 2016) and 62/393,401 (filed on 12 Sep. 2016). Other modifications have been developed for the 5′ end of oligonucleotides (see, e.g., Intl. Patent Application No. WO 2011/133871; U.S. Pat. No. 8,927,513; and Prakash et al., (2015) NUCLEIC ACIDS RES. 43:2993-3011).

As used herein, “reduced expression” of a target gene refers to a decrease in the amount or level of RNA transcript (e.g., target mRNA) or protein encoded by the target gene and/or a decrease in the amount or level of activity of the gene in a cell, a population of cells, a sample, or a subject, when compared to an appropriate reference (e.g., a reference cell, population of cells, sample, or subject). For example, the act of contacting a cell with an oligonucleotide or conjugate herein (e.g., an ligand-conjugated RNAi oligonucleotide comprising an antisense strand having a nucleotide sequence that is complementary to a nucleotide sequence comprising a target mRNA) may result in a decrease in the amount or level of target mRNA, protein encoded by a target gene, and/or target gene activity (e.g., via inactivation and/or degradation of target mRNA by the RNAi pathway) when compared to a cell that is not treated with the double-stranded oligonucleotide. Similarly, and as used herein, “reducing expression” refers to an act that results in reduced expression of a target gene.

As used herein, “region of complementarity” refers to a sequence of nucleotides of a nucleic acid (e.g., a dsRNA) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base. As used herein, “RNAi oligonucleotide” refers to either (a) a dsRNA having a sense strand (passenger) and antisense strand (guide), in which the antisense strand or part of the antisense strand is used by the Argonaute 2 (Ago2) endonuclease in the cleavage of a target mRNA or (b) a ss oligonucleotide having a single antisense strand, where that antisense strand (or part of that antisense strand) is used by the Ago2 endonuclease in the cleavage of a target mRNA. As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits, and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule or “moiety” (e.g., a carbohydrate, amino sugar, cholesterol, polypeptide, or lipid) that selectively binds to a cognate molecule (e.g., a receptor) of a tissue or cell of interest and/or that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “Tm-increasing nucleotide” refers to a nucleotide that increases the melting temperature (Tₘ) of an oligonucleotide duplex as compared to the oligonucleotide duplex without the Tₘ-increasing nucleotide. Tₘ-increasing nucleotides include, but are not limited to, bicyclic nucleotides, tricyclic nucleotides, a G-clamp, and analogues thereof, and hexitol nucleotides. Certain modified nucleotides having a modified sugar moiety, or a modified nucleobase can also be used to increase the Tₘ of an oligonucleotide duplex. As used herein, the term “Tₘ-increasing nucleotide” specifically excludes nucleotides modified at the 2′-position of the sugar moiety with 2′-OMe or 2′-F.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### Embodiments

Embodiment 1. A double-stranded oligonucleotide comprising an antisense strand of about 18-22 nucleotides in length and a sense strand of about 13-20 nucleotides in length, wherein the antisense and sense strands form a duplex region of about 13-20 base pairs, wherein the antisense strand comprises a 3′ overhang of at least one nucleotide, wherein the antisense strand comprises a region of complementarity to a liver mRNA target sequence, and wherein the sense strand and/or antisense strand comprise at least one nucleotide conjugated to a GalNAc molecule.
Embodiment 2. The double-stranded oligonucleotide of Embodiment 1, wherein the 3′ overhang is 1-7 nucleotides.
Embodiment 3. The double-stranded oligonucleotide of Embodiment 1, wherein the 3′ overhang is 3-7 nucleotides.
Embodiment 4. The double-stranded oligonucleotide of any one of Embodiments 1-3, comprising a blunt end.
Embodiment 5. The double-stranded oligonucleotide of Embodiment 4, wherein the blunt end comprises the 3′ end of the sense strand and the 5′ end of the antisense strand.
Embodiment 6. The double-stranded oligonucleotide of any one of Embodiments 1-3, wherein the antisense strand comprises a 5′ overhang of at least one nucleotide.
Embodiment 7. The double-stranded oligonucleotide of Embodiment 6, wherein the 3′ overhang is 2 nucleotides and the 5′ overhang is 2 nucleotides.
Embodiment 8. The double-stranded oligonucleotide of any one of Embodiments 1-7, wherein:
(i) the antisense strand is 22 nucleotides, the sense strand is 20 nucleotides, the duplex region is 20 base pairs, and the 3′ overhang is two nucleotides;
(ii) the antisense strand is 22 nucleotides, the sense strand is 18 nucleotides, the duplex region is 18 base pairs, and the 3′ overhang is two nucleotides;
(iii) the antisense strand is 22 nucleotides, the sense strand is 15 nucleotides, the duplex region is 15 base pairs, and the 3′ overhang is 7 nucleotides;
(iv) the antisense strand is 22 nucleotides, the sense strand is 13 nucleotides, the duplex region is 13 base pairs, and the 3′ overhang is 7 nucleotides;
(v) the antisense strand is 22 nucleotides, the sense strand is 16 nucleotides, the duplex region is 16 base pairs, and the 3′ overhang is 4 nucleotides; or
(vi) the antisense strand is 22 nucleotides, the sense strand is 17 nucleotides, the duplex region is 17 base pairs, and the 3′ overhang is 4 nucleotides.
   Embodiment 9. The double-stranded oligonucleotide of Embodiment 8, wherein the three 5′ terminal nucleotides of the sense strand are each conjugated to a GalNAc molecule.
   Embodiment 10. The double-stranded oligonucleotide of Embodiment 8, wherein the three 3′ terminal nucleotides of the sense strand are each conjugated to a GalNAc molecule.
   Embodiment 11. The double-stranded oligonucleotide of Embodiment 8, wherein the two 3′ terminal nucleotides of the sense strand are each conjugated to a GalNAc molecule.
   Embodiment 12. The double-stranded oligonucleotide of Embodiment 8, wherein two to four, or three consecutive nucleotides of the sense strand are each conjugated to a GalNAc molecule.
   Embodiment 13. The double-stranded oligonucleotide of Embodiment 12, wherein the three consecutive nucleotides of the sense strand are selected from (i) nucleotides at positions 4, 5 and 6; (ii) nucleotides at positions 9, 10 and 11; (iii) nucleotides at positions 12, 13 and 15; and (iv) nucleotides at positions 15, 16 and 17.
   Embodiment 14. The double-stranded oligonucleotide of Embodiment 12, wherein the three consecutive nucleotides of the sense strand are selected from (i) nucleotides at positions 2, 3 and 4; (ii) nucleotides at positions 5, 6 and 7; and (iii) nucleotides at positions 7, 8 and 9.
   Embodiment 15. The double-stranded oligonucleotide of Embodiment 8, wherein the two 5′ terminal nucleotides of the sense strand are each conjugated to a GalNAc molecule.
   Embodiment 16. The double-stranded oligonucleotide of Embodiment 8, wherein two consecutive nucleotides of the sense strand are each conjugated to a GalNAc molecule.
   Embodiment 17. The double-stranded oligonucleotide of Embodiment 12, wherein the two consecutive nucleotides of the sense strand are selected from (i) nucleotides at positions 8 and 9; and (ii) nucleotides at positions 12 and 13.
   Embodiment 18. The double-stranded oligonucleotide of Embodiment 8, wherein two to four consecutive nucleotides of the antisense strand are each conjugated to a GalNAc molecule.
   Embodiment 19. The double-stranded oligonucleotide of Embodiment 8, wherein nucleotides at positions 20 and 21 of the antisense strand are each conjugated to a GalNAc molecule.
   Embodiment 20. The double-stranded oligonucleotide of Embodiment 8, wherein the 5′ terminal nucleotide of the sense strand is conjugated to a GalNAc molecule, and wherein two consecutive nucleotides of the antisense strand are each conjugated to a GalNAc molecule.
   Embodiment 21. The double-stranded oligonucleotide of Embodiment 8, wherein the two 5′ terminal nucleotides of the sense strand are each conjugated to a GalNAc molecule, and wherein a nucleotide of the antisense strand is conjugated to a GalNAc molecule.
   Embodiment 22. The double-stranded oligonucleotide of Embodiment 21, wherein the nucleotide of the antisense strand conjugated to the GalNAc molecule is the nucleotide at position 20 of the antisense strand.
   Embodiment 23. The double-stranded oligonucleotide of Embodiment 8, wherein the two 5′ terminal nucleotides of the sense strand are each conjugated to a GalNAc molecule, and wherein positions 19 and 20 of the antisense strand are each conjugated to a GalNAc molecule.
   Embodiment 24. The double-stranded oligonucleotide of any one of Embodiments 1-7, wherein:
(i) the antisense strand is 21 nucleotides, the sense strand is 15 nucleotides, the duplex region is 15 base pairs, and the 3′ overhang is 6 nucleotides;
(ii) the antisense strand is 20 nucleotides, the sense strand is 15 nucleotides, the duplex region is 15 base pairs, and the 3′ overhang is 5 nucleotides;
(iii) the antisense strand is 19 nucleotides, the sense strand is 15 nucleotides, the duplex region is 15 base pairs, and the 3′ overhang is 4 nucleotides; or
(iv) the antisense strand is 18 nucleotides, the sense strand is 15 nucleotides, the duplex region is 15 base pairs, and the 3′ overhang is 3 nucleotides.
   Embodiment 25. The double-stranded oligonucleotide of Embodiment 24, wherein two to four consecutive nucleotides of the sense strand are each conjugated to a GalNAc molecule.
   Embodiment 26. The double-stranded oligonucleotide of Embodiment 24, wherein the three 3′ terminal nucleotides of the sense strand are each conjugated to a GalNAc molecule.
   Embodiment 27. The double-stranded oligonucleotide of any one of Embodiments 1-26, wherein the GalNAc molecule is a monovalent GalNAc moiety, a bivalent GalNAc moiety, a trivalent GalNAc moiety, or a tetravalent GalNAc moiety.
   Embodiment 28. The double-stranded oligonucleotide of Embodiment 27, wherein the GalNAc molecule is a monovalent GalNAc moiety.
   Embodiment 29. A double-stranded oligonucleotide comprising an antisense strand of about 20-22 nucleotides in length and a sense strand of about 18-20 nucleotides in length, wherein the antisense and sense strands form an asymmetric duplex region of about 18-20 base pairs comprising a 3′ terminal overhang of at least 2 nucleotides of the antisense strand, wherein the antisense strand comprises a region of complementarity to a liver mRNA target sequence, and wherein the sense strand comprises two to four consecutive nucleotides at the 5′ or 3′ terminus each conjugated to a monovalent GalNAc molecule.
   Embodiment 30. The double-stranded oligonucleotide of Embodiment 29, wherein the antisense strand is 22 nucleotides in length, the sense strand is 20 nucleotides in length, the duplex region is 20 base pairs, and the 3′ terminal overhang is 2 nucleotides.
   Embodiment 31. The double-stranded oligonucleotide of Embodiment 29 or 30, comprising a blunt end comprising the 3′ end of the sense strand and the 5′ end of the antisense strand.
   Embodiment 32. A double-stranded oligonucleotide comprising an antisense strand of about 18-22 nucleotides in length and a sense strand of about 15 nucleotides in length, wherein the antisense and sense strands form an asymmetric duplex region of about 15 base pairs comprising a 3′ terminal overhang of at least 2 nucleotides of the antisense strand, wherein the antisense strand comprises a region of complementarity to an mRNA target sequence, and wherein the sense strand comprises three consecutive nucleotides at the 5′ or 3′ terminus each conjugated to a monovalent GalNAc molecule.
   Embodiment 33. The double-stranded oligonucleotide of Embodiment 32, wherein:
(i) the antisense strand is 21 nucleotides, the sense strand is 15 nucleotides, the duplex region is 15 base pairs, and the 3′ terminal overhang is 6 nucleotides;
(ii) the antisense strand is 20 nucleotides, the sense strand is 15 nucleotides, the duplex region is 15 base pairs, and the 3′ terminal overhang is 5 nucleotides;
(iii) the antisense strand is 19 nucleotides, the sense strand is 15 nucleotides, the duplex region is 15 base pairs, and the 3′ terminal overhang is 4 nucleotides; or
(iv) the antisense strand is 18 nucleotides, the sense strand is 15 nucleotides, the duplex region is 15 base pairs, and the 3′ terminal overhang is 3 nucleotides.
   Embodiment 34. The double-stranded oligonucleotide of Embodiment 32 or 33, comprising a blunt end comprising the 3′ end of the sense strand and the 5′ end of the antisense strand.
   Embodiment 35. A double-stranded oligonucleotide comprising an antisense strand of about 20-22 nucleotides in length and a sense strand of about 18-20 nucleotides in length, wherein the antisense and sense strands form an asymmetric duplex region of about 18-20 base pairs comprising a 3′ terminal overhang of at least 2 nucleotides of the antisense strand, wherein the antisense strand comprises a region of complementarity to an mRNA target sequence, and wherein the antisense strand comprises two to four nucleotides each conjugated to a monovalent GalNAc molecule.
   Embodiment 36. The double-stranded oligonucleotide of Embodiment 35, wherein the antisense strand is 22 nucleotides, and wherein (i) nucleotides at positions 20 and 21 are each conjugated to the monovalent GalNAc molecule, (ii) nucleotides at positions 19 and 20 are each conjugated to the monovalent GalNAc molecule, or (iii) the nucleotide at position 20 is conjugated to the monovalent GalNAc molecule.
   Embodiment 37. The double-stranded oligonucleotide of Embodiment 35 or 36, wherein the sense strand comprises a monovalent GalNAc molecule conjugated to the 5′ terminal nucleotide.
   Embodiment 38. The double-stranded oligonucleotide of Embodiment 35 or 36, wherein the two 5′ terminal nucleotides of the sense strand are each conjugated to a monovalent GalNAc molecule.
   Embodiment 39. A double-stranded oligonucleotide comprising an antisense strand of about 20-22 nucleotides in length and a sense strand of about 13-18 nucleotides in length, wherein the antisense and sense strands form a duplex region of about 13-18 base pairs, wherein the antisense strand comprises an orientation of 5′ to 3′, wherein the antisense strand comprises a 3′ overhang of at least one nucleotide and a 5′ overhang of at least one nucleotide, wherein the antisense strand comprises a region of complementarity to an mRNA target sequence, and wherein the sense strand comprises two to four nucleotides each conjugated to a monovalent GalNAc molecule.
   Embodiment 40. The double-stranded oligonucleotide of Embodiment 39, wherein the antisense strand is 22 nucleotides, the sense strand is 18 nucleotides, the duplex region is 18 base pairs, the 3′ overhang is 2 nucleotides, and the 5′ overhang is two nucleotides.
   Embodiment 41. The double-stranded oligonucleotide of Embodiment 40, wherein the three consecutive 3′ terminal nucleotides of the sense strand are each conjugated to the monovalent GalNAc molecule.
   Embodiment 42. The double-stranded oligonucleotide of Embodiment 39, wherein the antisense strand is 22 nucleotides, the sense strand is 13 nucleotides, the duplex region is 13 base pairs, the 3′ overhang is 7 nucleotides, and the 5′ overhang is 2 nucleotides.
   Embodiment 43. The double-stranded oligonucleotide of Embodiment 42, wherein two to three consecutive nucleotides of the sense strand are each conjugated to the monovalent GalNAc molecule.
   Embodiment 44. The double-stranded oligonucleotide of Embodiment 39, wherein the antisense strand is 22 nucleotides, the sense strand is 16 nucleotides, the duplex region is 16 base pairs, the 3′ overhang is 4 nucleotides, and the 5′ overhang is 2 nucleotides.
   Embodiment 45. The double-stranded oligonucleotide of Embodiment 39, wherein the antisense strand is 22 nucleotides, the sense strand is 17 nucleotides, the duplex region is 17 base pairs, the 3′ overhang is 3 nucleotides, and the 5′ overhang is 2 nucleotides.
   Embodiment 46. The double-stranded oligonucleotide of Embodiment 44 or 45, wherein the three consecutive 5′ terminal nucleotides of the sense strand are each conjugated to the monovalent GalNAc molecule.
   Embodiment 47. A double-stranded oligonucleotide comprising an antisense strand of about 18-22 nucleotides in length and a sense strand of about 13-18 nucleotides in length, wherein the antisense and sense strands form a duplex region of about 13-18 base pairs, wherein the antisense strand comprises an orientation of 5′ to 3′, wherein the antisense strand comprises a 3′ overhang of at least one nucleotide and a 5′ overhang of at least one nucleotide, wherein the antisense strand comprises a region of complementarity to an mRNA target sequence, and wherein the sense strand comprises two to four nucleotides each conjugated to a monovalent GalNAc molecule.
   Embodiment 48. The double-stranded oligonucleotide of any one of Embodiments 1-47, wherein the oligonucleotide comprises at least one modified nucleotide.
   Embodiment 49. The double-stranded oligonucleotide of Embodiment 48, wherein the modified nucleotide comprises a 2′-modification.
   Embodiment 50. The double-stranded oligonucleotide of Embodiment 49, wherein each of the nucleotides of the sense strand and the antisense strand comprise a 2′-modification.
   Embodiment 51. The double-stranded oligonucleotide of Embodiment 49 or 50, wherein the 2′-modification is a modification selected from 2′-aminoethyl, 2′-fluoro, 2′-O-methyl, 2′-O-methoxyethyl, and 2′-deoxy-2′-fluoro-β-d-arabinonucleic acid.
   Embodiment 52. The double-stranded oligonucleotide of any one of Embodiments 1-51, wherein the sense strand comprises nucleotide positions numbered 5′ to 3′, and wherein position 8 comprises a 2′-fluor modification, or each of positions (i) 8-11; (ii) 3-6; (iii) 5-6; (iv) 3-4; (v) 6-9; or (vi) 7-10, comprise a 2′-fluoro modification.
   Embodiment 53. The double stranded oligonucleotide of any one of Embodiments 1-51, wherein the sense strand comprises a 2′-fluoro modification at each of nucleotides forming a base pair with nucleotides at positions 10-13, 12-13, or 10-11, of the antisense strand, wherein positions are numbered 5′ to 3′.
   Embodiment 54. The double-stranded oligonucleotide of any one of Embodiments 1-53, wherein the antisense strand comprises positions numbered 5′ to 3′, and wherein each of positions 2, 3, 4, 5, 7, 10 and 14 comprise a 2′-fluoro modification.
   Embodiment 55. The double-stranded oligonucleotide of any one of Embodiments 52-54, wherein the remaining nucleotides comprise a 2′-O-methyl modification, provided that the nucleotides conjugated to the GalNAc molecule does not comprise a 2′-O-methyl modification.
   Embodiment 56. The double-stranded oligonucleotide of any one of the preceding Embodiments, wherein the oligonucleotide comprises at least one modified internucleotide linkage.
   Embodiment 57. The double-stranded oligonucleotide of Embodiment 56, wherein the at least one modified internucleotide linkage is a phosphorothioate linkage.
   Embodiment 58. The double-stranded oligonucleotide of Embodiment 57, wherein the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′.
   Embodiment 59. The double-stranded oligonucleotide of Embodiment 57 or 58, wherein the antisense strand comprises a phosphorothioate linkage between the penultimate nucleotide and third nucleotide from the 3′ end, and between the penultimate nucleotide and ultimate nucleotide.
   Embodiment 60. The double-stranded oligonucleotide of Embodiment 57 or 58, wherein the antisense strand is 18 nucleotides in length, and wherein the antisense strand comprises a phosphorothioate linkage between positions 16 and 17 and between positions 17 and 18, wherein positions are numbered 1-18 from 5′ to 3′.
   Embodiment 61. The double-stranded oligonucleotide of any one of Embodiments 57-60, wherein the sense strand comprises a phosphorothioate linkage between positions 1 and 2, between the penultimate nucleotide and third nucleotide from the 3′ end, and between the penultimate nucleotide and ultimate nucleotide.
   Embodiment 62. The double-stranded oligonucleotide of any one of Embodiments 1-61, wherein the sense strand comprises at least one Tm-increasing nucleotide.
   Embodiment 63. The double-stranded oligonucleotide of Embodiment 62, wherein the sense strand comprises up to three Tm-increasing nucleotides.
   Embodiment 64. The double-stranded oligonucleotide of Embodiment 62, wherein the sense strand comprises three Tm-increasing nucleotides.
   Embodiment 65. The double-stranded oligonucleotide of any one of Embodiments 62-64, wherein the Tm-increasing nucleotide is a bicyclic nucleotide.
   Embodiment 66. The double-stranded oligonucleotide of any one of Embodiments 62-64, wherein the Tm-increasing nucleotide is a locked nucleic acid.
   Embodiment 67. The double-stranded oligonucleotide of any one of Embodiments 1-66, wherein the antisense strand comprises a phosphorylated nucleotide at the 5′ terminus, wherein the phosphorylated nucleotide is selected from uridine and adenosine.
   Embodiment 68. The double-stranded oligonucleotide of Embodiment 67, wherein the phosphorylated nucleotide is uridine.
   Embodiment 69. The double-stranded oligonucleotide of any one of Embodiments 67-68, wherein the phosphorylated nucleotide is 4′-O-monomethylphosphonate-2′-O-methyl uridine.
   Embodiment 70. The double-stranded oligonucleotide of any one of Embodiments 1-66, wherein the 4′-carbon of the sugar of the 5′-nucleotide of the antisense strand comprises a phosphate analog.
   Embodiment 71. The double-stranded oligonucleotide of Embodiment 70, wherein the phosphate analog is oxymethyl phosphonate, vinyl phosphonate or malonyl phosphonate.
   Embodiment 72. The double-stranded oligonucleotide of any one of Embodiments 1-31 and 48-71, wherein the region of complementarity is fully complementary to the liver mRNA target sequence.
   Embodiment 73. The double-stranded oligonucleotide of any one of Embodiments 1-31 and 48-71, wherein the region of complementarity is partially complementary to the liver mRNA target sequence.
   Embodiment 74. The double-stranded oligonucleotide of Embodiment 72, wherein the region of complementarity comprises no more than four mismatches to the liver mRNA target sequence.
   Embodiment 75. The double-stranded oligonucleotide of any one of Embodiments 32-71 wherein the region of complementarity is fully complementary to the mRNA target sequence.
   Embodiment 76. The double-stranded oligonucleotide of any one of Embodiments 32-71, wherein the region of complementarity is partially complementary to the mRNA target sequence.
   Embodiment 77. The double-stranded oligonucleotide of Embodiment 76, wherein the region of complementarity comprises no more than four mismatches to the mRNA target sequence.
   Embodiment 78. The double-stranded oligonucleotide of any one of Embodiments 1-76, wherein the oligonucleotide reduces expression of the liver mRNA target sequence or mRNA target sequence in a cell or population of cells in vitro and/or in vivo.
   Embodiment 79. A pharmaceutical composition comprising the oligonucleotide of any one of Embodiments 1-78, and a pharmaceutically acceptable carrier, delivery agent or excipient.
   Embodiment 80. A method for treating a subject having a disease, disorder or condition associated with expression of a liver mRNA or target mRNA, the method comprising administering to the subject a therapeutically effective amount of the double-stranded oligonucleotide of any one of Embodiments 1-78, or the pharmaceutical composition of Embodiment 79.
   Embodiment 81. A method of reducing expression of a liver mRNA or target mRNA in a subject, comprising administering to the subject the double-stranded oligonucleotide of any one of Embodiments 1-78, or the pharmaceutical composition of Embodiment 79.
   Embodiment 82. A kit comprising the double-stranded oligonucleotide of any one of Embodiments 1-78, an optional pharmaceutically acceptable carrier, and a package insert comprising instructions for administration to a subject having a disease, disorder or condition associated with the overexpression or reduction in expression of a liver mRNA or target mRNA.
   Embodiment 83. Use of the double-stranded oligonucleotide of any one of Embodiments 1-78, or the pharmaceutical composition of Embodiment 79, in the manufacture of a medicament for the treatment of a disease, disorder, or condition associated with the overexpression or reduction in expression of a liver mRNA or a target mRNA.
   Embodiment 84. The double-stranded oligonucleotide of any one of Embodiments 1-78, or the pharmaceutical composition of Embodiment 79, for use, or adaptable for use, in the treatment of a disease, disorder, or condition associated with overexpression or reduction in expression of a liver mRNA or target mRNA.
   Embodiment 85. A method of activating target-specific RNA interference (RNAi) in an organism comprising administering to said organism the double-stranded oligonucleotide of any one of Embodiments 1-78, said double-stranded oligonucleotide being administered in an amount sufficient for degradation of the liver mRNA or target mRNA to occur, thereby activating target-specific RNAi in the organism.
   Embodiment 86. The method of Embodiment 85, wherein the liver mRNA or target mRNA specifies the amino acid sequence of a protein involved or predicted to be involved in a human disease or disorder.
   Embodiment 87. The method of Embodiment 86, wherein the disease or disorder is selected from the group consisting of viral infections, bacterial infections, parasitic infections, cancers, allergies, autoimmune diseases, immunodeficiencies, and immunosuppression.

### EXAMPLES

### Example 1: Preparation of RNAi Oligonucleotides

### Oligonucleotide Synthesis and Purification

The oligonucleotides (RNAi oligonucleotides) described in the Examples were chemically synthesized using methods described herein. Generally, RNAi oligonucleotides were synthesized using solid phase oligonucleotide synthesis methods as described for 19-23mer RNAi oligonucleotides (see, e.g., Scaringe et al. (1990) NUCLEIC ACIDS RES. 18:5433-41 and Usman et al. (1987) J. AM. CHEM. SOC. 109:7845-45; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis methodologies (see, e.g., Hughes and Ellington (2017) COLD SPRING HARB PERSPECT BIOL. 9(1):a023812; Beaucage S. L., Caruthers M. H. STUDIES ON NUCLEOTIDE CHEMISTRY V: Deoxynucleoside Phosphoramidites—A New Class of Key Intermediates for Deoxypolynucleotide Synthesis, TETRAHEDRON LETT. 1981; 22:1859-62. doi: 10.1016/S0040-4039(01)90461-7); PCT application No. PCT/US2021/42469 (each of which is incorporated herein by this reference)).

Individual RNA strands were synthesized and HPLC purified according to standard methods (Integrated DNA Technologies; Coralville, IA). For example, RNA oligonucleotides were synthesized using solid phase phosphoramidite chemistry, deprotected and desalted on NAP-5 columns (Amersham Pharmacia Biotech; Piscataway, NJ) using standard techniques (Damha & Olgivie (1993) METHODS MOL. BIOL. 20:81-114; Wincott et al. (1995) NUCLEIC ACIDS RES. 23:2677-84) and the phosphoramidite synthesis as shown below:

### Synthesis of 2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy) ethan-1-ammonium formate (1-6)

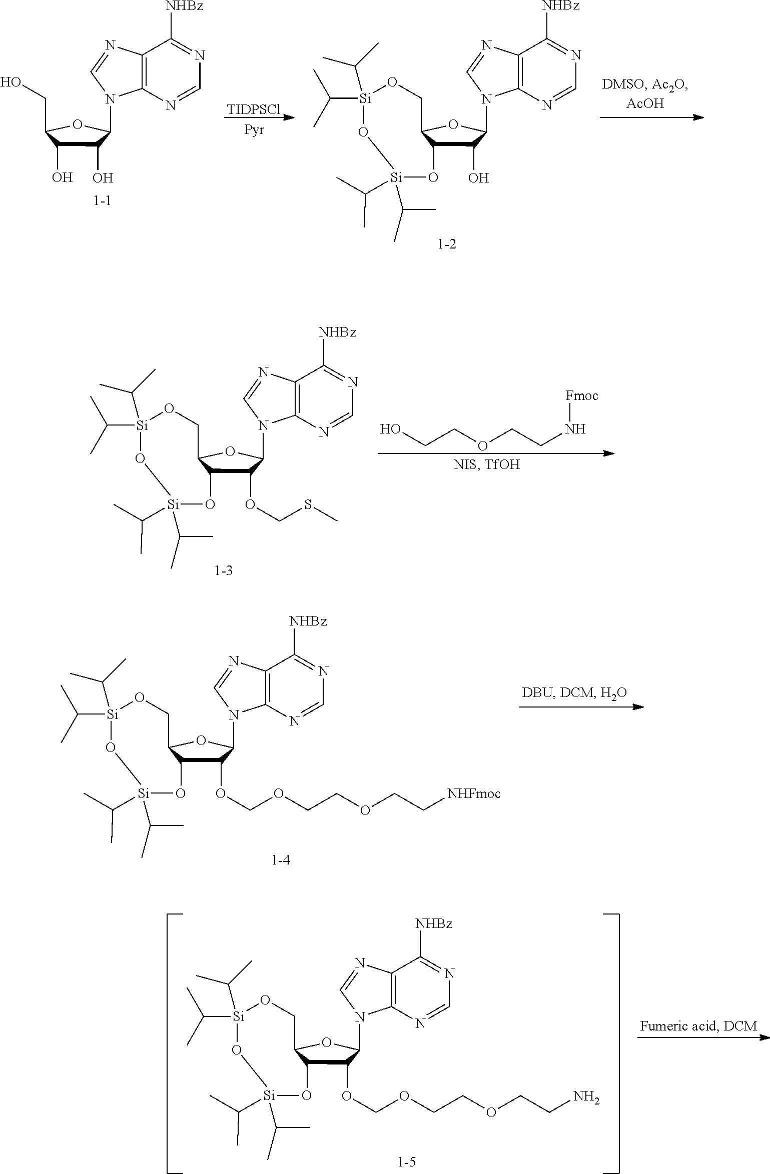

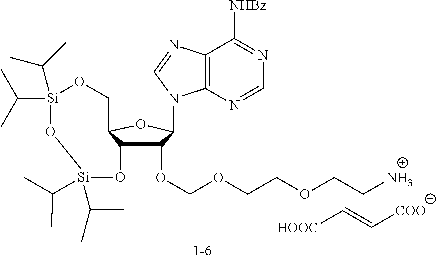

A solution of compound 1-1 (25.00 g, 67.38 mmol) in 20 mL of DMF was treated with pyridine (11 mL, 134.67 mmol) and tetraisopropyldisiloxane dichloride (22.63 mL, 70.75 mmol) at 10° C. The resulting mixture was stirred at 25° C. for 3 h and quenched with 20% citric acid (50 mL). The aqueous layer was extracted with EtOAc (3×50 mL) and the combined organic layers were concentrated in vacuo. The crude residue was recrystallized from a mixture of MTBE and n-heptane (1:15, 320 mL) to afford compound 1-2 (37.20 g, 90%) as a white oily solid.

A solution of compound 1-2 (37.00 g, 60.33 mmol) in 20 mL of DMSO was treated with AcOH (20 mL, 317.20 mmol) and Ac₂O (15 mL, 156.68 mmol). The mixture was stirred at 25° C. for 15 h. The reaction was diluted with EtOAc (100 mL) and quenched with sat. K₂CO₃ (50 mL). The aqueous layer was extracted with EtOAc (3×50 mL). The combined organic layers were concentrated and recrystallized with ACN (30 mL) to afford compound 1-3 (15.65 g, 38.4%) as a white solid.

A solution of compound 1-3 (20.00 g, 29.72 mmol) in 120 mL of DCM was treated with Fmoc-amino-ethoxy ethanol (11.67 g, 35.66 mmol) at 25° C. The mixture was stirred to afford a clear solution and then treated with 4 Å molecular sieves (20.0 g), N-iodosuccinimide (8.02 g, 35.66 mmol), and TfOH (5.25 mL, 59.44 mmol). The mixture was stirred at 30° C. until the HPLC analysis indicated >95% consumption of compound 1-3. The reaction was quenched with TEA (6 mL) and filtered. The filtrate was diluted with EtOAc, washed with sat. NaHCO₃ (2×100 mL), sat. Na₂SO₃ (2×100 mL), and water (2×100 mL) and concentrated in vacuo to afford crude compound 1-4 (26.34 g, 93.9%) as a yellow solid, which was used directly for the next step without further purification.

A solution of compound 1-4 (26.34 g, 27.62 mmol) in a mixture of DCM/water (10:7, 170 mL) was treated with DBU (7.00 mL, 45.08 mmol) at 5° C. The mixture was stirred at 5-25° C. for 1 h. The organic layer was then separated, washed with water (100 mL), and diluted with DCM (130 mL). The solution was treated with fumaric acid (7.05 g, 60.76 mmol) and 4 Å molecular sieves (26.34 g) in four portions. The mixture was stirred for 1 h, concentrated, and recrystallized from a mixture of MTBE and DCM (5:1) to afford compound 1-6 (14.74 g, 62.9%) as a white solid: ¹H NMR (400 MHz, d₆-DMSO) 8.73 (s, 1H), 8.58 (s, 1H), 8.15-8.02 (m, 2H), 7.65-7.60 (m, 1H), 7.59-7.51 (m, 2H), 6.52 (s, 2H), 6.15 (s, 1H), 5.08-4.90 (m, 3H), 4.83-4.78 (m, 1H), 4.15-3.90 (m, 3H), 3.79-3.65 (m, 2H), 2.98-2.85 (m, 6H), 1.20-0.95 (m, 28H).

### Synthesis of (2R,3R,4R,5R)-5-(6-benzamido-9H-purin-9-yl)-2-((bis(4-methoxyphenyl)(phenyl)methoxy)methyl)-4-((2-(2-[lipid]-amidoethoxy)ethoxy)methoxy) tetrahydrofuran-3-yl(2-cyanoethyl) diisopropylphosphoramidite (2-4a to 2-4e)

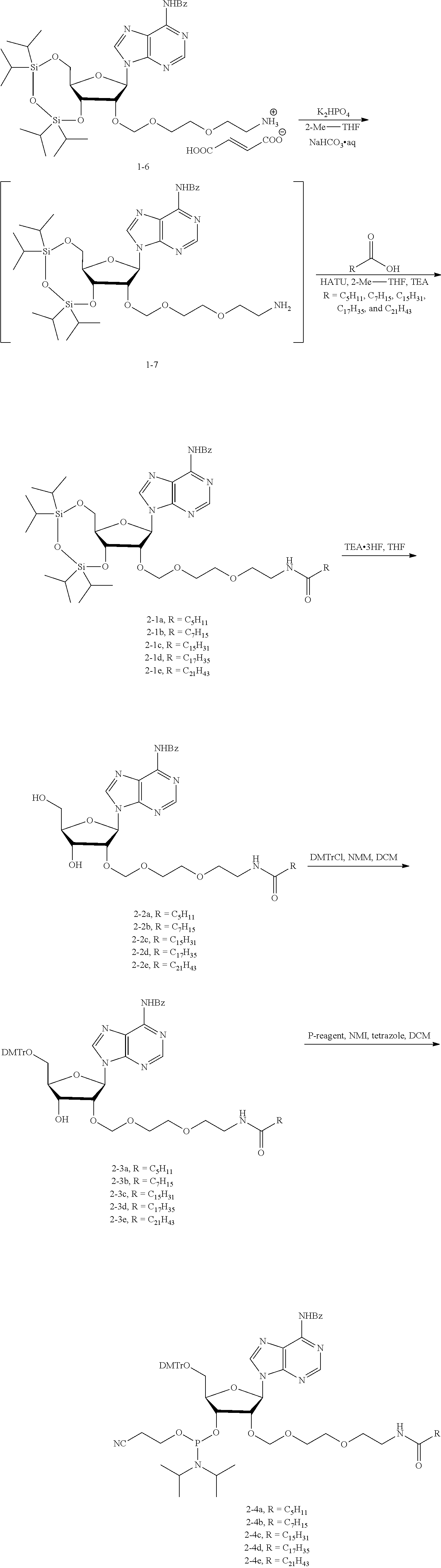

A solution of compound 1-6 (50.00 g, 59.01 mmol) in 150 mL of 2-methyltetrahydrofuran was washed with ice cold aqueous K₂HPO₄ (6%, 100 mL) and brine (20%, 2×100 mL). The organic layer was separated and treated with hexanoic acid (10.33 mL, 82.61 mmol), HATU (33.66 g, 88.52 mmol), and DMAP (10.81 g, 147.52 mmol) at 0° C. The resulting mixture was warmed to 25° C. and stirred for 1 h. The solution was washed with water (2×100 mL), brine (100 mL), and concentrated in vacuo to afford a crude residue. Flash chromatography on silica gel (1:1 hexanes/acetone) gave compound 2-1a (34.95 g, 71.5%) as a white solid.

A mixture of compound 2-1a (34.95 g, 42.19 mmol) and TEA (9.28 mL, 126.58 mmol) in 80 mL of THF was treated with triethylamine trihydrofluoride (20.61 mL, 126.58 mmol) dropwise at 10° C. The mixture was warmed to 25° C. and stirred for 2 h. The reaction was concentrated, dissolved in DCM (100 mL), and washed with sat. NaHCO₃ (5×20 mL) and brine (50 mL). The organic layer was concentrated in vacuo to afford crude compound 2-2a (24.72 g, 99%), which was used directly for the next step without further purification.

A solution of compound 2-2a (24.72 g, 42.18 mmol) in 50 mL of DCM was treated with N-methylmorpholine (18.54 mL, 168.67 mmol) and DMTr-Cl (15.69 g, 46.38 mmol). The mixture was stirred at 25° C. for 2 h and quenched with sat. NaHCO₃ (50 mL). The organic layer was separated, washed with water, concentrated to afford a slurry crude. Flash chromatography on silica gel (1:1 hexanes/acetone) gave compound 2-3a (30.05 g, 33.8 mmol, 79.9%) as a white solid.

A solution of compound 2-3a (25.00 g, 28.17 mmol) in 50 mL of DCM was treated with N-methylmorpholine (3.10 mL, 28.17 mmol) and tetrazole (0.67 mL, 14.09 mmol) under nitrogen atmosphere. Bis(diisopropylamino) chlorophosphine (9.02 g, 33.80 mmol) was added to the solution dropwise and the resulting mixture was stirred at 25° C. for 4 h. The reaction was quenched with water (15 mL), and the aqueous layer was extracted with DCM (3×50 mL). The combined organic layers were washed with sat. NaHCO₃ (50 mL), concentrated to afford a crude solid that was recrystallized from a mixture of DCM/MTBE/n-hexane (1:4:40) to afford compound 2-4a (25.52 g, 83.4%) as a white solid: ¹H NMR (400 MHz, d₆-DMSO) 11.25 (s, 1H), 8.65-8.60 (m, 2H), 8.09-8.02 (m, 2H), 7.71 (s, 1H), 7.67-7.60 (m, 1H), 7.59-7.51 (m, 2H), 7.38-7.34 (m, 2H), 7.30-7.25 (m, 7H), 6.85-6.79 (m, 4H), 6.23-6.20 (m, 1H), 5.23-5.14 (m, 1H), 4.80-4.69 (m, 3H), 4.33-4.23 (m, 2H), 3.90-3.78 (m, 1H), 3.75 (s, 6H), 3.74-3.52 (m, 3H), 3.50-3.20 (m, 6H), 3.14-3.09 (m, 2H), 3.09 (s, 1H), 2.82-2.80 (m, 1H), 2.65-2.60 (m, 1H), 2.05-1.96 (m, 2H), 1.50-1.39 (m, 2H), 1.31-1.10 (m, 14H), 1.08-1.05 (m, 2H), 0.85-0.79 (m, 3H); ³¹P NMR (162 MHz, d₆-DMSO) 149.43, 149.18.

Compound 2-4b, 2-4c, 2-4d, and 2-4e were prepared using similar procedures described above for compound 2-4a. Compound 2-4b was obtained (25.50 g, 85.4%) as a white solid: ¹H NMR (400 MHz, d₆-DMSO) 11.23 (s, 1H), 8.65-8.60 (m, 2H), 8.05-8.02 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.97 (m, 2H), 1.50-1.38 (m, 2H), 1.31-1.10 (m, 18H), 1.08-1.05 (m, 2H), 0.85-0.78 (m, 3H); ³¹P NMR (162 MHz, d₆-DMSO) 149.43, 149.19.

Compound 2-4c was obtained (36.60 g, 66.3%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.25-5.17 (m, 1H), 4.80-4.69 (m, 3H), 4.40-4.21 (m, 2H), 3.91-3.80 (m, 1H), 3.74 (s, 6H), 3.74-3.50 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.33-1.12 (m, 38H), 1.08-1.05 (m, 2H), 0.86-0.80 (m, 3H); ³¹P NMR (162 MHz, d₆-DMSO) 149.42, 149.17.

Compound 2-4d was obtained (26.60 g, 72.9%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.33 (m, 2H), 7.30-7.25 (m, 7H), 6.89-6.80 (m, 4H), 6.21-6.15 (m, 1H), 5.22-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.08 (m, 38H), 1.08-1.05 (m, 2H), 0.85-0.79 (m, 3H); ³¹P NMR (162 MHz, d₆-DMSO) 149.47, 149.22.

Compound 2-4e was obtained (38.10 g, 54.0%) as a white solid: ¹H NMR (400 MHz, do-DMSO) 11.21 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.73 (s, 6H), 3.74-3.52 (m, 3H), 3.47-3.22 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.06 (m, 46H), 1.08-1.06 (m, 2H), 0.85-0.77 (m, 3H); ³¹P NMR (162 MHz, d₆-DMSO) 149.41, 149.15.

The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.; Fullerton, CA). The CE capillaries have a 100 μm inner diameter and contain ssDNA 100R Gel (Beckman-Coulter). Typically, about 0.6 nmole of oligonucleotide was injected into a capillary, run in an electric field of 444 V/cm, and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DE™ Biospectometry Work Station (Applied Biosystems; Foster City, CA) following the manufacturer's recommended protocol. Relative molecular masses of all oligomers were obtained, often within 0.2% of expected molecular mass.

### Preparation of Duplexes

Single strand RNA oligomers were resuspended (e.g., at 100 μM concentration) in duplex buffer consisting of 100 mM potassium acetate, 30 mM HEPES, pH 7.5. Complementary sense and antisense strands were mixed in equal molar amounts to yield a final solution of, for example, 50 M duplex. Samples were heated to 100° C. for 5′ in RNA buffer (IDT) and were allowed to cool to room temperature before use. The RNAi oligonucleotides were stored at −20° C. Single strand RNA oligomers were stored lyophilized or in nuclease-free water at −80° C.

The synthesis methods described herein are used to generate the lipid-conjugated oligonucleotides described in the Examples below.

### Example 2: Low Molecular Weight GalNAc RNAi Oligonucleotides

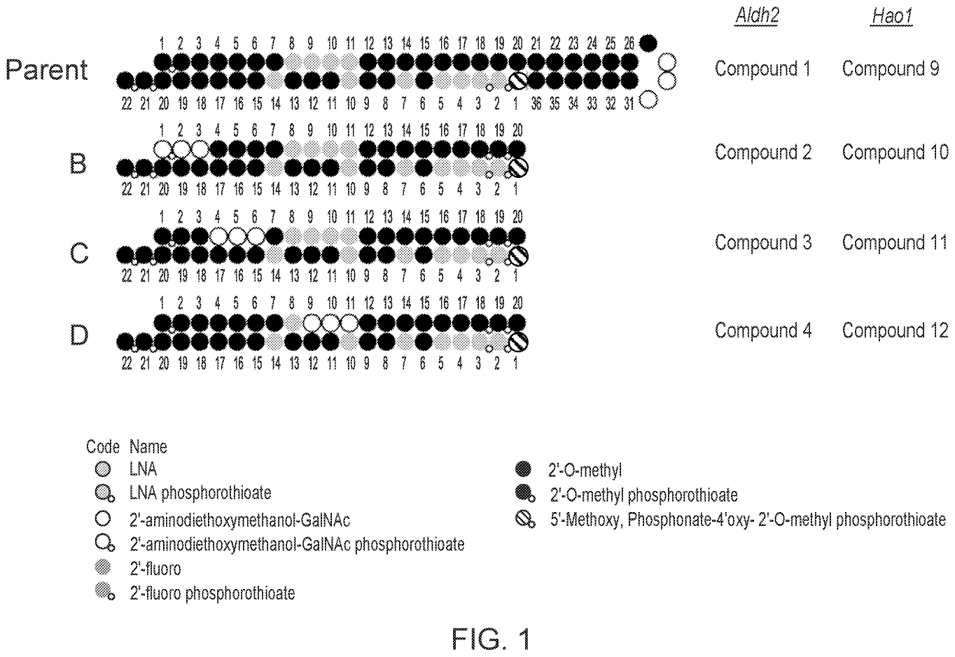

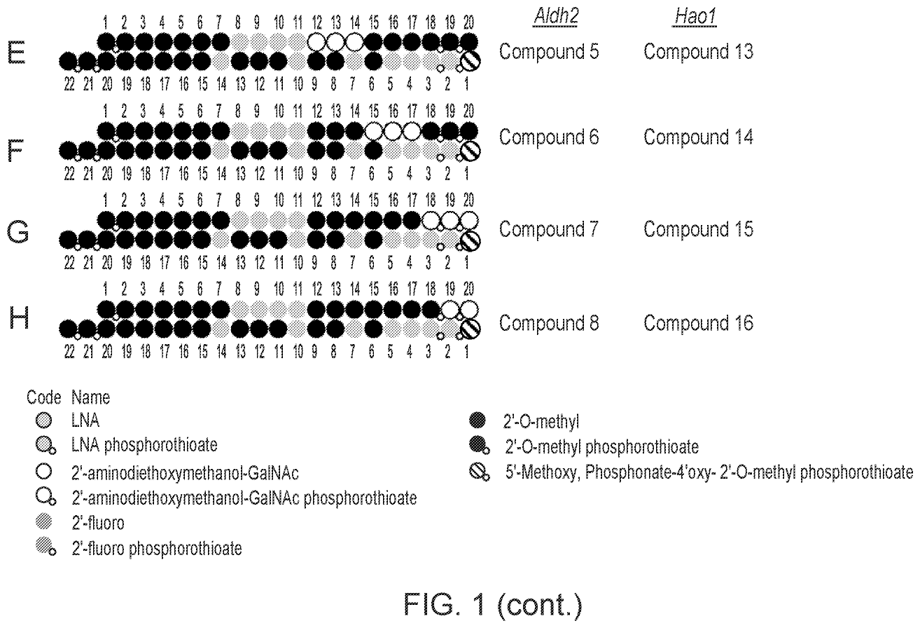

To test if target gene knockdown was retained in low molecular weight oligonucleotides, RNAi oligonucleotide-ligand conjugates were generated with GalNAc molecules conjugated to different positions of the sense strand using the methods described in Example 1. The oligonucleotides comprised an antisense strand having a region of complementarity to mRNA encoding Aldh2 (Table 1) or Hao1 (Table 2) with the modification pattern as shown in FIG. 1. Oligonucleotides were administered in a single subcutaneous dose at 0.5 mg/kg.

Comparison was made to a parent oligonucleotide-ligand conjugate with a 36-nucleotide (nt) sense strand and a 22-nt antisense strand, where the sense strand comprises a tetraloop (Compounds 1 and 9, FIG. 1).

RNAi oligonucleotide-ligand conjugates that were evaluated in comparison to Compounds 1 and 9 had structures according to Compounds 2-8 and 10-16 as shown in FIG. 1. Compounds 2-8 and 10-16 comprise a 20-nt sense strand, a 2-nt overhang at the 3′ terminus the antisense strand, and a blunt end at the 5′ terminus of the antisense strand. These Compounds comprise the additional features described below:
(i) Compounds 2 and 10 comprise a GalNAc molecule conjugated to Positions 1, 2, and 3 of the sense strand.
(ii) Compounds 3 and 11 comprise a GalNAc molecule conjugated to Positions 4, 5, and 6 of the sense strand.
(iii) Compounds 4 and 12 comprise a GalNAc molecule conjugated to Positions 9, 10, and 11 of the sense strand.
(iv) Compounds 5 and 13 comprise a GalNAc molecule conjugated to Positions 12, 13, and 14 of the sense strand.
(v) Compounds 6 and 14 comprise a GalNAc molecule conjugated to Positions 15, 16, and 17 of the sense strand.
(vi) Compounds 7 and 15 comprise a GalNAc molecule conjugated to Positions 18, 19, and 20 of the sense strand.
(vii) Compounds 8 and 16 comprise a GalNAc molecule conjugated to Positions 19 and 20 of the sense strand.

TABLE 1
Aldh2 RNAi Oligonucleotide-ligand Conjugates

| | Compound | Sense SEQ ID NOS | | Antisense SEQ ID NOS | |
|---|---|---|---|---|---|
| Group | ID | Unmodified | Modified | Unmodified | Modified |
| Parent | 1 | 1 | 21 | 15 | 67 |
| B | 2 | 2 | 22 | 15 | 67 |
| C | 3 | 2 | 23 | 15 | 67 |
| D | 4 | 2 | 24 | 15 | 67 |
| E | 5 | 2 | 25 | 15 | 67 |
| F | 6 | 2 | 26 | 15 | 67 |
| G | 7 | 2 | 27 | 15 | 67 |
| H | 8 | 2 | 28 | 15 | 67 |

TABLE 1
Hao1 RNAi Oligonucleotide-ligand Conjugates

| | Compound | Sense SEQ ID NOS | | Antisense SEQ ID NOS | |
|---|---|---|---|---|---|
| Group | ID | Unmodified | Modified | Unmodified | Modified |
| Parent | 9 | 3 | 29 | 16 | 68 |
| B | 10 | 4 | 30 | 16 | 68 |
| C | 11 | 4 | 31 | 16 | 68 |
| D | 12 | 4 | 32 | 16 | 68 |
| E | 13 | 4 | 33 | 16 | 68 |
| F | 14 | 4 | 34 | 16 | 68 |
| G | 15 | 4 | 35 | 16 | 68 |
| H | 16 | 4 | 36 | 16 | 68 |

Target knockdown was assessed 5 days post-injection. RNA was extracted from liver to determine murine Aldh2 and Hao1 mRNA levels by qPCR (normalized to endogenous housekeeping gene Ppib). The qPCR was performed using PrimeTime™ qPCR Probe Assays. The percentage of murine Aldh2 or Hao1 mRNA remaining in the samples from treated mice was determined using the 2-ΔΔCt (“delta-delta Ct”) method (Livak and Schmittgen (2001) METHODS 25:402-408).

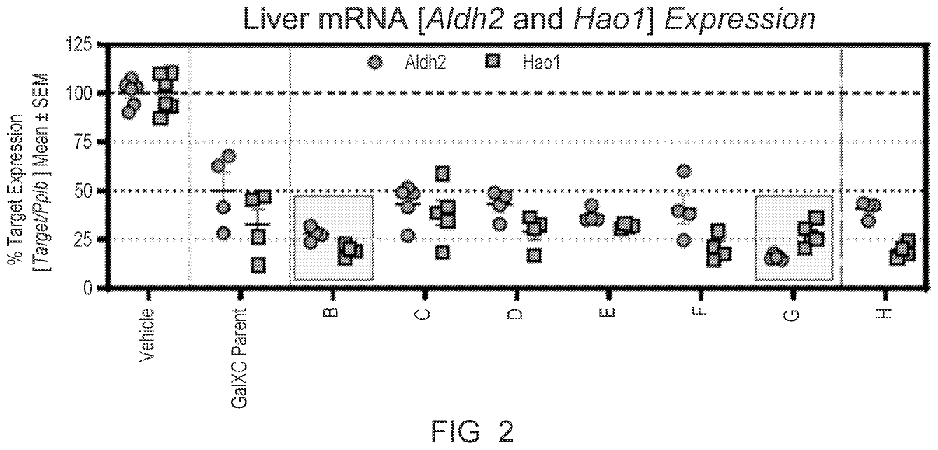

Percent Aldh2 and Hao1 mRNA expression remaining in liver is shown in FIG. 2. Low molecular weight RNAi oligonucleotide-ligand conjugates had similar or better activity for target gene knockdown relative to the parent molecule. Activity was observed regardless of GalNAc placement on the sense strand.

### Example 3: Target Gene Knockdown Activity is Retained in Truncated RNAi-Oligonucleotides with GalNAc Conjugation

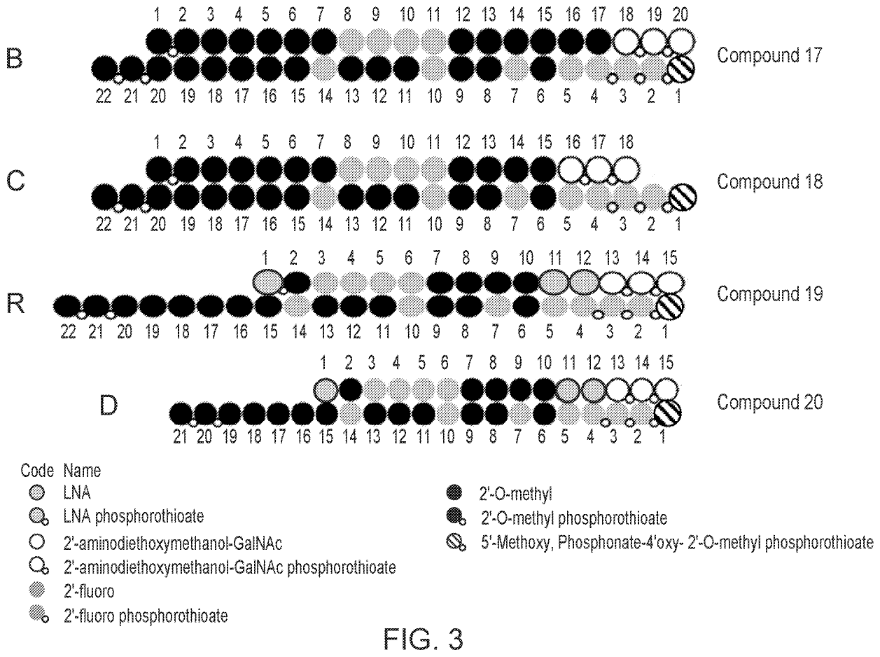

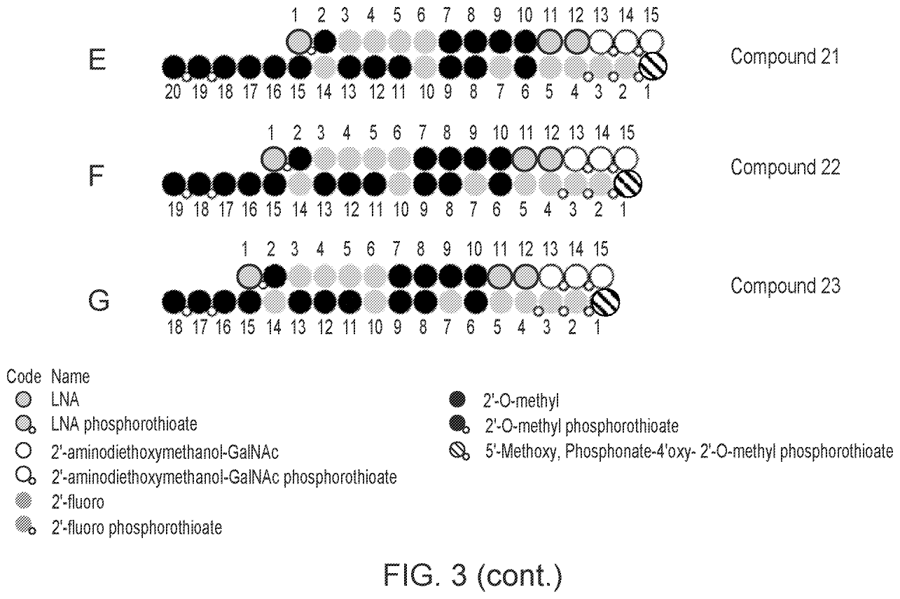

To test if target gene knockdown in truncated molecules was retained with GalNAc conjugated to the oligonucleotide, RNAi oligonucleotide-ligand conjugates were generated with GalNAc molecules conjugated to the three 3′ terminal nucleotides of the sense strand using the methods described in Example 1. The oligonucleotides comprised an antisense strand having a region of complementarity to mRNA encoding Aldh2 (Table 3) with the modification pattern as shown in FIG. 3. Oligonucleotides were administered in a single subcutaneous dose at 0.5 mg/kg.

Comparison was made to a parent oligonucleotide-ligand conjugate with a 20-nt sense strand and a 22-nt antisense strand (Compound 17, FIG. 3).

RNAi oligonucleotide-ligand conjugates that were evaluated in comparison to Compound 17 had structures according to Compounds 18-23 as shown in FIG. 3. These structures contain the features described below:
(i) Compound 18 comprises an 18-nt sense strand. The Compound comprises a 2-nt overhang at the 3′ terminus the antisense strand and a 2-nt overhang at the 5′ terminus of the antisense strand.
(ii) Compounds 19-23 comprise a 15-nt sense strand. The Compounds comprise a blunt end at the 3′ end of the sense strand. The sense strand comprises locked-nucleic acids at positions 1, 11, and 12. Compound 19 comprises a 22-nt antisense strand and a 7-nt overhang at the 3′ terminus of the antisense strand. Compound 20 comprises a 21-nt antisense strand and a 6-nt overhang at the 3′ terminus of the antisense strand. Compound 21 comprises a 20-nt antisense strand and a 5-nt overhang at the 3′ terminus of the antisense strand. Compound 22 comprises 1 19-nt antisense strand and a 4-nt overhang at the 3′ terminus of the antisense strand. Compound 23 comprises an 18-nt antisense strand and a 3-nt overhang at the 3′ terminus of the antisense strand.

TABLE 3
Aldh2 RNAi Oligonucleotide-ligand Conjugates

| | Compound | Sense SEQ ID NOS | | Antisense SEQ ID NOs | |
|---|---|---|---|---|---|
| Group | ID | Unmodified | Modified | Unmodified | Modified |
| B | 17 | 2 | 27 | 15 | 69 |
| C | 18 | 5 | 37 | 15 | 69 |
| R | 19 | 6 | 38 | 15 | 69 |
| D | 20 | 6 | 38 | 17 | 70 |
| E | 21 | 6 | 38 | 18 | 71 |
| F | 22 | 6 | 38 | 19 | 72 |
| G | 23 | 6 | 38 | 20 | 73 |

Target knockdown was assessed 7 days post-injection. RNA was extracted from liver to determine murine Aldh2 mRNA levels by qPCR as described in Example 2.

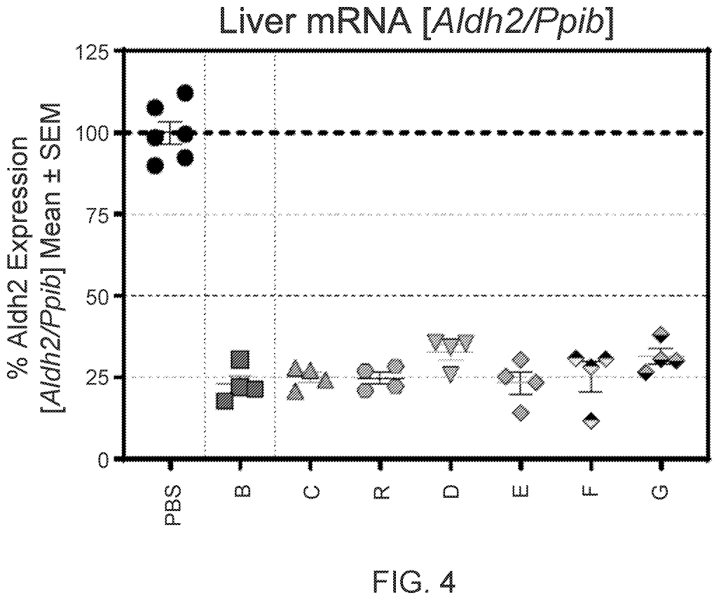

Percent Aldh2 mRNA expression remaining in liver is shown in FIG. 4. RNAi oligonucleotide-ligand conjugates with GalNAc conjugates and truncated antisense strands maintained target gene knockdown relative to the parent molecule.

### Example 4: Position of GalNAc Molecules on Antisense Strand Impact RNAi Oligonucleotide-Ligand Conjugate Activity

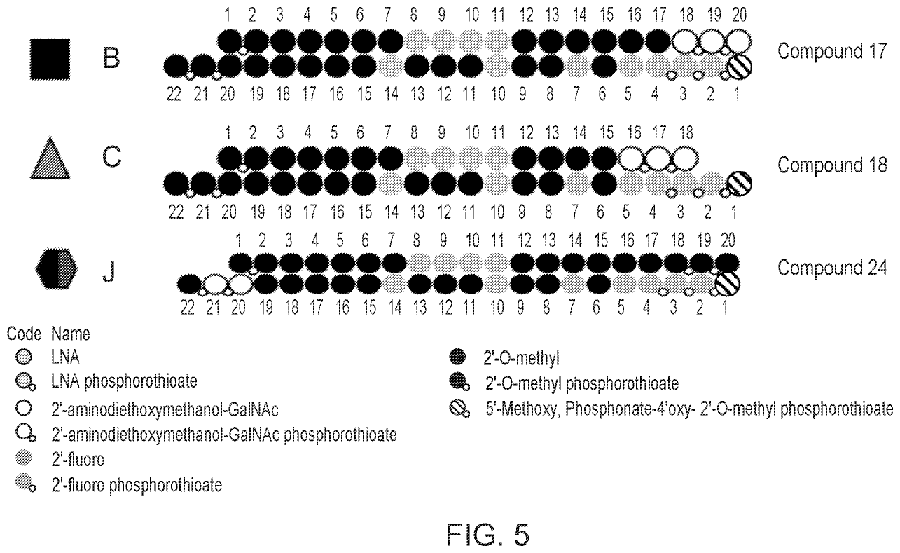

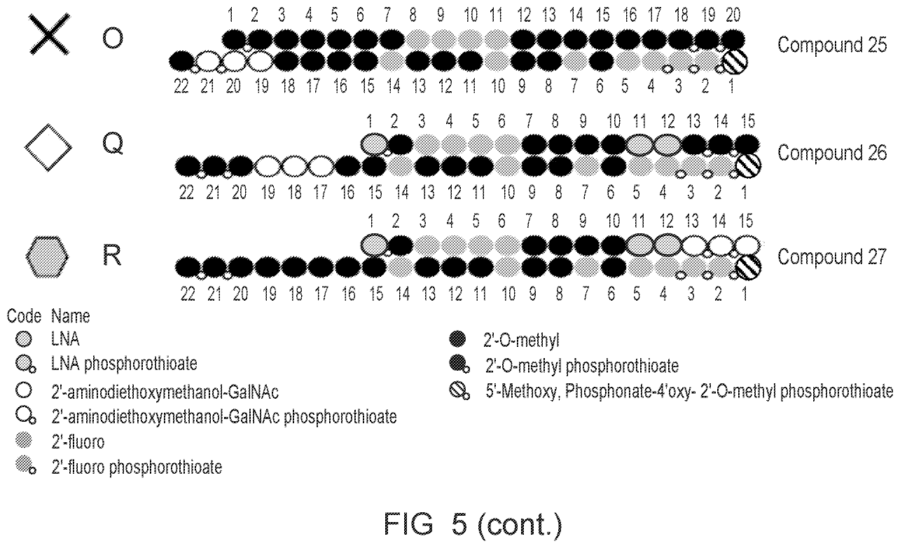

To test if target gene knockdown was impacted by incorporation of GalNAc molecules on the antisense strand, RNAi oligonucleotide-ligand conjugates comprising GalNAc molecules at different antisense strand positions were generated using the methods described in Example 1. The oligonucleotides comprised an antisense strand having a region of complementarity to mRNA encoding Aldh2 (Table 4) with the modification patterns as shown in FIG. 5. Oligonucleotides were administered in a single subcutaneous dose at 0.5 mg/kg.

Comparison was made to parent RNAi oligonucleotide-ligand conjugate Compounds 17 and 18 previously described in Example 3.

RNAi oligonucleotide-ligand conjugates that were evaluated in comparison to Compounds 17 and 18 had structures according to Compounds 24-27 as shown in FIG. 5. These structures contain the features described below:
(i) Compounds 24 and 25 comprise a 20-nt sense strand and a 22-nt antisense strand. The Compounds comprise a 2-nt overhang at the 3′ terminus of the antisense strand. Compound 24 comprises a GalNAc molecule conjugated to positions 20 and 21 of the antisense strand. Compound 25 comprises a GalNAc molecule conjugated to positions 19, 20, and 21 of the antisense strand.
(ii) Compounds 26 and 27 comprise a 15-nt sense strand and a 22-nt antisense strand. The Compounds comprise a 7-nt overhang at the 3′ terminus of the antisense strand. The Compounds comprise a locked nucleic acid at positions 1, 11, and 12 of the sense strand. Compound 26 comprises a GalNAc molecule conjugated to positions 17, 18, and 19 of the antisense strand. Compound 27 comprises a GalNAc molecule conjugated to positions 13, 14, and 15 of the sense strand.

TABLE 4
Aldh2 RNAi Oligonucleotide-ligand Conjugates

| | Compound | Sense SEQ ID NOS | | Antisense SEQ ID NOS | |
|---|---|---|---|---|---|
| Group | ID | Unmodified | Modified | Unmodified | Modified |
| B | 17 | 2 | 27 | 15 | 69 |
| C | 18 | 5 | 37 | 15 | 69 |
| J | 24 | 2 | 39 | 15 | 74 |
| 0 | 25 | 2 | 39 | 15 | 75 |
| Q | 26 | 6 | 40 | 15 | 76 |
| R | 27 | 6 | 38 | 15 | 69 |

Target knockdown was assessed 7 days post-injection. RNA was extracted from liver to determine murine Aldh2 mRNA levels by qPCR as described in Example 2.

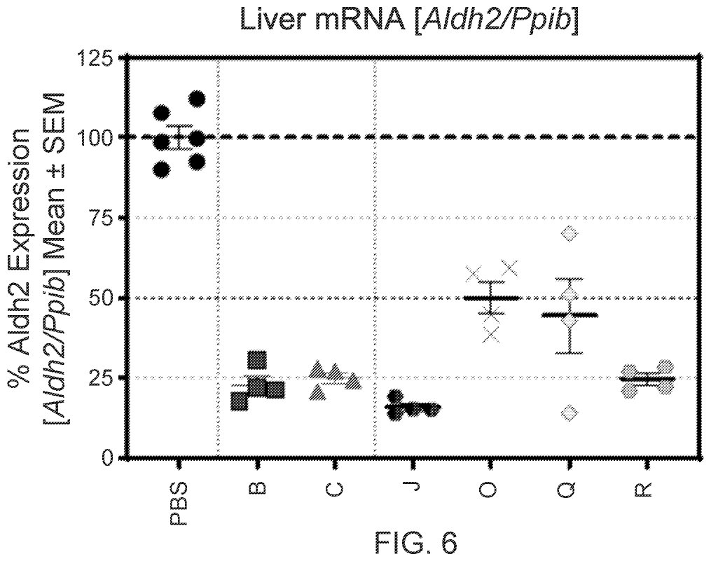

Percent Aldh2 mRNA expression remaining in liver is shown in FIG. 6. RNAi oligonucleotide-ligand conjugates with GalNAc conjugates and truncated antisense strands demonstrated target gene knockdown.

### Example 5: Distribution and Number of GalNAc Molecules Influence RNAi Oligonucleotide-Ligand Conjugate Activity

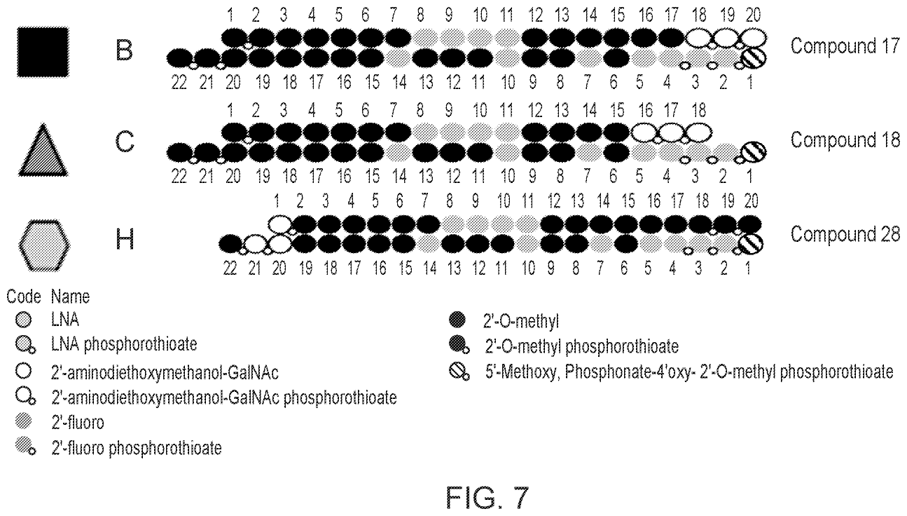

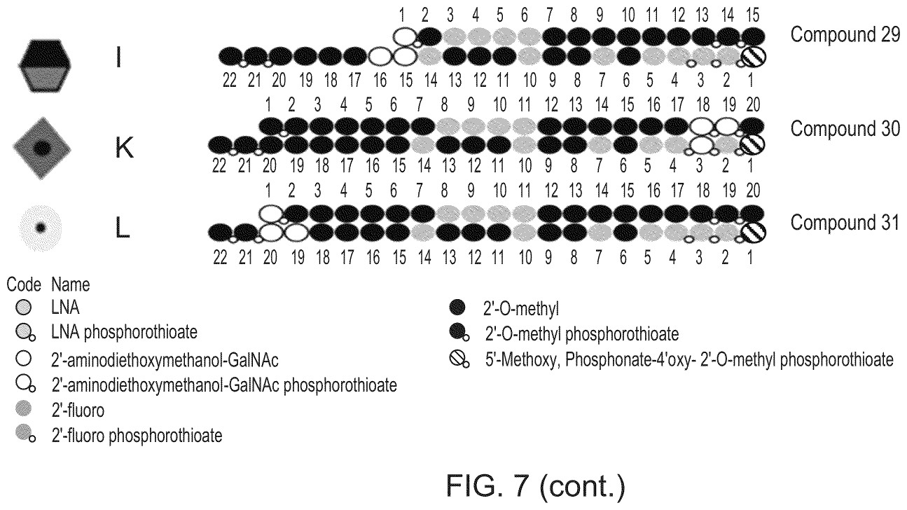

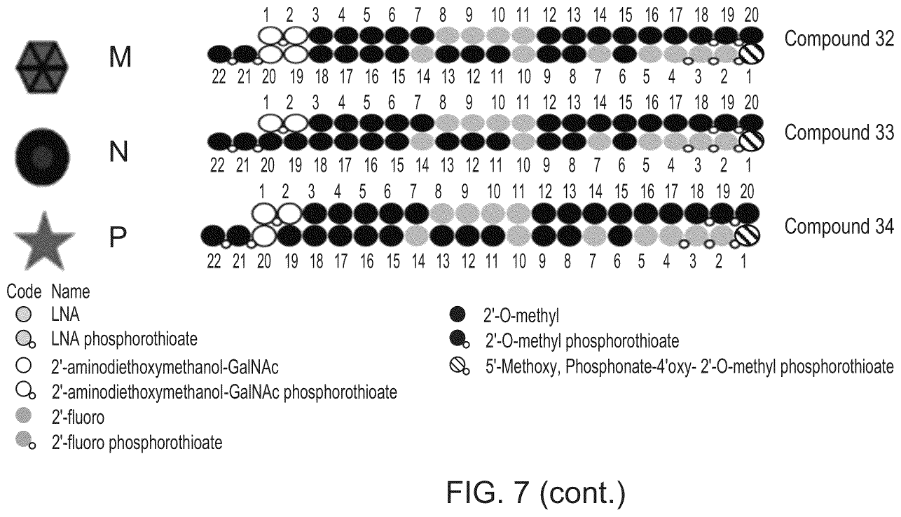

To test if the number of GalNAc molecules or distribution of the GalNAc molecules impacted target gene knockdown, RNAi oligonucleotide-ligand conjugates comprising GalNAc molecules at different positions were generated using the methods described in Example 1. The oligonucleotides comprised an antisense strand having a region of complementarity to mRNA encoding Aldh2 (Table 5) with the modification patterns as shown in FIG. 7. Oligonucleotides were administered in a single subcutaneous dose at 0.5 mg/kg.

Comparison was made to parent RNAi oligonucleotide-ligand conjugate Compounds 17 and 18 previously described in Example 3.

RNAi oligonucleotide-ligand conjugates that were evaluated in comparison to Compounds 17 and 18 had structures according to Compounds 28-34 as shown in FIG. 7. These structures contain the features described below:
(i) Compound 28 comprises a 20-nt sense strand and an 22-nt antisense strand. The Compound comprises a 2-nucleotide overhang at the 3′ terminus of the antisense strand. The sense strand comprises a GalNAc molecule at position 1. The antisense strand comprises a GalNAc compound at positions 20 and 21.
(ii) Compound 29 comprises a 15-nt sense strand and an 22-nt antisense strand. The Compound comprises a 7-nucleotide overhang at the 3′ terminus of the antisense strand. The sense strand comprises a GalNAc molecule at position 1. The antisense strand comprises a GalNAc compound at positions 15 and 16.
(iii) Compound 30 comprises a 20-nt sense strand and an 22-nt antisense strand. The Compound comprises a 2-nucleotide overhang at the 3′ terminus of the antisense strand. The sense strand comprises a GalNAc molecule at positions 18 and 19. The antisense strand comprises a GalNAc compound at position 3.
(iv) Compound 31 comprises a 20-nt sense strand and an 22-nt antisense strand. The Compound comprises a 2-nucleotide overhang at the 3′ terminus of the antisense strand. The sense strand comprises a GalNAc molecule at position 1. The antisense strand comprises a GalNAc compound at positions 19 and 20.
(v) Compound 32 comprises a 20-nt sense strand and an 22-nt antisense strand. The Compound comprises a 2-nucleotide overhang at the 3′ terminus of the antisense strand. The sense strand comprises a GalNAc molecule at positions 1 and 2. The antisense strand comprises a GalNAc compound at positions 19 and 20.
(vi) Compound 33 comprises a 20-nt sense strand and an 22-nt antisense strand. The Compound comprises a 2-nucleotide overhang at the 3′ terminus of the antisense strand. The sense strand comprises a GalNAc molecule at positions 1 and 2.
(vii) Compound 34 comprises a 20-nt sense strand and an 22-nt antisense strand. The Compound comprises a 2-nucleotide overhang at the 3′ terminus of the antisense strand. The sense strand comprises a GalNAc molecule at positions 1 and 2. The antisense strand comprises a GalNAc compound at position 20.

TABLE 5
Aldh2 RNAi Oligonucleotide-ligand Conjugates

| | Compound | Sense SEQ ID NOS | | Antisense SEQ ID NOS | |
|---|---|---|---|---|---|
| Group | ID | Unmodified | Modified | Unmodified | Modified |
| B | 17 | 2 | 27 | 15 | 69 |
| C | 18 | 5 | 37 | 15 | 69 |
| H | 28 | 2 | 41 | 15 | 74 |
| I | 29 | 7 | 42 | 15 | 77 |
| K | 30 | 2 | 43 | 15 | 78 |
| L | 31 | 2 | 41 | 15 | 79 |
| M | 32 | 2 | 44 | 15 | 79 |
| N | 33 | 2 | 44 | 15 | 69 |
| P | 34 | 2 | 44 | 15 | 80 |

Target knockdown was assessed 7 days post-injection. RNA was extracted from liver to determine murine Aldh2 mRNA levels by qPCR as described in Example 2.

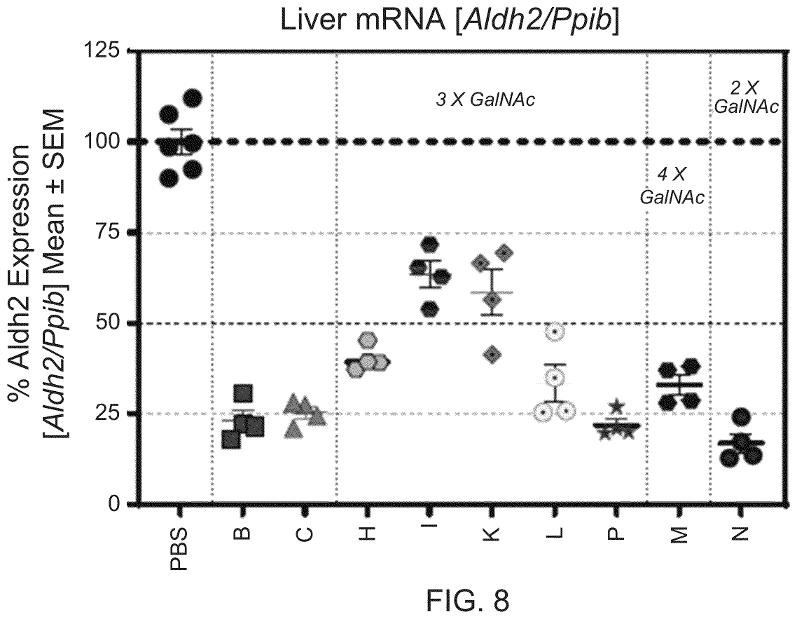

Percent Aldh2 mRNA expression remaining in liver is shown in FIG. 8. RNAi oligonucleotide-ligand conjugates with GalNAc conjugates on the sense and antisense strand impacted target gene knockdown relative to the parent molecule.

### Example 6: 5′ Position of GalNAc on Truncated Sense Strand does not Impact Target Gene Knockdown Activity

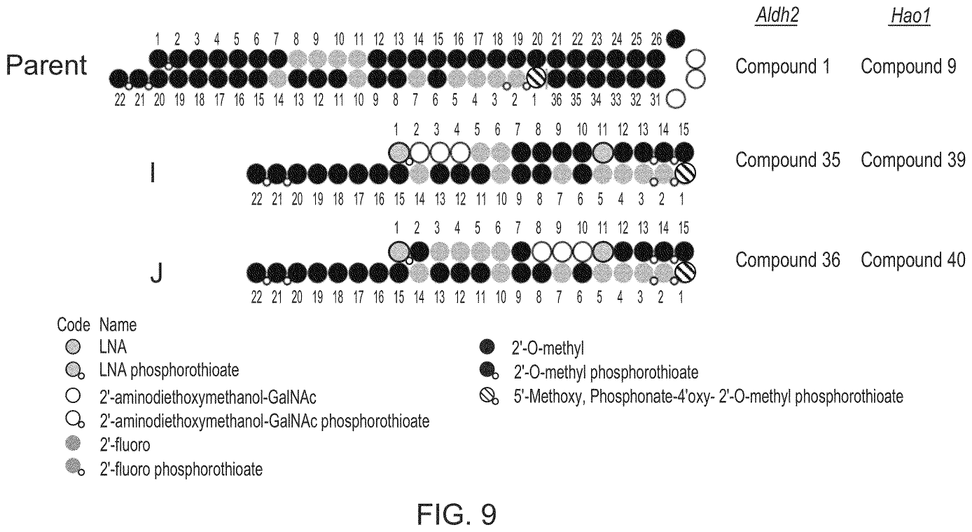

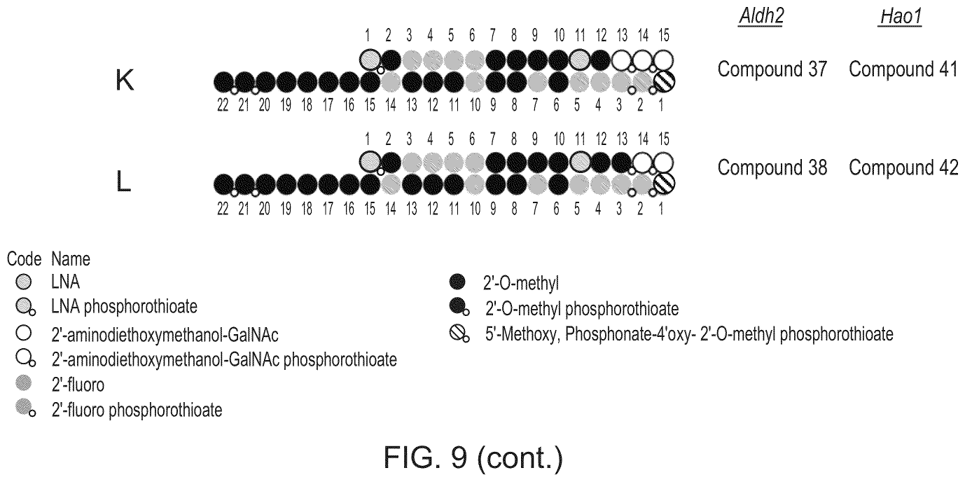

To test if target gene knockdown was impacted by the location of GalNAc molecules on a 5′ truncated sense strand, RNAi oligonucleotide-ligand conjugates comprising a 5′ sense strand truncation and GalNAc molecules at different strand positions were generated using the methods described in Example 1. The oligonucleotides comprised an antisense strand having a region of complementarity to mRNA encoding Aldh2 (Table 6) or Hao1 (Table 7) with the modification patterns as shown in FIG. 9. Oligonucleotides were administered in a single subcutaneous dose at 0.5 mg/kg.

Comparison was made to parent RNAi oligonucleotide-ligand conjugate Compounds 1 and 9 previously described in Example 2.

RNAi oligonucleotide-ligand conjugates that were evaluated in comparison to Compounds 1 and 9 had structures according to Compounds 35-42 as shown in FIG. 9. Compounds 35-42 comprise a 15-nt sense strand, a 7-nt overhang at the 3′ terminus the antisense strand, and a blunt end at the 5′ terminus of the antisense strand. The compounds further comprise locked nucleic acids at positions 1 and 11 of the sense strand. These Compounds comprise the additional features described below:
(i) Compounds 35 and 39 comprise a GalNAc molecule conjugated to positions 2, 3, and 4 of the sense strand.
(ii) Compounds 36 and 40 comprise a GalNAc molecule conjugated to positions 8, 9, and 10 of the sense strand.
(iii) Compounds 37 and 41 comprise a GalNAc molecule conjugated to positions 13, 14, and 15 of the sense strand.
(iv) Compounds 38 and 42 comprise a GalNAc molecule conjugated to positions 14 and 15 of the sense strand.

TABLE 6
Aldh2 RNAi Oligonucleotide-ligand Conjugates

| | Compound | Sense SEQ ID NOS | | Antisense SEQ ID NOS | |
|---|---|---|---|---|---|
| Group | ID | Unmodified | Modified | Unmodified | Modified |
| Parent | 1 | 1 | 21 | 15 | 67 |
| I | 35 | 7 | 45 | 15 | 67 |
| J | 36 | 7 | 46 | 15 | 67 |
| K | 37 | 7 | 47 | 15 | 67 |
| L | 38 | 7 | 48 | 15 | 67 |

TABLE 7
Haol RNAi Oligonucleotide-ligand Conjugates

| | Compound | Sense SEQ ID NOS | | Antisense SEQ ID NOS | |
|---|---|---|---|---|---|
| Group | ID | Unmodified | Modified | Unmodified | Modified |
| Parent | 9 | 1 | 29 | 16 | 68 |
| I | 39 | 8 | 49 | 16 | 68 |
| J | 40 | 8 | 50 | 16 | 68 |
| K | 41 | 8 | 51 | 16 | 68 |
| L | 42 | 8 | 52 | 16 | 68 |

Target knockdown was assessed 5 days post-injection. RNA was extracted from liver to determine murine Aldh2 or Hao1 mRNA levels by qPCR as described in Example 2.

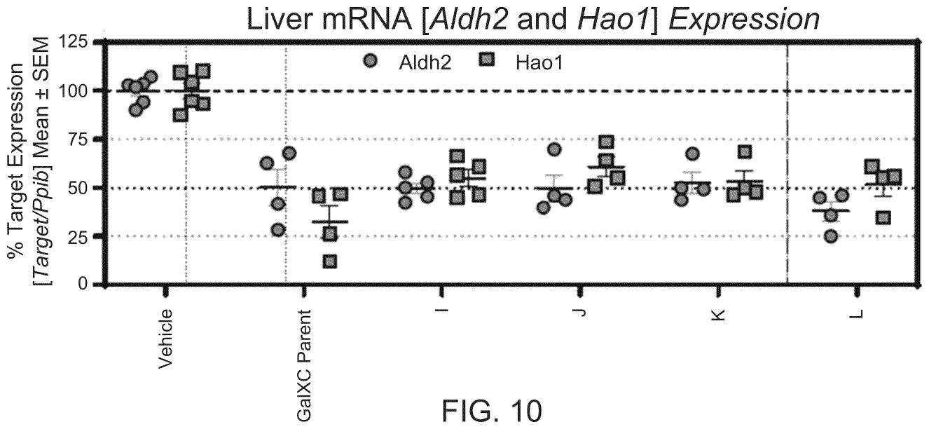

Percent Aldh2 and Hao1 mRNA expression remaining in liver is shown in FIG. 10. RNAi oligonucleotide-ligand conjugates with a 5′ truncated sense strand maintained target gene knockdown relative to the parent molecule.

### Example 7: Effect of Antisense Strand 3′ Overhang on RNAi Oligonucleotide-Ligand Conjugate Activity

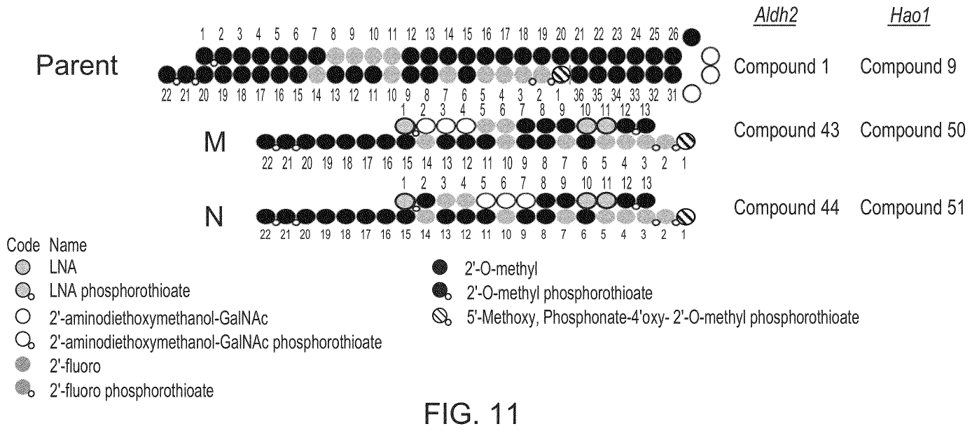

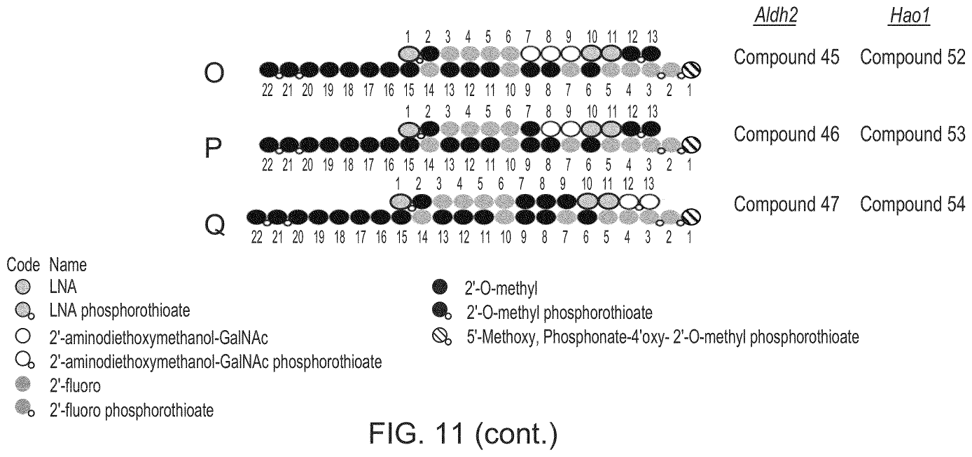

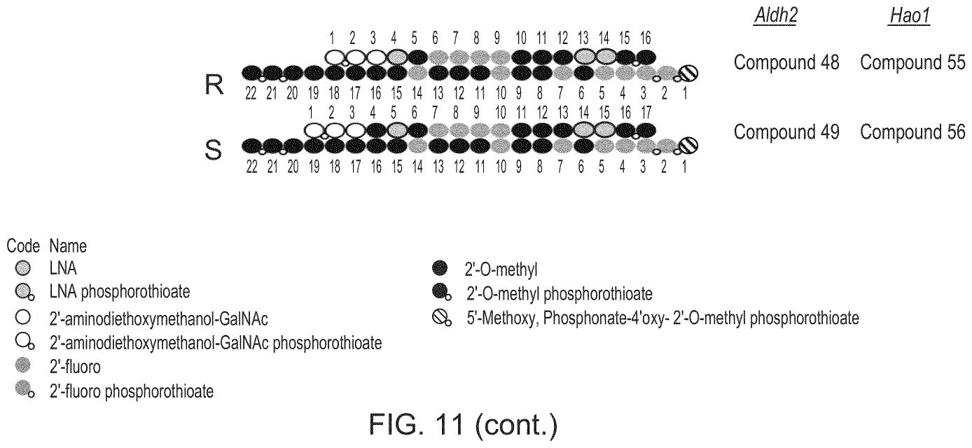

To test if inclusion of a 3′ overhang impacted target gene knockdown, RNAi oligonucleotide-ligand conjugates with a 3′ antisense strand overhang and GalNAc molecules at different sense strand positions were generated using the methods described in Example 1. The oligonucleotides comprised an antisense strand having a region of complementarity to mRNA encoding Aldh2 (Table 8) or Hao1 (Table 9) with the modification patterns as shown in FIG. 11. Oligonucleotides were administered in a single subcutaneous dose at 0.5 mg/kg.

Comparison was made to parent RNAi oligonucleotide-ligand conjugate Compounds 1 and 9 previously described in Example 2.

RNAi oligonucleotide-ligand conjugates that were evaluated in comparison to Compounds 1 and 9 had structures according to Compounds 43-56 as shown in FIG. 11. These structures contain the features described below:
(i) Compounds 43-47 and 50-54 comprise a 13-nt sense strand and a 22-nt antisense strand. The Compounds comprise a 7-nucleotide overhang at the 3′ terminus of the antisense strand and a 2-nt overhang at the 5′ terminus of the antisense strand. The compounds comprise locked nucleic acids at positions 1, 10, and 11 of the sense strand. Compounds 43 and 50 comprise a GalNAc molecule conjugated to positions 2, 3, and 4 of the sense strand. Compounds 44 and 51 comprise a GalNAc molecule conjugated to positions 5, 6, and 7 of the sense strand. Compounds 45 and 52 comprise a GalNAc molecule conjugated to positions 7, 8, and 9 of the sense strand. Compounds 46 and 53 comprise a GalNAc molecule conjugated to positions 8 and 9 of the sense strand. Compounds 47 and 54 comprise a GalNAc molecule conjugated to positions 12 and 13 of the sense strand.
(ii) Compounds 48 and 55 comprise a 16-nt sense strand and a 22-nt antisense strand. The Compounds comprise a 4-nucleotide overhang at the 3′ terminus of the antisense strand and a 2-nt overhang at the 5′ terminus of the antisense strand. The Compounds comprise a locked nucleic acid at positions 4, 13, and 14 of the sense strand. The compounds comprise a GalNAc molecule conjugated to positions 1, 2, and 3 of the sense strand.
(iii) Compounds 49 and 56 comprise a 17-nt sense strand and a 22-nt antisense strand. The Compounds comprise a 3-nucleotide overhang at the 3′ terminus of the antisense strand and a 2-nt overhang at the 5′ terminus of the antisense strand. The compounds comprise a locked nucleic acid at positions 5, 14, and 15. The compounds comprise a GalNAc molecule conjugated to positions 1, 2, and 3 of the sense strand.

TABLE 8
Aldh2 RNAi Oligonucleotide-ligand Conjugates

| | Compound | Sense SEQ ID NOS | | Antisense SEQ ID NOS | |
|---|---|---|---|---|---|
| Group | ID | Unmodified | Modified | Unmodified | Modified |
| Parent | 1 | 1 | 21 | 15 | 67 |
| M | 43 | 9 | 53 | 15 | 67 |
| N | 44 | 9 | 54 | 15 | 67 |
| 0 | 45 | 9 | 55 | 15 | 67 |
| P | 46 | 9 | 56 | 15 | 67 |
| Q | 47 | 9 | 57 | 15 | 67 |
| R | 48 | 10 | 58 | 15 | 67 |
| S | 49 | 11 | 59 | 15 | 67 |

TABLE 9
Hao1 RNAi Oligonucleotide-ligand Conjugates

| | Compound | Sense SEQ ID NOS | | Antisense SEQ ID NOS | |
|---|---|---|---|---|---|
| Group | ID | Unmodified | Modified | Unmodified | Modified |
| Parent | 9 | 3 | 29 | 16 | 68 |
| M | 50 | 12 | 60 | 16 | 68 |
| N | 51 | 12 | 61 | 16 | 68 |
| 0 | 52 | 12 | 62 | 16 | 68 |
| P | 53 | 12 | 63 | 16 | 68 |
| Q | 54 | 12 | 64 | 16 | 68 |
| R | 55 | 13 | 65 | 16 | 68 |
| S | 56 | 14 | 66 | 16 | 68 |

Target knockdown was assessed 5 days post-injection. RNA was extracted from liver to determine murine Aldh2 or Hao1 mRNA levels by qPCR as described in Example 2.

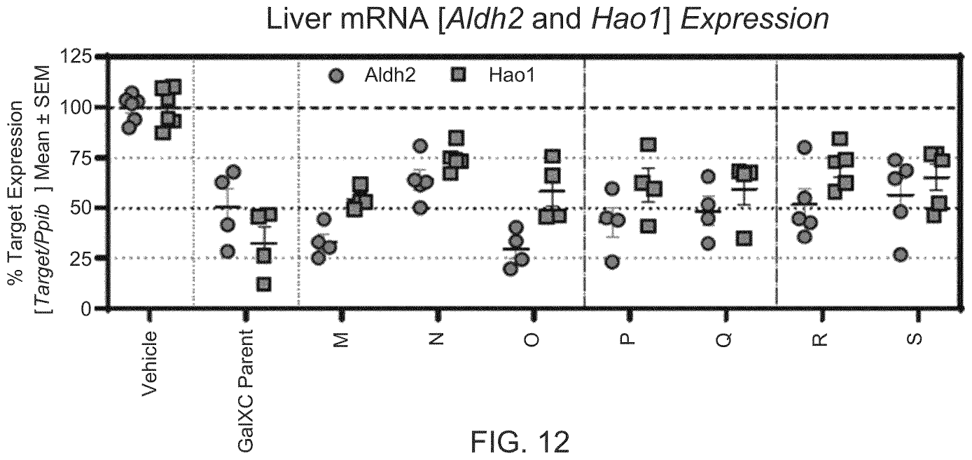

Percent Aldh2 and Hao1 mRNA expression remaining in liver is shown in FIG. 12. RNAi oligonucleotide-ligand conjugates with GalNAc conjugates and truncated antisense strands maintained target gene knockdown relative to the parent molecule.

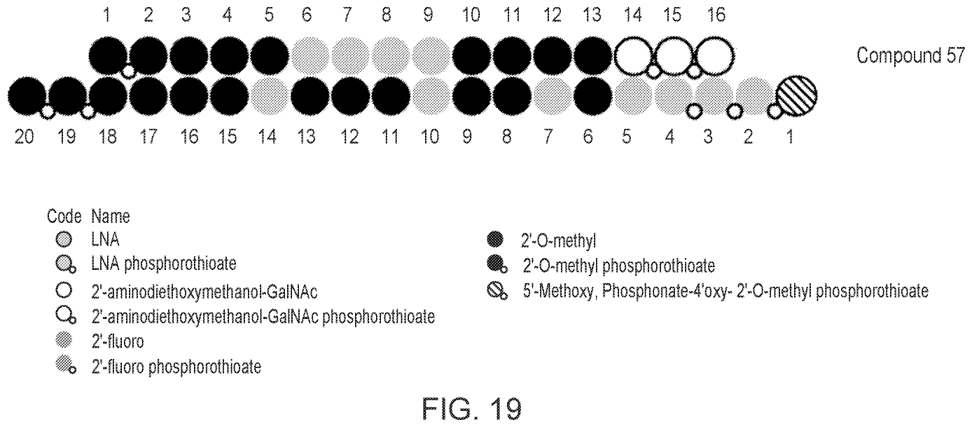

An additional RNAi oligonucleotide-ligand conjugate having a 3′ antisense strand overhang and GalNAc molecules on the sense strand is generated using the methods described in Example 1. The oligonucleotide has a modification pattern as shown in FIG. 19 and has an antisense strand having a region of complementarity to a target mRNA (e.g., mRNA encoding Aldh2 or Hao1). Compound 57 comprises a 2-nt overhang at the 3′ terminus the antisense strand and a 2-nt overhang at the 5′ terminus of the antisense strand, and comprises a GalNAc molecule conjugated to Positions 14, 15, and 16 of the sense strand. Oligonucleotide is administered to a mouse in a single subcutaneous dose at 0.5 mg/kg. Comparison is made to parent RNAi oligonucleotide-ligand conjugate Compounds 1 and 9 described in Example 2. Expression of the target mRNA is evaluated.

### Example 8: Effect of Dose on RNAi Oligonucleotide-Ligand Conjugate Activity

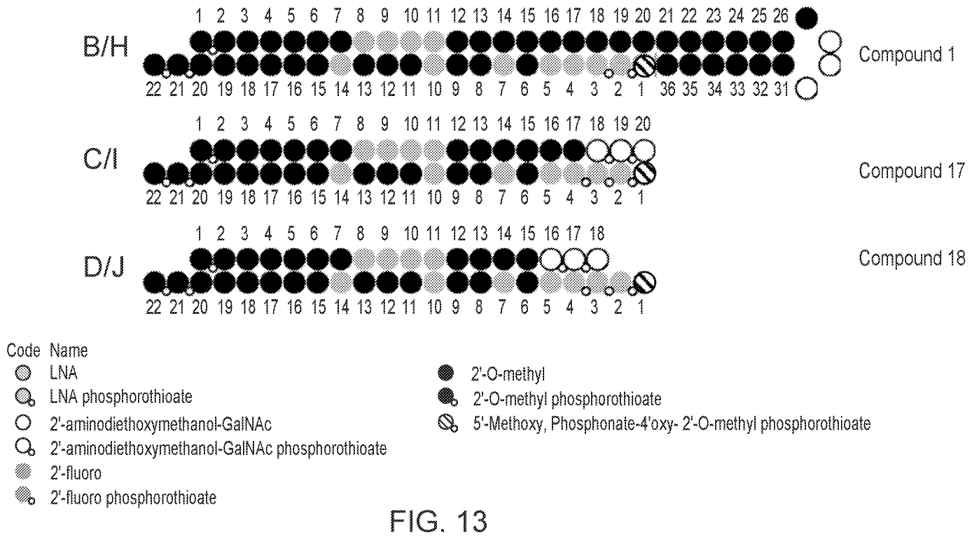

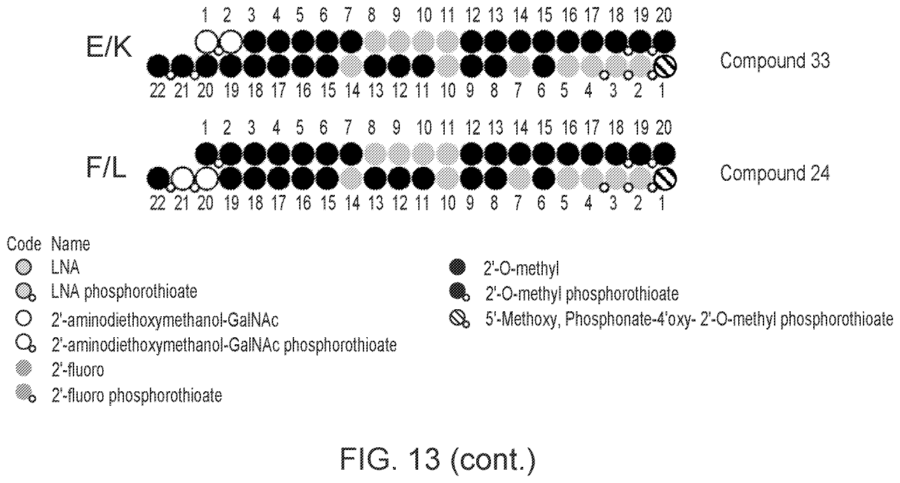

To test if dose of the RNAi oligonucleotide ligand conjugate impacted target gene knockdown activity, RNAi oligonucleotides tested in the examples above were evaluated for target gene knockdown after administration in mice at 0.25 mg/kg or 0.5 mg/kg. The oligonucleotides comprised an antisense strand having a region of complementarity to mRNA encoding Aldh2 (Table 10) with the modification patterns as shown in FIG. 13. Oligonucleotides were administered in a single subcutaneous dose.

Comparison was made to parent RNAi oligonucleotide-ligand conjugate Compound 1 previously described in Example 2.

RNAi oligonucleotide-ligand conjugates that were evaluated in comparison to Compound 1 had structures according to Compounds 17, 18, 33, and 24 as shown in FIG. 13. These structures are described in Examples 3, 4, and 5.

TABLE 10
Aldh2 RNAi Oligonucleotide-ligand Conjugates

| | Compound | Sense SEQ ID NOS | | Antisense SEQ ID NOS | |
|---|---|---|---|---|---|
| Group | ID | Unmodified | Modified | Unmodified | Modified |
| B/H | 1 | 1 | 21 | 15 | 67 |
| C/I | 17 | 2 | 27 | 15 | 69 |
| D/J | 18 | 5 | 37 | 15 | 69 |
| E/K | 33 | 2 | 39 | 15 | 69 |
| F/L | 24 | 2 | 44 | 15 | 74 |

Target knockdown was assessed 7 days post-injection. RNA was extracted from liver to determine murine Aldh2 mRNA levels by qPCR as described in Example 2.

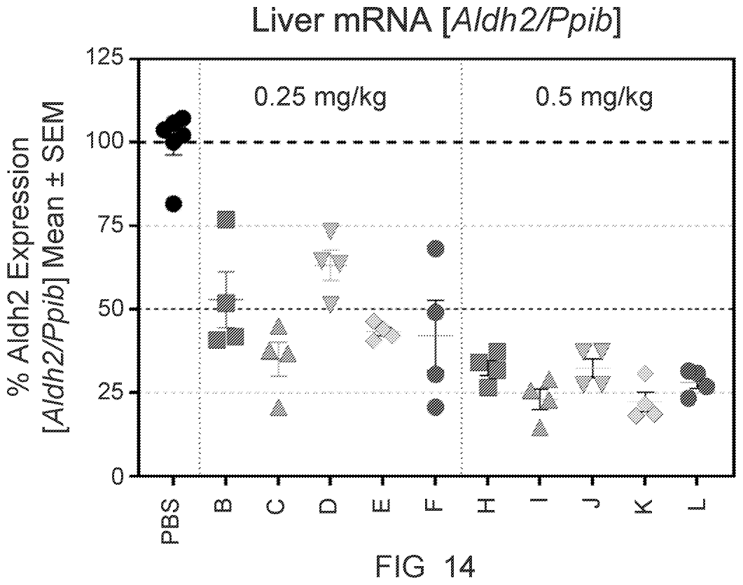

Percent Aldh2 mRNA expression remaining in liver is shown in FIG. 14. RNAi oligonucleotide-ligand conjugates demonstrated target gene knockdown at both 0.25 mg/kg and 0.5 mg/kg with an increase in knockdown in the 0.5 mg/kg dose.

### Example 9: Duration of Target Gene Knockdown with Low Molecular Weight RNAi Oligonucleotide-Ligand Conjugate

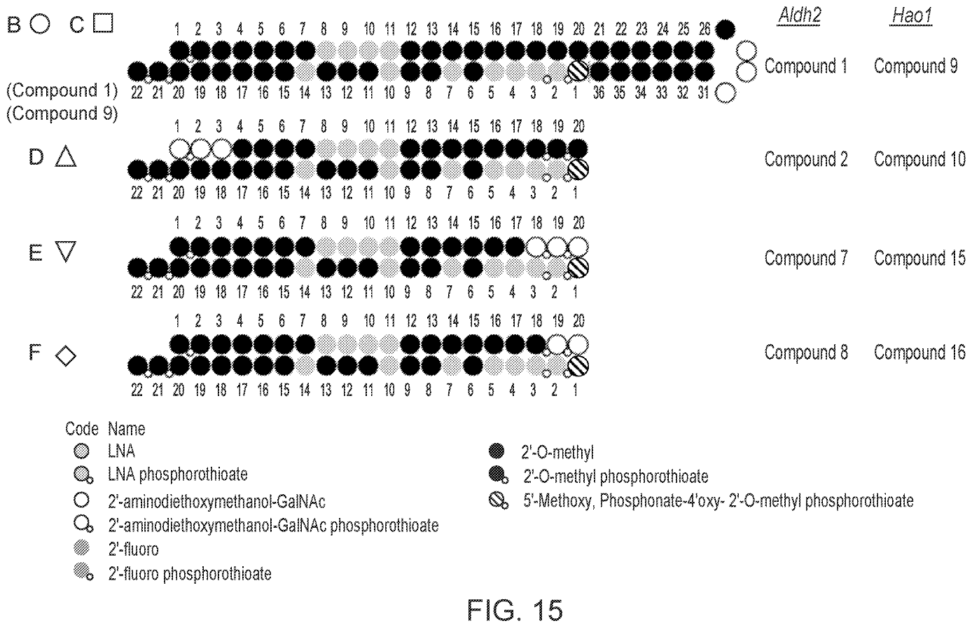

To test the duration of target gene knockdown with low molecular weight RNAi oligonucleotide ligand conjugates, RNAi oligonucleotides tested in the examples above were evaluated for target gene knockdown 7 days, 14 days, and 21 days after administration in mice. The oligonucleotides comprised an antisense strand having a region of complementarity to mRNA encoding Aldh2 (Table 11) or Hao1 (Table 12) with the modification patterns as shown in FIG. 15. Oligonucleotides were administered in a single subcutaneous dose.

Comparison was made to parent RNAi oligonucleotide-ligand conjugate Compounds 1 and 9 previously described in Example 2.

RNAi oligonucleotide-ligand conjugates that were evaluated in comparison to Compounds 1 and 9 had structures according to Compounds 2, 7, 8, 10, 15, and 16 as shown in FIG. 15. These structures are described in Example 2.

TABLE 11
Aldh2 RNAi Oligonucleotide-ligand Conjugates

| | Compound | Sense SEQ ID NOS | | Antisense SEQ ID NOS | |
|---|---|---|---|---|---|
| Group | ID | Unmodified | Modified | Unmodified | Modified |
| B | 1 | 1 | 21 | 15 | 67 |
| D | 2 | 2 | 22 | 15 | 67 |
| E | 7 | 2 | 27 | 15 | 67 |
| F | 8 | 2 | 28 | 15 | 67 |

TABLE 12
Haol RNAi Oligonucleotide-ligand Conjugates

| | Compound | Sense SEQ ID NOS | | Antisense SEQ ID NOS | |
|---|---|---|---|---|---|
| Group | ID | Unmodified | Modified | Unmodified | Modified |
| C | 9 | 3 | 29 | 16 | 68 |
| D | 10 | 4 | 30 | 16 | 68 |
| E | 15 | 4 | 35 | 16 | 68 |
| F | 16 | 4 | 36 | 16 | 68 |

Target knockdown was assessed 7, 14, and 21-days post-injection. RNA was extracted from liver to determine murine Aldh2 and Hao1 mRNA levels by qPCR as described in Example 2.

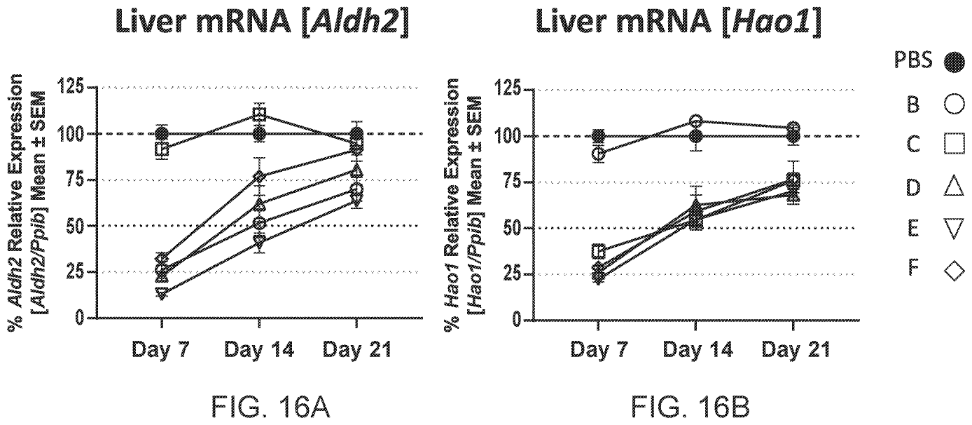

Percent Aldh2 mRNA expression remaining in liver is shown in FIG. 16A. RNAi oligonucleotide-ligand conjugates demonstrated knockdown through day 21. Percent Hao1 mRNA expression remaining in liver is shown in FIG. 16B. RNAi oligonucleotide-ligand conjugates demonstrated knockdown through day 21.

### Example 10: Duration of Target Gene Knockdown with RNAi Oligonucleotide-Ligand Conjugate in Non-Human Primates

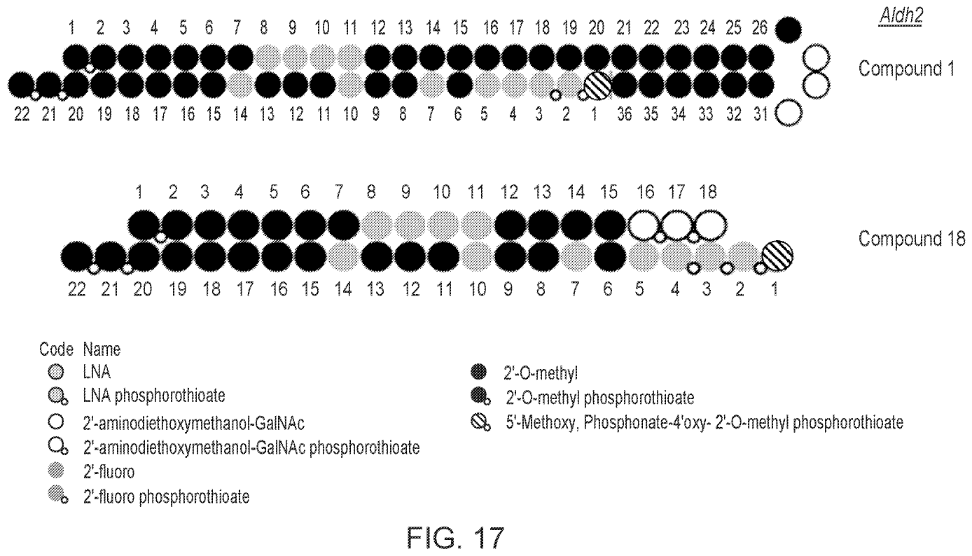

To test the efficacy of the RNAi oligonucleotide ligand conjugates in non-human primates (NHPs), animals were subcutaneously administered a single 3 mg/kg dose of oligonucleotide and gene knockdown activity was evaluated. The oligonucleotides comprised an antisense strand having a region of complementarity to mRNA encoding Aldh2 (Table 13) with the modification patterns as shown in FIG. 17. Oligonucleotides were administered in a subcutaneous dose.

Comparison was made to parent RNAi oligonucleotide-ligand conjugate Compound 1 previously described in Example 2.

The RNAi oligonucleotide-ligand conjugate evaluated in comparison to Compound 1 had the structure according to Compound 18 as shown in FIG. 7. This structure is described in Example 3.

TABLE 13
Aldh2 RNAi Oligonucleotide-ligand Conjugates

| Compound | Sense SEQ ID NOS | | Antisense SEQ ID NOS | |
|---|---|---|---|---|
| ID | Unmodified | Modified | Unmodified | Modified |
| 1 | 1 | 21 | 15 | 67 |
| 18 | 5 | 37 | 15 | 69 |

Target knockdown was assessed on days 28, 56, 84, and 112 post-injection. RNA was extracted from liver biopsy to determine NHP Aldh2 mRNA levels by qPCR as described in Example 2.

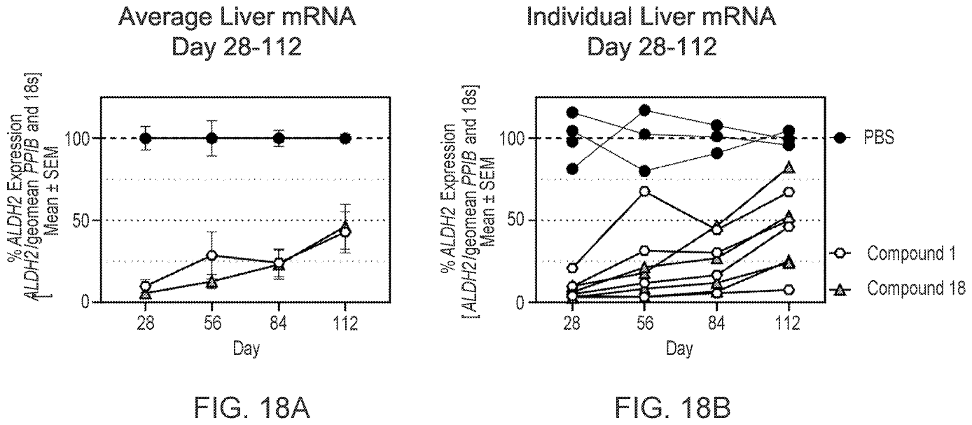

Percent Aldh2 mRNA expression remaining in liver is shown as an average of all treated NHPs in FIG. 18A and individual animals are shown in FIG. 18B. RNAi oligonucleotide-ligand conjugates demonstrated similar target gene knockdown for the duration of the study using both Compound 1 and Compound 18.

SEQUENCE LISTING

| | | SEQ |
|---|---|---|
| Description | Sequence | ID NO |
| Unmodified Sense | GGUGGAUGAAACUCAGUUUAGCAGCCGAAAGGCU | 1 |
| Strand | GC | |
| Unmodified Sense | GGUGGAUGAAACUCAGUUUA | 2 |
| Strand | | |
| Unmodified Sense | AUAUUUUCCCAUCUGUAUUAGCAGCCGAAAGGCU | 3 |
| Strand | GC | |
| Unmodified Sense | AUAUUUUCCCAUCUGUAUUA | 4 |
| Strand | | |
| Unmodified Sense | GGUGGAUGAAACUCAGUU | 5 |
| Strand | | |
| Unmodified Sense | AUGAAACUCAGTUUA | 6 |
| Strand | | |
| Unmodified Sense | AUGAAACUCAGUUUA | 7 |
| Strand | | |
| Unmodified Sense | TUCCCAUCUGTAUUA | 8 |
| Strand | | |
| Unmodified Sense | AUGAAACUCAGUU | 9 |
| Strand | | |
| Unmodified Sense | AAAAUGAAACUCAGUU | 10 |
| Strand | | |
| Unmodified Sense | AAAAAUGAAACUCAGUU | 11 |
| Strand | | |
| Unmodified Sense | TUCCCAUCUGTAU | 12 |
| Strand | | |
| Unmodified Sense | AAATUCCCAUCUGTAU | 13 |
| Strand | | |
| Unmodified Sense | AAAATUCCCAUCUGTAU | 14 |
| Strand | | |
| Unmodified Antisense | UAAACUGAGUUUCAUCCACCGG | 15 |
| Strand | | |
| Unmodified Antisense | UAAUACAGAUGGGAAAAUAUGG | 16 |
| Strand | | |
| Unmodified Antisense | UAAACUGAGUUUCAUCCACCG | 17 |
| Strand | | |
| Unmodified Antisense | UAAACUGAGUUUCAUCCACC | 18 |
| Strand | | |
| Unmodified Antisense | UAAACUGAGUUUCAUCCAC | 19 |
| Strand | | |
| Unmodified Antisense | UAAACUGAGUUUCAUCCA | 20 |
| Strand | | |
| Modified Sense Strand | [mGs][mG][mU][mG][mG][mA][mU][fG][fA][fA][fA][mC | 21 |
| | ][mU][mC][mA][mG][mU][mU][mU][mA][mG][mC][mA] | |
| | [mG][mC][mC][mG][ademA-GalNAc][ademA- | |
| | GalNAc][ademA-GalNAc][mG][mG][mC][mU][mG][mC] | |
| Modified Sense Strand | [ademGs-GalNAc][ademG-GalNAc][ademU- | 22 |
| | GalNAc][mG][mG][mA][mU][fG][fA][fA][fA][mC][mU][ | |
| | mC][mA][mG][mU][mUs][mUs][mA] | |
| Modified Sense Strand | [mGs][mG][mU][ademG-GalNAc][ademG- | 23 |
| | GalNAc][ademA- | |
| | GalNAc][mU][fG][fA][fA][fA][mC][mU][mC][mA][mG][ | |
| | mU][mUs][mUs][mA] | |
| Modified Sense Strand | [mGs][mG][mU][mG][mG][mA][mU][fG][ademA- | 24 |
| | GalNAc][ademA-GalNAc][ademA- | |
| | GalNAc][mC][mU][mC][mA][mG][mU][mUs][mUs][mA] | |
| Modified Sense Strand | [mGs][mG][mU][mG][mG][mA][mU][fG][fA][fA][fA][ade | 25 |
| | mC-GalNAc][ademU-GalNAc][ademC- | |
| | GalNAc][mA][mG][mU][mUs][mUs][mA] | |
| Modified Sense Strand | [mGs][mG][mU][mG][mG][mA][mU][fG][fA][fA][fA][mC | 26 |
| | ][mU][mC][ademA-GalNAc][ademG-GalNAc][ademU- | |
| | GalNAc][mUs][mUs][mA] | |
| Modified Sense Strand | [mGs][mG][mU][mG][mG][mA][mU][fG][fA][A][fA][mC | 27 |
| | ][mU][mC][mA][mG][mU][ademUs-GalNAc][ademUs- | |
| | GalNAc][ademA-GalNAc] | |
| Modified Sense Strand | [mGs][mG][mU][mG][mG][mA][mU][fG][fA][fA][fA][mC | 28 |
| | ][mU][mC][mA][mG][mU][mUs][ademUs- | |
| | GalNAc][ademA-GalNAc] | |
| Modified Sense Strand | [mAs][mU][mA][mU][mU][mU][mU][fC][fC][fC][fA][mU | 29 |
| | ][mC][mU][mG][mU][mA][mU][mU][mA][mG][mC][mA] | |
| | [mG][mC][mC][mG][ademA-GalNAc][ademA- | |
| | GalNAc][ademA-GalNAc][mG][mG][mC][mU][mG][mC] | |
| Modified Sense Strand | [ademAs-GalNAc][ademU-GalNAc][ademA- | 30 |
| | GalNAc][mU][mU][mU][mU][fC][fC][fC][fA][mU][mC][ | |
| | mU][mG][mU][mA][mUs][mUs][mA] | |
| Modified Sense Strand | [mAs][mU][mA][ademU-GalNAc][ademU- | 31 |
| | GalNAc][ademU- | |
| | GalNAc][mU][fC][fC][fC][fA][mU][mC][mU][mG][mU][ | |
| | mA][mUs][mUs][mA] | |
| Modified Sense Strand | [mAs][mU][mA][mU][mU][mU][mU][fC][ademC- | 32 |
| | GalNAc][ademC-GalNAc][ademA- | |
| | GalNAc][mU][mC][mU][mG][mU][mA][mUs][mUs][mA] | |
| Modified Sense Strand | [mAs][mU][mA][mU][mU][mU][mU][fC][fC][fC][fA][ade | 33 |
| | mU-GalNAc][ademC-GalNAc][ademU- | |
| | GalNAc][mG][mU][mA][mUs][mUs][mA] | |
| Modified Sense Strand | [mAs][mU][mA][mU][mU][mU][mU][fC][fC][fC][fA][mU | 34 |
| | ][mC][mU][ademG-GalNAc][ademU-GalNAc][ademA- | |
| | GalNAc][mUs][mUs][mA] | |
| Modified Sense Strand | [mAs][mU][mA][mU][mU][mU][mU][fC][fC][fC][fA][mU | 35 |
| | ][mC][mU][mG][mU][mA][ademUs-GalNAc][ademUs- | |
| | GalNAc][ademA-GalNAc] | |
| Modified Sense Strand | [mAs][mU][mA][mU][mU][mU][mU][fC][fC][fC][fA][mU | 36 |
| | ][mC][mU][mG][mU][mA][mUs][ademUs- | |
| | GalNAc][ademA-GalNAc] | |
| Modified Sense Strand | [mGs][mG][mU][mG][mG][mA][mU][fG][fA][fA][fA][mC | 37 |
| | ][mU][mC][mA][ademGs-GalNAc][ademUs- | |
| | GalNAc][ademU-GalNAc] | |
| Modified Sense Strand | [+As][mU][fG][fA][fA][fA][mC][mU][mC][mA][+G][+T][ | 38 |
| | ademUs-GalNAc][ademUs-GalNAc][ademA-GalNAc] | |
| Modified Sense Strand | [mGs][mG][mU][mG][mG][mA][mU][fG][fA][fA][fA][mC | 39 |
| | ][mU][mC][mA][mG][mU][mUs][mUs][mA] | |
| Modified Sense Strand | [+As][mU][fG][fA][fA][fA][mC][mU][mC][mA][+G][+T][ | 40 |
| | mUs][mUs][mA] | |
| Modified Sense Strand | [ademGs- | 41 |
| | GalNAc][mG][mU][mG][mG][mA][mU][fG][fA][fA][fA][ | |
| | mC][mU][mC][mA][mG][mU][mUs][mUs][mA] | |
| Modified Sense Strand | [ademAs- | 42 |
| | GalNAc][mU][fG][fA][fA][fA][mC][mU][mC][mA][mG][ | |
| | mU][mUs][mUs][mA] | |
| Modified Sense Strand | [mGs][mG][mU][mG][mG][mA][mU][fG][fA][fA][fA][mC | 43 |
| | ][mU][mC][mA][mG][mU][ademUs-GalNAc][ademUs- | |
| | GalNAc][mA] | |
| Modified Sense Strand | [ademGs-GalNAc][ademG- | 44 |
| | GalNAc][mU][mG][mG][mA][mU][fG][fA][fA][fA][mC][ | |
| | mU][mC][mA][mG][mU][mUs][mUs][mA] | |
| Modified Sense Strand | [+As][ademU-GalNAc][ademG-GalNAc][ademA- | 45 |
| | GalNAc][fA][fA][mC][mU][mC][mA][+G][mU][mUs][mU | |
| | s][mA] | |
| Modified Sense Strand | [+As][mU][fG][fA][fA][fA][mC][ademU-GalNAc][ademC- | 46 |
| | GalNAc][ademA-GalNAc][+G][mU][mUs][mUs][mA] | |
| Modified Sense Strand | [+As][mU][fG][fA][fA][fA][mC][mU][mC][mA][+G][mU] | 47 |
| | [ademUs-GalNAc][ademUs-GalNAc][ademA-GalNAc] | |
| Modified Sense Strand | [+As][mU][fG][fA][fA][fA][mC][mU][mC][mA][+G][mU] | 48 |
| | [mUs][ademUs-GalNAc][ademA-GalNAc] | |
| Modified Sense Strand | [+Ts][ademU-GalNAc][ademC-GalNAc][ademC- | 49 |
| | GalNAc][fC][fA][mU][mC][mU][mG][+T][mA][mUs][mU | |
| | s][mA] | |
| Modified Sense Strand | [+Ts][mU][fC][fC][fC][fA][mU][ademC-GalNAc][ademU- | 50 |
| | GalNAc][ademG-GalNAc][+T][mA][mUs][mUs][mA] | |
| Modified Sense Strand | [+Ts][mU][fC][fC][fC][fA][mU][mC][mU][mG][+T][mA][ | 51 |
| | ademUs-GalNAc] [ademUs-GalNAc][ademA-GalNAc] | |
| Modified Sense Strand | [+Ts][mU][fC][fC][fC][fA][mU][mC][mU][mG][+T][mA][ | 52 |
| | mUs][ademUs-GalNAc][ademA-GalNAc] | |
| Modified Sense Strand | [+As][ademU-GalNAc][ademG-GalNAc][ademA- | 53 |
| | GalNAc][fA][fA][mC][mU][mC][+A][+Gs][mUs][mU] | |
| Modified Sense Strand | [+As][mU][fG][fA][ademA-GalNAc][ademA- | 54 |
| | GalNAc][ademC-GalNAc][mU][mC][+A][+Gs][mUs][mU] | |
| Modified Sense Strand | [+As][mU][fG][fA][fA][fA][ademC-GalNAc][ademU- | 55 |
| | GalNAc][ademC-GalNAc][+A][+Gs][mUs][mU] | |
| Modified Sense Strand | [+As][mU][fG][fA][fA][fA][mC][ademU-GalNAc][ademC- | 56 |
| | GalNAc][+A][+Gs][mUs][mU] | |
| Modified Sense Strand | [+As][mU][fG][fA][fA][fA][mC][mU][mC][+A][+Gs][ade | 57 |
| | mUs-GalNAc][ademU-GalNAc] | |
| Modified Sense Strand | [ademAs-GalNAc][ademA-GalNAc][ademA- | 58 |
| | GalNAc][+A][mU][fG][fA][fA][fA][mC][mU][mC][+A][+ | |
| | Gs][mUs][mU] | |
| Modified Sense Strand | [ademAs-GalNAc][ademA-GalNAc][ademA- | 59 |
| | GalNAc][mA][+A][mU][fG][fA][fA][fA][mC][mU][mC][+ | |
| | A][+Gs][mUs][mU] | |
| Modified Sense Strand | [+Ts][ademU-GalNAc][ademC-GalNAc][ademC- | 60 |
| | GalNAc][fC][fA][mU][mC][mU][+G][+Ts][mAs][mU] | |
| Modified Sense Strand | [+Ts][mU][fC][fC][ademC-GalNAc][ademA- | 61 |
| | GalNAc][ademU-GalNAc][mC][mU][+G][+Ts][mAs][mU] | |
| Modified Sense Strand | [+Ts][mU][fC][fC][fC][fA][ademU-GalNAc][ademC- | 62 |
| | GalNAc][ademU-GalNAc][+G][+Ts][mAs][mU] | |
| Modified Sense Strand | [+Ts][mU][fC][fC][fC][fA][mU][ademC-GalNAc][ademU- | 63 |
| | GalNAc][+G][+Ts][mAs][mU] | |
| Modified Sense Strand | [+Ts][mU][fC][fC][fC][fA][mU][mC][mU][+G][+Ts][ade | 64 |
| | mAs-GalNAc][ademU-GalNAc] | |
| Modified Sense Strand | [ademAs-GalNAc][ademA-GalNAc][ademA- | 65 |
| | GalNAc][+T][mU][fC][fC][fC][fA][mU][mC][mU][+G][+ | |
| | Ts][mAs][mU] | |
| Modified Sense Strand | [ademAs-GalNAc][ademA-GalNAc][ademA- | 66 |
| | GalNAc][mA][+T][mU][fC][fC][fC][fA][mU][mC][mU][+ | |
| | G][+Ts][mAs][mU] | |
| Modified Antisense | [MePhosphonate-4O- | 67 |
| Strand | mUs][fAs][fA][fA][fC][mU][fG][mA][mG][fU][mU][mU][ | |
| | mC][fA][mU][mC][mC][mA][mC][mCs][mGs][mG] | |
| Modified Antisense | [MePhosphonate-4O- | 68 |
| Strand | mUs][fAs][fA][fU][fA][mC][fA][mG][mA][fU][mG][mG][ | |
| | mG][fA][mA][mA][mA][mU][mA][mUs][mGs][mG] | |
| Modified Antisense | [MePhosphonate-4O- | 69 |
| Strand | mUs][fAs][fAs][fA][fC][mU][fG][mA][mG][fU][mU][mU] | |
| | [mC][fA][mU][mC][mC][mA][mC][mCs][mGs][mG] | |
| Modified Antisense | [MePhosphonate-4O- | 70 |
| Strand | mUs][fAs][fAs][fA][fC][mU][fG][mA][mG][fU][mU][mU] | |
| | [mC][fA][mU][mC][mC][mA][mCs][mCs][mG] | |
| Modified Antisense | [MePhosphonate-4O- | 71 |
| Strand | mUs][fAs][fAs][fA][fC][mU][fG][mA][mG][fU][mU][mU] | |
| | [mC][fA][mU][mC][mC][mAs][mCs][mC] | |
| Modified Antisense | [MePhosphonate-4O- | 72 |
| Strand | mUs][fAs][fAs][fA][fC][mU][fG][mA][mG][fU][mU][mU] | |
| | [mC][fA][mU][mC][mCs][mAs][mC] | |
| Modified Antisense | [MePhosphonate-4O- | 73 |
| Strand | mUs][fAs][fAs][fA][fC][mU][fG][mA][mG][fU][mU][mU] | |
| | [mC][fA][mU][mCs][mCs][mA] | |
| Modified Antisense | [MePhosphonate-4O- | 74 |
| Strand | mUs][fAs][fAs][fA][fC][mU][fG][mA][mG][fU][mU][mU] | |
| | [mC][fA][mU][mC][mC][mA][mC][ademCs- | |
| | GalNAc][ademGs-GalNAc][mG] | |
| Modified Antisense | [MePhosphonate-4O- | 75 |
| Strand | mUs][fAs][fAs][fA][fC][mU][fG][mA][mG][fU][mU][mU] | |
| | [mC][fA][mU][mC][mC][mA][ademC-GalNAc][ademCs- | |
| | GalNAc][ademGs-GalNAc][mG] | |
| Modified Antisense | [MePhosphonate-4O- | 76 |
| Strand | mUs][fAs][fAs][fA][fC][mU][fG][mA][mG][fU][mU][mU] | |
| | [mC][fA][mU][mC][ademC-GalNAc][ademA- | |
| | GalNAc][ademC-GalNAc][mCs] [mGs][mG] | |
| Modified Antisense | [MePhosphonate-4O- | 77 |
| Strand | mUs][fAs][fAs][fA][fC][mU][fG][mA][mG][fU][mU][mU] | |
| | [mC][fA][ademU-GalNAc][ademC- | |
| | GalNAc][mC][mA][mC][mCs][mGs][mG] | |
| Modified Antisense | [MePhosphonate-4O-mUs][fAs][ademAs- | 78 |
| Strand | GalNAc][fA][fC][mU][fG][mA][mG][fU][mU][mU][mC][f | |
| | A][mU][mC][mC][mA][mC][mCs][mGs][mG] | |
| Modified Antisense | [MePhosphonate-4O- | 79 |
| Strand | mUs][fAs][fAs][fA][fC][mU][fG][mA][mG][fU][mU][mU] | |
| | [mC][fA][mU][mC][mC][mA][ademC-GalNAc][ademCs- | |
| | GalNAc][mGs][mG] | |
| Modified Antisense | [MePhosphonate-4O- | 80 |
| Strand | mUs][fAs][fAs][fA][fC][mU][fG][mA][mG][fU][mU][mU] | |
| | [mC][fA][mU][mC][mC][mA][mC][ademCs- | |
| | GalNAc][mGs][mG] | |

1. A double-stranded oligonucleotide comprising an antisense strand of about 20-22 nucleotides in length and a sense strand of about 16-20 nucleotides in length, wherein:
(i) the antisense and sense strands form a duplex region of about 16-20 base pairs,
(ii) the antisense strand comprises a 3′ overhang of at least two nucleotides,
(iii) the antisense strand comprises a region of complementarity to a liver mRNA target sequence,
(iv) the sense strand and antisense strand each comprise at least two nucleotides modified with a 2′-modification, wherein the 2′-modification is 2′-fluoro and 2′-O-methyl, and
(v) the sense strand comprises one to four nucleotides conjugated to a GalNAc molecule.

2. The double-stranded oligonucleotide of claim 1, wherein the antisense strand comprises a 5′ overhang of at least two nucleotides.

3. The double-stranded oligonucleotide of claim 1, wherein the oligonucleotide comprises a blunt end comprising the 5′ end of the antisense strand and the 3′ end of the sense strand.

4. The double-stranded oligonucleotide of claim 1, wherein the antisense strand is 22 nucleotides and the sense strand is 18-20 nucleotides.

5. The double-stranded oligonucleotide of claim 4, wherein the antisense strand comprises nucleotide positions numbered 5′ to 3′, and wherein positions 2, 3, 4, 5, 7 and 14 each comprise a 2′-fluoro modification.

6. The double-stranded oligonucleotide of claim 5, wherein positions 6, 8, 9, 11-13 and 15-22 each comprise a 2′-O-methyl modification.

7. The double-stranded oligonucleotide of any one of claims 4-6, wherein the sense strand is 18 nucleotides, and wherein the antisense comprises a 5′ overhang of at least two nucleotides.

8. The double-stranded oligonucleotide of any one of claims 4-6, wherein the sense strand is 20 nucleotides, and wherein the oligonucleotide comprises a blunt end comprising the 5′ end of the antisense strand and the 3′ end of the sense strand.

9. The double-stranded oligonucleotide of any one of claims 4-8, wherein the sense strand comprises three nucleotides conjugated to a GalNAc molecule.

10. The double-stranded oligonucleotide of claim 9, wherein the three nucleotides are at the 3′ end of the sense strand.

11. The double-stranded oligonucleotide of any one of claims 4-10, wherein the sense strand comprises nucleotide positions numbered 5′ to 3′, and wherein positions 8-11 each comprise a 2′-fluoro modification.

12. The double-stranded oligonucleotide of claim 11, wherein the remaining nucleotides of the sense strand comprise a 2′-O-methyl modification, provided the nucleotide is not conjugated to the GalNAc molecule.

13. The double-stranded oligonucleotide of claim 1, wherein the antisense strand is 20 nucleotides and the sense strand is 16-18 nucleotides.

14. The double-stranded oligonucleotide of claim 13, wherein the antisense strand comprises nucleotide positions numbered 5′ to 3′, and wherein positions 2, 3, 4, 5, 7 and 14 each comprise a 2′-fluoro modification.

15. The double-stranded oligonucleotide of claim 14, wherein positions 6, 8, 9, 11-13 and 15-20 each comprise a 2′-O-methyl modification.

16. The double-stranded oligonucleotide of any one of claims 13-15, wherein the sense strand is 16 nucleotides, and wherein the antisense comprises a 5′ overhang of at least two nucleotides.

17. The double-stranded oligonucleotide of any one of claims 13-16, wherein the sense strand comprises three nucleotides conjugated to a GalNAc molecule.

18. The double-stranded oligonucleotide of claim 17, wherein the three nucleotides are at the 3′ end of the sense strand.

19. The double-stranded oligonucleotide of any one of claims 13-18, wherein the sense strand comprises nucleotide positions numbered 5′ to 3′, and wherein positions 6-9 each comprise a 2′-fluoro modification.

20. The double-stranded oligonucleotide of claim 19, wherein the remaining nucleotides of the sense strand comprise a 2′-O-methyl modification, provided the nucleotide is not conjugated to the GalNAc molecule.

21. The double-stranded oligonucleotide of any one of claims 1-20, wherein the antisense strand comprises a phosphorylated nucleotide at the 5′ terminus, wherein the phosphorylated nucleotide is selected from uridine and adenosine.

22. The double-stranded oligonucleotide of claim 21, wherein the phosphorylated nucleotide is uridine.

23. The double-stranded oligonucleotide of any one of claims 21-22, wherein the phosphorylated nucleotide is 4′-O-monomethylphosphonate-2′-O-methyl uridine.

24. The double-stranded oligonucleotide of any one of claims 1-20, wherein the 4′-carbon of the sugar of the 5′-nucleotide of the antisense strand comprises a phosphate analog.

25. The double-stranded oligonucleotide of claim 24, wherein the phosphate analog is oxymethyl phosphonate, vinyl phosphonate or malonyl phosphonate.

26. The double-stranded oligonucleotide of any one of the preceding claims, wherein the oligonucleotide comprises at least one modified internucleotide linkage.

27. The double-stranded oligonucleotide of claim 26, wherein the at least one modified internucleotide linkage is a phosphorothioate linkage.

28. The double-stranded oligonucleotide of claim 27, wherein the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′.

29. The double-stranded oligonucleotide of claim 27 or 28, wherein the antisense strand comprises a phosphorothioate linkage between the penultimate nucleotide and third nucleotide from the 3′ end, and between the penultimate nucleotide and ultimate nucleotide.

30. The double-stranded oligonucleotide of any one of claims 27-29, wherein the sense strand comprises a phosphorothioate linkage between positions 1 and 2, between the penultimate nucleotide and third nucleotide from the 3′ end, and between the penultimate nucleotide and ultimate nucleotide.

31. The double-stranded oligonucleotide of any one of claims 1-30, wherein the oligonucleotide reduces expression of the liver mRNA target sequence in a cell or population of cells in vitro and/or in vivo.

32. A pharmaceutical composition comprising the oligonucleotide of any one of claims 1-31, and a pharmaceutically acceptable carrier, delivery agent or excipient.

33. A method for treating a subject having a disease, disorder or condition associated with expression of a liver mRNA, the method comprising administering to the subject a therapeutically effective amount of the double-stranded oligonucleotide of any one of claims 1-31, or the pharmaceutical composition of claim 32.

34. A method of reducing expression of a liver mRNA in a subject, comprising administering to the subject the double-stranded oligonucleotide of any one of claims 1-31, or the pharmaceutical composition of claim 32.
